(19) United States
(12) Patent Application Publication
(10) Pub. No.: US 2014/0349989 A1
(43) Pub. Date: Nov. 27, 2014

(54) COMPOUNDS AND THERAPEUTIC USES THEREOF

(71) Applicant: Alzheimer's Institute of America, Kansas City, KS (US)

(72) Inventors: Dange Vijay Kumar, San Diego, CA (US); Paul M. Slattum, Cottonwood Heights, UT (US); Kraig M. Yager, Muray, UT (US); Mark D. Shenderovich, Salt Lake City, UT (US); Rajendra Tangallapally, Germantown, TN (US); Se-Ho Kim, Cottonwood Heights, CA (US)

(73) Assignee: ALZHEIMER'S INSTITUTE OF AMERICA, Kansas City, KS (US)

(21) Appl. No.: 14/136,698

(22) Filed: Dec. 20, 2013

Related U.S. Application Data
(63) Continuation of application No. PCT/US2012/043376, filed on Jun. 20, 2012.
(60) Provisional application No. 61/578,065, filed on Dec. 20, 2011.
(60) Provisional application No. 61/499,012, filed on Jun. 20, 2011.

(51) Int. Cl. C07D 471/04; A61K 31/4439; A61K 31/444; A61K 45/06; A61K 31/496; A61K 31/437; A61K 31/4545; A61K 31/541; C07D 401/12; C07D 401/14

(52) U.S. Cl. CPC C07D 471/04; C07D 401/12; A61K 31/4439; A61K 31/444; C07D 401/14; A61K 31/496; A61K 31/437; A61K 31/4545; A61K 31/541; A61K 45/06
USPC 514/210.18; 546/275.4; 514/341; 546/278.1; 514/339; 546/194; 514/318; 546/275.7; 514/338; 544/364; 546/113; 514/300; 546/187; 514/316; 544/124; 514/235.2; 514/210.01

(57) ABSTRACT

The invention relates to compounds, pharmaceutical compositions and methods useful for treating cancer, systemic or chronic inflammation, rheumatoid arthritis, diabetes, obesity, T-cell mediated autoimmune disease, ischemia, and complications associated with these diseases and disorders.

### CROSS-REFERENCE TO RELATED APPLICATIONS

This application is a continuation of international application PCT/US2012/043376, filed Jun. 20, 2012 and published as WO 2012/177782 on Dec. 27, 2012, which claims the benefit of U.S. provisional application Ser. No. 61/578,065, filed Dec. 20, 2011 and U.S. provisional application Ser. No. 61/499,012, filed Jun. 20, 2011, the contents of all of which are hereby incorporated by reference in their entirety.

### FIELD OF THE INVENTION

The present invention relates generally to the field of medicinal chemistry. Specifically, the present invention provides compounds that inhibit Nicotinamide phosphoribosyltransferase (Nampt). The invention also provides methods for making these compounds, pharmaceutical compositions comprising these compounds, and methods for treating diseases with these compounds; particularly cancer, systemic or chronic inflammation, rheumatoid arthritis, diabetes, obesity, T-cell mediated autoimmune disease, ischemia, and other complications associated with these diseases and disorders, that respond favorably to the inhibition of Nampt.

### BACKGROUND OF THE INVENTION

Nicotinamide phosphoribosyltransferase (Nampt; also know as visfatin and pre-B-cell colony-enhancing factor 1 (PBEF)) catalyzes the condensation of nicotinamide (NaM) with 5-phosphoribosyl-1-pyrophosphate to yield nicotinamide mononucleotide. This is the first and rate-limiting step in one biosynthetic pathway that cells use to make nicotinamide adenine dinucleotide (NAD⁺).

NAD⁺ has many important cellular functions. Classically, it plays a role as a key coenzyme in metabolic pathways, where it continually cycles between its oxidized form (NAD⁺) and its reduced form (NADH). More recently, NAD⁺ has been shown to be involved in genome integrity maintainence, stress response, and Ca²⁺ signaling, where it is consumed by enzymes including poly(ADP-ribose) polymerases (PARPs), sirtuins, and cADP-ribose synthases, respectively. (Reviewed in Belenky, P. et al., NAD⁺ metabolism in health and disease. Trends Biochem. Sci. 32, 12-19 (2007).)

As a critical coenzyme in redox reactions, NAD⁺ is required in glycolysis and the citric acid cycle; where it accepts the high energy electrons produced and, as NADH, passes these electrons on to the electron transport chain. The NADH-mediated supply of high energy electrons is the driving force behind oxidative phosphorylation, the process by which the majority of ATP is generated in aerobic cells. Consequently, having sufficient levels of NAD⁺ available in the cell is critical for the maintenance of proper ATP levels in the cell. Understandably, reduction in cellular NAD⁺ levels by Nampt inhibition can be expected to eventually lead to depletion of ATP and, ultimately, cell death.

In view of the above, it is perhaps not surprising that inhibitors of Nampt are being developed as chemotherapeutic agents for the treatment of cancer. In fact, there are currently two Nampt inhibitors in clinical trials for the treatment of cancer (Holen, K. et al. The pharmacokinetics, toxicities, and biologic effects of FK866, a nicotinamide adenine dinucleotide biosynthesis inhibitor. Invest. New Drugs. 26, 45-51 (2008); Hovstadius, P. et al. A Phase I study of CHS 828 in patients with solid tumor malignancy. Clin. Cancer Res. 8, 2843-2850 (2002); Ravaud, A. et al., Phase I study and pharmacokinetic of CHS-828, a guanidino-containing compound, administered orally as a single dose every 3 weeks in solid tumours: an ECSG/EORTC study. Eur. J. Cancer. 41, 702-707 (2005); and von Heideman, A. et al. Safety and efficacy of NAD depleting cancer drugs: results of a phase I clinical trial of CHS 828 and overview of published data. Cancer Chemother. Pharmacol. (2009) Sep. 30 [Epub ahead of print]).

Consequently, there is a clear need for compounds that inhibit Nampt, which can not only be used in the treatment of cancer, but can also be used in the treatment of systemic or chronic inflammation, rheumatoid arthritis, diabetes, obesity, T-cell mediated autoimmune disease, ischemia, and other complications associated with these diseases and disorders.

### BRIEF SUMMARY OF THE INVENTION

The present invention provides chemical compounds that inhibit the activity of Nampt. These compounds can be used in the treatment of cancer, systemic or chronic inflammation, rheumatoid arthritis, diabetes, obesity, T-cell mediated autoimmune disease, ischemia, and other complications associated with these diseases and disorders.

Specifically, the present invention provides compounds of Formula I

J-K-L-E-Q-P Formula I

and pharmaceutically-acceptable salts and solvates thereof; wherein J, K, L, E, Q, and P are as defined herein below.

Additionally, the present invention provides compounds of Formula II

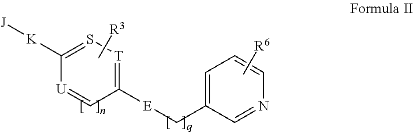

and pharmaceutically-acceptable salts and solvates thereof; wherein J, K, E, S, T, U, n, q, R³ and R⁶ are as defined herein below.

Additionally, the present invention provides compounds of Formula III

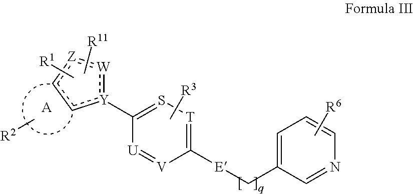

and pharmaceutically-acceptable salts and solvates thereof; wherein A, E′, S, T, U, V, W, Y, Z, q, R¹, R², R³, R⁴, R⁵, and R⁶ are as defined herein below.

Additionally, the present invention provides compounds of Formula IV

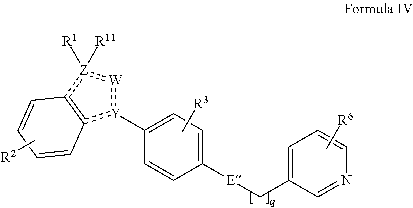

and pharmaceutically-acceptable salts and solvates thereof; wherein E″, W, Y, Z, q, R¹, R², R³, R⁶, and R¹¹ are as defined herein below.

Additionally, the present invention provides compounds of Formula IVa

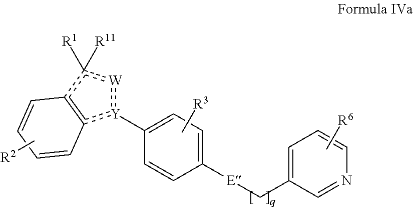

and pharmaceutically-acceptable salts and solvates thereof; wherein E¹¹, W, q, R¹, R², R³, R⁶, and R¹¹ are as defined herein below.

Additionally, the present invention provides compounds of Formula IVb

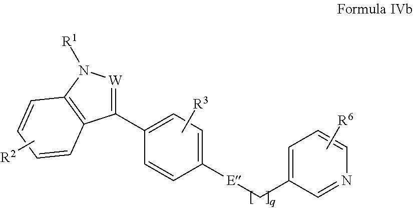

and pharmaceutically-acceptable salts and solvates thereof; wherein E¹¹, W, q, R¹, R², R³, and R⁶ are as defined herein below.

As noted above, the present invention provides chemical compounds that inhibit the activity of Nampt, and therefore can be used in the treatment of cancer, systemic or chronic inflammation, rheumatoid arthritis, diabetes, obesity, T-cell mediated autoimmune disease, ischemia, and other complications associated with these diseases and disorders. Thus, in a related aspect, the present invention also provides methods for treating cancer, systemic or chronic inflammation, rheumatoid arthritis, diabetes, obesity, T-cell mediated autoimmune disease, ischemia, and other complications associated with these diseases and disorders, by administering to a patient in need of such treatment a therapeutically effective amount of one or more of the compounds of the present invention.

Also provided is the use of the compounds of the present invention for the manufacture of a medicament useful for therapy, particularly for the treatment of cancer, systemic or chronic inflammation, rheumatoid arthritis, diabetes, obesity, T-cell mediated autoimmune disease, ischemia, and other complications associated with these diseases and disorders. In addition, the present invention also provides a pharmaceutical composition having one or more of the compounds of the present invention and one or more pharmaceutically acceptable excipients. Further, methods for the treatment of cancer, systemic or chronic inflammation, rheumatoid arthritis, diabetes, obesity, T-cell mediated autoimmune disease, ischemia, and other complications associated with these diseases and disorders, by administering to a patient in need of such treatment, a pharmaceutical composition of the present invention, is also encompassed.

In addition, the present invention further provides methods for treating or delaying the onset of the symptoms associated with cancer, systemic or chronic inflammation, rheumatoid arthritis, type 2 diabetes, obesity, T-cell mediated autoimmune disease, ischemia, and other complications associated with these diseases and disorders. These methods comprise administering an effective amount of one or more of the compounds of the present invention, preferably in the form of a pharmaceutical composition or medicament, to an individual having, or at risk of developing, cancer, systemic or chronic inflammation, rheumatoid arthritis, type 2 diabetes, obesity, T-cell mediated autoimmune disease, ischemia, and other complications associated with these diseases and disorders.

The compounds of the present invention can be used in combination therapies. Thus, combination therapy methods are also provided for treating or delaying the onset of the symptoms associated with cancer, systemic or chronic inflammation, rheumatoid arthritis, type 2 diabetes, obesity, T-cell mediated autoimmune disease, ischemia, and other complications associated with these diseases and disorders. Such methods comprise administering to a patient in need thereof one or more of the compounds of the present invention and, together or separately, at least one other anti-cancer, anti-inflammation, anti-rheumatoid arthritis, anti-type 2 diabetes, anti-obesity, anti-T-cell mediated autoimmune disease, or anti-ischemia therapy.

The foregoing and other advantages and features of the embodiments of the present invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only, and are not intended to be limiting.

Other features and advantages of the invention will be apparent to one of skill in the art from the following detailed description, and from the claims below.

### DETAILED DESCRIPTION OF THE INVENTION

### 1. Definitions

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group can consist of 1, 2 or 3 carbon atoms, or more carbon atoms, up to a total of 20). An alkyl group can be in an unsubstituted form or substituted form with one or more substituents (generally one to three substitutents can be present except in the case of halogen substituents, e.g., perchloro). For example, a C₁₋₆ alkyl group refers to a straight or branched aliphatic group containing 1 to 6 carbon atoms (e.g., include methyl, ethyl, propyl, isopropyl, butyl, sec-butyl, tert-butyl, 3-pentyl, hexyl, etc.), which can be optionally substituted.

As used herein, “lower alkyl” refers to an alkyl group having from 1 to 6 carbon atoms.

The term “alkylene” as used herein means a saturated aliphatic hydrocarbon straight chain or branched chain group having from 1 to 20 carbon atoms having two connecting points (i.e., a “divalent” chain). For example, “ethylene” represents the group —CH₂—CH₂— and “methylene” represents the group —CH₂—. Alkylene chain groups can also be thought of as multiple methylene groups. For example, ethylene contains two methylene groups. Alkylene groups can also be in an unsubstituted form or substituted form with one or more substituents.

The term “alkenyl” as employed herein by itself or as part of another group means a straight or branched divalent chain radical of 2-10 carbon atoms (unless the chain length is otherwise specified), including at least one double bond between two of the carbon atoms in the chain. The alkenyl group can also be in an unsubstituted form or substituted form with one or more substituents (generally one to three substitutents except in the case of halogen substituents, e.g., perchloro or perfluoroalkyls). For example, a C₂₋₆ alkenyl group refers to a straight or branched chain radical containing 2 to 6 carbon atoms and having at least one double bond between two of the carbon atoms in the chain (e.g., ethenyl, 1-propenyl, 2-propenyl, 2-methyl-1-propenyl, 1-butenyl and 2-butenyl, which can be optionally substituted).

The term “alkenylene” as used herein means an alkenyl group having two connecting points. For example, “ethenylene” represents the group —CH═CH—. Alkenylene groups can also be in an unsubstituted form or substituted form with one or more substituents.

The term “alkynyl” as used herein by itself or as part of another group means a straight or branched chain radical of 2-10 carbon atoms (unless the chain length is otherwise specified), wherein at least one triple bond occurs between two of the carbon atoms in the chain. The alkynyl group can be in an unsubstituted form or substituted form with one or more substituents (generally one to three substitutents except in the case of halogen substituents, e.g., perchloro or perfluoroalkyls). For example, a C₂₋₆ alkynyl group refers to a straight or branched chain radical containing 2 to 6 carbon atoms, which can be optionally substituted, and having at least one triple bond between two of the carbon atoms in the chain (e.g., ethynyl, 1-propynyl, 1-methyl-2-propynyl, 2-propynyl, 1-butynyl and 2-butynyl).

The term “alkynylene” as used herein means an alkynyl having two connecting points. For example, “ethynylene” represents the group —C≡C—. Alkynylene groups can also be in an unsubstituted form or substituted form with one or more substituents.

The term “carbocycle” as used herein by itself or as part of another group means cycloalkyl and non-aromatic partially saturated carbocyclic groups such as cycloalkenyl and cycloalkynyl. A carbocycle can be in an unsubstituted form or substituted form with one or more substituents so long as the resulting compound is sufficiently stable and suitable for use in the embodiments of the present invention.

The term “cycloalkyl” as used herein by itself or as part of another group refers to a fully saturated 3- to 8-membered cyclic hydrocarbon ring (i.e., a cyclic form of an alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with other such rings) (“polycyclic cycloalkyl”). Thus, a cycloalkyl can exist as a monocyclic ring, bicyclic ring, or a spiral ring. When a cycloalkyl is referred to as a Cₓ cycloalkyl, this means a cycloalkyl in which the fully saturated cyclic hydrocarbon ring (which may or may not be fused to another ring) has x number of carbon atoms. When a cycloalkyl is recited as a substituent on a chemical entity, it is intended that the cycloalkyl moiety is attached to the entity through a single carbon atom within the fully saturated cyclic hydrocarbon ring of the cycloalkyl. In contrast, a substituent on a cycloalkyl can be attached to any carbon atom of the cycloalkyl. A cycloalkyl group can be unsubstituted or substituted with one or more substitutents so long as the resulting compound is sufficiently stable and suitable for use in the embodiments of the present invention. Examples of cycloalkyl groups include, for example, cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring having a double bond therein (i.e., a cyclic form of an alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl can exist as a monocyclic ring, bicyclic ring, polycyclic or a spiral ring. When a cycloalkenyl is referred to as a Cₓ cycloalkenyl, this means a cycloalkenyl in which the non-aromatic partially saturated cyclic hydrocarbon ring (which may or may not be fused to another ring) has x number of carbon atoms. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group can be in an unsubstituted form or substituted form with one or more substitutents. Examples of cycloalkenyl groups include cyclopentenyl, cycloheptenyl and cyclooctenyl.

The term “heterocycle” (or “heterocyclyl” or “heterocyclic” or “heterocyclo”) as used herein by itself or as part of another group means a saturated or partially saturated 3-7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of atoms with such other rings) (“polycyclic heterocycle”). Thus, a heterocycle can exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle can be in an unsubstituted form or substituted form with one or more substituents so long as the resulting compound is sufficiently stable and suitable for use in the embodiments of the present invention.

Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is referred to as a Cₓ aryl, this means an aryl in which the all-carbon aromatic ring (which may or may not be fused to another ring) has x number of carbon atoms. When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl can be in an unsubstituted form or substituted form with one or more substituents so long as the resulting compound is sufficiently stable and suitable for use in the embodiments of the present invention.

The term “heteroaryl” as employed herein refers to a stable aromatic ring having up to 7 ring atoms with 1, 2, 3 or 4 hetero ring actoms in the ring which are oxygen, nitrogen or sulfur or a combination thereof (“monocylic heteroaryl”). In addition to monocyclic heteroaromatic rings, the term “heteroaryl” also encompasses a group having the monocyclic heteroaromatic ring above fused to another cycloalkyl, cycloalkynyl, cycloalkenyl, heterocycle, aryl or heteroaryl ring (i.e., sharing an adjacent pair of atoms with such other rings) (“polycyclic heteroaryl”). When a heteroaryl is recited as a substituent on a chemical entity, it is intended that the heteroaryl moiety is attached to the entity through an atom within the heteroaromatic ring of the heteroaryl. In contrast, a substituent on a heteroaryl can be attached to any suitable atom of the heteroaryl. A heteroaryl can be in an unsubstituted form or substituted form with one or more substituents so long as the resulting compound is sufficiently stable and suitable for use in the embodiments of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-c]pyrimidin-4-one, pyrazolo[1,5-c]pyrimidinyl, including without limitation pyrazolo[1,5-c]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom can be in the form of an N-oxide, e.g., a pyridyl N-oxide, pyrazinyl N-oxide and pyrimidinyl N-oxide.

As used herein, the term “halo” refers to chloro, fluoro, bromo, or iodo substitutents.

As used herein, the term “hydro” refers to a bound hydrogen atom (—H group).

As used herein, the term “hydroxyl” refers to an —OH group.

As used herein, the term “alkoxy” refers to an —O—(C₁₋₁₂ alkyl). Lower alkoxy refers to —O-(lower alkyl) groups.

As used herein, the term “alkynyloxy” refers to an —O—(C₂₋₁₂ alkynyl).

As used herein, the term “cycloalkyloxy” refers to an —O-cycloalkyl group.

As used herein, the term “heterocycloxy” refers to an —O-heterocycle group.

As used herein, the term “aryloxy” refers to an —O-aryl group. Examples of aryloxy groups include, but are not limited to, phenoxy and 4-methylphenoxy.

The term “heteroaryloxy” refers to an —O-heteroaryl group.

The terms “arylalkoxy” and “heteroarylalkoxy” are used herein to mean alkoxy group substituted with an aryl group and a heteroaryl group, respectively. Examples of arylalkoxy groups include, but are not limited to, benzyloxy and phenethyloxy.

As used herein, the term “mercapto” or “thiol” group refers to an —SH group.

The term “alkylthio” group refers to an —S-alkyl group.

The term “arylthio” group refers to an —S-aryl group.

The term “arylalkyl” is used herein to mean above-defined alkyl group substituted by an aryl group defined above. Examples of arylalkyl groups include benzyl, phenethyl and naphthylmethyl, etc. An arylalkyl group can be unsubstituted or substituted with one or more substituents so long as the resulting compound is sufficiently stable and suitable for use in the embodiments of the present invention.

The term “heteroarylalkyl” is used herein to mean an alkyl group, as defined above, substituted by any heteroaryl group. A heteroarylalkyl can be unsubstituted or substituted with one or more substituents, so long as the resulting compound is sufficiently stable and suitable for use in the embodiments of the present invention.

The term “heteroarylalkenyl” is used herein to mean any of the above-defined alkenyl groups substituted by any of the above-defined heteroaryl groups.

The term “arylalkynyl” is used herein to mean any of the above-defined alkynyl groups substituted by any of the above-defined aryl groups.

The term “heteroarylalkenyl” is used herein to mean any of the above-defined alkenyl groups substituted by any of the above-defined heteroaryl groups.

The term “arylalkoxy” is used herein to mean alkoxy group substituted by an aryl group as defined above.

“Heteroarylalkoxy” is used herein to mean any of the above-defined alkoxy groups substituted by any of the above-defined heteroaryl groups.

“Haloalkyl” means an alkyl group that is substituted with one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has an oxygen doubly-bonded to it; i.e. one of the ring carbon atoms is a —C(═O) group.

As used herein, the term “thiocarbonyl” group refers to a —C(═S)R″ group, with R″ as defined herein.

“Alkanoyl” refers to an —C(═O)-alkyl group.

The term “heterocyclonoyl” group refers to a heterocyclo group linked to the alkyl chain of an alkanoyl group.

The term “acetyl” group refers to a —C(═O)CH₃ group.

“Alkylthiocarbonyl” refers to an —C(═S)-alkyl group.

The term “cycloketone” refers to a carbocycle or heterocycle group in which one of the carbon atoms which form the ring has an oxygen doubly-bonded to it; i.e., one of the ring carbon atoms is a —C(═O) group.

The term “O-carboxy” group refers to a —OC(═O)R″group, where R″ is as defined herein.

The term “C-carboxy” group refers to a —C(═O)OR″ groups where R″ is as defined herein.

As used herein, the term “carboxylic acid” refers to a C-carboxy group in which R″ is hydro. In other words, the term “carboxylic acid” refers to —COOH.

As used herein, the term “ester” is a C-carboxy group, as defined herein, wherein R″ is as defined above, except that it is not hydro (e.g., it is methyl, ethyl, or lower alkyl).

As used herein, the term “C-carboxy salt” refers to a —C(═O)O⁻ M⁺ group wherein M⁺ is selected from the group consisting of lithium, sodium, magnesium, calcium, potassium, barium, iron, zinc and quaternary ammonium.

The term “carboxyalkyl” refers to —C₁₋₆ alkylene—C(═O)OR″ (that is, a C₁₋₆ alkyl group connected to the main structure wherein the alkyl group is substituted with —C(═O)OR″ with R″ being defined herein). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene—C(═O)OR″ with R″ being defined herein.

The term “carboxyalkyl salt” refers to a —(CH₂)ᵣC(═O)O⁻ M⁺ wherein M⁺ is selected from the group consisting of lithium, sodium, potassium, calcium, magnesium, barium, iron, zinc and quaternary ammonium, and wherein r is 1-6.

The term “carboxyalkoxy” refers to —O—(CH₂)ᵣC(═O)OR″ wherein r is 1-6, and R″ is as defined herein.

“Cₓ carboxyalkanoyl” means a carbonyl group (—O(O═)C—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—(O═)C—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene—O—C₁₋₆ alkylene—C(═O)—, R″ is as defined herein.

“Amino” refers to an —NR^{x}R^{y} group, with R^{x} and R^{y} as defined herein.

“Alkylamino” means an amino group with a substituent being a C₁₋₆ alkyl.

“Aminoalkyl” means an alkyl group connected to the main structure of a molecule where the alkyl group has a substituent being amino.

“Quaternary ammonium” refers to a —⁺N(R^{x})(R^{y})(R^{z}) group wherein R^{x}, R^{y}, and R^{z} are as defined herein.

The term “nitro” refers to a —NO₂ group.

The term “O-carbamyl” refers to a —OC(═O)N(R^{x})(R^{y}) group with R^{x} and R^{y} as defined herein.

The term “N-carbamyl” refers to a R^{y}OC(═O)N(R^{x})— group, with R^{x} and R^{y} as defined herein.

The term “O-thiocarbamyl” refers to a —OC(═S)N(R^{x})(R^{y}) group with R^{x} and R^{y} as defined herein.

The term “N-thiocarbamyl” refers to a R^{x}OC(═S)NR^{y}— group, with R^{x} and R^{y} as defined herein.

“C-amido” refers to a —C(═O)N(R^{x})(R^{y}) group with R^{x} and R^{y} as defined herein.

“N-amido” refers to a R^{x}C(═O)N(R^{y})— group with R^{x} and R^{y} as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R^{x})(alkoxy) group with R^{x} as defined herein.

The terms “cyano” and “cyanyl” refer to a —C≡N group.

The term “nitrile” group, as used herein, refers to a —C≡N substituent.

The term “cyanato” refers to a —CNO group.

The term “isocyanato” refers to a —NCO group.

The term “thiocyanato” refers to a —CNS group.

The term “isothiocyanato” refers to a —NCS group.

The term “oxo” refers to a —C(═O)— group.

The term “sulfinyl” refers to a —S(═O)R″ group, where R″ is as defined herein.

The term “sulfonyl” refers to a —S(═O)₂R″ group, where R″ is as defined herein.

The term “sulfonamide” refers to a —(R^{x})N—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{y})N—S(═O)₂—O— group with R^{x} and R^{y} as defined herein.

“Sulfonamidecarbonyl” means R″—S(═O)₂—N(R^{x})—C(═O)— with R″ and R^{x} as defined herein.

“Alkanoylaminosulfonyl” refers to an alkyl—C(═O)—N(R^{x})—S(═O)₂— group with R^{x} as defined herein.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is selected from the group consisting of hydro, alkyl, cycloalkyl, aryl, heteroaryl and heterocycle, each being optionally substituted.

R^{x}, R^{y}, and R^{z} are independently selected from the group consisting of hydro and optionally substituted alkyl.

The term “methylenedioxy” refers to a —OCH₂O— group wherein the oxygen atoms are bonded to adjacent ring carbon atoms.

The term “ethylenedioxy” refers to a —OCH₂CH₂O— group wherein the oxygen atoms are bonded to adjacent ring carbon atoms.

The symbol “═ ═” in a chemical structure refers to a bond that can be either a “double” or a “single” bond, as those terms are used in the art.

As used herein, the phrase “optionally substituted” means substituted or unsubstituted.

Unless specifically stated otherwise or indicated by a bond symbol (dash, double dash, or triple dash), the connecting point to a recited group will be on the right-most stated group. Thus, for example, a hydroxyalkyl group is connected to the main structure through the alkyl and the hydroxyl is a substituent on the alkyl.

### 2. Therapeutic Compounds

The present invention provides chemical compounds that selectively inhibit the activity of Nampt. These compounds can be used in the treatment of cancer, systemic or chronic inflammation, rheumatoid arthritis, diabetes, obesity, T-cell mediated autoimmune disease, ischemia, and complications associated with these diseases and disorders.

In some embodiments, the present invention provides compounds of Formula I

J-K-L-E-Q-P Formula I

and pharmaceutically-acceptable salts and solvates thereof; wherein:

J is selected from: alkyl, nitro, cyano, alkoxy, C-amido, N-amido, haloalkyl, C-carboxy, O-carboxy, sulfonamide, amino, hydroxyl, mercapto, alkylthio, sulfonyl, sulfinyl, carbocycle, spiro-linked (i.e., two adjacent atoms of J are linked to one atom of K) carbocycle, cycloalkyl, spiro-linked cycloalkyl, cycloalkenyl, spiro-linked cycloalkenyl, heterocycle, spiro-linked heterocycle, heterocyclonoyl, aryl, spiro-linked aryl, heteroaryl, spiro-linked heteroaryl, carbocycloalkyl, heterocyclylalkyl, arylalkyl, arylalkenyl, heteroarylalkyl, heteroarylalkenyl, heteroarylalkynyl, or arylalkynyl, wherein any of the foregoing groups are optionally substituted at least once with alkyl, alkylene, alkenyl, alkenylene, alkynyl, alkynylene, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyamino carbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide, wherein any of the foregoing optional substituents are themselves optionally substituted;

K is an optionally further substituted 5-membered heteroaryl or heterocyclic ring;

L is either (i) an optionally-substituted phenyl or an optionally-substituted 5- or 6-membered heteroaryl ring, (ii) optionally-substituted 5- or 6-membered cycloalkyl, (iii) optionally-substituted alkyl, (iv) optionally-substituted alkenyl, or (v) optionally-substituted alkynyl;

E is either (i) —C₀₋₂ alkylene-N(H)—C(═X)—N(H)— or (ii) -M-C(═X′)—N(H)—, wherein X is O, S, or N—C≡N, wherein M is optionally-substituted ethenylene or optionally-substituted ethylene, and wherein X′ is O or S;

Q is optionally present and if present is optionally-substituted ethylene or optionally-substituted methylene;

P is an optionally-substituted pyridinyl ring;

with the proviso that when L is optionally-substituted alkyl, then K is an optionally-substituted 5-membered bicyclic heteroaryl or bicyclic heterocyclic ring (i.e., K comprises a 5-membered heteroaryl or heterocyclic ring fused to a second ring, wherein attachment to J and L is via the 5-membered heteroaryl or heterocyclic ring); and

with the proviso that when E is -M-C(═X′)—N(H)—, then K is not xanthine; and also

with the proviso that when E is —C₀₋₂ alkylene-N(H)—C(═X)—N(H)—, then either K is an optionally-substituted 5-membered bicyclic heteroaryl or bicyclic heterocyclic ring (i.e., K comprises a 5-membered heteroaryl or heterocyclic ring fused to a second ring, wherein attachment to J and L is via the 5-membered heteroaryl or heterocyclic ring) or J is a spiro-linked moiety (i.e., two adjacent atoms of J are linked to one atom of K), such as, for example, spiro-linked carbocycle, spiro-linked cycloalkyl, spiro-linked cycloalkenyl, spiro-linked heterocycle, spiro-linked aryl, and spiro-linked heteroaryl; and

with the proviso that the compound is not:
Urea, N-(6-chloro-3-pyridinyl)-N′-[2-[4-(5-methyl-3-oxo-1H-imidazo[1,5-c]imidazol-2(3H)-yl)-1-piperidinyl]-2-oxo-1-phenylethyl]-;
Urea, N-[2-(3′-chloro[1,1′-biphenyl]-4-yl)-2-(1-cyclopentyl-4-piperidinyl)ethyl]-N′-3-pyridinyl-;
Urea, N-[2-(3′-cyano[1,1′-biphenyl]-4-yl)-2-(1-cyclopentyl-4-piperidinyl)ethyl]-N′-3-pyridinyl-;
2H-Pyrazino[2,1-c][1,2,4]triazine-1(6H)-carboxamide,hexahydro-6-[(4-hydroxyphenyl)methyl]-8-[[1-methyl-3-[4-[[[[6-(4-methyl-1-piperazinyl)-3-pyridinyl]amino]carbonyl]amino]phenyl]-1H-indol-7-yl]methyl]-4,7-dioxo-N-(phenylmethyl)-2-(2-propen-1-yl)-, (6S,9aS)-; or
2H-Pyrazino[2,1-c][1,2,4]triazine-1(6H)-carboxamide,hexahydro-6-[(4-hydroxyphenyl)methyl]-8-[[3-[4-[[[(6-methoxy-3-pyridinyl)amino]carbonyl]amino]phenyl]-1-methyl-1H-indol-7-yl]methyl]-4,7-dioxo-N-(phenylmethyl)-2-(2-propen-1-yl)-(6S,9aS)-.

In some embodiments of the compounds of Formula I, L is selected from phenyl, thienyl (thiophenyl), furyl (furanyl), pyrrolyl (including without limitation 2H-pyrrolyl), imidazolyl, pyrazolyl, isothiazolyl, thiazolyl, isoxazolyl, oxadiazolyl, oxazolyl, furazanyl, pyridinyl, pyrimidinyl, pyrazinyl, pyridazinyl, triazinyl, pyranyl, thiopyranyl, silinyl, phosphininyl, arsininyl, thiazinyl, dioxinyl, dithiinyl, or tetrazinyl.

In some embodiments of the compounds of Formula I, L is selected from cyclohexyl or cyclopentyl.

In some embodiments of the compounds of Formula I, Q is methylene or ethylene. In some of such embodiments, the methylene or ethylene is substituted one or more times with C₁₋₄ alkyl, halo, C₁₋₄ haloalkyl, or C₃ or C₄ cycloalkyl. In other embodiments the methylene or ethylene is unsubstituted.

In some embodiments of the compounds of Formula I, P is 3-pyridinyl. In some embodiments of the compounds of Formula I, P is 4-pyridinyl. In some embodiments of the compounds of Formula I, P is not substituted or is substituted one, two, three, or four times. In some embodiments of the compounds of Formula I, any substituent of P is halo (such as, for example, fluoro), methyl, nitro, cyano, trihalomethyl, methoxy, amino, hydroxyl, or mercapto. In some embodiments of the compounds of Formula I, P is unsubstituted 3-pyridinyl or is 3-pyridinyl substituted at the 4 position with NH₂.

In some embodiments, the present invention provides compounds of Formula II

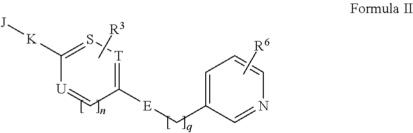

and pharmaceutically-acceptable salts and solvates thereof; wherein:

J and K are each as defined for Formula I;

S, T, and U are each independently carbon or nitrogen, provided that when any of S, T, or

U is nitrogen, then there is no substituent on the nitrogen;

n is 0 or 1;

R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

E is either (i) —C₀₋₂ alkylene-N(H)—C(═X)—N(H)— or (ii) -M-C(═X′)—N(H)—, wherein X is O, S, or N—C≡N, wherein M is optionally-substituted ethenylene or optionally-substituted ethylene, and wherein X′ is O or S;

q is 0, 1, or 2, wherein any methylene group of the q region is optionally independently substituted with C₁₋₄ alkyl, halo, C₁₋₄ haloalkyl, or C₃ or C₄ cycloalkyl;

R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl; and

with the proviso that when E is -M-C(═X′)—N(H)—, then K is not xanthine; and also with the proviso that when E is —C₀₋₂ alkylene-N(H)—C(═X)—N(H)—, then either K is an optionally-substituted 5-membered bicyclic heteroaryl or bicyclic heterocyclic ring (i.e., K comprises a 5-membered heteroaryl or heterocyclic ring fused to a second ring, wherein attachment to J and L is via the 5-membered heteroaryl or heterocyclic ring) or J is a spiro-linked moiety (i.e., two adjacent atoms of J are linked to one atom of K), such as, for example, spiro-linked carbocycle, spiro-linked cycloalkyl, spiro-linked cycloalkenyl, spiro-linked heterocycle, spiro-linked aryl, and spiro-linked heteroaryl; and,

with the proviso that the compound is not:
2H-Pyrazino[2,1-c][1,2,4]triazine-1(6H)-carboxamide,hexahydro-6-[(4-hydroxyphenyl)methyl]-8-[[3-[4-[[[(6-methoxy-3-pyridinyl)amino]carbonyl]amino]phenyl]-1-methyl-1H-indol-7-yl]methyl]-4,7-dioxo-N-(phenylmethyl)-2-(2-propen-1-yl)-, (6S,9aS)—;
Benzenepropanamide, 4-(2-methyl-3H-imidazo[4,5-b]pyridin-3-yl)-N-(3-pyridinylmethyl)-;
Pentanamide, 5-chloro-N-[(5-chloro-2-methyl-3-pyridinyl)methyl]-2-[[3-methoxy-4-(4-methyl-1H-imidazol-1-yl)phenyl]methylene]-,(2E)-; or
Pentanamide, 5-chloro-2-[[3-methoxy-4-(4-methyl-1H-imidazol-1-yl)phenyl]methylene]-N-[[6-(4-morpholinyl)-3-pyridinyl]methyl]-,(2E)-.

In some embodiments of the compounds of each of Formulae I and II, E is -M-C(═X′)—N(H)—. In some of such embodiments M is optionally-substituted ethenylene, including unsubstituted ethenylene. In others of such embodiments M is optionally-substituted ethylene, including unsubstituted ethylene. In some of such embodiments X′ is oxygen. In others of such embodiments X′ is sulfur.

In some embodiments of the compounds of each of Formula I and II, the ethenylene or ethylene group of M is substituted one or more times with hydroxyl, C₁₋₄ alkyl, C₁₋₄ alkoxy, halo, C₁₋₄ haloalkyl, C≡N, or C₃ or C₄ cycloalkyl.

In some embodiments of the compounds of each of Formulae I and II, E is —C₀₋₂ alkylene-N(H)—C(═X)—N(H)—. In some of such embodiments E is —N(H)—C(═X)—N(H)—. In some of such embodiments X is oxygen. In others of such embodiments X is sulfur. In yet others of such embodiments X is N—C≡N.

In some embodiments of the compounds of each of Formulae I and II, J comprises a nitrogen atom.

In some embodiments of the compounds of each of Formulae I and II, J is selected from the following:

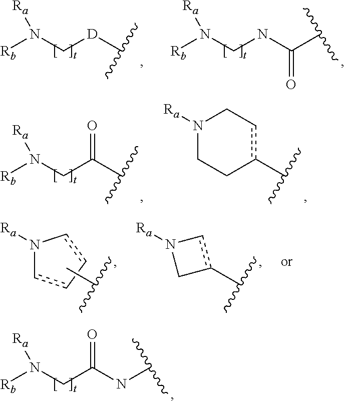

wherein t is 0, 1, 2, 3, or 4; D is N(H), O, C(H)₂, or S; and Rₐ and R_{b} are each independently hydro, C₃₋₆ cycloalkyl, optionally-substituted C₃₋₆ heterocyclo, or C₁₋₆ alkyl, or Rₐ and R_{b}, together with the linking nitrogen between them, form a first C₃₋₆ heterocyclo, and wherein the first C₃₋₆ heterocyclo is optionally substituted with C₁₋₆ alkyl, amino, or a second C₃₋₆ heterocyclo.

In some embodiments of the compounds of each of Formulae I and II, J is selected from the following:

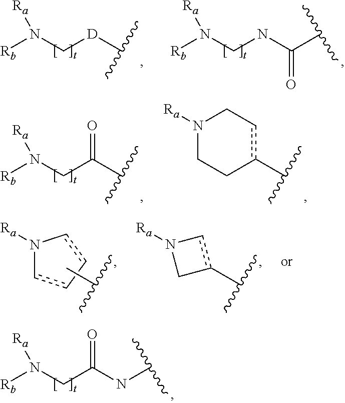

wherein t is 0, 1, 2, 3, or 4; D is N(H), O, C(H)₂, or S; and Rₐ and R_{b} are each independently hydro, C₃₋₆ cycloalkyl, C₁₋₆ alkyl, optionally-substituted morpholine, optionally-substituted piperazine, optionally-substituted azetidine, optionally-substituted pyrrolidine, or optionally-substituted piperidine; or Rₐ and R_{b}, together with the linking nitrogen between them, form a first ring selected from morpholine, piperazine, azetidine, pyrrolidine, or piperidine, wherein the first ring is optionally substituted with C₁₋₆ alkyl, amino, or a second ring selected from optionally-substituted morpholine, optionally-substituted piperazine, optionally-substituted azetidine, optionally-substituted pyrrolidine, or optionally-substituted piperidine.

In some embodiments of the compounds of each of Formulae I and II, J is selected from the following:

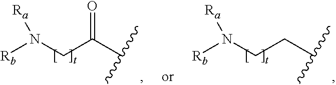

wherein t is 0, 1, 2, 3, or 4; and Rₐ and R_{b} are each independently hydro, C₃₋₆ cycloalkyl, C₁₋₆ alkyl, optionally-substituted morpholine, optionally-substituted piperazine, optionally-substituted azetidine, optionally-substituted pyrrolidine, or optionally-substituted piperidine; or Rₐ and R_{b}, together with the linking nitrogen between them, form a first ring selected from morpholine, piperazine, azetidine, pyrrolidine, and piperidine, wherein the first ring is optionally substituted with C₁₋₆ alkyl, amino, or a second ring selected from optionally-substituted morpholine, optionally-substituted piperazine, optionally-substituted azetidine, optionally-substituted pyrrolidine, or optionally-substituted piperidine.

In some embodiments of the compounds of each of Formulae I and II, J is selected from the following: spiro-linked carbocycle, spiro-linked cycloalkyl, spiro-linked cycloalkenyl, spiro-linked heterocycle, spiro-linked aryl, or spiro-linked heteroaryl, wherein any of the foregoing groups are optionally substituted at least once with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, sulfonyl, or the following:

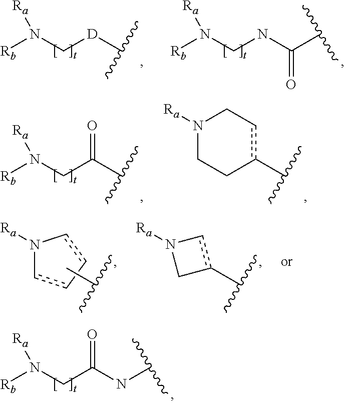

wherein t is 0, 1, 2, 3, or 4 and any methylene group of the t region is optionally-substituted one or more times with C₁₋₃ alkyl; D is N(H), O, C(H)₂, or S; and Rₐ and R_{b} are each independently hydro, C₃₋₆ cycloalkyl, optionally-substituted C₃₋₆ heterocyclo, or C₁₋₆ alkyl, or Rₐ and R_{b}, together with the linking nitrogen between them, form a first C₃₋₆ heterocyclo, wherein the first C₃₋₆ heterocyclo is optionally substituted with C₁₋₆ alkyl, amino, or a second C₃₋₆ heterocyclo.

In some embodiments of the compounds of each of Formulae I and II, J is a spiro-linked heterocycle, optionally substituted at the heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, sulfonyl, or one of the following:

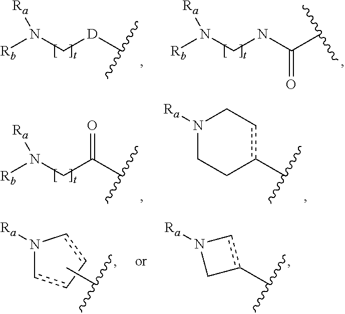

wherein t is 0, 1, 2, 3, or 4 and any methylene group of the t region is optionally-substituted one or more times with C₁₋₃ alkyl; D is O, C(H)₂, or S; and Rₐ and R_{b} are each independently hydro, C₃₋₆ cycloalkyl, optionally-substituted C₃₋₆ heterocyclo, or C₁₋₆ alkyl, or Rₐ and R_{b}, together with the linking nitrogen between them, form a first C₃₋₆ heterocyclo, wherein the first C₃₋₆ heterocyclo is optionally substituted with C₁₋₆ alkyl, amino, or a second C₃₋₆ heterocyclo.

In some embodiments of the compounds of each of Formulae I and II, J together with a ring carbon of K forms

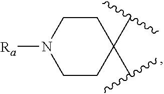

wherein Rₐ is selected from halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl.

In some embodiments of the compounds of each of Formulae I and II, K is an optionally-substituted 5-membered monocyclic heteroaryl ring, such as, for example, thienyl (thiophenyl), furyl (furanyl), pyrrolyl (including without limitation 2H-pyrrolyl), imidazolyl, pyrazolyl, isothiazolyl, thiazolyl, isoxazolyl, oxazolyl, and furazanyl.

In some embodiments of the compounds of each of Formulae I and II, K is an optionally-substituted 5-membered bicyclic heteroaryl ring (i.e., K comprises a 5-membered heteroaryl ring fused to a second ring, wherein attachment to J and L is via the 5-membered heteroaryl ring), such as, for example, benzo[b]thienyl, benzo[b]furanyl, isobenzofuranyl, isobenzothiophenyl, indolizinyl, isoindolyl, 3H-indolyl, indolyl, indazolyl, purinyl, pyrazolopyrazinyl, imidazopyrazinyl, pyrazolopyridazinyl, imidazopyridazinyl, imidazopyrimidinyl, pyrazolopyrimidinyl, isoxazolopyrazinyl, oxazolopyrazinyl, isoxazolopyridazinyl, oxazolopyridazinyl, oxazolopyrimidinyl, isoxazolopyrimidinyl, isothiazolopyrazinyl, thiazolopyrazinyl, isothiazolopyridazinyl, thiazolopyridazinyl, thiazolopyrimidinyl, isothiazolopyrimidinyl, pyrazolo[1,5-c]pyrimidinyl, including without limitation pyrazolo[1,5-c]pyrimidin-3-yl, pyrazolo[1,5-a]pyridinyl, isoxazolo[2,3-a]pyridinyl, isothiazolo[2,3-a]pyridinyl, imidazo[1,5-a]pyridinyl, oxazolo[3,4-a]pyridinyl, thiazolo[3,4-a]pyridinyl, imidazo[1,2-a]pyridinyl, oxazolo[3,2-a]pyridinyl, thiazolo[3,2-c]pyridinyl, benzoisoxazolyl, benzoxazolyl, 1,2-benzoisoxazol-3-yl, benzimidazolyl, benzthiazolyl, benzisothiazolyl, 2-oxindolyl, and 2-oxobenzimidazolyl.

In some embodiments of the compounds of Formula II, at least one of S, T, and U is nitrogen. In some embodiments of the compounds of Formula II, at least two of S, T, and U are nitrogen. In some embodiments of the compounds of Formula II, only S is nitrogen. In some embodiments of the compounds of Formula II, only T is nitrogen. In some embodiments of the compounds of Formula II, only U is nitrogen. In some embodiments of the compounds of Formula I, S and U are nitrogen. In some embodiments of the compounds of Formula II, S, T, and U are all carbon.

In some embodiments of the compounds of Formula II, n is 0. In some embodiments of the compounds of Formula II, n is 1.

In some embodiments, the present invention provides compounds of Formula III

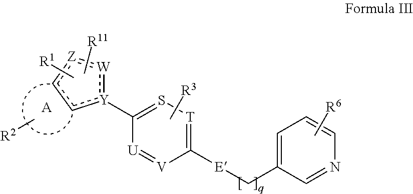

and pharmaceutically-acceptable salts and solvates thereof; wherein:

R¹ substitutes for a hydrogen and is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, and optionally-substituted sulfinyl;

R¹¹ is optionally present, and if present, substitutes a hydrogen and together with R¹ forms a spiro-linked heterocycle (i.e., R¹ and R¹¹ both attach to the same ring carbon atom) optionally substituted at a heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, or sulfonyl;

A is optionally present and when present is cycloalkyl, heterocycle, aryl, or heteroaryl;

R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl; with the proviso that R² is only present if A is present;

W, Y, and Z are each independently carbon or nitrogen, provided that at least one, but not both, of Y and Z is nitrogen;

S, T, U, and V are each independently carbon or nitrogen, provided that when any of S, T, U, or V is nitrogen, then there is no substituent on the nitrogen;

R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

E′ is either —C₀₋₂ alkylene-N(H)—C(═O)—N(H)— or

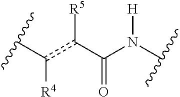

wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;

q is 0, 1, or 2, wherein any methylene group of the q region is optionally independently substituted with C₁₋₄ alkyl, halo, C₁₋₄ haloalkyl, or C₃ or C₄ cycloalkyl;

R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl;

with the proviso that when E′ is —C₀₋₂ alkylene-N(H)—C(═O)—N(H)—, then A is present; and

with the proviso that the compound is not:
Benzenepropanamide, 4-(2-methyl-3H-imidazo[4,5-b]pyridin-3-yl)-N-(3-pyridinylmethyl)-; and

with the proviso that when E is -M-C(═X′)—N(H)—, then K is not xanthine.

In some embodiments of the compounds of Formula III, at least one of S, T, U, and V is nitrogen. In some embodiments of the compounds of Formula III, at least two of S, T, and U, and V are nitrogen. In some embodiments of the compounds of Formula III, only S is nitrogen. In some embodiments of the compounds of Formula III, only T is nitrogen. In some embodiments of the compounds of Formula III, only U is nitrogen. In some embodiments of the compounds of Formula III, only V is nitrogen. In some embodiments of the compounds of Formula III, T and V are nitrogen. In some embodiments of the compounds of Formula III, S and U are nitrogen. In some embodiments of the compounds of Formula I, S, T, U, and V are all carbon.

In some embodiments of the compounds of Formula III, E′ is

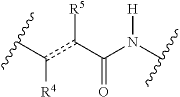

wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl. In some of such embodiments, “═ ═” is a double bond, while in other embodiments “═ ═” is a single bond.

In some embodiments of the compounds of each of Formulae I and II, E′ is —C₀₋₂ alkylene-N(H)—C(═O)—N(H)—. In some of such embodiments E′ is —N(H)—C(═O)—N(H)—.

In some embodiments of the compounds of Formula III, R¹ is a substituent of Z. In some embodiments of the compounds of Formula III, R¹ is a substituent of W.

In some embodiments of the compounds of Formula III, A is present and is a cycloalkyl ring.

In some embodiments of the compounds of Formula III, A is present and is a heterocycle ring.

In some embodiments of the compounds of Formula III, A is present and is an aryl ring.

In some embodiments of the compounds of Formula III, A is present and is an heteroaryl ring.

In some embodiments of the compounds of Formula III, A is present and is a cyclopentyl ring.

In some embodiments of the compounds of Formula III, A is present and is a cyclohexyl ring.

In some embodiments of the compounds of Formula III, A is present and is a cycloheptyl ring.

In some embodiments of the compounds of Formula III, A is present and is a pyridine ring, such as a 2-pyridine ring, a 3-pyridine ring, or a 4-pyridine ring.

In some embodiments of the compounds of Formula III, A is present and is a pyrimidine ring.

In some embodiments of the compounds of Formula III, A is present and is a pyrazine ring.

In some embodiments of the compounds of Formula III, A is present and is a pyridazine ring.

In some embodiments of the compounds of Formula III, A is not present.

In some embodiments, the present invention provides compounds of Formula IV

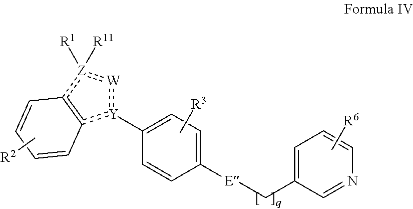

and pharmaceutically-acceptable salts and solvates thereof; wherein:

R¹ is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl;

R¹¹ is optionally present, and if present, substitutes a hydrogen and together with R¹ forms a spiro-linked heterocycle (i.e., R¹ and R¹¹ both attach to the same ring carbon atom) optionally substituted at a heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, or sulfonyl;

R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

W, Y, and Z are each independently carbon or nitrogen, provided that at least one, but not both, of Y and Z is nitrogen;

R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

E″ is either —N(H)—C(═O)—N(H)— or

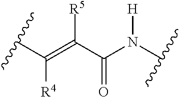

wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;

q is 0, 1, or 2, wherein any methylene group of the q region is optionally independently substituted with C₁₋₄ alkyl, halo, C₁₋₄ haloalkyl, or C₃ or C₄ cycloalkyl; and

R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl.

In some embodiments of the compounds of each of Formulae III and IV, the ring comprising W, Y, and Z is aromatic.

In some embodiments of the compounds of each of Formulae III and IV, the ring comprising W, Y, and Z is alicyclic. In some of such embodiments, the ring comprising W, Y, and Z contains only single bonds.

In some embodiments, the present invention provides compounds of Formula IVa

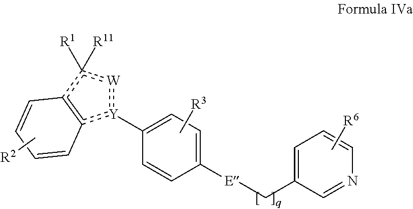

and pharmaceutically-acceptable salts and solvates thereof; wherein:

R¹ is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl;

R¹¹ is optionally present, and if present, substitutes a hydrogen and together with R¹ forms a spiro-linked heterocycle (i.e., R¹ and R¹¹ both attach to the same ring carbon atom) optionally substituted at a heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, or sulfonyl;

R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

W is carbon or nitrogen;

R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

E″ is either —N(H)—C(═O)—N(H)— or

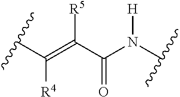

wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;

q is 1 or 2, wherein any methylene group of the q region is optionally independently substituted with C₁₋₄ alkyl, halo, C₁₋₄ haloalkyl, or C₃ or C₄ cycloalkyl; and

R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl.

In some embodiments of the compounds of Formula IV, the ring comprising W is aromatic.

In some embodiments of the compounds of Formula IV, the ring comprising W is alicyclic. In some of such embodiments, the ring comprising W contains only single bonds.

In some embodiments, the present invention provides compounds of Formula IVb

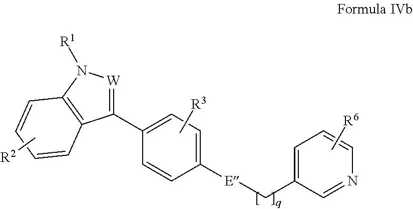

and pharmaceutically-acceptable salts and solvates thereof; wherein:

R¹ is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl;

R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

W is carbon or nitrogen;

R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;

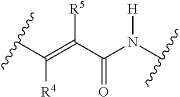

E″ is either —N(H)—C(═O)—N(H)— or wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;

q is 1 or 2, wherein any methylene group of the q region is optionally independently substituted with C₁₋₄ alkyl, halo, C₁₋₄ haloalkyl, or C₃ or C₄ cycloalkyl; and

R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl.

In some embodiments of the compounds of each of Formulae IV, IVa, and IVb, E″ is

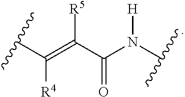

In some embodiments of the compounds of each of Formulae IV, IVa, and IVb, E″ is —N(H)—C(═O)—N(H)—.

In some embodiments of the compounds of each of Formulae III, IV, IVa, and IVb, R¹ is selected from C₁₋₅ alkyl, C₁₋₅ alkoxy, C-amido, N-amido, amino, aminoalkyl, or alkylthio, each further substituted with heterocyclo, cycloalkyl, or amino.

In some embodiments of the compounds of each of Formulae III, IV, IVa, and IVb, R¹ is selected from the following:

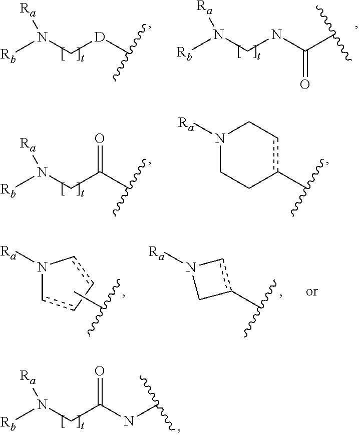

wherein t is 0, 1, 2, 3, or 4; D is N(H), O, C(H)₂, or S; and Rₐ and R_{b} are each independently hydro, C₃₋₆ cycloalkyl, optionally-substituted C₃₋₆ heterocyclo, or C₁₋₆ alkyl, or Rₐ and R_{b}, together with the linking nitrogen between them, form a first C₃₋₆ heterocyclo, wherein the first C₃₋₆ heterocyclo is optionally substituted with C₁₋₆ alkyl, amino, or a second C₃₋₆ heterocyclo.

In some embodiments of the compounds of each of Formulae III, IV, IVa, and IVb, R¹ is selected from the following:

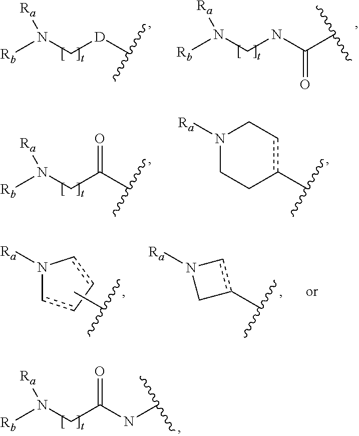

wherein t is 0, 1, 2, 3, or 4; D is N(H), O, C(H)₂, or S; and Rₐ and R_{b} are each independently hydro, C₃₋₆ cycloalkyl, C₁₋₆ alkyl, optionally-substituted morpholine, optionally-substituted piperazine, optionally-substituted azetidine, optionally-substituted pyrrolidine, or optionally-substituted piperidine; or Rₐ and R_{b}, together with the linking nitrogen between them, form a first ring selected from morpholine, piperazine, azetidine, pyrrolidine, or piperidine, wherein the first ring is optionally substituted with C₁₋₆ alkyl, amino, or a second ring selected from optionally-substituted morpholine, optionally-substituted piperazine, optionally-substituted azetidine, optionally-substituted pyrrolidine, or optionally-substituted piperidine.

In some embodiments of the compounds of each of Formulae III, IV, IVa, and IVb, R¹ is:

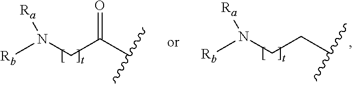

wherein t is 0, 1, 2, 3, or 4; and Rₐ and R_{b} are each independently hydro, C₃₋₆ cycloalkyl, C₁₋₆ alkyl, optionally-substituted morpholine, optionally-substituted piperazine, optionally-substituted azetidine, optionally-substituted pyrrolidine, or optionally-substituted piperidine; or Rₐ and R_{b}, together with the linking nitrogen between them, form a first ring selected from morpholine, piperazine, azetidine, pyrrolidine, and piperidine, wherein the first ring is optionally substituted with C₁₋₆ alkyl, amino, or a second ring selected from optionally-substituted morpholine, optionally-substituted piperazine, optionally-substituted azetidine, optionally-substituted pyrrolidine, or optionally-substituted piperidine.

In some embodiments of the compounds of each of Formulae III, IV, and IVa, R¹ and R¹¹ together form a spiro-linked carbocycle, spiro-linked cycloalkyl, spiro-linked cycloalkenyl, spiro-linked heterocycle, spiro-linked aryl, and spiro-linked heteroaryl, wherein any of the foregoing groups are optionally substituted at least once with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, sulfonyl, or the following:

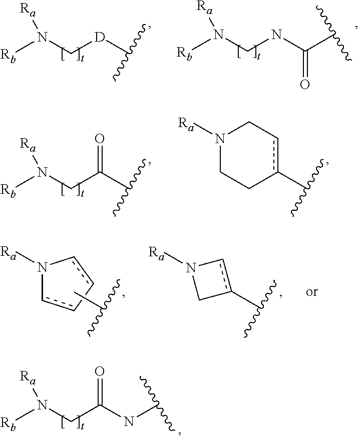

wherein t is 0, 1, 2, 3, or 4 and any methylene group of the t region is optionally-substituted one or more times with C₁₋₃ alkyl; D is N(H), O, C(H)₂, or S; and Rₐ and R_{b} are each independently hydro, C₃₋₆ cycloalkyl, optionally-substituted C₃₋₆ heterocyclo, or C₁₋₆ alkyl, or Rₐ and R_{b}, together with the linking nitrogen between them, form a first C₃₋₆ heterocyclo, wherein the first C₃₋₆ heterocyclo is optionally substituted with C₁₋₆ alkyl, amino, or a second C₃₋₆ heterocyclo.

In some embodiments of the compounds of each of Formulae III, IV, and IVa, R¹ and R¹¹ together form a spiro-linked heterocycle, optionally substituted at the heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, sulfonyl, or one of the following:

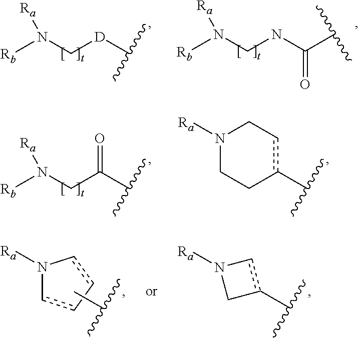

wherein t is 0, 1, 2, 3, or 4 and any methylene group of the t region is optionally-substituted one or more times with C₁₋₃ alkyl; D is O, C(H)₂, or S; and Rₐ and R_{b} are each independently hydro, C₃₋₆ cycloalkyl, optionally-substituted C₃₋₆ heterocyclo, or C₁₋₆ alkyl, or Rₐ and R_{b}, together with the linking nitrogen between them, form a first C₃₋₆ heterocyclo, wherein the first C₃₋₆ heterocyclo is optionally substituted with C₁₋₆ alkyl, amino, or a second C₃₋₆ heterocyclo.

In some embodiments of the compounds of each of Formulae III, IV, and IVa, R¹ and R¹¹ together with the same ring carbon form

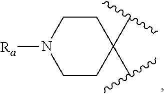

wherein Rₐ is selected from halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl.

In some embodiments of the compounds of each of Formulae III, IV, IVa, and IVb, R² is not present, or is present one, two, three, or four times. In some of such embodiments, R² is not present or is fluoro, methyl, or trifluormethyl. In some of such embodiments, R² is not present.

In some embodiments of the compounds of each of Formulae III, IV, IVa, and IVb, W is carbon.

In some embodiments of the compounds of each of Formulae III, IV, IVa, and IVb, W is nitrogen.

In some embodiments of the compounds of each of Formulae III, IV, IVa, and IVb, R³ is not present, or is present one, two, three, or four times. In some of such embodiments, R³ is not present or is fluoro, chloro, methyl, or trifluormethyl. In some of such embodiments, R³ is not present.

In some embodiments of the compounds of each of Formulae II, III, IV, IVa, and IVb, when present, R⁴ is hydro or hydroxyl. In some embodiments of the compounds of each of Formulae II, III, IV, IVa, and IVb, when present, R⁴ is hydro.

In some embodiments of the compounds of each of Formulae II, III, IV, IVa, and IVb, when present, R⁵ is hydro, fluoro, or hydroxyl. In some embodiments of the compounds of each of Formulae II, III, IV, IVa, and IVb, when present, R⁵ is hydro.

In some embodiments of the compounds of each of Formulae II, III, IV, IVa, and IVb, q is 1. In some embodiments of the compounds of each of Formulae II, III, IV, IVa, and IVb, q is 2. In some embodiments of the compounds of each of Formulae II, III, IV, IVa, and IVb, any methylene groups of the q region are optionally substituted with fluoro or methyl. In some embodiments of the compounds of each of Formulae II, III, IV, IVa, and IVb, any methylene groups of the q region are all fully saturated.

In some embodiments of the compounds of each of Formulae II, III, IV, IVa, and IVb, R⁶ is not present or is present one, two, three, or four times. In some of such embodiments, R⁶ is halo (such as, for example, fluoro), methyl, nitro, cyano, trihalomethyl, methoxy, amino, hydroxyl, or mercapto. In some embodiments of the compounds of II, III, IV, IVa, and IVb, R⁶ is not present or is NH₂ at the 4-position of the 3-pyridinyl ring.

In some embodiments of the compounds of each of Formulae II, III, IV, IVa, and IVb, R⁶ is not present and q is 1.

In some embodiments of the compounds of each of Formulae II, III, IV, IVa, and IVb, R⁶ is not present, q is 1, and the methylene group of q is fully saturated.

In some embodiments of the compounds of each of Formulae III, IV, IVa, and IVb, R⁶ is not present, q is 1, the methylene group of q is fully saturated, and R³ is not present.

In some embodiments of the compounds of each of Formulae III, IV, IVa, and IVb, R⁶ is not present, q is 1, the methylene group of q is fully saturated, and R² and R³ are not present.

In some embodiments of the compounds of each of Formulae III, IV, IVa, and IVb, R⁶ is not present, q is 1, the methylene group of q is fully saturated, R² and R³ are not present, and W is carbon.

In some embodiments of the compounds of each of Formulae III, IV, IVa, and IVb, R⁶ is not present, q is 1, the methylene group of q is fully saturated, R² and R³ are not present, and W is nitrogen.

The compounds of the present invention include the compounds of Formulae I, II, III, IV, IVa, and IVb, as illustrated herein, and the compounds of Tables 1-9, as well as for any of the foregoing their stereochemically isomeric forms thereof. The compounds of the present invention also include pharmaceutically-acceptable salts, prodrugs, N-oxide forms, quaternary amines, and solvates of the compounds of Formulae I, II, III, IV, IVa, and IVb, as illustrated herein, and the compounds of Tables 1-9.

For therapeutic use, salts of the compounds of Formulae I, II, III, IV, IVa, and IVb, as illustrated herein, and the compounds of Tables 1-9, are those particular salts wherein the counterion is pharmaceutically-acceptable. However, salts of acids and bases which are non-pharmaceutically-acceptable can also find use, for example, in the preparation or purification of a pharmaceutically-acceptable compound. All salts, whether pharmaceutically-acceptable or not, are within the ambit of the present invention.

The pharmaceutically-acceptable addition salts as mentioned herein are meant to comprise the therapeutically active non-toxic acid addition salt forms which the compounds of Formulae I, II, III, IV, IVa, and IVb, as illustrated herein, and the compounds of Tables 1-9, are able to form. The salts can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of Formulae I, II, III, IV, IVa, and IVb, as illustrated herein, and the compounds of Tables 1-9, containing acidic protons can be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanedi-ol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of Formulae I, II, III, IV, IVa, and IVb, as illustrated herein, and the compounds of Tables 1-9, are able to form. Examples of such forms are e.g. hydrates, alcoholates and the like.

The term “quaternary amine” as used herein defines the quaternary ammonium salts which the compounds of Formulae I, II, III, IV, IVa, and IVb, as illustrated herein, and the compounds of Tables 1-9, are able to form by reaction between a basic nitrogen of one of the compounds of Formulae I, II, III, IV, IVa, and IVb, as illustrated herein, and the compounds of Tables 1-9, and an appropriate quaternizing agent, such as, for example, an optionally substituted alkylhalide, arylhalide or arylalkylhalide, e.g. methyliodide or benzyliodide. Other reactants with good leaving groups can also be used, such as, for example, alkyl trifluoromethanesulfonates, alkyl methanesulfonates, and alkyl p-toluenesulfonates. A quaternary amine has a positively-charged nitrogen. Pharmaceutically-acceptable counterions include chloro, bromo, iodo, trifluoroacetate and acetate. The counterion of choice can be introduced using ion exchange resins.

Pharmaceutically-acceptable salts of the compounds of Formulae I, II, III, IV, IVa, and IVb, as illustrated herein, and the compounds of Tables 1-9, include all salts are exemplified by alkaline salts with an inorganic acid and/or a salt with an organic acid that are known in the art. In addition, pharmaceutically-acceptable salts include acid salts of inorganic bases, as well as acid salts of organic bases. Their hydrates, solvates, and the like are also encompassed in the present invention. In addition, N-oxide compounds are also encompassed in the present invention.

It will be appreciated that some of the compounds of Formulae I, II, III, IV, IVa, and IVb, as illustrated herein, and the compounds of Tables 1-9, and their N-oxides, addition salts, quaternary amines and stereochemically isomeric forms can contain one or more centers of chirality and exist as stereochemically isomeric forms.

The term “stereochemically isomeric forms” as used hereinbefore defines all the possible stereoisomeric forms which the compounds of Formulae I, II, III, IV, IVa, and IVb, as illustrated herein, and the compounds of Tables 1-9, and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of the compounds of Formulae I, II, III, IV, IVa, and IVb, as illustrated herein, and the compounds of Tables 1-9, and their N-oxides, salts, solvates or quaternary amines substantially free, i.e. associated with less than 10%, preferably less than 5%, in particular less than 2% and most preferably less than 1% of the other isomers. In particular, stereogenic centers can have the R- or S-configuration; substituents on bivalent cyclic (partially) saturated radicals can have either the cis- or trans-configuration. Compounds encompassing double bonds can have an E- or Z-stereochemistry at said double bond. Stereochemically isomeric forms of the compounds of Formulae I, II, III, IV, IVa, and IVb, as illustrated herein, and the compounds of Tables 1-9, are fully intended to be embraced within the scope of this invention.

“N-oxides” are meant to comprise the compounds of Formulae I, II, III, IV, IVa, and IVb, as illustrated herein, and the compounds of Tables 1-9, or a pharmaceutically-acceptable salt thereof, wherein one or several nitrogen atoms are oxidized to the so-called N-oxide.

Some of the compounds of Formulae I, II, III, IV, IVa, and IVb, as illustrated herein, and the compounds of Tables 1-9, or a pharmaceutically-acceptable salt thereof, can also exist in their tautomeric form. Such forms although not explicitly indicated in the above formula are intended to be included within the scope of the present invention.

In preferred embodiments, compounds of the present invention are provided having an IC₅₀ of less than about 100 nM, as determined in the cytotoxicity assays as described in the Examples below (i.e., Cytotoxicity Assays).

In all compounds of the present invention, such as, for example, the compounds of Formulae I, II, III, IV, IVa, and IVb, as illustrated herein, and the compounds of Tables 1-9, or a pharmaceutically-acceptable salt thereof, reference to any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039, which are incorporated by reference herein their entirety. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms slows the catabolism of the deuterated compound, relative to the non-deuterated compound, such that the deuterated compound exhibits a longer half-life in the bodies of individuals administered such compounds. This is particularly so when the catabolism of the hydrogenated compound is mediated by cytochrome P450 systems. See Kushner et al., Can. J. Physiol. Pharmacol. (1999) 77:79-88, which is incorporated by reference herein its entirety.

### 3. Pharmaceutical Compositions and Formulations

Additionally, the present invention provides a composition for use as a medicament or a pharmaceutical composition comprising one of the compounds of the present invention, such as, for example, the compounds of Formulae I, II, III, IV, IVa, and IVb, as illustrated herein, and the compounds of Tables 1-9, or a pharmaceutically-acceptable salt thereof, and a pharmaceutically-acceptable excipient. In some of such embodiments, the medicament or pharmaceutical composition comprises a therapeutically or prophylactically effective amount of at least one of the compounds of Formulae I, II, III, IV, IVa, and IVb, as illustrated herein, and the compounds of Tables 1-9.

In some of such embodiments, the composition or pharmaceutical composition is for use in treating cancer, systemic or chronic inflammation, rheumatoid arthritis, diabetes, obesity, T-cell mediated autoimmune disease, ischemia, and other complications associated with these diseases and disorders. In some of such embodiments, the composition or pharmaceutical composition is for use in treating cancer.

Typically, one of the compounds of the present invention, such as, for example, the compounds of Formulae I, II, III, IV, IVa, and IVb, as illustrated herein, and the compounds of Tables 1-9, or a pharmaceutically-acceptable salt thereof, can be effective at an amount of from about 0.01 μg/kg to about 100 mg/kg per day based on total body weight. The active ingredient can be administered at once, or can be divided into a number of smaller doses to be administered at predetermined intervals of time. The suitable dosage unit for each administration can be, e.g., from about 1 μg to about 2000 mg, preferably from about 5 μg to about 1000 mg. The pharmacology and toxicology of many of such other anticancer compounds are known in the art. See e.g., Physicians Desk References, Medical Economics, Montvale, N.J.; and The Merck Index, Merck & Co., Rahway, N.J. The therapeutically-effective amounts and suitable unit dosage ranges of such compounds used in art can be applicable to the compounds of the present invention, such as, for example, the compounds of Formulae I, II, III, IV, IVa, and IVb, as illustrated herein, and the compounds of Tables 1-9, or a pharmaceutically-acceptable salt thereof.

It should be understood that the dosage ranges set forth above are exemplary only and are not intended to limit the scope of this invention. The therapeutically-effective amount for individual compounds of the present invention, such as, for example, the compounds of Formulae I, II, III, IV, IVa, and IVb, as illustrated herein, and the compounds of Tables 1-9, or a pharmaceutically-acceptable salt thereof, can vary with factors including but not limited to the activity of the compound used, the stability of the compound used in the patient's body, the severity of the conditions to be alleviated, the total weight of the patient treated, the route of administration, the ease of absorption, distribution, and excretion of the compound by the body, the age and sensitivity of the patient to be treated, and the like, as will be apparent to a skilled artisan. The amount of administration can be adjusted as the various factors change over time.

In the pharmaceutical compositions, the compounds of the present invention, such as, for example, the compounds of Formulae I, II, III, IV, IVa, and IVb, illustrated herein, and the compounds of Tables 1-9, can be in any pharmaceutically-acceptable salt form, as described above.

For oral delivery, the compounds of the present invention, such as, for example, the compounds of Formulae I, II, III, IV, IVa, and IVb, as illustrated herein, and the compounds of Tables 1-9, or a pharmaceutically-acceptable salt thereof, can be incorporated into a formulation that includes pharmaceutically-acceptable excipients or carriers such as, for example, binders, lubricants, disintegrating agents, and sweetening or flavoring agents, all known in the art. The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as, for example, fatty oil can also be included in capsules.

Suitable oral formulations can also be in the form of a solution, suspension, syrup, chewing gum, wafer, elixir, and the like. If desired, conventional agents for modifying flavors, tastes, colors, and shapes of the special forms can also be included.

The compounds of the present invention, such as, for example, the compounds of Formulae I, II, III, IV, IVa, and IVb, as illustrated herein, and the compounds of Tables 1-9, or a pharmaceutically-acceptable salt thereof, can also be administered parenterally in the form of a solution or suspension, or in a lyophilized form capable of conversion into a solution or suspension form before use. In such formulations, diluents or pharmaceutically-acceptable carriers such as, for example, sterile water and physiological saline buffer can be used. Other conventional solvents, pH buffers, stabilizers, anti-bacteria agents, surfactants, and antioxidants can all be included. The parenteral formulations can be stored in any conventional containers such as, for example, vials and ampoules.

Routes of topical administration include dermal, nasal, bucal, mucosal, rectal, vaginal, or occular applications. For topical administration, the compounds of the present invention, such as, for example, the compounds of Formulae I, II, III, IV, IVa, and IVb, as illustrated herein, and the compounds of Tables 1-9, or a pharmaceutically-acceptable salt thereof, can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches that can be used with the compounds of the present invention, such as, for example, the compounds of Formulae I, II, III, IV, IVa, and IVb, as illustrated herein, and the compounds of Tables 1-9, or a pharmaceutically-acceptable salt thereof, are disclosed, e.g., in Brown, et al., Annual Review of Medicine, 39:221-229 (1988), which is incorporated herein by reference.

Subcutaneous implantation for sustained release of the compounds of the present invention, such as, for example, the compounds of Formulae I, II, III, IV, IVa, and IVb, as illustrated herein, and the compounds of Tables 1-9, or a pharmaceutically-acceptable salt thereof, can also be a suitable route of administration. This entails surgical procedures for implanting one or more of the compounds of the present invention, such as, for example, the compounds of Formulae I, II, III, IV, IVa, and IVb, as illustrated herein, and the compounds of Tables 1-9, or a pharmaceutically-acceptable salt thereof, in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the compounds of the present invention, such as, for example, the compounds of Formulae I, II, III, IV, IVa, and IVb, as illustrated herein, and the compounds of Tables 1-9, or a pharmaceutically-acceptable salt thereof. Hydrogels are generally known in the art. They are typically made by crosslinking high molecular weight biocompatible polymers into a network, which swells in water to form a gel-like material. Preferably, hydrogels are biodegradable or biosorbable. See, e.g., Phillips et al., J. Pharmaceut. Sci., 73:1718-1720 (1984).

The compounds of the present invention, such as, for example, the compounds of Formulae I, II, III, IV, IVa, and IVb, as illustrated herein, and the compounds of Tables 1-9, or a pharmaceutically-acceptable salt thereof, can also be conjugated, to a water soluble, non-immunogenic, non-peptidic, high molecular weight polymer to form a polymer conjugate. For example, one or more of the compounds of the present invention, such as, for example, the compounds of Formulae I, II, III, IV, IVa, and IVb, as illustrated herein, and the compounds of Tables 1-9, or a pharmaceutically-acceptable salt thereof, is covalently linked to polyethylene glycol to form a conjugate. Typically, such a conjugate exhibits improved solubility, stability, and reduced toxicity and immunogenicity. Thus, when administered to a patient, compounds of the present invention, such as, for example, the compounds of Formulae I, II, III, IV, IVa, and IVb, as illustrated herein, and the compounds of Tables 1-9, or a pharmaceutically-acceptable salt thereof, in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL).

It is preferred that the covalent linkage between the polymer and one or more of the compounds of the present invention, such as, for example, the compounds of Formulae I, II, III, IV, IVa, and IVb, as illustrated herein, and the compounds of Tables 1-9, or a pharmaceutically-acceptable salt thereof, and/or the polymer itself is hydrolytically degradable under physiological conditions. Such conjugates can readily release the compounds of the present invention, such as, for example, the compounds of Formulae I, II, III, IV, IVa, and IVb, as illustrated herein, and the compounds of Tables 1-9, or a pharmaceutically-acceptable salt thereof, inside the body. Controlled release of the compounds of the present invention, such as, for example, the compounds of Formulae I, II, III, IV, IVa, and IVb, as illustrated herein, and the compounds of Tables 1-9, or a pharmaceutically-acceptable salt thereof, can also be achieved by incorporating one or more of the compounds of the present invention into microcapsules, nanocapsules, or hydrogels that are generally known in the art.

Liposomes can also be used as carriers for the compounds of the present invention, such as, for example, the compounds of Formulae I, II, III, IV, IVa, and IVb, as illustrated herein, and the compounds of Tables 1-9, or a pharmaceutically-acceptable salt thereof. Liposomes are micelles made of various lipids such as, for example, cholesterol, phospholipids, fatty acids, and derivatives thereof. Various modified lipids can also be used. Liposomes can reduce toxicity of the compounds of the present invention, and can increase their stability. Methods for preparing liposomal suspensions containing active ingredients therein are generally known in the art, and, thus, can be used with the compounds of the present invention. See, e.g., U.S. Pat. No. 4,522,811; Prescott, Ed., Methods in Cell Biology, Volume XIV, Academic Press, New York, N.Y. (1976).

### 4. Therapeutic Methods

The present invention provides therapeutic methods for treating diseases and disorders that will respond favorably to therapy with a Nampt inhibitor. Consequently, the present invention provides therapeutic methods for treating cancer, systemic or chronic inflammation, rheumatoid arthritis, diabetes, obesity, T-cell mediated autoimmune disease, ischemia, and other complications associated with these diseases and disorders. These therapeutic methods involve treating a patient (either a human or another animal) in need of such treatment, with a therapeutically-effective amount of one or more of the compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising a therapeutically-effective amount of one or more of the compounds of the present invention.

Additionally, the present invention provides the use of the compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising a therapeutically-effective amount of one or more of the compounds of the present invention, for the manufacture of a medicament useful for human therapy.

In some of such embodiments, the therapy comprises therapy for the treatment of cancer, systemic or chronic inflammation, rheumatoid arthritis, diabetes, obesity, T-cell mediated autoimmune disease, ischemia, and other complications associated with these diseases and disorders, in a human patient.

In some of such embodiments, the therapy comprises therapy for the delaying the onset of, or reducing the symptoms of, cancer, systemic or chronic inflammation, rheumatoid arthritis, diabetes, obesity, T-cell mediated autoimmune disease, ischemia, and other complications associated with these diseases and disorders, in a human patient.

The present invention also comprises treating isolated cells with a therapeutically-effective amount of one or more of the compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising a therapeutically-effective amount of one or more of the compounds of the present invention.

As used herein, the phrase “treating . . . with . . . a compound” means either administering one or more of the compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more of the compounds of the present invention, directly to isolated cells or to an animal, or administering to isolated cells or an animal another agent to cause the presence or formation of one or more of the compounds of the present invention inside the cells or the animal.

In some embodiments, the present invention provides a method of inhibiting the activity of Nampt in human cells comprising, contacting the cells with a compound of the present invention, such as, for example, a compound of I, II, III, IV, IVa, and IVb, as illustrated herein, and a compounds of Table 1, or a pharmaceutically-acceptable salt thereof. In some of such embodiments, the cells are with the body of a human patient.

Preferably, the methods of the present invention comprise administering to cells in vitro or to a warm-blood animal, particularly a mammal, and more particularly a human, a pharmaceutical composition comprising an effective amount of one or more of the compounds of the present invention, such as, for example, the compounds of Formulae I, II, III, IV, IVa, and IVb, as illustrated herein, and the compounds of Tables 1-9, or a pharmaceutically-acceptable salt thereof, or another agent to cause the presence or formation of one or more of the compounds of the present invention inside the cells or the animal.

As would be appreciated by the skilled artisan, one or more of the compounds of the present invention, such as, for example, the compounds of Formulae I, II, III, IV, IVa, and IVb, as illustrated herein, and the compounds of Tables 1-9, or a pharmaceutically-acceptable salt thereof, can be administered in one dose at one time, or can be divided into a number of smaller doses to be administered at predetermined intervals of time. The suitable dosage unit for each administration can be determined based on the effective daily amount and the pharmacokinetics of the compounds.

a. Treating Cancer:

In particular embodiments, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient in need of such treatment.

In some embodiments, the patient is a human patient.

In some embodiments, the method comprises identifying a patient in need of such treatment. A patient having cancer can be identified by conventional diagnostic techniques known in the art, as well as by those methods discussed in International Patent Application No. PCT/US11/26752, filed Mar. 1, 2011, the entire contents of which are incorporated herein by reference.

As noted previously, Nampt catalyzes the first and rate-limiting step in the generation of NAD⁺ from NaM, and NAD⁺ is critical for the generation of cellular ATP by glycolysis, the citric acid cycle, and oxidative phosphorylation. By these mechanisms and others, reduction in cellular NAD⁺ levels by Nampt inhibition causes depletion of cellular ATP and, ultimately, cell death. Tumor cells are thought to be more sensitive to NAD⁺ and ATP loss than normal cells due to their higher energy needs and an increased reliance on glycolysis. Known as the “Warburg effect” (Warburg, O. On respiratory impairment in cancer cells. Science 124, 269-270 (1956)), a wide spectrum of cancer cells exhibit increased glycolysis relative to oxidative phosphorylation, despite the availability of oxygen. The shift from oxidative phosphorylation to a reliance on glycolysis is thought to result from mitochondrial damage and/or a hypoxic tumor microenvironment (reviewed in Hsu, P. P and Sabatini, D. M. Cancer cell metabolism: Warburg and beyond. Cell 134, 703-707 (2008)) and/or cellular reprogramming by oncogenes and/or tumor suppressors (reviewed in Levine, A. J. and Puzio-Kuter A. M. Science. 330, 1340-1344 (2010)). With regards to depleting energy levels in tumor cells, Nampt inhibitors would be analogous to inhibitors of other glycolytic enzymes, several of which are in cancer preclinical or clinical trials (reviewed in Pelicano H. et al. Glycolysis inhibition for anticancer treatment. Oncogene 25, 4633-4646 (2006)).

In addition to increased energy needs, tumor cells are more susceptible to NAD⁺ loss due to a higher turnover of NAD⁺ in response to DNA damage and genomic instability. According to this model, poly(ADP-ribose) polymerases (PARPs) consume NAD⁺ as they generate poly(ADP-ribose) to repair DNA in response to alkylating agents, ionizing radiation, and oxidative stress (reviewed in GallíM. et al. The nicotinamide phosphoribosyltransferase: a molecular link between metabolism, inflammation, and cancer. Cancer Res. 70, 8-11 (2010)). Indeed, an inability to replenish this NAD⁺ loss, either by reducing Nampt expression or inhibiting Nampt activity, sensitizes cells to PARP activation (Rongvaux, et al. Nicotinamide phosphoribosyl transferase/pre-B cell colony-enhancing factor/visfatin is required for lymphocyte development and cellular resistance to genotoxic stress. J. Immunol. 181, 4685-4695 (2008)).

The increased metabolic demands of cancer cells (Luo et al., Cell. 136(5):823-37 (2009). Erratum in: Cell., 2009 Aug. 21; 138(4):807.)) suggests that they should require NAD⁺ in sufficient levels to maintain cellular pools of ATP. This requirement, and the critical role played by Nampt in NAD⁺ synthesis further suggests that cancer cells have a critical need for adequate Nampt activity. Consistent with this hypothesis are reports of Nampt over-expression in colon cancers (Hufton et al., FEBS Lett. 463(1-2):77-82 (1999), Van Beijnum et al., Int. J. Cancer. 101(2):118-27 (2002)), ovarian cancers (Shackelford et al., Int J. Clin. Exp. Pathol. 3(5): 522-527 (2010)), prostate cancers (Wang et al., Oncogene 30: 907-921 (2011)) and GBM cancers (Reddy et al., Cancer Biol. Ther. 7(5):663-8 (2008)), and suggestions of the amplification of the gene encoding Nampt in multiple other cancers. Immunohistochemistry analyses suggest strong expression of Nampt occurs in greater than 20% of biopsies of: breast, lung, malignant lymphoma, ovarian, pancreatic, prostate and testicular cancers (www.proteinatlas.org). In addition to the role played by NAD⁺ as a cofactor in redox reactions, NAD⁺ also serves as a substrate for mono and poly-ADP ribosyltransferases (PARPs), class III histone deacetylases (sirtuins) and ADP-ribose cyclases. PARPs appear to be major consumers of cellular NAD⁺ (Paine et al., Biochem. J. 202(2):551-3 (1982)), and evidence exists for increased polyADP-ribosylation activity in oral cancer (Das, B. R., Cancer Lett. 73(1):29-34 (1993)), hepatocellular carcinoma (Shiobara et al., J. Gastroenterol. Hepatol. 16(3):338-44 (2001), Nomura et al., J Gastroenterol. Hepatol. 15(5):529-35 (2000)), rectal cancer (Yalcintepe et al., Braz. J. Med. Biol. Res. 38(3):361-5 (2005); Epub 2005, Mar. 8.), and leukemia and ovarian cancers (Singh N, Cancer Lett. 58(1-2):131-5 (1991)). Increased ADP-ribosylation in cancer can reflect PARPs' role in DNA repair (Durkacz et al., Nature. 283(5747):593-6 (1980); deMurcia et al., Proc. Natl. Acad. Sci. U.S.A. 94(14):7303-7 (1997), Simbulan-Rosenthal et al., Proc. Natl. Acad. Sci. U.S.A. 96(23):13191-6 (1999)) and the need to maintain genome integrity in the face of genomic instability and the resulting accumulation of point mutations, deletions, chromosomal rearrangement and aneuploidy (Hartwell and Kastan, Science. 266(5192):1821-8 (1994)). PARP-1 itself is reported to be over-expressed in breast cancer, where its expression inversely correlates with genomic instability (Biechi et al., Clin. Cancer Res. 2(7):1163-7 (1996)).

Furthermore, the Nampt transcript is known to be upregulated in colon cancers (van Beijnum J R, et al. Target validation for genomics using peptide-specific phage antibodies: a study of five gene products overexpressed in colorectal cancer. Int. J. Cancer. 101, 118-127 (2002); and Hufton S E, et al. A profile of differentially expressed genes in primary colorectal cancer using suppression subtractive hybridization. FEBS Lett. 463, 77-82 (1999)) and glioblastoma cancers (Reddy P S, et al. PBEF1/NAmPRTase/Visfatin: a potential malignant astrocytoma/glioblastoma serum marker with prognostic value. Cancer Biol. Ther. 7, 663-668 (2008)), and it remains possible that the Nampt gene is amplified in other cancers.

However, without wishing to be bound by theory, cancers that express low levels of the Nampt enzyme may be more sensitive to treatment with a Nampt inhibitor, than a cancer that expresses high levels of the Nampt enzyme. International Patent Application No. PCT/US11/26752, filed Mar. 1, 2011, the entire contents of which are incorporated herein by reference, discloses, among other things, that Nampt expression may inversely correlate with tumoricidal and NAD depletion potency and may directly correlate with basal NAD levels. Accordingly, the present invention includes methods of treating cancer, comprising first identifying a cancer exhibiting a low level of Nampt expression. The methods further comprise administering to a patient having a cancer exhibiting low levels of Nampt expression, a therapeutically-effective dose of a compound of Formulae I, II, III, IV, IVa, and IVb or a compound of Table 1, or a pharmaceutically-acceptable salt thereof.

In view of the above, it is believed that inhibition of Nampt activity would be effective in treating a wide range of cancers. Support for this assertion is found in the Examples section below and in the Examples of International Patent Application No. PCT/US11/26752, filed Mar. 1, 2011, the entire contents of which are incorporated herein by reference.

Thus, in one embodiment, the present invention provides a method of treating colon cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating prostate cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating breast cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating non-small-cell lung cancer (NSCLC), comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating sarcoma cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating pancreatic cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating SCLC cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating gastric cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating myeloma cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating ovarian cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating lymphoma cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

Thus, in one embodiment, the present invention provides a method of treating glioma cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient.

As used herein, the term “cancer” has its conventional meaning in the art. Cancer includes any condition of the animal or human body characterized by abnormal cellular proliferation. The cancers to be treated comprise a group of diseases characterized by the uncontrolled growth and spread of abnormal cells. Compounds of the present invention have been shown to be effective in a variety of standard cancer models, and are thus thought to have utility in treating a broad range of cancers. However, preferred methods of the invention involve treating cancers that have been found to respond favorably to treatment with Nampt inhibitors. Further, “treating cancer” should be understood as encompassing treating a patient who is at any one of the several stages of cancer, including diagnosed but as yet asymptomatic cancer.

Specific cancers that can be treated by the methods of the invention are those cancers that respond favorably to treatment with a Nampt inhibitor. Such cancers include, but are not limited to, Hodgkin's disease, non-Hodgkin's lymphoma, acute lymphocytic leukemia, chronic lymphocytic leukemia, acute myeloid leukemia, mantle-cell lymphoma, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

NAD⁺ can be generated by several Nampt-independent pathways as well, including: (1) de novo synthesis from L-tryptophan via the kynurenine pathway; (2) from nicotinic acid (NA) via the Preiss-Handler pathway; and (3) from nicotinamide riboside or nicotinic acid riboside via nicotinamide/nicotinic acid riboside kinases (reviewed in Khan, J. A. et al., Nicotinamide adenine dinucleotide metabolism as an attractive target for drug discovery. Expert Opin. Ther. Targets. 11(5):695-705 (2007)). However, these different routes of NAD⁺ synthesis are generally tissue specific: The de novo pathway is present in liver, brain, and immune cells, the Priess-Handler pathway is primarily active in the liver, kidney, and heart, and Nrk2, of the nicotinamide riboside kinase pathway, is expressed in brain, heart, and skeletal muscle (Bogan, K. L. and Brenner, C. Nicotinic acid, nicotinamide, and nicotinamide riboside: a molecular evaluation of NAD⁺ precursor vitamins in human nutrition. Annu. Rev. Nutr. 28:115-30 (2008) and Tempel, W. et al., Nicotinamide riboside kinase structures reveal new pathways to NAD⁺. PLoS Biol. 5(10):e263 (2007)).

Of these alternative pathways of NAD⁺ synthesis, the Preiss-Handler pathway is perhaps the most important for cancer cells. The first and rate-limiting step of this pathway, the conversion of nicotinic acid (NA) to nicotinic acid mononucleotide (NAMN), is catalyzed by the enzyme Naprt1.

While not wishing to be bound by theory it follows, therefore, that one way to stratify patients and to potentially expand the therapeutic window of the compounds of the present invention would be to identify those patients having cancers with reduced or absent levels of Naprt1 expression. Such cancers would theoretically be less able to replace cellular NAD⁺ through this alternative pathway, while being treated with Nampt inhibitors. Hence, they should be more sensitive to treatment by the compounds of the present invention, such as, for example, a compound of Formula I, II, III, IV, IVa, and IVb, as illustrated herein, and a compound of Table 1, or a pharmaceutically-acceptable salt thereof.

Accordingly, the present invention includes methods of treating cancer, comprising first identifying a patient having a cancer exhibiting a low level of Naprt expression. These methods further comprise administering to a patient having a cancer exhibiting low levels of Naprt1 expression, a therapeutically-effective dose of a compound of Formulae I, II, III, IV, IVa, and IVb or a compound of Table 1, or a pharmaceutically-acceptable salt thereof.

In some embodiments, identifying a patient having a cancer exhibiting a low level of Naprt1 expression comprises determining the level of expression of Naprt1 protein within cancer cells from the patient. In some of such embodiments, determining the level of expression of Naprt1 protein is by way of a Western Blot and/or an Enzyme-Linked Immunosorbant Assay (ELISA).

In some embodiments, identifying a patient having a cancer exhibiting a low level of Naprt1 expression comprises determining the level of expression of the mRNA transcript encoding the Naprt1 protein within cancer cells from the patient. In some of such embodiments, determining the level of expression of the mRNA transcript encoding the Naprt1 protein is by way of a Northern Blot and/or by quantitative RT-PCR (qRT-PCT).

In some embodiments, identifying a patient having a cancer exhibiting a low level of Naprt1 expression further comprises determining whether such cancer expresses low levels of the Nampt enzyme within cancer cells from the patient.

International Patent Application No. PCT/US11/26752, filed Mar. 1, 2011, the entire contents of which are incorporated herein by reference, discloses cell lines treated with Nampt inhibitors and screened for NA rescue and Naprt1 expression by immunoblotting and quantitative RT-PCRNaprt1 expression was least in brain cancers, lung cancers, lymphoma, myeloma and osteosarcoma. Further, glioblastoma and sarcoma cell lines that are reported to be resistant to NA rescue have been found to have reduced Naprt1 expression (Watson, et al. Mol. Cell. Biol. 29(21):5872-88 (2009)).

Thus, in one embodiment, the present invention provides a method of treating brain cancer, such as, for example, glioblastoma, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient in need of such treatment.

Thus, in one embodiment, the present invention provides a method of treating lung cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient in need of such treatment.

Thus, in one embodiment, the present invention provides a method of treating osteosarcoma cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, to a patient in need of such treatment.

Those cancers with reduced or absent levels of Naprt1 expression should be more susceptible to treatment with compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, such as, for example, the compounds of Formulae I, II, III, IV, IVa, and IVb, as illustrated herein, and the compounds of Tables 1, or a pharmaceutically-acceptable salt thereof. Co-administration of nicotinic acid (“NA”) to patients having such cancers could prevent toxicity in other tissues associated with Nampt inhibition. This phenomenon is referred to in the art as “NA rescue.” Cells and/or cancers that are capable of NA rescue are also referred to herein as “exhibiting the NA Rescue Phenotype.” International Patent Application No. PCT/US11/26752, filed Mar. 1, 2011, the entire contents of which are incorporated herein by reference, discloses the results of studies that indicate that the level of expression of Naprt1 is correlated with the ability of the cell lines to be rescued from Nampt inhibitor-induced cytotoxicity by NA.

Accordingly, in some embodiments, the methods of treating cancer disclosed herein further comprise administering nicotinic acid, or a compound capable of forming nicotinic acid in vivo, to the patient in addition to administering a compound of the present invention, such as, for example, a compound of Formulae I, II, III, IV, IVa, and IVb, as illustrated herein, and a compound of Table 1, or a pharmaceutically-acceptable salt thereof. In some of such embodiments, the compound of the present invention is able to be administered at dose that exceeds the maximum tolerated dose for that particular compound of the present invention as determined for mono-therapy.

In some of such embodiments, administering NA may include administering NA prior to administering one or more of the compounds of the present invention, co-administering NA with one or more of the compounds of the present invention, or first treating the patient with one or more of the compounds of the present invention, followed by thereafter administering NA.

b. Treating Systemic or Chronic Inflammation

Nampt expression in visceral adipose tissue has been found to correlate with the expression of proinflammatory genes, CD68 and TNFα (Chang et al.; Metabolism. 59(1):93-9 (2010)). Several studies have noted an increase in reactive oxygen species and activation of NF-kappaB in response to Nampt expression (Oita et al.; Pflugers Arch. (2009); Romacho et al.; Diabetologia. 52(11):2455-63 (2009)). Nampt serum levels were found to have been increased in patients with inflammatory bowel diseases and correlated with disease activity (Moschen et al.; Mutat. Res. (2009)). One study has even suggested a specific mechanism for Nampt in inflammation: High levels of Nampt increase cellular NAD⁺ levels leading to a post-transcriptional upregulation of TNF via the NAD-dependent deacetylase, SirT6 (Van Gool et al. Nat. Med. 15(2):206-10 (2009)). Further, inhibition of Nampt reduced levels of inflammatory cytokines IL-6 and TNF-α (Busso et al. PLoS One. 21; 3(5):e2267 (2008)). In another study, Nampt inhibition was found to prevent TNF-α and IFN-γ production in T-lymphocytes (Bruzzone et al.; PLoS One.; 4(11):e7897 (2009)).

In view of the above, it is believed that inhibition of Nampt activity would be effective in treating systemic or chronic inflammation resulting from a wide range of causes. Consequently, the present invention provides methods of treating systemic or chronic inflammation by administering therapeutically-effective amounts of one or more of the compounds of the present invention to a patient in need of such treament.

c. Treating Rheumatoid Arthritis

Nampt levels increased in a mouse model of arthritis and treatment of these mice with a Nampt inhibitor reduced the arthritis symptoms (Busso et al. PLoS One. 21; 3(5):e2267 (2008)). Also, because Nampt inhibition can decrease the activity of poly(ADP ribose) polymerases (PARPs) through the dependence of PARPs on NAD as a substrate, Nampt inhibitors, either alone or in combination with PARP inhibitors can be efficacious in any ailment treatable by PARP inhibitors. In this regard, PARP inhibitors have shown efficacy in models of arthritis (Kroger et al. Inflammation. 20(2):203-215 (1996)).

In view of the above, it is believed that inhibition of Nampt activity would be effective in treating RA. Consequently, the present invention provides methods of treating RA by administering therapeutically-effective amounts of one or more of the compounds of the present invention, either alone, or in combination with a PARP inhibitor, to a patient in need of such treament.

d. Treating Obesity and Diabetes

Nampt, also known as visfatin, was described as an adipokine found in visceral fat that acted as an insulin mimetic (Fukuhara et al. Science 307:426-30 (2007)). This paper was eventually retracted and other groups have failed to confirm that Nampt binds the insulin receptor. Nevertheless, many subsequent papers continue to report correlations between Nampt expression and obesity and/or diabetes. In one, increased expression of Nampt and levels of circulating Nampt were seen in obese patients (Catalan et al.; Nutr. Metab. Cardiovasc. Dis. (2010)), although a different study found that the correlation was specific only to obese patients with type 2 diabetes (Laudes, et al.; Horm. Metab. Res. (2010)). Yet another study reported a correlation between BMI and body fat mass and Nampt plasma levels, but an inverse correlation with cerebrospinal fluid levels of Nampt (Hallschmid et al.; Diabetes. 58(3):637-40 (2009)). Following bariatric surgery, patients with pronounced weight loss showed decreased levels of Nampt mRNA in liver (Moschen et al.; J. Hepatol. 51(4):765-77 (2009)). Finally, a rare single nucleotide polymorphism was identified in Nampt that correlated with severe obesity (Blakemore, et al.; Obesity 17(8):1549-53 (2009)). In contrast to these reports, Nampt levels were not altered in rat models of obesity (Mercader et al.; Horm. Metab. Res. 40(7):467-72 (2008)). Further, circulating levels of Nampt correlated with HDL-cholesterol and inversely with triglycerides (Wang et al.; Pflugers Arch. 454(6):971-6 2007)), arguing against Nampt involvement in obesity. Finally Nampt has been show to be a positive regulator of insulin secretion by beta-cells (Revollo et al. Cell Metab. 6(5):363-75 (2007)). This effect seems to require the enzymatic activity of Nampt and can be mimicked in cell culture models by exogenous addition of NaMN.

Because Nampt inhibition can decrease the activity of poly(ADP ribose) polymerases (PARPs) through the dependence of PARPs on NAD as a substrate, Nampt inhibitor, either alone or in combination with PARP inhibitors can be efficacious in any ailment treatable by PARP inhibitors. In this regard, PARP inhibitors have shown efficacy in models of type I diabetes (Drel et al. Endocrinology. 2009 December; 150(12):5273-83. Epub 2009 Oct. 23).

In view of the above, and despite the contrasting results mentioned, it is believed that inhibition of Nampt activity would be effective in treating obesity and diabetes, and other complications associated with these, and other, metabolic diseases and disorders. Consequently, the present invention provides methods of treating obesity and diabetes, and other complications associated with these, and other, metabolic diseases and disorders, by administering therapeutically-effective amounts of one or more of the compounds of the present invention, to a patient in need of such treament.

e. Treating T-cell Mediated Autoimmune Disease

Nampt expression has been shown to be upregulated in activated T-cells (Rongavaux et al.; J. Immunol. 181(7):4685-95 2008)) and Phase I clinical trials report lymphopenia in patients treated with Nampt inhibitors (reviewed in von Heideman et al.; Cancer Chemother. Pharmacol. (2009)). Additionally, in a mouse model of a T-cell autoimmune disease, experimental autoimmune encephalomyelitis (EAE), Nampt inhibition reduced the clinical disease score and demyelination in the spinal cord (Bruzzone et al.; PLoS One. 4(11):e7897 (2009)).

In view of the above, it is believed that inhibition of Nampt activity would be effective in treating T-cell mediated autoimmune disease, and other complications associated with diseases and disorders. Consequently, the present invention provides methods of treating T-cell mediated autoimmune disease, and other complications associated with these diseases and disorders, by administering therapeutically-effective amounts of one or more of the compounds of the present invention, to a patient in need of such treament.

f. Treating Ischemia

Because Nampt inhibition can decrease the activity of poly(ADP ribose) polymerases (PARPs) through the dependence of PARPs on NAD as a substrate, Nampt inhibitor, either alone or in combination with PARP inhibitors can be efficacious in any ailment treatable by PARP inhibitors. The PARP inhibitor FR247304 has been shown to attenuate neuronal damage in vitro and in vivo models of cerebral ischemia (Iwashita, et al. J. Pharmacol Exp. Ther. 310(2):425-36 (2004). Epub 2004 Apr. 9). Similarly there are suggestions that PARP inhibitors could be efficacious in clinical management of chronic hypoperfusion-induced neurodegenerative diseases including ocular ischemic syndrome (Mester et al. Neurotox. Res. 16(1):68-76 (2009) Epub 2009 Apr. 9) or ischemia reperfusion (Crawford et al. Surgery. 2010 Feb. 2. [Epub ahead of print]).

In view of the above, it is believed that inhibition of Nampt activity would be effective in treating ischemia and other complications associated with this condition. Consequently, the present invention provides methods of treating ischemia and other complications associated with this condition, by administering therapeutically-effective amounts of one or more of the compounds of the present invention, either alone, or in combination with a PARP inhibitor, to a patient in need of such treament.

### 5. Combination Therapy

Additionally, the present invention provides methods for combination therapy for treating cancer, systemic or chronic inflammation, rheumatoid arthritis, diabetes, obesity, T-cell mediated autoimmune disease, ischemia, and other complications associated with these diseases and disorders, by treating a patient in need thereof, with a therapeutically-effective amount of one of the compounds of the present invention together with a therapeutically-effective amount of one or more other compounds that have been shown to be effective in the treatment of cancer, systemic or chronic inflammation, rheumatoid arthritis, diabetes, obesity, T-cell mediated autoimmune disease, ischemia, and other complications associated with these diseases and disorders.

In some embodiments, the present invention provides methods for combination therapy for treating cancer by treating a patient (either a human or another animal) in need of such treatment with one of the compounds of the present invention together with one or more other anti-cancer therapies. Such other anti-cancer therapies include traditional chemotherapy agents, targeted agents, radiation therapy, surgery, hormone therapy, immune adjuvants, etc. In the combination therapy, one of the compounds of the present invention, such as, for example, a compound of Formulae I, II, III, IV, IVa, and IVb, as illustrated herein, and a compound of Table 1, or a pharmaceutically-acceptable salt thereof, can be administered separately from, or together with the one or more other anti-cancer therapies.

Specifically, Nampt inhibition has been shown to sensitize cells to the effects of various chemotherapeutic or cytotoxic agents. Specifically, Nampt inhibition has been shown to sensitize cells to amiloride, mitomycin C, N-methyl-N′-nitro-N-nitrosoguanidine (MNNG), melphalan, daunorubicin, cytarabine (Ara-C), and etoposide (Ekelund, S. et al. Chemotherapy 48:196-204 (2002); Rongvaux, A. et al. The Journal of Immunology 181(7):4685-95 (2008); Martinsson, P. et al. British Journal of Pharmacology 137:568-73 (2002); Pogrebniak, A. et al. European Journal of Medical Research 11(8):313-21 (2006)). It is also thought that lactate dehydrogenase A inhibitors, prostaglandin H2 synthase 2 (PGHS-2) inhibitors, combined with Nampt inhibitors would be effective cancer treatments. Without wishing to be bound by theory, Nampt inhibition may cause a drop in cellular levels of NAD⁺ at doses and times of exposure that are not overtly toxic to the cell. Without wishing to be bound by theory, it is believed that sub-lethal NAD⁺ drops render cells vulnerable to other cytotoxic agents, and particularly to compounds which activate the DNA repair enzyme poly(ADP-ribose) polymerase (PARP), since PARP requires NAD⁺ as a substrate and consumes NAD⁺ during its enzymatic action.

Accordingly, in some embodiments, the present invention provides that the methods of treating cancer disclosed herein further comprise administering a therapeutically-effective amount of a PARP activator to the patient in addition to administering a compound of the present invention, such as, for example, a compound of Formulae I, II, III, IV, IVa, and IVb, as illustrated herein, and a compound of Table 1, or a pharmaceutically-acceptable salt thereof.

Additionally, in some of such embodiments, the cells of the cancer have functional homologous recombination (HR) systems. Also, in some of such embodiments, the methods further comprise identifying the cells of the cancer as having functional HR systems. Methods of performing such identification are known in the art. Furthermore, in addition to a PARP activator, in some embodiments, the methods of treating cancer disclosed herein further comprise administering a therapeutically-effective amount of a non-DNA damaging agent to the patient, wherein the non-DNA damaging agent is not a PARP activator and not a compound of the present invention. For example, where the cancer has functional HR systems for repairing DNA damage, then an additional chemotherapeutic could be administered that does not rely on DNA damage for efficacy. Chemotherapeutics the do not damage DNA are known in the art.

Agents or treatments that may be capable of activating the PARP enzyme include but are not limited to: alkylating agents (methyl methane sulfonate (MMS), N-methyl-N′ nitro-N-nitrosoguanidine (MNNG), Nitrosoureas (N-methyl-N-nitrosourea (MNU), streptozotocin, carmustine, lomustine), Nitrogen mustards (melphalan, cyclophosphamide, uramustine, ifosfamide, clorambucil, mechlorethamine), alkyl sulfonates (busulfan), platins (cisplatin, oxaliplatin, carboplatin, nedaplatin, satraplatin, triplatin tetranitrate), non-classical DNA alkylating agents (temozolomide, dacarbazine, mitozolamide, procarbazine, altretamine)), radiation (X-rays, gamma rays, charged particles, UV, systemic or targeted radioisotope therapy), and other DNA damaging agents such as: topoisomerase inhibitors (camptothecin, beta-lapachone, irinotecan, etoposide), anthracyclines (doxorubicin, daunorubicin, epirubicin, idarubicin, valrubicin, mitoxantrone), reactive oxygen generators (menadione, peroxynitrite), and anti-metabolites (5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, floxuridine).

It is further believed that tumors or tumor cell lines treated with compounds that directly or indirectly inhibit the enzyme thymidylate synthase (TS) can also be more susceptible to Nampt inhibitors, such as, for example, compounds of the present invention.

Accordingly, in some embodiments, the present invention provides the methods of treating cancer disclosed herein further comprise administering a therapeutically-effective amount of a thymidylate synthase inhibitor to the patient in need of such treatment, in addition to administering a compound of the present invention, such as, for example, a compound of Formulae I, II, III, IV, IVa, and IVb, as illustrated herein, and a compound of Table 1, or a pharmaceutically-acceptable salt thereof.

In some embodiments, the thymidylate synthase inhibitor directly or indirectly inhibits thymidylate synthase. Thymidylate synthase inhibitors include 5-FU, raltitrexed, pemetrexed, and other TS inhibitors developed over the past decades.

It is further believed that agents that promote aberrant uracil incorporation into DNA can also make subjects being administered such agents more susceptible to Nampt inhibitors, such as, for example, compounds of the present invention. Any inhibitor of thymidylate synthase (TS) would cause uracil incorporation into DNA. Other agents, such as, for example, inhibitors of dihydrofolate reductase (e.g. methotrexate) have also been shown to cause uracil to aberrantly incorporate into DNA.

Accordingly, in some embodiments, the present invention provides the methods of treating cancer disclosed herein further comprise administering a therapeutically-effective amount of agents that promote aberrant uracil incorporation into DNA, to the patient in need of such treatment, in addition to administering a compound of the present invention, such as, for example, a compound of Formulae I, II, III, IV, IVa, and IVb, as illustrated herein, and a compound of Table 1, or a pharmaceutically-acceptable salt thereof.

In view of the above, some embodiments of the present invention comprises the use of the compounds of the present invention with a second chemotherapeutic agent that has been discovered to work synergistically with one or more of the compounds of the present invention, such as, for example, compounds or treatments that activate PARP, induce DNA damage, inhibit TS, and/or promote aberrant uracil incorporation into DNA, or inhibit proteasomes or specific kinases.

In certain of such embodiments, the second chemotherapeutic agent is selected from, at least, methyl methanesulfonate (MMS), mechlorethamine, streptozotocin, 5-fluorouracil (5-FU), raltitrexed, methotrexate, bortezomib, PI-103, and dasatinib.

In cells that have lost the function of BRCA tumor suppressors, HR function is compromised, and these cells are killed by PARP inhibitors (Ashworth A. (2008) Journal of Clinical Oncology 26(22):3785-90). International Patent Application No. PCT/US11/26752, filed Mar. 1, 2011, the entire contents of which are incorporated herein by reference, discloses that Nampt inhibitors and the PARP inhibitor olaparib synergized in causing cell death. This result is particularly encouraging as it suggests that the drug combination of one of the compounds of the present invention plus a PARP inhibitor would be antagonistic in normal cells, but synergistic in cells that do not have functional HR systems, such as, for example, cells that have lost BRCA tumor suppressor function.

Other routes of HR deficiency in oncogenesis (other than BRCA sequence mutation) could also lead to sensitivity to PARP inhibition plus Nampt inhibitor combination therapy. These additional mutations, which lead to a “BRCAness” phenotype, include, as documented in ovarian cancers, BRCA1 promoter methylation and upregulation of BRCA inhibitors, such as, for example, the protein EMSY (Bast R. C. and Mills G. B. Journal of Clinical Oncology 28(22):3545-8 (2010)). Further studies have demonstrated that mutation of the tumor suppressor gene phosphatase and tensin homolog (PTEN), a gene frequently mutated in a variety of cancers, reduces HR function and sensitizes cells to PARP inhibitors (Mendes-Pereira A. M. et al. EMBO Molecular Medicine 1:315-322 (2009)). Providing more evidence for the BRCAness model of PARP inhibitor sensitivity, in a cell biological study using RNA interference, mutation of any of 12 different genes functionally important for HR sensitized cells to PARP inhibitors (McCabe et al. Cancer Research 66(16): 8109-15 (2006)). Finally, a recent paper has demonstrated that cells in hypoxic conditions, such as those found in the center of virtually all solid tumors, are selectively killed by PARP inhibitors (Chan et al. Cancer Research 70(2): 8045-54 (2010)).

Accordingly, in some embodiments, the present invention provides the methods of treating cancer disclosed herein further comprise administering a therapeutically-effective amount of a PARP inhibitor to the patient in need of such treatment, in addition to administering a compound of the present invention, such as, for example, a compound of Formulae I, II, III, IV, IVa, and IVb, as illustrated herein, and a compound of Table 1, or a pharmaceutically-acceptable salt thereof.

In some of such embodiments, the cells of the cancer do not have functional homologous recombination (HR) systems. In some of such embodiments, the methods of treating cancer further comprise identifying the cells of the cancer as not having functional HR systems. Methods of performing such identification are known in the art.

In some of such embodiments, the PARP inhibitor is olaparib, AG014699/PF-01367338, INO-1001, ABT-888, Iniparib, BSI-410, CEP-9722, MK4827, or E7016.

In some of such embodiments, the methods further comprise administering a therapeutically-effective amount of a DNA damaging agent to the patient in need of such treatment, wherein the DNA damaging agent is other than a PARP inhibitor. DNA damaging agents are known in the art and include topoisomerase inhibitors (camptothecin, beta-lapachone, irinotecan, etoposide), anthracyclines (doxorubicin, daunorubicin, epirubicin, idarubicin, valrubicin, mitoxantrone), reactive oxygen generators (menadione, peroxynitrite), and anti-metabolites (5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, floxuridine).

Another specific example of an active agent with which the compounds of the present invention can be co-administered is the immune adjuvant L-1-methyl tryptophan (L-1MT). In studies of co-administration of L-1MT with another inhibitor of Nampt, APO866 (also known as FK866 or WK175), the combination was shown to provide an additive inhibitory effect on tumor growth of murine gastric and bladder tumors in immune-competent mice (Yang et al. Exp. Biol. Med. 235:869-76 (2010)).

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of temozolomide, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of 4HC, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of 5-FU, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of L-1MT, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of methyl methanesulfonate (MMS), to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of mechlorethamine, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of streptozotocin, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of raltitrexed, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of methotrexate, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of bortezomib, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of PI-103, to a patient in need of such treatment.

Thus, in one embodiment, the present invention provides a method of treating cancer, comprising administering a therapeutically-effective amount of one or more compounds of the present invention, such as, for example, the compounds of Formulae I, II, III, IV, IVa, and IVb, as illustrated herein, and the compounds of Tables 1-9, or a pharmaceutically-acceptable salt thereof, or a pharmaceutical composition comprising one or more compounds of the present invention, and administering a therapeutically-effective amount of dasatinib, to a patient in need of such treatment.

In the case of combination therapy, a therapeutically-effective amount of one or more other therapeutically-effective compounds can be administered in a separate pharmaceutical composition, or alternatively included in the same pharmaceutical composition of the present invention which contains one of the compounds of the present invention. One or more of the compounds of the present invention can be administered together in the same formulation with the one or more other compounds that have been shown to be effective in the treatment of cancer, systemic or chronic inflammation, rheumatoid arthritis, diabetes, obesity, T-cell mediated autoimmune disease, ischemia, and other complications associated with these diseases and disorders, in the same formulation or dosage form. Thus, the present invention also provides pharmaceutical compositions or medicaments for combination therapy, comprising an effective amount of one or more of the compounds of the present invention, and an effective amount of at least one other compound that has been shown to be effective in the treatment of cancer, systemic or chronic inflammation, rheumatoid arthritis, diabetes, obesity, T-cell mediated autoimmune disease, ischemia, and other complications associated with these diseases and disorders.

The compounds of the present invention can also be administered in combination with another active agent that synergistically treats or prevents the same symptoms or is effective for another disease or symptom in the patient being treated, so long as the other active agent does not interfere with, or adversely affect, the effects of the compounds of the present invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, immune adjuvants, and the like.

### 6. Methods of Making the Compounds of the Present Invention

Additionally, the present invention provides methods of the making the compounds of the present invention. Embodiments of methods of making the compounds of the present invention, and intermediates used in their synthesis, are provided in General Synthetic Method A and General Synthetic Method B below. Specific methods of making some of the compounds of the present invention are illustrated in Synthetic Methods A-Z and 1 through 2. General Synthetic Method A and Synthetic Methods A-Z illustrate methods of making particular compounds of the present invention containing a urea-moiety (i.e., —N(H)—C(═O)—N(H)—). General Synthetic Method B and Synthetic Methods 1 and 2 illustrate methods of making particular compounds of the present invention containing a —C(H)═C(H)—C(═O)—N(H)— moiety. It should be understood that to the extent a portion of a General Synthetic Method or Synthetic Method does not relate specifically to formation of a urea moiety or a —C(H)═C(H)—C(═O)—N(H)— moiety, then that portion of the General Synthetic Method or Synthetic Method may be used to make compounds containing either of such moieties.

General Synthetic Method A

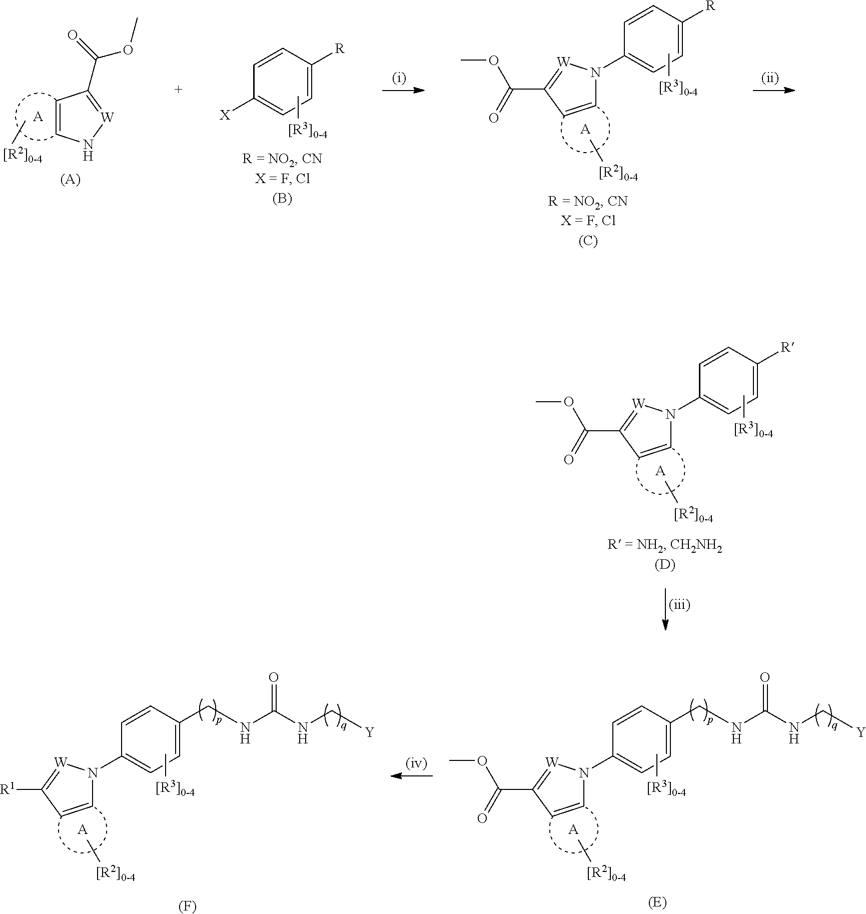

For example, compounds of the present invention can be prepared starting from an appropriately substituted ester (A), such as from a commercially available ester or acid. If an acid is used, then the acid can be converted to the corresponding ester (A) using a conventional acid (such as, for example, HCl, H₂SO₄, etc.) under catalyzed esterification conditions in alcoholic solvents (such as, for example, methanol or ethanol) at room temperature or thermal conditions (60-80° C.). Ester (A) can be converted to the intermediate (C) via nucleophilic displacement (i) of the halogen (such as, for example, fluorine or chlorine) in an appropriately substituted halo-arene (B) using a base (such as, for example, sodium hydride or cesium carbonate, etc.) in a solvent (such as DMF, DMSO, etc.) at either room temperature or thermal conditions (40-60° C.) for 1-4 hours. Nitro or the cyano group in the intermediate (C) can be reduced (ii) using appropriate reducing agents (such as, for example, 10% Pd/C, Zn, Fe, Sn, etc.) in a solvent (such as, for example, MeOH, EtOH, acetic acid, HCl, etc.) to an aniline or alkyl amino derivative (D). Intermediate (D) which in turn can be converted (iii) to a desired urea derivative (E) using an appropriate heteroaryl amine or heteroaryl alkyl amine with coupling reagents (such as, for example, diphosgene, triphosgene, CDI, etc.) in a solvent (such as dichloromethane, dioxane, pyridine, etc) at 0° C. to room temperature over 4-8 hours. Finally, the ester in the urea derivative (E) can be hydrolyzed to an acid (not shown) using a base (such as, for example, sodium hydroxide, potassium hydroxide, cesium carbonate, etc.) in solvent (such as, for example, methanol, ethanol, etc.). The resulting acid (not shown) can be coupled (iv) with an appropriate amine using standing coupling conditions using reagents (such as, for example, HATU, EDCI, HOBT, etc.) in a solvent (such as, for example, DMF, THF, etc.) at room temperature for 8-16 hours to form R¹.

Certain embodiments of methods of making the compounds of the present invention, and intermediates used in their synthesis, are provided in General Synthetic Method B below.

General Synthetic Method B

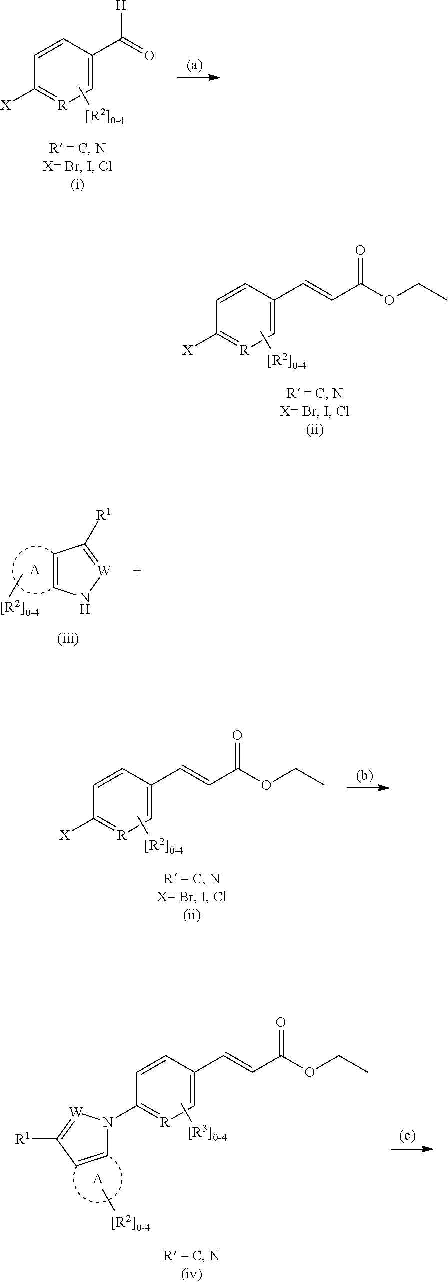

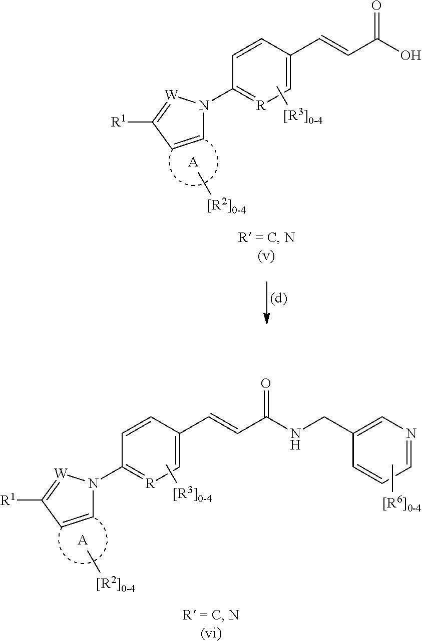

For example, many compounds of the present invention can be prepared starting from an appropriately substituted aldehyde of 5 or 6 membered aromatic groups such as aryl or heteroaryls (i), which are commercially available. Aldehyde (i) can be converted to the α,β-unsaturated ester derivative (ii) employing standard Horner-Emmons reaction conditions with reagent such as, for example, ethyl phosphonoacetate and a base (such as, for example, lithium hydroxide or sodium hydride) in solvents (such as, for example, THF, DME, etc.), or by using Wittig reaction conditions with reagents (such as, for example, (2-methoxy-2-oxoethyledene)triphenylphosphorane) in solvents (such as, for example, toluene, THF, etc.) at either room temperature or in refluxing conditions. α,β-unsaturated ester derivative (ii) can be coupled with intermediate (iii) employing standard Buchwald conditions using either palladium or copper catalyst with ligands (such as, for example, trans-cyclohexyldiamine, etc.) in solvents (such as, for example, DMF, toluene, etc.) at 100-110° C. to yield intermediate (iv). Intermediate (iii), which is an optionally substituted mono or bicyclic heteroaryl, is commercially available. Intermediate (iv) can be hydrolyzed using alkaline hydrolytic conditions and a base (such as, for example, aqueous sodium hydroxide, potassium hydroxide or lithium hydroxide, etc.) in solvents (such as, for example, methanol, THF, etc.) at either room temperature or at 40-80° C. for 3-7 hours to furnish acid derivative (v). The acid derivative (v) can be coupled with an appropriate amine (described in the claims) employing standard coupling reagents (such as, for example, HATU, EDC, HOBT, etc.) in solvents (such as, for example, THF, DMF, DMA, etc.) at the room temperature.

Synthetic Method A:

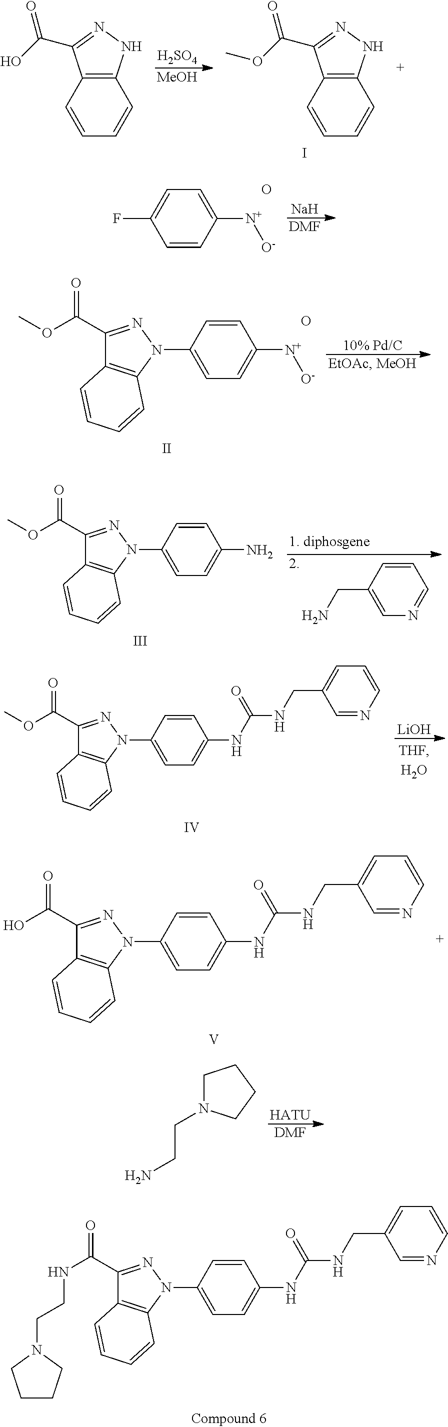

### Methyl 1H-indazole-3-carboxylate (I)

1H-indazole-3-carboxylic acid (2.4 g, 14.8 mmol) was dissolved in 100 mL methanol with 0.20 mL H₂SO₄ and heated to 80° C. for 16 hours. Methanol was removed on rotary evaporator and the resulting residue was dissolved in 100 mL EtOAc. The organic solution was washed with water, saturated NaHCO₃ and brine, dried over Na₂SO₄ and concentrated to yield product (2.37 g, 13.5 mmol, 90.1%). Product was identified by GC/MS.

### Methyl 1-(4-nitrophenyl)indazole-3-carboxylate (II)

Methyl 1H-indazole-3-carboxylate (4.0 g, 22.7 mmol) was dissolved in 100 mL DMF and chilled to 0° C. NaH (0.82 g, 34.1 mmol) was added portion wise and stirred for 30 minutes at room temperature. 1-fluoro-4-nitro-benzene (3.84 g, 27.2 mmol) was added and the reaction was stirred for an additional 3 hours at room temperature. Product was isolated by filtration following precipitation with 100 mL H₂O yielding (2.43 g, 8.18 mmol, 36%). Product was identified by LC/MS.

### Methyl 1-(4-aminophenyl)indazole-3-carboxylate (III)

Methyl 1-(4-nitrophenyl)indazole-3-carboxylate (2.43 g, 8.18 mmol) was dissolved in 200 mL EtOAc, 250 mL MeOH, and 2.0 mL CH₃CO₂H. To this solution was added 10% Pd/C (300 mg) and placed under balloon pressure H₂ for 16 hours. Note not all starting material was soluble initially, but did go into solution during the course of the reaction. Pd/C was removed by celite filtration and solvent removed on rotary evaporator. The reaction residue was taken up in EtOAc and washed with saturated NaHCO₃ and brine, dried over Na₂SO₄ and concentrated to yield product (1.76 g, 6.59 mmol, 80.6%). Product was identified by LC/MS.

### Methyl 1-[4-(3-pyridylmethylcarbamoylamino)phenyl]indazole-3-carboxylate (IV)

Methyl 1-(4-aminophenyl)indazole-3-carboxylate (1.76 g, 6.59 mmol) was dissolved in 33 mL CH₂Cl₂ and chilled to 0° C. on an ice bath. Diphosgene (782 mg, 0.476 mL, 3.95 mmol) was added dropwise via syringe, followed by triethylamine (799 mg, 7.91 mmol, 1.10 mL) in the same fashion. The reaction was stirred for 30 minutes at 0° C. 3-Pyridylmethanamine (1.42 g, 13.2 mmol, 1.34 mL) was added dropwise via syringe, followed by triethylamine (799 mg, 7.91 mmol, 1.10 mL) in the same fashion. The reaction was stirred for 3 hours while coming to room temperature. The solution was washed with water, saturated ammonium chloride, saturated sodium bicarbonate and brine, dried over Na₂SO₄ and concentrated to yield product (2.52 g, 6.28 mmol, 95%). Product was identified by LCMS.

### 1-[4-(3-pyridylmethylcarbamoylamino)phenyl]indazole-3-carboxylic acid (V)

Methyl 1-[4-(3-pyridylmethylcarbamoylamino)phenyl]indazole-3-carboxylate (2.52 g, 6.28 mmol) was dissolved in THF (30 mL), MeOH (2.0 mL), and H₂O (2.0 mL). LiOH.H₂O (789 mg, 18.8 mmol) was added and the mixture was heated at 70° C. for 12 hours. The reaction mixture was evaporated to dryness on a rotary evaporator, the resiude was taken up in 30 mL H₂O and neutralized with 5 N HCl. The product was isolated by filtration and vacuum dried yielding (1.79 g, 4.62 mmol, 73.6%). Product was identified by LC/MS.

### 1-[4-(3-pyridylmethylcarbamoylamino)phenyl]-N-(2-pyrrolidin-1-ylethyl)indazole-3-carboxamide (Compound 7)

1-[4-(3-pyridylmethylcarbamoylamino)phenyl]indazole-3-carboxylic acid (75 mg, 0.194 mmol), 2-pyrrolidin-1-ylethanamine (26.6 mg, 0.233 mmol, 29.0 μL) and HATU (111 mg, 0.291 mmol) were combined in DMF (1.0 mL) and stirred at room temperature for 1.5 hours. The reaction mixture was deposited on celite and vacuum dried. Purification was done my MPLC [13 g C-18: 20→40% MeOH/H₂O, 0.1% TFA]. Product was identified by H¹ NMR: δ9.64 (bs, 1H), 9.39 (s, 1H), 8.81 (t, 5.84 Hz, 1H), 8.73 (s, 1H), 8.67 (s, 1H), 8.30 (d, 8.14 Hz, 1H), 8.14 (d, 7.89 Hz, 1H), 7.78-7.64 (m, 5H), 7.54 (t, 7.89 Hz, 1H), 7.39 (t, 8.20 Hz, 1H), 7.32 (t, 5.30 Hz, 1H), 4.45 (d, 5.64 Hz, 2H), 3.71-3.64 (m, 4H), 3.43-3.37 (m, 2H), 3.12-3.03 (m, 2H), 2.06-1.98 (m, 2H), 1.91-1.83 (m, 2H). Mass Spectrometry confirmed structure calculated mass 483.2383 found mass 483.2405.

Synthetic Method B

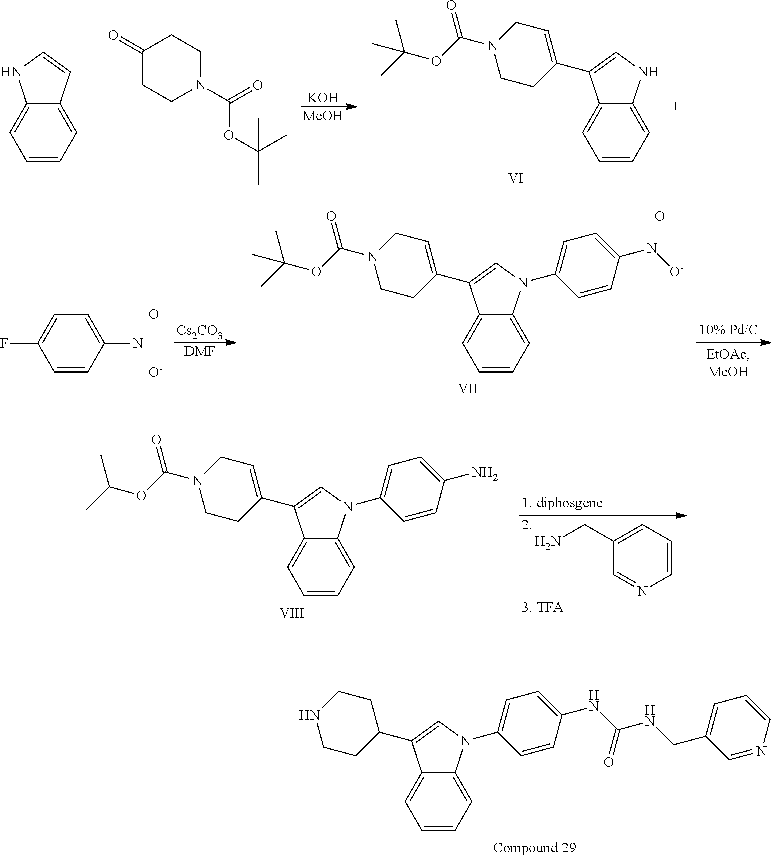

### tert-butyl 4-(1H-indol-3-yl)-3,6-dihydro-2H-pyridine-1-carboxylate (VI)

Indole (1.17 g, 1.0 mmol) and tert-butyl 4-oxopiperidine-1-carboxylate (2.39 g, 1.2 mmol) were combined with KOH (1.12 g, 20 mmol) in 50 mL MeOH and heated to 60° C. for 18 hours. Following solvent removal the reaction residue was purified by MPLC [40 g silica: 0→48→52% EtOAc/hexane]. Product eluted at 52% MeOH. Removal of mobile phase yielded product (1.82 g, 6.11 mmol, 61%) product. Product was identified by LC/MS.

### tert-butyl 4-[1-(4-nitrophenyl)indol-3-yl]-3,6-dihydro-2H-pyridine-1-carboxylate (VII)

tert-butyl 4-(1H-indol-3-yl)-3,6-dihydro-2H-pyridine-1-carboxylate (1.82 g, 6.11 mmol), 1-fluoro-4-nitro-benzene (1.03 g, 7.32 mmol) and Cs₂CO₃ (3.97 g, 12.2 mmol) were combined in 20 mL DMF and heated to 40° C. for 18 hours. The reaction mixture was poured into 200 mL water and extracted into EtOAc (3×75 mL). Solvent was removed from the combined organic extracts and the residue was purified by MPLC [40 g silica: 0→30% EtOAC/Hexane]. Removal of mobile phase yielded product (1.44 g, 3.44 mmol, 56%). Product was identified by LC/MS.

### tert-butyl 4-[1-(4-aminophenyl)indol-3-yl]piperidine-1-carboxylate (VIII)

tert-butyl 4-[1-(4-nitrophenyl)indol-3-yl]-3,6-dihydro-2H-pyridine-1-carboxylate (1.44 g, 3.44 mmol) was dissolved in 100 mL MeOH and 50 mL EtOAc. To this solution was added 10% Pd/C (approximately 300 mg) and placed under 30 p.s.i. H₂ on Parr Shaker for 16 hours. Pd/C was removed by celite filtration and solvent removed on rotary evaporator. The reaction residue was taken up in EtOAc and washed with saturated NaHCO₃ and brine, dried over Na₂SO₄ and concentrated. The reaction residue was purified by MPLC [40 g silica: 0→50% EtOAC/Hexane] to yield product (1.04 g, 2.67 mmol, 77%). Product was identified by LC/MS.

### 1-[4-[3-(4-piperidyl)indol-1-yl]phenyl]-3-(3-pyridylmethyl)urea (Compound 29)

This compound was prepared in the same manner as 1-[4-(3-pyridylmethylcarbamoylamino)phenyl]-N-(2-pyrrolidin-1-ylethyl)indazole-3-carboxamide was prepared in Method A above. Following formation of the urea the BOC group was removed in TFA/CH₂Cl₂ and purified by MPLC [13 g C-18: 5→35% MeOH/H₂O, 0.1% TFA]. Product was identified by H¹ NMR: δ 9.16 (s, 1H), 8.73 (bs, 2H), 8.67 (bs, 1H), 8.55-8.45 (m, 1H), 8.15 (d, 7.93 Hz, 1H), 7.76-7.72 (m, 2H), 7.61 (d, 8.63 Hz, 2H), 7.47-7.38 (m, 4H), 7.21-7.10 (m, 3H), 4.45 (d, 5.81 Hz, 2H), 3.41 (d, 12.6 Hz, 2H), 3.22-3.05 (m, 3H), 2.16 (d, 12.6 Hz, 2H), 1.98-1.87 (m, 2H). Mass Spectrometry confirmed structure calculated mass 426.22884 found mass 426.23414.

Synthetic Method C:

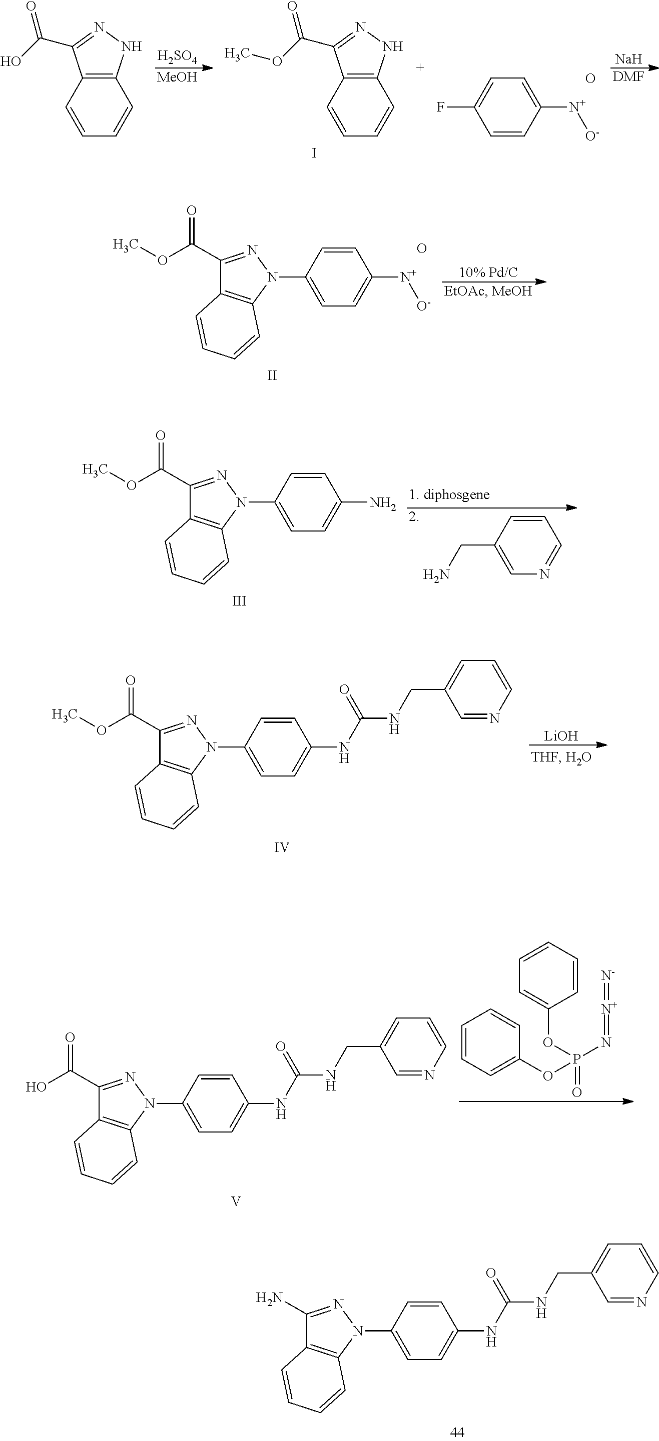

### Methyl 1H-indazole-3-carboxylate (I)

1H-indazole-3-carboxylic acid (2.4 g, 14.8 mmol) was dissolved in 100 mL methanol with 0.20 mL H₂SO₄ and heated to 80° C. for 16 hours. Methanol was removed on rotary evaporator and the resulting residue was dissolved in 100 mL EtOAc. The organic solution was washed with water, saturated NaHCO₃ and brine, dried over Na₂SO₄ and concentrated to yield product (2.37 g, 13.5 mmol, 90.1%). Product was identified by GC/MS.

### Methyl 1-(4-nitrophenyl)indazole-3-carboxylate (II)

Methyl 1H-indazole-3-carboxylate (I) (4.0 g, 22.7 mmol) was dissolved in 100 mL DMF and chilled to 0° C. NaH (60%, 0.82 g, 34.1 mmol) was added portion wise and stirred for 30 minutes at room temperature. 1-fluoro-4-nitro-benzene (3.84 g, 27.2 mmol) was added and the reaction was stirred for an additional 3 hours at room temperature. Product was isolated by filtration following precipitation with 100 mL H₂O yielding (2.43 g, 8.18 mmol, 36%). Product was identified by LC/MS.

### Methyl 1-(4-aminophenyl)indazole-3-carboxylate (III)

Methyl 1-(4-nitrophenyl)indazole-3-carboxylate (II) (2.43 g, 8.18 mmol) was dissolved in 200 mL EtOAc, 250 mL MeOH, and 2.0 mL CH₃CO₂H. To this solution was added 10% Pd/C (300 mg) and placed under balloon pressure H₂ for 16 hours. Note not all starting material was soluble initially, but did go into solution during the course of the reaction. Pd/C was removed by celite filtration and solvent removed on rotary evaporator. The reaction residue was taken up in EtOAc and washed with saturated NaHCO₃ and brine, dried over Na₂SO₄ and concentrated to yield product (1.76 g, 6.59 mmol, 80.6%). Product was identified by LC/MS.

### Methyl 1-[4-(3-pyridylmethylcarbamoylamino)phenyl]indazole-3-carboxylate(IV)

Methyl 1-(4-aminophenyl)indazole-3-carboxylate (III) (1.76 g, 6.59 mmol) was dissolved in 33 mL CH₂Cl₂ and chilled to 0° C. on an ice bath. Diphosgene (782 mg, 0.476 mL, 3.95 mmol) was added dropwise via syringe, followed by triethylamine (799 mg, 7.91 mmol, 1.10 mL) in the same fashion. The reaction was stirred for 30 minutes at 0° C. 3-Pyridylmethanamine (1.42 g, 13.2 mmol, 1.34 mL) was added dropwise via syringe, followed by triethylamine (799 mg, 7.91 mmol, 1.10 mL) in the same fashion. The reaction was stirred for 3 hours while coming to room temperature. The solution was washed with water, saturated ammonium chloride, saturated sodium bicarbonate and brine, dried over Na₂SO₄ and concentrated to yield product (2.52 g, 6.28 mmol, 95%). Product was identified by LCMS.

### 1-[4-(3-pyridylmethylcarbamoylamino)phenyl]indazole-3-carboxylic acid (V)

Methyl 1-[4-(3-pyridylmethylcarbamoylamino)phenyl]indazole-3-carboxylate (IV) (2.52 g, 6.28 mmol) was dissolved in THF (30 mL), MeOH (2.0 mL), and H₂O (2.0 mL). LiOH.H₂O (789 mg, 18.8 mmol) was added and the mixture was heated at 70° C. for 12 hours. The reaction mixture was evaporated to dryness on a rotary evaporator, the residue was taken up in 30 mL H₂O and neutralized with 5 N HCl. The product was isolated by filtration and vacuum dried yielding (1.79 g, 4.62 mmol, 73.6%). Product was identified by LC/MS.

1-[4-(3-aminoindazol-1-yl)phenyl]-3-(3-pyridylmethyl)urea (44): To a suspension of 1-[4-(3-pyridylmethylcarbamoylamino)phenyl]indazole-3-carboxylic acid (V) (387 mg, 1.0 mmol) lin DMF (8 mL) was successively added a solution of triethylamine (152 mg, 1.5 mmol) dissolved in DMF (1.8 mL) and diphenylphosphoryl azide (413 mg, 1.5 mmol) dissolved in DMF (1.8 mL). The reaction was stirred at RT for 2.5 hours. Water (1.0 mL) was added and the reaction was heated to 100° C. for 1 hour. The product was filtered as a ppt and vacuum dried. Product was identified by LC/MS and H¹-NMR.

Synthetic Method D:

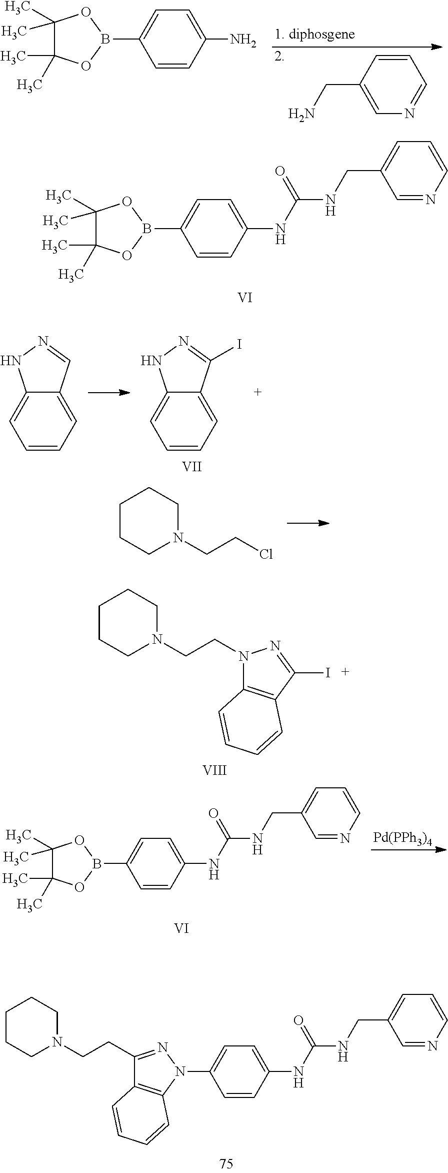

### 1-(3-pyridylmethyl)-3-[4-(4,4,5,5-tetramethyl-1,3,2-dioxaborolan-2-yl)phenyl]urea (VI)

4-(4,4,5,5-tetramethyl-1,3,2-dioxaborolan-2-yl)aniline (2.0 g, 9.1 mmol) was dissolved in 47 mL CH₂Cl₂ and chilled to 0° C. on an ice bath. Diphosgene (1.08 g, 0.66 mL, 5.46 mmol) was added dropwise via syringe, followed by triethylamine (1.11 g, 1.85 mL, 11.0 mmol) in the same fashion. The reaction was stirred for 30 minutes at 0° C. 3-Pyridylmethanamine (1.97 g, 1.85 mL, 18.2 mmol) was added dropwise via syringe, followed by triethylamine (1.11 g, 1.85 mL, 11.0 mmol) in the same fashion. The reaction was stirred for 3 hours while coming to room temperature. The solution was washed with water, saturated ammonium chloride, saturated sodium bicarbonate and brine, dried over Na₂SO₄ and concentrated to yield product (2.86 g, 8.1 mmol, 89%). Product was identified by LCMS.

### 3-iodo-1H-indazole (VII)

Indazole (1.0 g, 8.47 mmol) and K₂CO₃ (1.71 g, 12.4 mmol) were combined in DMF (5 mL) and chilled to 0° C. I₂ (2.70 g, 1.3 mmol) dissolved in DMF (2 mL) was added dropwise over a one hour time period, then stirred 18 hours at room temperature. The reaction was then poured into a solution of sodiumthiosulfate (2.0 g) and K₂CO₃ (10 mg) in 10 mL water. A white precipitate formed and was stirred at room temperature for 1.5 hours. Product was isolated by filtration and indentified by LCMS yielding (1.87 g, 7.68 mmol, 91%).

### 3-iodo-1-[2-(1-piperidyl)ethyl]indazole (VIII)

3-iodo-1H-indazole (VII) (488 mg, 2.0 mmol) was dissolved in DMF (8.0 mL) and chilled to 0° C. NaH (60%, 168 mg, 4.2 mmol) was added to reaction and stirred for 20 minutes. 1-(2-chloroethyl)piperidine hydrochloride (386 mg, 2.1 mmol) was added in one portion, the reaction was stirred 18 hours at room temperature. The reaction was poured into 40 mL water and the product was isolated by filtration and vacuum drying. The product was identified by LCMS and yielded 637 mg (89%).

### 1-[4-[1-[2-(1-piperidyl)ethyl]indazol-3-yl]phenyl]-3-(3-pyridylmethyl)urea (75)

1-(3-pyridylmethyl)-3-[4-(4,4,5,5-tetramethyl-1,3,2-dioxaborolan-2-yl)phenyl]urea (VI) (354 mg, 0.99 mmol), 3-iodo-1-[2-(1-piperidyl)ethyl]indazole (VIII) (175 mg, 0.49 mmol) and tetrakis(triphenylphosphine) palladium(0) (85.0 mg, 0.074 mmol) were combined in dimethoxyethane (6.0 mL), ethanol (1.2 mL), and saturated NaHCO₃ (1.2 mL). The reaction mixture was degassed and heated to 100° C. for 18 hours. The reaction residue was purified by MPLC [13 g C-18: 15→50→95% MeOH/H₂O, 0.1% TFA] to yield product (1.04 g, 2.67 mmol, 77%). Product was identified by LC/MS.

Synthetic Method E:

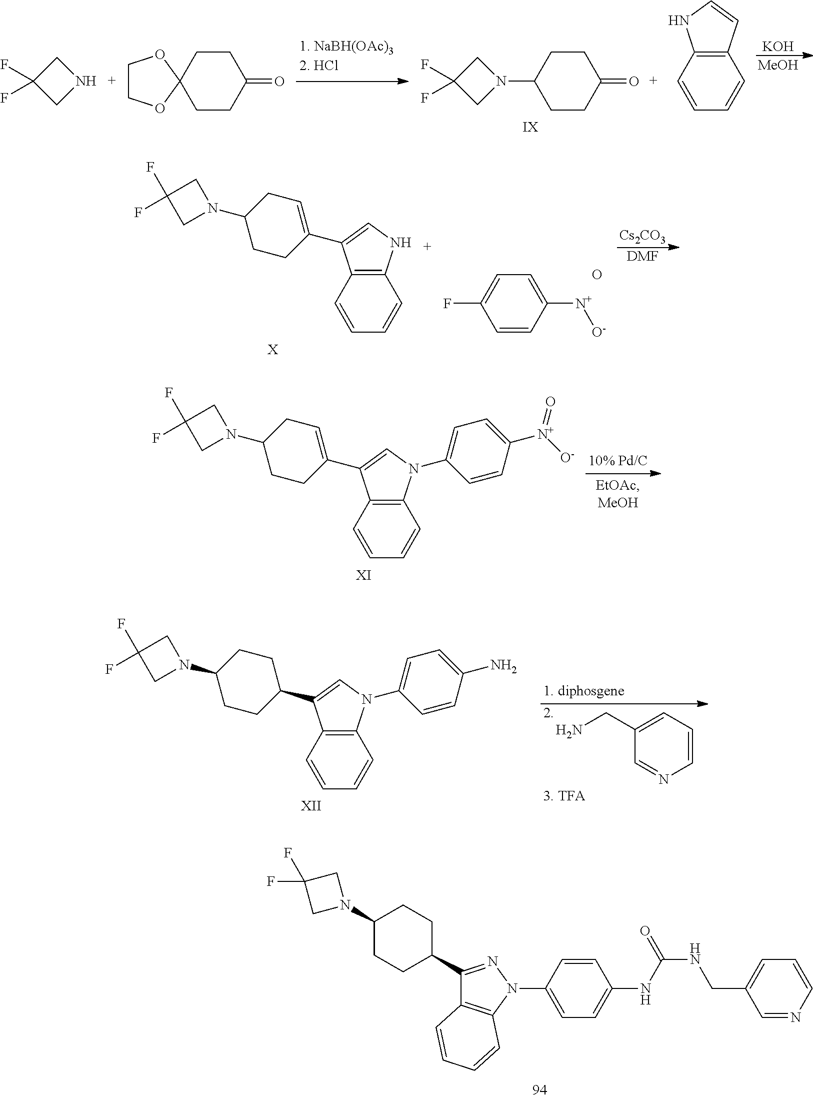

### 4-(3,3-difluoroazetidin-1-yl)cyclohexanone (IX)

3,3-difluoro azetidine hydrochloride (500 mg, 3.87 mmol), 1,4-dioxaspiro[4.5]decan-8-one (604 mg, 3.87 mmol), and diisopropylethylamine (499 mg, 0.742 mL, 3.87 mmol) were combined in MeOH (20 mL). Sodium triacetoxyborohydride (2.04 g, 9.68 mmol) was added, the reaction was stirred at room temperature for 18 hours. Reaction solvent was removed under vacuum, the residue was dissolved in ethyl acetate and washed with saturated NaHCO₃, H₂O and brine. The organic phase was dried over Na₂SO₄ and concentrated, the residue was dissolved in 5 N HCl (10 mL) and stirred at room temperature for 3 hours. The aqueous reaction mixture was extracted 3× with ethyl acetate. The combined organic layers were washed with brine and dried over Na₂SO₄. Solvent removal afforded desired product which was identified by GC/MS and used without further purification.

### 3-[4-(3,3-difluoroazetidin-1-yl)cyclohexen-1-yl]-1H-indole (X)

4-(3,3-difluoroazetidin-1-yl)cyclohexanone (IX) (300 mg, 1.59 mmol) and indole (155 mg, 1.32 mmol) were combined with KOH (147 mg, 2.64 mmol) in MeOH (6.6 mL) and heated to 60° C. for 18 hours in a sealed tube. The reaction mixture was deposited on silica and purified by MPLC [12 g silica: 0→60% ethyl acetate/hexane] to yield product (0.10 g, 0.35 mmol, 22%). Product was identified by LC/MS.

### 3-[4-(3,3-difluoroazetidin-1-yl)cyclohexen-1-yl]-1-(4-nitrophenyl)indole (XI)

3-[4-(3,3-difluoroazetidin-1-yl)cyclohexen-1-yl]-1H-indole (X) (100 mg, 0.347 mmol), 1-fluoro-4-nitro-benzene (53.9 mg, 0.382 mmol) and Cs₂CO₃ (169 mg, 0.521 mmol) were combined in 2.0 mL DMF and heated to 60° C. for 18 hours. The reaction mixture was poured into 200 mL water and extracted into EtOAc (3×75 mL). Solvent was removed from the combined organic extracts and the residue was purified by MPLC [12 g silica: 0 hold 5 min→30% MeOH/CH₂Cl₂]. Removal of mobile phase yielded product (140 mg, 0.34 mmol, 99%). Product was identified by LC/MS.

### 4-[3-[4-(3,3-difluoroazetidin-1-yl)cyclohexyl]indol-1-yl]aniline (XII)

3-[4-(3,3-difluoroazetidin-1-yl)cyclohexen-1-yl]-1-(4-nitrophenyl)indole (XI) (140 mg, 0.34 mmol) was dissolved in 25 mL MeOH. To this solution was added 10% Pd/C (approximately 30 mg) and placed under 40 p.s.i. of H₂ on Parr Shaker for 18 hours. Pd/C was removed by celite filtration and solvent removed on rotary evaporator. The reaction residue was taken up in EtOAc and washed with saturated NaHCO₃ and brine, dried over Na₂SO₄ and concentrated. Both nitro and alkene were reduced under the conditions used. The reaction residue was purified by MPLC [40 g silica: 0→23→28→40% EtOAC/Hexane] to yield product (20 mg, 2.67 mmol, 15%).

Product was identified as the cis isomer by H¹-NMR, mass was confirmed by LC/MS. A small amount of trans isomer was isolated but not used in subsequent reactions.

### 1-[4-[3-[4-(3,3-difluoroazetidin-1-yl)cyclohexyl]indol-1-yl]phenyl]-3-(3-pyridylmethyl)urea (94)

4-[3-[4-(3,3-difluoroazetidin-1-yl)cyclohexyl]indol-1-yl]aniline (XII) (20.0 mg, 0.052 mmol) was dissolved in 1.0 mL CH₂Cl₂ and chilled to 0° C. on an ice bath. Diphosgene (6.2 mg, 0.011 mL, 0.031 mmol) was added dropwise via syringe, followed by triethylamine (6.3 mg, 0.009 mL, 0.062 mmol) in the same fashion. The reaction was stirred for 30 minutes at 0° C. 3-Pyridylmethanamine (11.2 mg, 0.011 mL, 0.104 mmol) was added dropwise via syringe, followed by triethylamine (6.3 mg, 0.009 mL, 0.062 mmol) in the same fashion. The reaction was stirred for 3 hours while coming to room temperature. The solution was washed with water, saturated ammonium chloride, saturated sodium bicarbonate and brine, dried over Na₂SO₄ and concentrated. The residue was deposited on celite and purified by MPLC [13 g C18: 5→55% MeOH/H₂O, 0.1% TFA] to yield product (12 mg, 0.023 mmol, 45%). Product was identified by LCMS and confirmed by H¹-NMR.

Synthetic Method F:

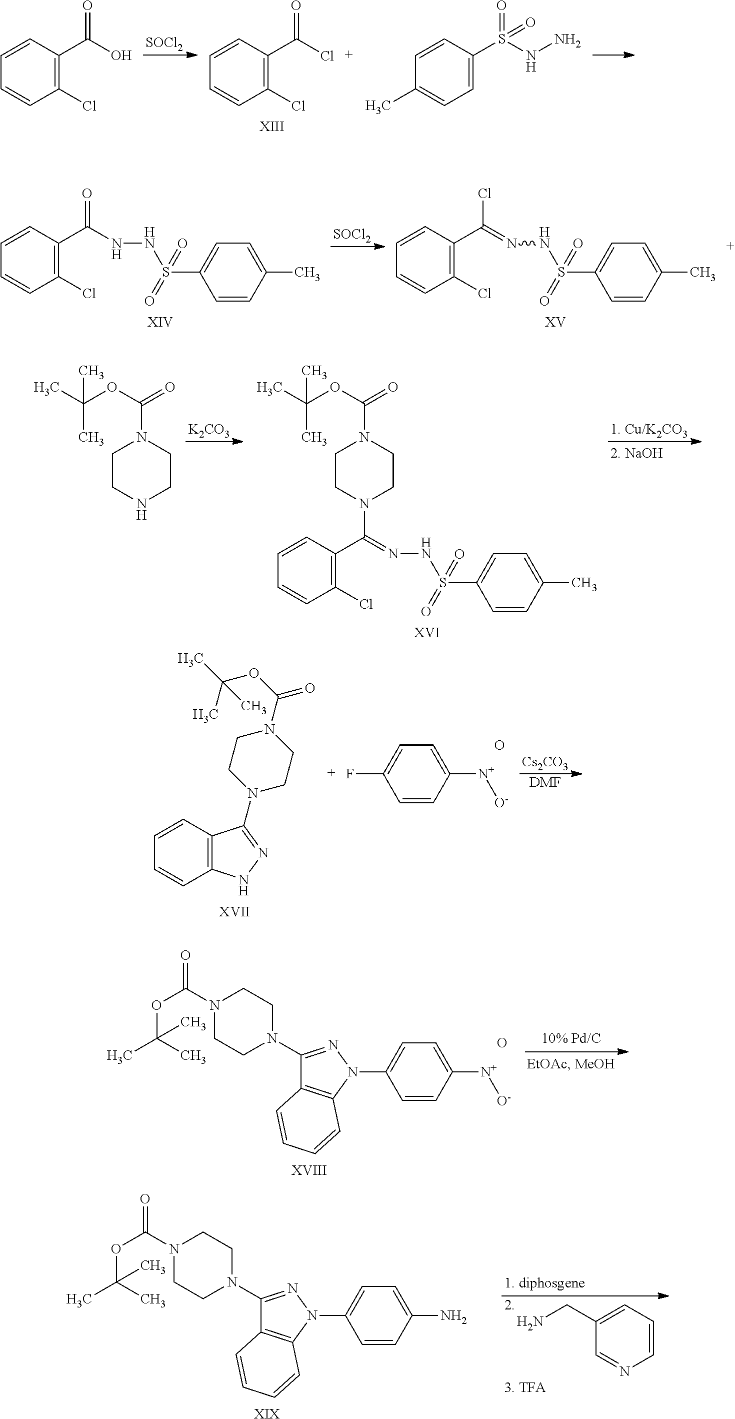

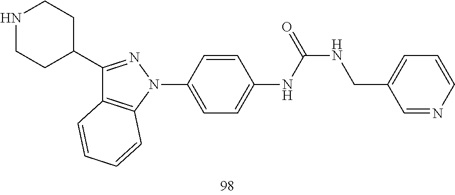 [SMILES: O=C(NCc1cccnc1)Nc1ccc(-n2nc(C3CCNCC3)c3ccccc32)cc1]

### 2-chlorobenzoyl chloride (XIII)

2-chlorobenzoic acid (1.0 g, 6.39 mmol) and thionyl chloride (792 mg, 6.71 mmol) were combined in toluene (7.5 mL) and heated to reflux for 18 hours. Toluene and excess thionyl chloride were removed from reaction mixture under reduced pressure; the reaction residue was dried under vacuum and carried on without further purification. Product identity confirmed by GC/MS.

### 2-chloro-N′-(p-tolylsulfonyl)benzohydrazide (XIV)

2-chlorobenzoyl chloride (XIII) (1.10 g, 6.39 mmol) and 4-methylbenzenesulfonohydrazide (1.19 g, 6.39 mmol) were combined in toluene (10 mL) and heated to 75° C. 18 hours. The resulting white precipitate was filtered, rinsed with toluene, and vacuum dried. Product (2.07 g, 6.39 mmol, 100%) was identified by LC/MS.

### (1Z)-2-chloro-N-(p-tolylsulfonyl)benzohydrazonoyl chloride (XV)

2-chloro-N′-(p-tolylsulfonyl)benzohydrazide (XIV) (1.04 g, 3.20 mmol) was combined with thionyl chloride (4.33 g, 36.7 mmol, 2.64 mL) and heated neat at 75° C. for 1.5 hours. The reaction was cooled to 60° C. and an additional portion of (XIV) (1.04 g, 3.20 mmol) was added and the reaction heated back to 75° C. for 2 hours. The reaction was quenched with hexane (50 mL). The resulting white precipitate was isolated by filtration and vacuum dried yielding product (1.83 g, 5.36 mmol, 84%).

### tert-butyl 4-[(Z)—C-(2-chlorophenyl)-N-(p-tolylsulfonylamino)carbonimidoyl]piperazine-1-carboxylate (XVI)

To a solution of tert-butyl piperazine-1-carboxylate (435 mg, 2.34 mmol) in NMP (3.7 mL) was added dropwise a solution of (1Z)-2-chloro-N-(p-tolylsulfonyl)benzohydrazonoyl chloride (XV) (400 mg, 1.17 mmol) in NMP (3.7 mL). The resulting solution was stirred for 40 minutes at room temperature. A portion of K₂CO₃ (242 mg, 1.76 mmol) was added and the reaction was heated to 40° C. for 3 hours. The reaction was quenched with 20 mL ice/water mixture. The resulting precipitate was isolated by filtration and vacuum dried yielding (576 mg, 1.17 mmol, 100%) product as an off-white solid. Product identity was confirmed by LC/MS.

### tert-butyl 4-(1H-indazol-3-yl)piperazine-1-carboxylate (XVII)

tert-butyl 4-[(Z)—C-(2-chlorophenyl)-N-(p-tolylsulfonylamino)carbonimidoyl]piperazine-1-carboxylate (XVI)

(576 mg, 1.17 mmol), Cu₍₀₎ (63.5 mg, 0.59 mmol) and K₂CO₃ (161 mg, 1.17 mmol) were combined in DMF (8.4 mL) and heated to reflux for 1 hour. The reaction was cooled to room temperature and quenched with H₂O (50 mL). The product was extracted into EtOAc (100 mL). The organic layer was washed with brine and dried over Na2SO4. Solvent removal afforded the tosyl protected product. The tosylated product was dissolved in methanol (50 mL) and 10 N NaOH (1 mL) and heated to reflux for 6 hours. Methanol was removed under reduced pressure. The residue was dissolved in ethyl acetate (100 mL), washed with H20 and brine, then dried over Na2SO4. Solvent removal afforded product (131 mg, 0.43 mmol, 37%). Product identity was confirmed by LC/MS.

### tert-butyl 4-[1-(4-nitrophenyl)indazol-3-yl]piperazine-1-carboxylate (XVIII)

tert-butyl 4-(1H-indazol-3-yl)piperazine-1-carboxylate (XVII) (131 mg, 0.43 mmol), 1-fluoro-4-nitro-benzene (66.0 mg, 0.47 mmol) and Cs₂CO₃ (280 mg, 0.86 mmol) were combined in 2.0 mL DMF and heated to 60° C. for 18 hours. The reaction mixture was poured into 100 mL water and the resulting precipitate was isolated by filtration. Product was identified by LC/MS and used without further purification.

### tert-butyl 4-[1-(4-aminophenyl)indazol-3-yl]piperazine-1-carboxylate (XIX)

tert-butyl 4-[1-(4-nitrophenyl)indazol-3-yl]piperazine-1-carboxylate (XVIII) (residue from above reaction) was dissolved in methanol (50 mL). 10% Pd/C (˜50 mg) was added and reaction was placed under a H₂ balloon with magnetic stirring for 18 hours. Pd/C was removed by filtration over pad of celite and methanol was removed under reduced pressure. The reaction residue was deposited on silica gel and purified by MPLC [12 g silica: 0→100% EtOAc/hexane]. Solvent was removed affording product (93 mg, 0.23 mmol, 55% over previous two steps). Product identity was confirmed by LC/MS.

### 1-[4-(3-piperazin-1-ylindazol-1-yl)phenyl]-3-(3-pyridylmethyl)urea (98)

tert-butyl 4-[1-(4-aminophenyl)indazol-3-yl]piperazine-1-carboxylate (XIX) (93.0 mg, 0.234 mmol) was dissolved in 1.0 mL CH₂Cl₂ and chilled to 0° C. on an ice bath. Diphosgene (27.8 mg, 0.14 mmol, 0.017 mL) was added dropwise via syringe, followed by triethylamine (28.4 mg, 0.28 mmol, 0.039 mL) in the same fashion. The reaction was stirred for 30 minutes at 0° C. 3-Pyridylmethanamine (50.1 mg, 0.464 mmol, 0.048 mL) was added dropwise via syringe, followed by triethylamine (28.4 mg, 0.28 mmol, 0.039 mL mmol) in the same fashion. The reaction was stirred for 3 hours while coming to room temperature. The reaction residue was deposited on celite and purified by MPLC [13 g C18: 5→95% MeOH/H₂O, 0.1% TFA] to yield BOC protected product. BOC protected product was dissolved in 1N HCl and allowed to stand at Rt 18 hours. The deprotected product was extracted into EtOAc. The organic layer was deposited on celite and purified by MPLC [13 g C18: 15→25% MeOH/H₂O, 0.1% TFA] Product (27.0 mg, 0.063 mmol, 26.9%) was identified by LC/MS and confirmed by H¹-NMR.

Synthetic Method G:

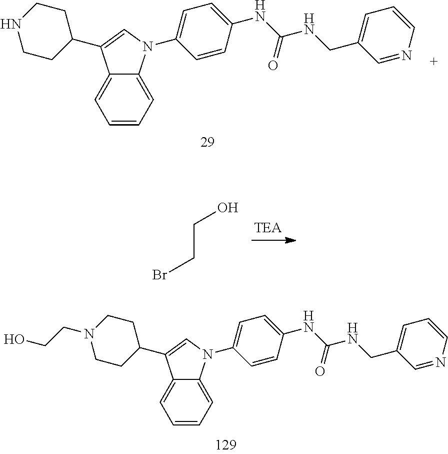

### 1-[4-[3-[1-(2-hydroxyethyl)-4-piperidyl]indol-1-yl]phenyl]-3-(3-pyridylmethyl)urea (129)

1-[4-[3-(4-piperidyl)indol-1-yl]phenyl]-3-(3-pyridylmethyl)urea (80.0 mg, 0.187 mmol), 2-bromoethanol (27.8 mg, 0.234 mmol) and triethylamine (377 mg, 0.374 mmol, 0.052 mL) were combined in DMF (1.0 mL) and heated to 50° C. 18 hours. The reaction mixture was deposited on celite and purified by MPLC [4.3 g C-18: 5→25% acetonitrile/water, 0.1% TFA]. Product (85.0 mg, 0.145 mmol, 78%) was identified by LC/MS and confirmed by H¹-NMR.

Synthetic Method H:

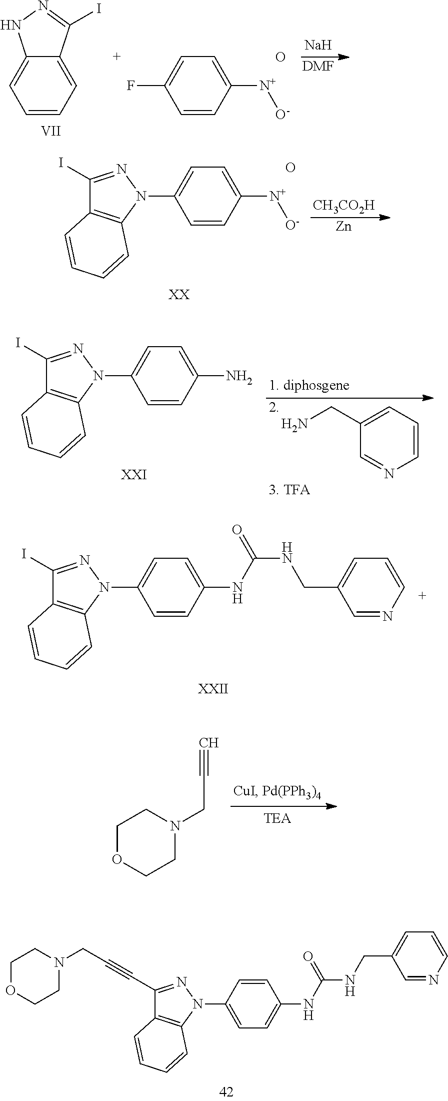

### 3-iodo-1-(4-nitrophenyl)indazole (XX)

3-iodo-1H-indazole (VII) (1.46 g, 6.0 mmol) was dissolved in 12 mL DMF and chilled to 0° C. NaH (60%, 0.48 g, 12.0 mmol) was added portion wise and stirred for 30 minutes at room temperature. 1-fluoro-4-nitro-benzene (0.89 g, 6.3 mmol) was added and the reaction was stirred for an additional 2 hours at room temperature. Product was isolated by filtration following precipitation with 50 mL H₂O yielding product (assumed quantitative yield). Product was identified by LC/MS and carried on without further purification.

### 4-(3-iodoindazol-1-yl)aniline (XXI)

3-iodo-1-(4-nitrophenyl)indazole (XX) (assumed 6.0 mmol) was dissolved in methanol (35 mL) and glacial acetic acid (35 mL) and chilled to 0° C. Zn dust (1.95 g, 30 mmol) was added and the reaction was stirred 3 hrs. Solvent was removed under reduced pressure. The residue was dissolved in ethyl acetate and washed with saturated sodium bicarbonate and brine. The organic layer was dried over Na2SO4 and concentrated to yield product (1.70 g, 5.05 mmol, 84% over 2 steps).

### 1-[4-(3-iodoindazol-1-yl)phenyl]-3-(3-pyridylmethyl)urea (XXII)

4-(3-iodoindazol-1-yl)aniline (XXI) (1.70 g, 5.05 mmol) was dissolved in CH₂Cl₂ (16 mL) and chilled to 0° C. on an ice bath. Diphosgene (599 mg, 3.03 mmol, 0.365 mL) was added dropwise via syringe, followed by triethylamine (612 mg, 6.06 mmol, 0.843 mL) in the same fashion. The reaction was stirred for 30 minutes at 0° C. 3-Pyridylmethanamine (708 mg, 6.56 mmol, 0.667 mL) was added dropwise via syringe, followed by triethylamine (612 mg, 6.06 mmol, 0.843 mL) in the same fashion. The reaction was stirred for 2 hours while coming to room temperature. The reaction residue was deposited on silica gel and purified by MPLC [12 g silica: 0→10% MeOH/CH₂Cl₂]. Solvent removal yielded product (2.07 g, 4.40 mmol, 80.2%) was identified by LC/MS.

### 1-[4-[3-(3-morpholinoprop-1-ynyl)indazol-1-yl]phenyl]-3-(3-pyridylmethyl)urea (42)

1-[4-(3-iodoindazol-1-yl)phenyl]-3-(3-pyridylmethyl)urea (XXII) (300 mg, 0.63 mmol) and 4-prop-2-ynylmorpholine (96.0 mg, 0.77 mmol) were combined in DMF (3.1 mL) along with CuI (2.4 mg, 0.013 mmol), tetrakis(triphenylphosphine)palladium(0) (29.1 mg, 0.025 mmol) and triethylamine (636 mg, 6.3 mmol, 0.876 mL). The reaction mixture was degassed by bubbling N₂ through reaction for 2 minutes and heated at 50° C. 18 hours. The reaction mixture was deposited on silica gel and purified by MPLC [12 g silica: 0→20% MeOH/CH₂Cl₂]. The product containing fractions were combined, deposited on celite and purified by MPLC [13 g C18: 5→65% acetonitrile/H₂O, 0.1% TFA]. Product (58.9 mg, 0.126 mmol, 20%) was identified by LC/MS and confirmed by H¹-NMR.

Synthetic Method I:

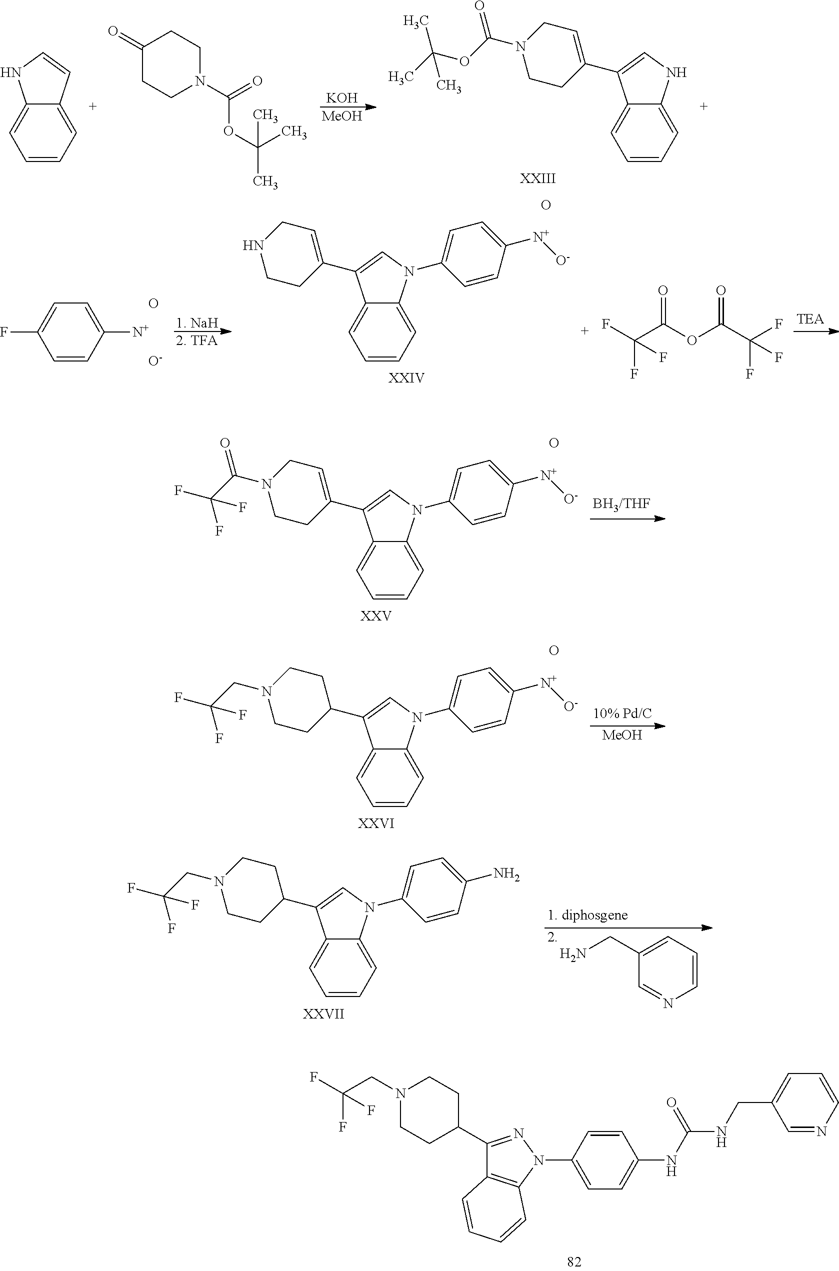

### tert-butyl 4-(1H-indol-3-yl)-3,6-dihydro-2H-pyridine-1-carboxylate (XXIII)

Indole (3.51 g, 30 mmol) and tert-butyl 4-oxopiperidine-1-carboxylate (7.16 g, 36 mmol) were combined with KOH (3.36 g, 60 mmol) in 150 mL MeOH and heated to reflux for 18 hours. Following solvent removal the reaction residue was purified by MPLC [40 g silica: 0→100% EtOAc/hexane. Removal of mobile phase yielded product (3.87 g, 13.0 mmol, 36%) product. Product was identified by LC/MS.

1-(4-nitrophenyl)-3-(1,2,3,6-tetrahydropyridin-4-yl)indole (XXIV): tert-butyl 4-(1H-indol-3-yl)-3,6-dihydro-2H-pyridine-1-carboxylate (XXIII) (600 mg, 2.0 mmol) was dissolved in 10 mL DMF and chilled to 0° C. NaH (60%, 160 mg, 4.0 mmol) was added portion wise and stirred for 30 minutes at room temperature. 1-fluoro-4-nitro-benzene (298 mg, 2.1 mmol) was added and the reaction was stirred for an additional 2 hours at room temperature. Product was isolated by filtration following precipitation with 50 mL H₂O yielding BOC protected product. BOC protected product was taken up in 5 mL CH₂Cl₂ and 1.0 mL TFA and incubated at room temperature for 1 hour. Solvent was removed from the reaction under reduced pressure and the residue was taken up in ethyl acetate. The organic solution was washed with saturated sodium bicarbonate and brine, dried over Na₂SO₄ and concentrated to give product (590 mg, 1.84 mmol, 92%). Product was identified by LC/MS and carried on without further purification.

### 2,2,2-trifluoro-1-[4-[1-(4-nitrophenyl)indol-3-yl]-3,6-dihydro-2H-pyridin-1-yl]ethanone (XXV)

1-(4-nitrophenyl)-3-(1,2,3,6-tetrahydropyridin-4-yl)indole (XXIV) (150 mg, 0.358 mmol) and triethylamine (72.0 mg, 0.716 mmol, 0.100 mL) were combined in CH₂Cl₂ (3.5 mL). Trifluoroacetic anhydride (90.2 mg, 0.429 mmol, 0.060 mL) was added and stirred at room temperature 72 hours (less time is probably sufficient). The reaction was diluted into CH₂Cl₂ (75 mL), washed with saturated sodium bicarbonate and brine, dried over Na2SO4 and concentrated to yield product (assumed quantitative yield). Product was identified by LC/MS and used without further purification.

### 4-[3-[1-(2,2,2-trifluoroethyl)-3,6-dihydro-2H-pyridin-4-yl]indol-1-yl]aniline (XXVII)

2,2,2-trifluoro-1-[4-[1-(4-nitrophenyl)indol-3-yl]-3,6-dihydro-2H-pyridin-1-yl]ethanone (XXV) (assume 0.358 mmol from XXV) was dissolve in THF (3.5 mL). BH₃ in THF (1.0M, 35 mmol) was added. The reaction was allowed to come to room temperature and was then heated to reflux 18 hours. The reaction was cooled to room temperature quenched with MeOH (1.0 mL) and evaporated to dryness. The residue (mainly XXVI) was taken up in MeOH (50 mL) 10% Pd/C (˜50 mg) was added. The reaction was placed on a parr shaker at 50 p.s.i. H₂ gas for 72 hours (less time is probably sufficient). Pd/C was removed by filtration over a bed of celite. Solvent was removed and the residue was purified by MPLC [12 g silica: 0→100% EtOAc/hexane]. Purification yielded product (67.7 mg, 0.181 mmol, 51%).

### 1-(3-pyridylmethyl)-3-[4-[3-[1-(2,2,2-trifluoroethyl)-4-piperidyl]indol-1-yl]phenyl]urea (82)

Compound 82 was prepared in the same manner as described before using 4-[3-[1-(2,2,2-trifluoroethyl)-3,6-dihydro-2H-pyridin-4-yl]indol-1-yl]aniline (XXVII) as the starting material.

Synthetic Method J:

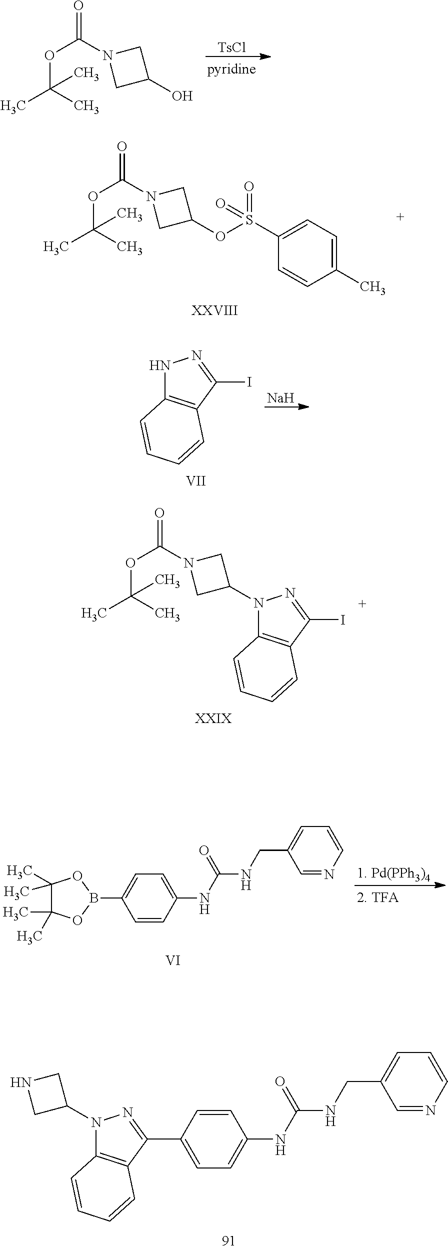

### tert-butyl 3-(p-tolylsulfonyloxy)azetidine-1-carboxylate (XXVIII)

tert-butyl 3-hydroxyazetidine-1-carboxylate (1.73 g, 10.0 mmol) was dissolved in pyridine (10 mL). To this solution was added p-tolylsulfonyl chloride (2.39 g, 12.0 mmol) and placed at −20° C. and incubated for 18 hours. Pyridine.HCl was removed by filtration and the remaining pyridine was removed under reduced pressure. The residue was purified by MPLC [40 g silica: 0→50% EtOAc/hexane]. Solvent removal afforded product (2.27 g, 6.94 mmol, 69%). Product identity was confirmed by GC/MS.

### tert-butyl 3-(3-iodoindazol-1-yl)azetidine-1-carboxylate (XXIX)

3-iodo-1H-indazole (VII) (488 mg, 2.0 mmol) was dissolved in DMF (8.0 mL) and chilled to 0° C. NaH (60%, 393 mg, 9.84 mmol) was added to reaction and stirred for 20 minutes. 1 tert-butyl 3-(p-tolylsulfonyloxy)azetidine-1-carboxylate (XXVIII) (2.44 g, 7.46 mmol) was added in one portion, the reaction was stirred 18 hours at room temperature. The reaction was poured into 40 mL water and the product was isolated by filtration and vacuum drying. The precipitate was purified by MPLC [80 g silica: 0→25% EtOAc/hexane], followed by recrystallization from acetonitrile/chloroform. The product (1.80 g, 4.5 mmol, 61%) was identified by LCMS.

### 1-[4-[1-(azetidin-3-yl)indazol-3-yl]phenyl]-3-(3-pyridylmethyl)urea (91)

Compound 91 was prepared in the same manner as described before, followed by removal of BOC group with TFA.

Synthetic Method K:

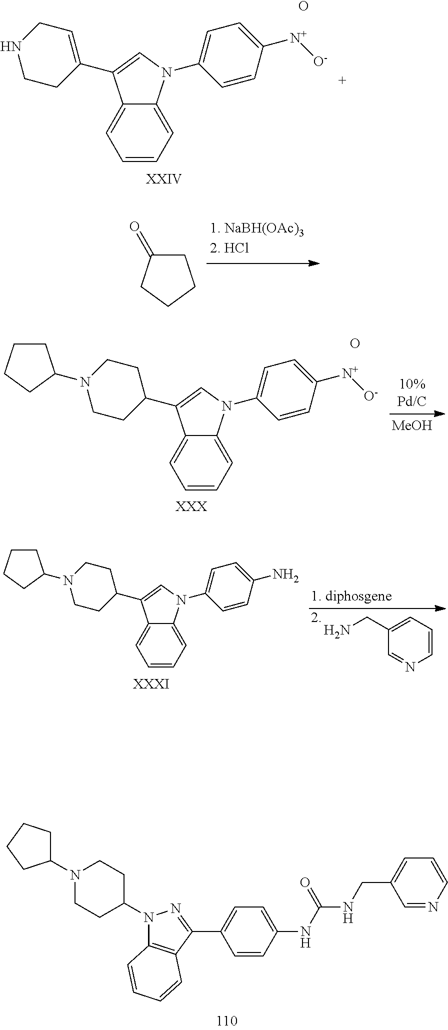

### 3-(1-cyclopentyl-3,6-dihydro-2H-pyridin-4-yl)-1-(4-nitrophenyl)indole (XXX)

1-(4-nitrophenyl)-3-(1,2,3,6-tetrahydropyridin-4-yl)indole (XXIV) (100 mg, 0.313 mmol) and cyclopentane (52.6 mg, 0.625 mmol) were combined in MeOH (5.0 mL). Sodium triacetoxyborohydride (166 mg, 0.783 mmol) was added, the reaction was stirred at room temperature for 18 hours. Reaction solvent was removed under vacuum, the residue was dissolved in ethyl acetate and washed with 1N NaOH, H₂O and brine. The organic phase was dried over Na₂SO₄. Solvent removal afforded desired product which was identified by GC/MS and used without further purification.

### 4-[3-(1-cyclopentyl-4-piperidyl)indol-1-yl]aniline (XXXI)

3-(1-cyclopentyl-3,6-dihydro-2H-pyridin-4-yl)-1-(4-nitrophenyl)indole (XXX) (assume 0.313 mmol) was dissolved in methanol (30 mL) along with CH₃CO₂H (0.20 mL). 10% Pd/C (˜50 mg) was added and reaction was placed in a Parr shaker under 50 p.s.i. H₂ gas for 18 hours. Pd/C was removed by filtration over pad of celite and methanol was removed under reduced pressure. The residue was partitioned between ethyl acetate and 1 N NaOH. The organic layer was washed with water and brine and dried over Na₂SO₄. Removal of solvent afforded product (75.0 mg, 0.209 mmol, 67% over 2 steps).

### 1-[4-[3-(1-cyclopentyl-4-piperidyl)indol-1-yl]phenyl]-3-(3-pyridylmethyl)urea (110)

Compound 110 was prepared in the same manner as before using 4-[3-(1-cyclopentyl-4-piperidyl)indol-1-yl]aniline (XXXI) as the starting material.

Synthetic Method L:

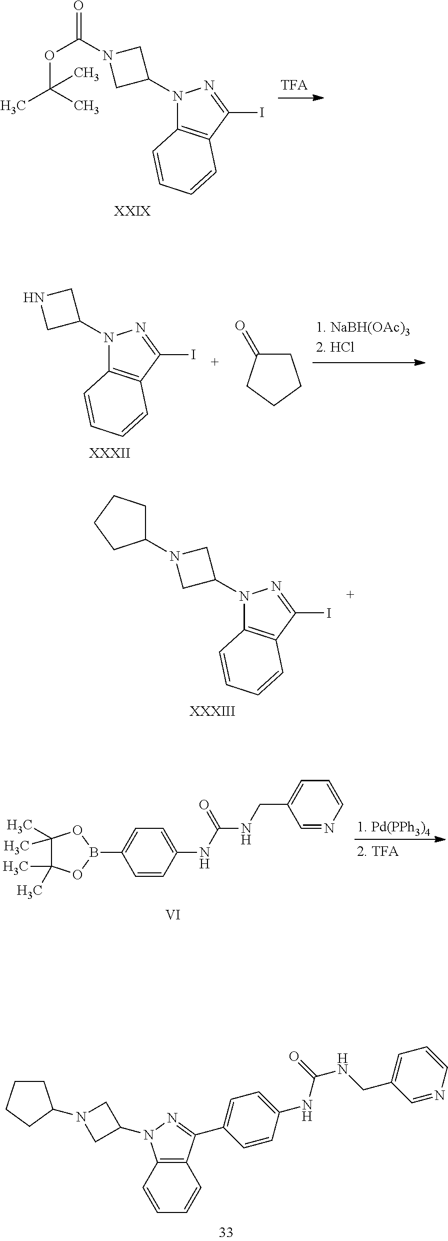

### 1-(1-cyclopentylazetidin-3-yl)-3-iodo-indazole (XXXIII)

XXXIII was prepared by deprotecting the BOC group with TFA to give XXXII and performing reductive amination as was done in the preparation of XXX.

1-[4-[1-(1-cyclopentylazetidin-3-yl)indazol-3-yl]phenyl]-3-(3-pyridylmethyl)urea (33): Compound 33 was prepared in the same manner as before in Synthetic Method B.

Synthetic Method M-1:

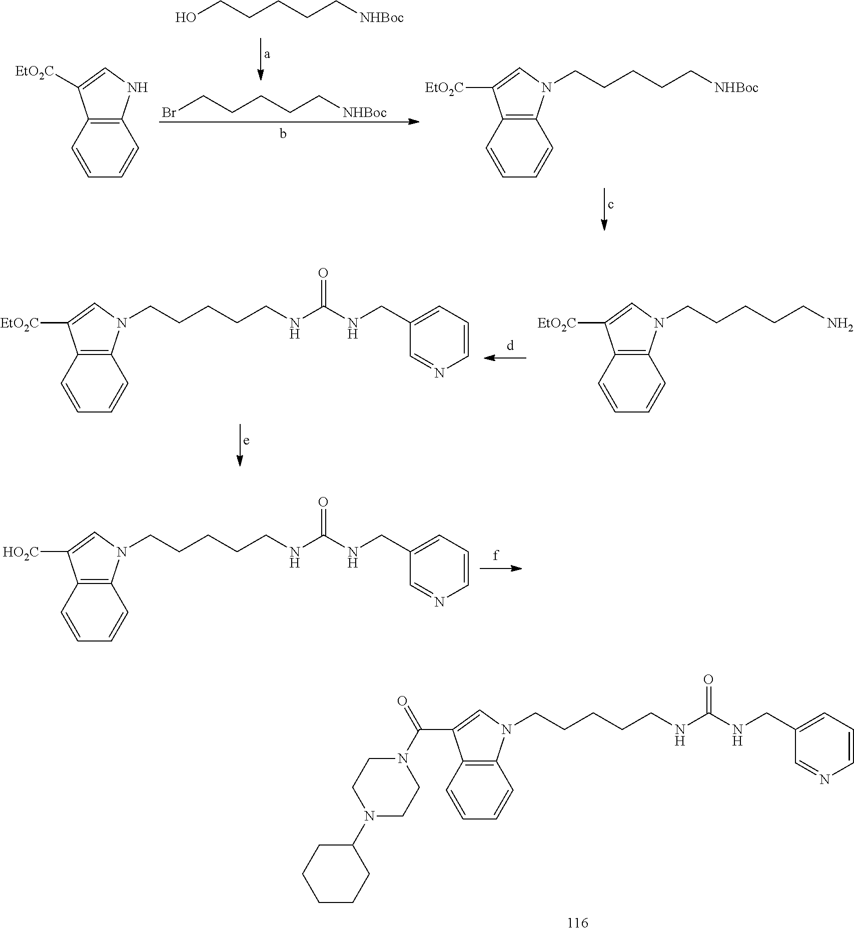

Reagents and conditions: (a) PPh₃, CBr₄, THF, 0° C. to rt; (b) NaH, Bu₄NI, DMF, 0° C. to rt; (c) 4M HCl in dioxane, CH₂Cl₂; (d) diphosgene, NEt₃, CH₂Cl₂, −10° C.; 3-pyridylmethanamine, rt; (e) LiOH monohydrate, THF/MeOH/H₂O, 60° C., 10 h; (f) 1-cyclohexylpiperazine, HATU, Hünig base, DMF, rt, 10 h.

### Step a: tert-butyl N-(5-bromopentyl)carbamate

A solution of 5-bromopentan-1-ol (3.00 g, 14.8 mmol) in THF (41 mL) was cooled to 0° C. and CBr₄ (7.34 g, 22.1 mmol) and PPh₃ (5.96 g, 22.7 mmol) were added. After stirring for 1 h at 0° C., additional CBr₄ (2.55 g, 7.68 mmol) and PPh₃ (2.17 g, 8.27 mmol) were added. The mixture was then stirred for 14 h at rt, diluted with 30% EtOAc/hexane, filtered, and washed with 30% EtOAc/hexane. The combined filtrates were concentrated in vacuo and the residue was purified by column chromatograph (SiO₂, CHCl₃/hexane, 20 to 100%) to afford the target product (3.2 g, 81%).

### Step b: ethyl 1-[5-(tert-butoxycarbonylamino)pentyl]indole-3-carboxylate

To a solution of ethyl 1H-indole-3-carboxylate (1.00 g, 5.28 mmol) in DMF (35 mL) was added NaH (275 mg, 6.87 mmol) at 0° C. After stirring for 0.5 h at the same temperature, Bu₄NI (1.95 g, 5.28 mmol) and tert-butyl N-(5-bromopentyl)carbamate (2.39 g, 8.98 mmol) were added. The mixture was stirred for overnight at rt, quenched with satd. NH₄Cl, extracted with EtOAc, washed with brine, dried (Na₂SO₄), filtered, and concentrated in vacuo. Purification of the crude by column chromatography (SiO₂, EtOAc/hexane, 0 to 100%) provided the product (1.81 g, 87%); LC/MS [M+Na]⁺ 397.3.

### Step c: ethyl 1-(5-aminopentyl)indole-3-carboxylate

To a solution of ethyl 1-[5-(tert-butoxycarbonylamino)pentyl]indole-3-carboxylate (1.81 g, 4.83 mmol) in CH₂Cl₂ (22 mL) was added 4 M HCl in dioxane (22 mL). After stirring for 6 h at rt, the mixture was concentrated in vacuo and the residue was triturated with EA, filtered, and dried to afford the product as a HCl salt (1.4 g, 93%).

### Step d: ethyl 1-[5-(3-pyridylmethylcarbamoylamino)pentyl]indole-3-carboxylate

To a solution of diphosgene (163 mL, 1.35 mmol) in CH₂Cl₂ (10 mL) was added a solution of ethyl 1-(5-aminopentyl)indole-3-carboxylate (700 mg, 2.25 mmol) and NEt₃ (942 ml) in CH₂Cl₂ (12 mL) at −10˜−20° C. and the mixture was stirred for 0.5 h at the same temperature before addition of 3-pyridylmethanamine (458 mL, 4.50 mmol) and NEt₃ (376 ml, 2.76 mmol). The mixture was warmed up to rt, stirred for overnight, quenched with satd. NaHCO₃, washed with brine, dried (Na₂SO₄), filtered, and concentrated in vacuo. Purification of the residue by column chromatography (SiO₂, MeOH/CH₂Cl₂, 1 to 8%) afforded the target product (741 mg, 81%).

### Step e: 1-[5-(3-pyridylmethylcarbamoylamino)pentyl]indole-3-carboxylic acid

To a solution of ethyl 1-[5-(3-pyridylmethylcarbamoylamino)pentyl]indole-3-carboxylate (741 mg, 1.81 mmol) in THF (6 mL) and MeOH (2 mL) was added a solution of LiOH monohydrate (304 mmg, 7.26 mmol) in water (2 mL). The mixture was heated at 60° C. for ˜10 h. After completion, the mixture was concentrated, diluted with water, and washed with Et₂O. The aqueous layer was acidified to pH=˜6 with 1 N HCl and the resulting precipitates were filtered, washed with water and air-dried, yielding the product (585 mg, 85%).

### Step f: 1-[5-[3-(4-cyclohexylpiperazine-1-carbonyl)indol-1-yl]pentyl]-3-(3-pyridylmethyl)urea (116)

To a solution of 1-[5-(3-pyridylmethylcarbamoylamino)pentyl]indole-3-carboxylic acid (80 mg, 0.21 mmol) and HATU (88 mg, 0.23 mmol) in DMF (1.5 mL) was added Hünig base (40 μL, 0.23 mmol) and the mixture was stirred for 5 min at rt before adding 1-cycicohexylpiperizine (39 mg, 0.23 mmol). After stirring for 10 h at rt, the mixture was concentrated in vacuo, diluted with CH₂Cl₂, washed with satd. NaHCO₃, brine, dried (Na₂SO₄), filtered, and concentrated in vacuo. Purification of the crude by column chromatography (SiO₂, MeOH/CH₂Cl₂, 0 to 10%) afforded the product (50 mg, 45%).

Synthetic Method M-2:

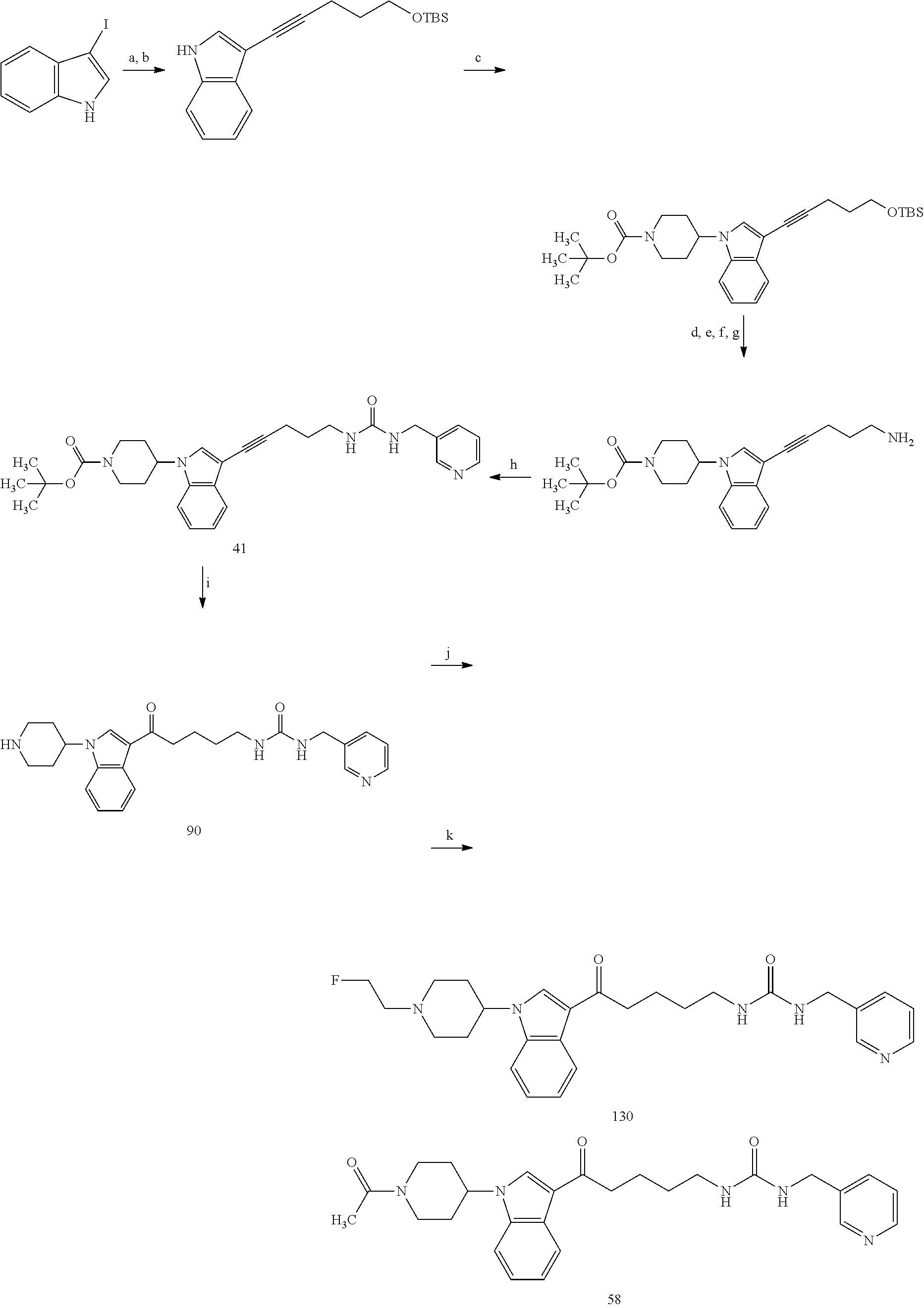

Reagents and conditions: (a) pentyn-4-yn-1-ol, PdCl₂(PPh₃)₂, CuI, NEt₃, rt, 10 h; (b) TBSCl, imidazole, DMF, rt, 10 h; (c) tert-butyl 4-methylsulfonyloxypiperidine-1-carboxylate, NaH, DMF, 95° C.; (d) TBAF (1.0 M in THF), THF, rt; (e) MsCl, NEt₃, CH₂Cl₂, rt; (f) NaN₃, DMF, rt; (g) PPh₃, THF, H₂O, rt; (h) 1,1′-carbonyldiimidazole, 3-aminomethylpyridine, THF, rt; (i) 4 M HCl in dioxane, CH₂Cl₂; (j) 1-bromo-2-fluoroethane, K₂CO₃, CH₃CN, 65° C.; (k) AcOH, HATU, Hünig base, DMF.

### Steps a-b: tert-butyl-[5-(1H-indol-3-yl)pent-4-ynoxy]-dimethyl-silane

To a mixture of 3-iodo-1H-indole (1.35 g, 5.55 mmol) and pentyn-4-yn-1-ol (620 μL, 6.67 mmol) in NEt₃ (18 mL) was added PdCl₂(PPh₃)₂ (156 mg, 0.222 mmol) and CuI (21 mg, 0.11 mmol). After stirring for 10 h, the mixture was diluted with EtOAc, washed with water and brine, dried (Na₂SO₄), filtered and concentrated in vacuo. Purification of the crude by column chromatograpy (SiO₂, EtOAc/hexane, 0 to 80%) provided 5-(1H-indol-3-yl)pent-4-yn-1-ol (170 mg). To a solution of the product in DMF (5 mL) was added imidazole (1.12 g, 16.5 mmol) and TBSC1 (463 mg, 3.07 mmol) and the mixture was stirred at rt for 10 h. After removal of solvent under reduced pressure, the residue was diluted with EtOAc, washed with water and brine, dried (Na₂SO₄), filtered, and concentrated in vacuo. Purification of the residue by column chromatography (SiO₂, EtOAc/hexane, 0 to 50%) afforded the title compound (650 mg).

### Step c: tert-butyl 4-[3-[5-[tert-butyl(dimethyl)silyl]oxypent-1-ynyl]indol-1-yl]piperidine-1-carboxylate

To a solution of tert-butyl-[5-(1H-indol-3-yl)pent-4-ynoxy]-dimethylsilane (650 mg, 2.07 mmol) in DMF (8 mL) was treated with NaH (108 mg, 2.70 mmol, 60% in oil) at 0° C. and the mixture was stirred for 20 min at rt, followed by addition of tert-butyl 4-methylsulfonyloxypiperidine-1-carboxylate (695 mg, 2.49 mmol). The mixture was heated at 95° C. for overnight. LC/MS indicated ˜40% conversion of starting material. The mixture was quenched with satd. NH₄Cl solution, washed with brine, dried (Na₂SO₄), filtered, and concentrated in vacuo. The residue was subjected to the above condition once more, followed by work-up. Purification of the crude by column chromatography (SiO₂, EtOAc/hexane, 0 to 50%) provided the title compound (690 mg).

### Steps d-g: tert-butyl 4-[3-(5-aminopent-1-ynyl)indol-1-yl]piperidine-1-carboxylate

tert-butyl 4-[3-[5-[tert-butyl(dimethyl)silyl]oxypent-1-ynyl]indol-1-yl]piperidine-1-carboxylate

To a solution of tert-butyl 4-[3-[5-[tert-butyl(dimethyl)silyl]oxypent-1-ynyl]indol-1-yl]piperidine-1-carboxylate (690 mg, 1.39 mmol) in THF (15 mL) was treated with TBAF (1.39 mL, 1.39 mmol, 1.0 M in THF). After stirring for 2 h at rt, the mixture was concentrated in vacuo and purified by column chromatography on SiO₂ using a gradient (EtOAc/Hexane, 0 to 50%) to provide the free alcohol (282 mg). To a solution of the alcohol (266 mg, 0.695 mmol) in CH₂Cl₂ (3 mL) was added NEt₃ (290 μL, 2.09 mmol), followed by addition of MsCl (70 μL, 0.90 mmol). After stirring for 1 h at rt, the mixture was diluted with CH₂Cl₂, washed with water and brine, dried (Na₂SO₄), and concentrated in vacuo. The crude was used for the next step without further purification. The crude mesylate (˜0.695 mmol) was dissolved in DMF (2 mL) and added NaN₃ (136 mg, 2.09 mmol). After stirring for 10 h, the mixture was concentrated in vacuo and the residue was diluted with EtOAc, washed with water, brine, dried (Na₂SO₄), and concentrated in vacuo. Purification of the residue by column chromatography (SiO₂, EtOAc/Hex, 0 to 50%) provided the corresponding azide (187 mg). To a solution of the azide (187 mg, 0.460 mmol) in THF (2.3 mL) was added PPh₃ (144 mg, 0.550 mmol). After stirring for 10 min, H₂O (200 μL, 0.920 mmol) was added and the mixture was stirred for overnight. Upon completion, the mixture was concentrated in vacuo and the crude amine was used for the next step without further purification.

### Step h: tert-butyl 4-[3-[5-(3-pyridylmethylcarbamoylamino)pent-1-ynyl]indol-1-yl]piperidine-1-carboxylate

To a solution of 1,1′-carbonyldiimidazole (112 mg, 0.700 mmol) in THF (2 mL) was added 3-aminomethylpyridine (70 μL, 0.70 mmol). After stirring for 10 min, a solution of tert-butyl 4-[3-(5-aminopent-1-ynyl)indol-1-yl]piperidine-1-carboxylate (crude, ˜0.46 mmol) was added and the mixture was stirred for 2 h. Upon completion, the mixture was concentrated, diluted with CH₂Cl₂, washed with water and brine, dried (Na₂SO₄), and concentrated in vacuum. Purification of the residue by column chromatography (SiO₂, MeOH/EtOAc, 0 to 10%) provided the title compound (162 mg).

### Step i: 1-[5-oxo-5-[1-(4-piperidyl)indol-3-yl]pentyl]-3-(3-pyridylmethyl)urea

To a solution of tert-butyl 4-[3-[5-(3-pyridylmethylcarbamoylamino)pent-1-ynyl]indol-1-yl]piperidine-1-carboxylate (160 mg, 0.310 mmol) in CH₂Cl₂ (2 mL) was treated with 4 M HCl (1.2 mL, 4.8 mmol) dropwise at 0° C. After the mixture had been stirred at rt for 6 h, the solvent was decanted and the residue was triturated with EtOAc (20 mL)/THF (1 mL). The solid was collected by filtration, washed with EA and hexane, and air-dried to give the title compound (164 mg); LC/MS [M+H]+ 434.3.

### Step j: 1-[5-[1-[1-(2-fluoroethyl)-4-piperidyl]indol-3-yl]-5-oxo-pentyl]-3-(3-pyridylmethyl)urea

A mixture of 1-[5-oxo-5-[1-(4-piperidyl)indol-3-yl]pentyl]-3-(3-pyridylmethyl)urea (50 mg, 0.10 mmol), 1-bromo-2-fluoroethane (13 μL, 0.19 mmol), and K₂CO₃ (141 mg, 1.02 mmol) in CH₃CN (1.5 mL) was heated at 65° C. for 10 h. After cooling, the mixture was diluted with CH₂Cl₂, washed with brine, dried (Na₂SO₄), filtered and concentrated in vacuo. Purification of the residue by column chromatography (SiO₂, MeOH/CH₂Cl₂, 0 to 15%) provided the title compound (25 mg).

### Step k: 1-[5-[1-(1-acetyl-4-piperidyl)indol-3-yl]-5-oxo-pentyl]-3-(3-pyridylmethyl)urea

To a solution of acetic acid (6.8 μL, 0.12 mmol) and HATU (54 mg, 0.14 mmol) in DMF (0.5 mL) was added Hünig base (25 μL, 4). After stirring for 5 min, a solution of 1-[5-oxo-5-[1-(4-piperidyl)indol-3-yl]pentyl]-3-(3-pyridylmethyl)urea (60 mg, 0.12 mmol) and NEt₃ (33 μL, 0.24 mmol) in DMF (1 mL) was added and the mixture was stirred for overnight. Upon completion, the mixture was concentrated in vacuum, washed with satd. NaHCO₃, brine, dried (Na₂SO₄), filtered, and concentrated in vacuo. Purification of the residue by column chromatography (SiO₂, MeOH/CH₂Cl₂, 0 to 10%) provided the title compound (35 mg).

Synthetic Method M-3

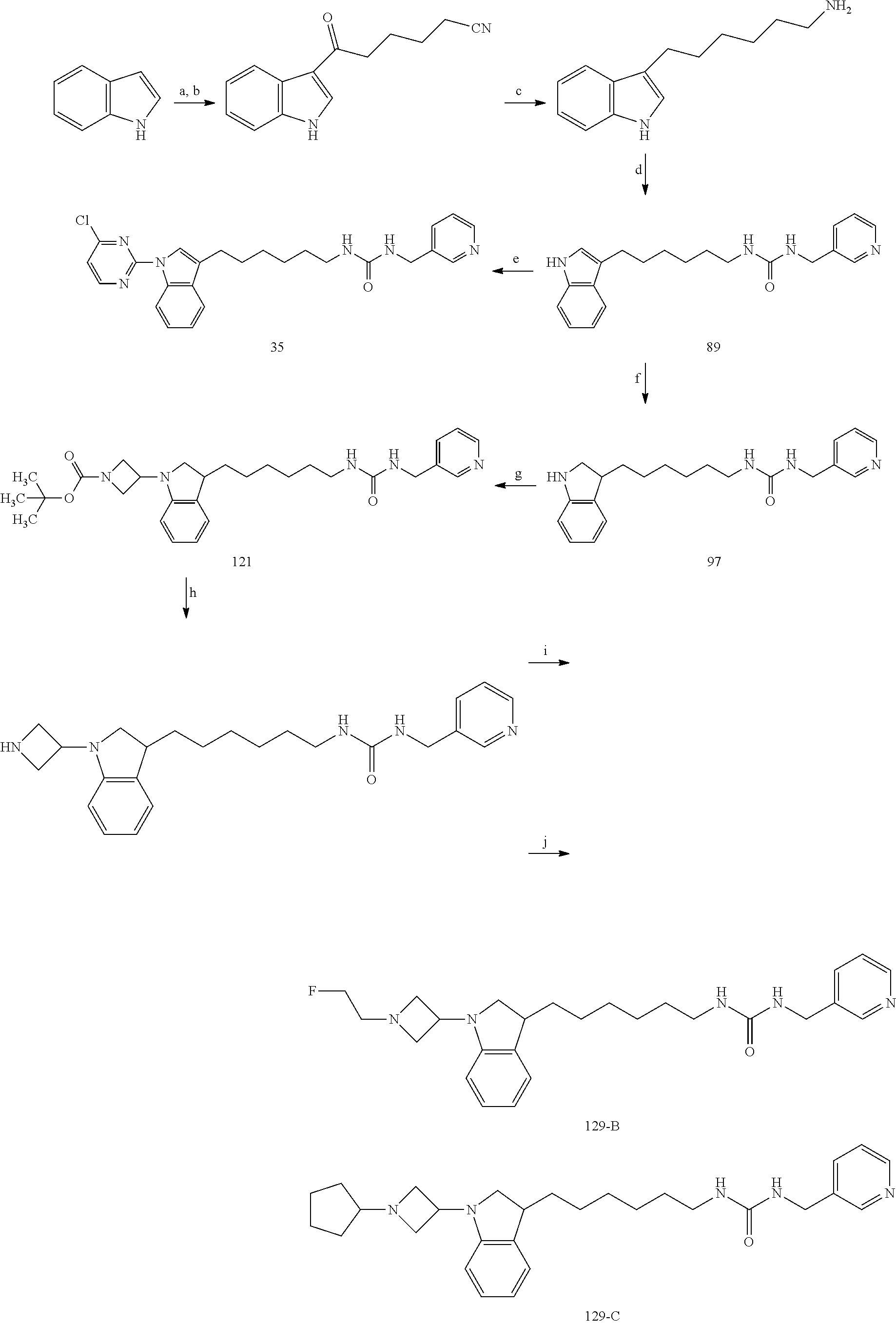

Reagents and Conditions:

(a) 5-chloropentanolyl chloride, A1Cl₃, CH₂Cl₂, 0° C.; (b) NaCN, DMF, 60° C.; (c) LAH, THF, reflux; (d) 1,1′-carbonyldiimidazole, 3-aminomethylpyridine, THF, rt; (e) 2,4-dichloropyrimidine, NaH, DMF, 95° C.; (0 NaBH₃CN, AcOH, rt; (g) t-butyl-3-oxoazetidine-1-carboxylate, NaB(OAc)₃H, AcOH, CH₂Cl₂; (h) 4 M HCl in dioxane, CH₂Cl₂; (i) 1-bromo-2-fluoroethane, K₂CO₃, CH₃CN, 65° C.; (j) cyclopentanone, NaBH(OAc)₃, MeOH, AcOH.

### Steps a-b: 6-(1H-indol-3-yl)-6-oxo-hexanenitrile

To a suspension of AlCl₃ (11.38 g, 85.36 mmol) in CH₂Cl₂ (200 mL) was slowly added 5-chloropentanolyl chloride (11.03 mL, 85.36 mmol) at 0° C. and the mixture was stirred for 0.5 h at 0° C. Indole (10.0 g, 85.4 mmol) was then added portionwise and the mixture was stirred for an additional hour. The mixture was poured into a mixture of c-HCl (63 mL) and cold water (180 mL). The precipitates was collected by filtration and air-dried to give 5-chloro-1-(1H-indol-3-yl)pentan-1-one (14 g).

To a solution of 5-chloro-1-(1H-indol-3-yl)pentan-1-one (3.00 g, 12.7 mmol) in DMF (16 mL) was added NaCN (1.87 g, 38.2 mmol). After heating at 60° C. for 10 h, the mixture was cooled to rt, concentrated in vacuo, and diluted with EtOAc and water. The precipitated solid was collected by filtration, washed with 80:20 EtOAc/hexane and dried to give the title compound (1.5 g).

### Step c: 6-(1H-indol-3-yl)hexan-1-amine

To a solution of 6-(1H-indol-3-yl)-6-oxo-hexanenitrile (1.50 g, 6.63 mmol) in THF (70 mL) was added LAH (1.00 g, 26.5 mmol) at 0° C. The mixture was warmed up to rt, stirred for 10 min, and heated at reflux for 6 h. After cooling to 0° C., the mixture was quenched with H₂O (1 mL), 15% NaOH (1 mL), H₂O (1.5 mL), and diluted with THF (50 mL). After stirring for overnight, the mixture was dried (Na₂SO₄), filtered, washed with THF/CH₂Cl₂, and concentrated in vacuo to give the title compound (1.3 g).

### Step d: 1-[6-(1H-indol-3-yl)hexyl]-3-(3-pyridylmethyl)urea

To a solution of 1,1′-carbonyldiimidazole (1.08 g, 6.63 mmol) in THF (20 mL) was added 3-aminomethylpyridine (675 μL, 6.63 mmol). After stirring for 10 min, a solution of 6-(1H-indol-3-yl)hexan-1-amine (crude, ˜6.63 mmol) in THF (13 mL) was added and the mixture was stirred for 2 h. Upon completion, the mixture was concentrated in vacuo and the residue was purified by column chromatography (SiO₂, MeOH/EtOAc, 0 to 10%) to give the title compound (2.0 g).

### Step e: 1-[6-[1-(4-chloropyrimidin-2-yl)indol-3-yl]hexyl]-3-(3-pyridylmethyl)urea

To a solution of 1-[6-(1H-indol-3-yl)hexyl]-3-(3-pyridylmethyl)urea (70 mg, 0.20 mmol) in DMF (2 mL) was treated with NaH (18 mg, 0.24 mmol). After stirring for 10 min, 2,4-dichloropyrimidine (36 mg, 0.24 mmol) was added and the mixture was heated at 70° C. for 10 h. Upon completion, the mixture was quenched with satd. NH₄Cl. The product portion was extracted with CH₂Cl₂, washed with brine, dried (Na₂SO₄), filtered, and concentrated in vacuo. Purification of the residue by column chromatography (SiO₂, MeOH/EA, 0 to 10%) provided the title compound (34 mg) along with a regioisomer (3 mg) as a minor product.

### Step f: 1-(6-indolin-3-ylhexyl)-3-(3-pyridylmethyl)urea

To a solution of 1-[6-(1H-indol-3-yl)hexyl]-3-(3-pyridylmethyl)urea (200 mg, 0.571 mmol) in AcOH (1.5 mL) was added NaBH₃CN (72 mg, 1.1 mmol) portionwise at 10° C. After stirring for overnight at rt, the mixture was diluted with water and basified with 10 N NaOH. The product portion was extracted with CH₂Cl₂, washed with water and brine, dried (Na₂SO₄), filtered and concentrated in vacuo. Purification of the residue by column chromatography (SiO₂, MeOH/EtOAc, 0 to 10%) provided the title compound (100 mg).

### Step g: tert-butyl 3-[3-[6-(3-pyridylmethylcarbamoylamino)hexyl]indolin-1-yl]azetidine-1-carboxylate

To a solution of 1-(6-indolin-3-ylhexyl)-3-(3-pyridylmethyl)urea (224 mg, 0.636 mmol) in CH₂Cl₂ (3 mL) was added t-butyl-3-oxoazetidine-1-carboxylate (120 mg, 0.699 mmol). After stirring for 20 min at rt, NaBH(OAc)₃ (297 mg, 1.399 mmol) and AcOH (36 μL) were added and the mixture was stirred for 10 h. Upon completion, the mixture was quenched with water, extracted with CH₂Cl₂, dried (Na₂SO₄), filtered and concentrated in vacuo. Purification of the residue by column chromatography (SiO₂, MeOH/CH₂Cl₂, 0 to 10%) provided the title compound (195 mg).

### Step h: 1-[6-[1-(azetidin-3-yl)indolin-3-yl]hexyl]-3-(3-pyridylmethyl)urea

To a solution of tert-butyl 3-[3-[6-(3-pyridylmethylcarbamoylamino)hexyl]indolin-1-yl]azetidine-1-carboxylate (170 mg, 0.335 mmol) in CH₂Cl₂ (3 mL) was added 4 M HCl (1.7 mL) and the mixture was stirred for 2 h at rt. The solvent was decanted and the semi-solid was washed with EA, followed by hexane, and dried to give the title compound (160 mg).

### Step i: 1-[6-[1-[1-(2-fluoroethyl)azetidin-3-yl]indolin-3-yl]hexyl]-3-(3-pyridylmethyl)urea

A mixture of 1-[6-[1-(azetidin-3-yl)indolin-3-yl]hexyl]-3-(3-pyridylmethyl)urea (crude 130 mg, 0.168 mmol) and K₂CO₃ (232 mg, 1.68 mmol) in CH₃CN (2 mL) was heated at 65° C. for overnight. After cooling to rt, the mixture was diluted with CH₂Cl₂, washed with brine, dried (Na₂SO₄), filtered, and concentrated in vacuo. Purification of the residue by column chromatography (SiO₂, MeOH/CH₂Cl₂, 0 to 10%) provided the title compound (35 mg).

### Step j: 1-[6-[1-(1-cyclopentylazetidin-3-yl)indolin-3-yl]hexyl]-3-(3-pyridylmethyl)urea

To a solution of 1-[6-[1-(azetidin-3-yl)indolin-3-yl]hexyl]-3-(3-pyridylmethyl)urea (crude, ˜0.182 mmol) in MeOH (3 mL) was treated with NEt₃ (54 μL), followed by cyclopentanone (19 μL) and AcOH (22 μL). After the mixture had been stirred for 20 min, NaBH(OAc)₃ (81 mg, 0.38 mmol) was added and the mixture was further stirred for 10 h. Upon completion, the mixture was concentrated in vacuo and the residue was purified by column chromatography (SiO₂, MeOH/CH₂Cl₂, 0 to 10%) to give the title compound (42 mg).

Synthetic Method N:

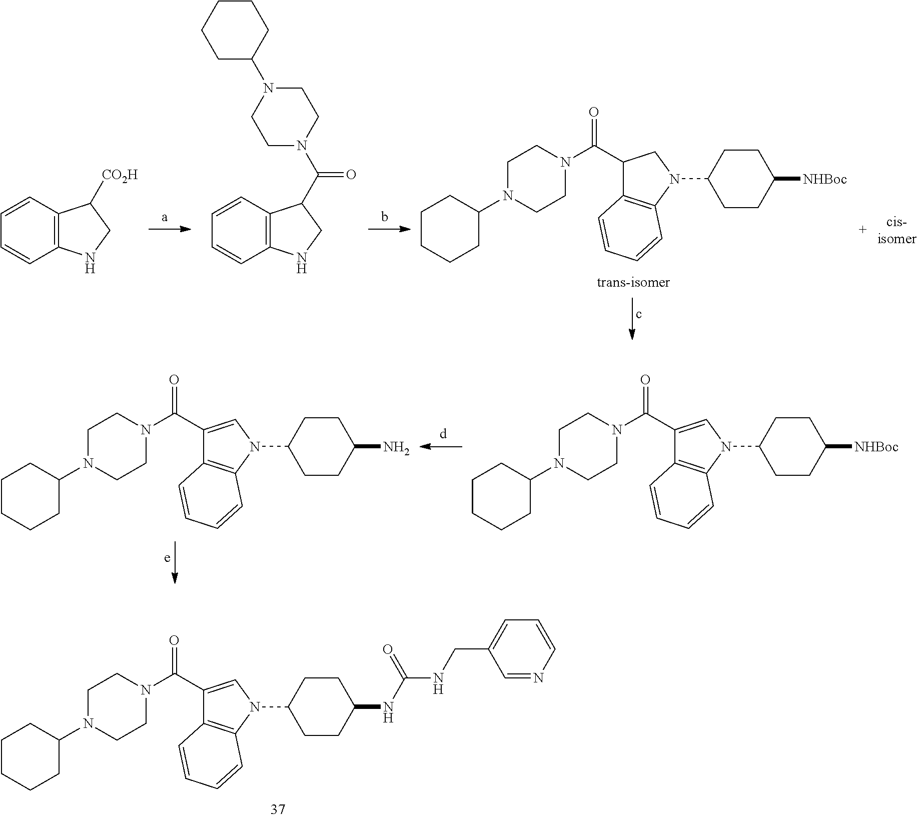

Reagents and conditions: (a) 1-cyclcohexylpiperizine, EDCI, HOBt, NEt₃, DMF, rt, 10 h; (b) tert-butyl N-(4-oxocyclohexyl)carbamate, NaBH(OAc)₃, AcOH, MeOH, rt, 10 h; (b) DDQ, CH₂Cl₂, rt, 2 h; (c) 4 M HCl in dioxane, CH₂Cl₂, rt, 2 h; (d)) diphosgene, NEt₃, CH₂Cl₂, −10° C.; 3-pyridylmethanamine, rt.

### Step a: (4-cyclohexylpiperazin-1-yl)-indolin-3-yl-methanone

To a solution of indoline-3-carboxylic acid (1.00 g, 6.13 mmol), 1-cyclcohexylpiperizine (1.13 g, 6.74 mmol), and HOBt (1.29 g, 6.74 mmol) in DMF (31 mL) was added EDCI (1.29 g, 6.74 mmol), followed by NEt₃ (939 μL). After stirring for 10 h, the mixture was concentrated in vacuo, diluted with CH₂Cl₂, washed with satd. NaHCO₃, brine, dried (Na₂SO₄), filtered, and concentrated in vacuo. Purification of the crude by column chromatography (SiO₂, MeOH/CH₂Cl₂, 0 to 10%) provided the product (1.14 g, 58%).

### Step b: tert-butyl N-[trans-4-[3-(4-cyclohexylpiperazine-1-carbonyl)indolin-1-yl]cyclohexyl]carbamate

To a solution of (4-cyclohexylpiperazin-1-yl)-indolin-3-yl-methanone (573 mg, 1.83 mmol) in MeOH (3 mL) was added tert-butyl N-(4-oxocyclohexyl)carbamate (469 mg, 2.20 mmol) and acetic acid (220 μL, 3.84 mmol) at 0° C. After stirring for 10 min, NaBH(OAc)₃ (815 mg, 3.84 mmol) was added. The resulting mixture was slowly warmed up to rt and stirred for 10 h. The reaction under this condition gave a ˜1:1 mixture of trans and cis-isomers. Upon completion, the mixture was concentrated in vacuo and the crude was purified by column chromatography (SiO₂, MeOH/EtOA, 0 to 3%) to provide the trans-isomer (100 mg), the cis-isomer (120 mg), and a mixture of trans/cis (238 mg).

### Step c: tert-butyl N-[4-[3-(4-cyclohexylpiperazine-1-carbonyl)indol-1-yl]cyclohexyl]carbamate

To a solution of tert-butyl N-[4-[3-(4-cyclohexylpiperazine-1-carbonyl)indolin-1-yl]cyclohexyl]carbamate (95 mg, 0.19 mmol) in CH₂Cl₂ (3 mL) was added DDQ (55 mg, 0.242 mmol) at 0° C. and the mixture was stirred at rt for 2 h. Upon completion, the mixture was diluted with CH₂Cl₂, washed with satd. NaHCO₃, brine, dried (Na₂SO₄), filtered, and concentrated in vacuo. Purification of the crude by column chromatography (SiO₂, MeOH/EtOAc, 0 to 5%) afforded the product (85 mg, 88%).

### Step d: [1-(4-aminocyclohexyl)indol-3-yl]-(4-cyclohexylpiperazin-1-yl)methanone

To a solution of tert-butyl N-[4-[3-(4-cyclohexylpiperazine-1-carbonyl)indol-1-yl]cyclohexyl]carbamate (82 mg, 0.1 6 mmol) in CH₂Cl₂ (2 mL) was added 4 M HCl (1 mL). After stirring for 2 h at rt, the mixture was concentrated in vacuo and the crude was used for the next step.

### Step e: 1-[trans-4-[3-(4-cyclohexylpiperazine-1-carbonyl)indol-1-yl]cyclohexyl]-3-(3-pyridylmethyl)urea (37)

The product (20 mg, 23%) was obtained using [1-(4-aminocyclohexyl)indol-3-yl]-(4-cyclohexylpiperazin-1-yl)methanone (0.16 mmol) as a starting material in a similar manner as shown in General Synthetic Method A (step e).

Compound (1-(cis-4-{3-[(4-Cyclohexylpiperazin-1-yl)carbonyl]-1H-indol-1-yl}cyclohexyl)-3-(pyridin-3-ylmethyl)urea) 34 was synthesized using tert-butyl N-[cis-4-[3-(4-cyclohexylpiperazine-1-carbonyl)indolin-1-yl]cyclohexyl]carbamate in a similar manner to that shown above.

Synthetic Method O:

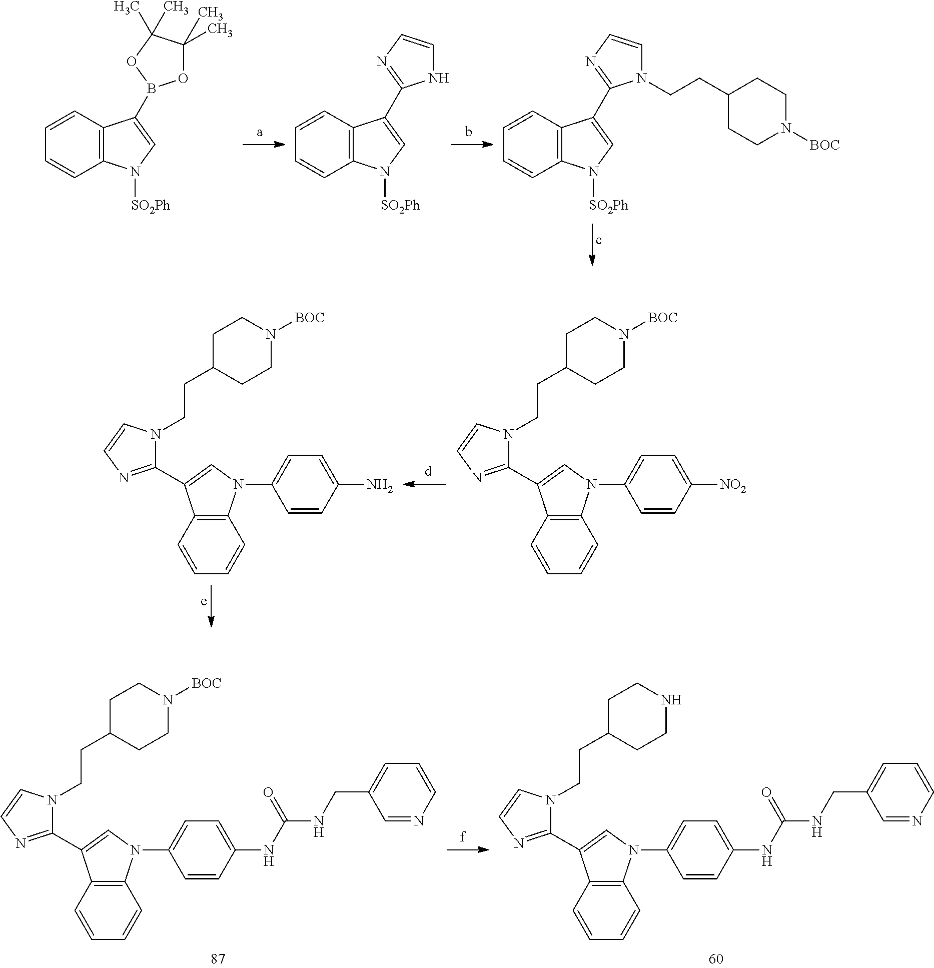

Reagents and conditions: (a) 2-bromo-1H-imidazole, Pd(PPh₃)₄, Na₂CO₃, dioxane/H₂O, 100° C., 10 h; (b) tert-butyl 4-(2-methylsulfonyloxyethyl)piperidine-1-carboxylate, K₂CO₃, DMF, 80° C., 10 h; (c) NaO-tBu, dioxane, 80° C., 2 h; then, 1-fluoro-4-nitro-benzene; (d) Fe, FeSO₄, MeOH, satd. NH₄Cl; (e) diphosgene, NEt₃, 0° C., then 3-pyridylmethanamine, NEt₃, rt; (f) 4M HCl in dioxane, CH₂Cl₂.

### Step a: 1-(benzenesulfonyl)-3-(1H-imidazol-2-yl)indole

1-(Benzenesulfonyl)-3-(4,4,5,5-tetramethyl-1,3,2-dioxaborolan-2-yl)indole (3.10 g, 8.09 mmol), 2-bromo-1H-imidazole (991 mmg, 6.74 mmol) and Na₂CO₃ (2.86 g, 27.0 mmol) were placed in a round flask and a mixture of dioxane (15 mL) and water (7 mL) was charged. After stirring for 10 h at 100° C., the mixture was cooled to rt, diluted with EA, washed with brine, dried (Na₂SO₄), filtered, and concentrated in vacuo. The crude was purified by column chromatography (SiO₂, EA/hexane, 0 to 80%) to afford the product (1.34 g, 74%); LC/MS [M+H]+ 324.1.

### Step b: tert-butyl 4-[2-[2-[1-(benzenesulfonyl)indol-3-yl]imidazol-1-yl]ethyl]piperidine-1-carboxylate

A mixture of 1-(benzenesulfonyl)-3-(1H-imidazol-2-yl)indole (300 mg, 0.928 mmol), tert-butyl 4-(2-methylsulfonyloxyethyl)piperidine-1-carboxylate (570 mg, 1.86 mmol) and K₂CO₃ (385 mg, 2.78 mmol) in DMF (3.5 mL) was heated at 80° C. for 10 h. After concentration, the residue was diluted with EA, washed with water and brine, dried (N₂SO₄), filtered, and concentrated in vacuo. Purification of the crude by column chromatography (SiO₂, EA/hexane, 30 to 100%) provided the product (145 mg, 29%); LC/MS [M+H]+ 534.4.

### Step c: tert-butyl 4-[2-[2-[1-(4-nitrophenyl)indol-3-yl]imidazol-1-yl]ethyl]piperidine-1-carboxylate

To a solution of tert-butyl 4-[2-[2-[1-(benzenesulfonyl)indol-3-yl]imidazol-1-yl]ethyl]piperidine-1-carboxylate (145 mg, 0.271 mmol) in dioxane (2.7 mL) was added NaO-tBu (104 mg, 1.09 mmol) and the mixture was heated at 80° C. for 2 h. After complete deprotection, 1-fluoro-4-nitro-benzene (55 mg, 0.38 mmol) was added and the mixture was stirred at 80° C. for 2-3 h. Upon completion, the mixture was cooled to rt, diluted with EA, washed with brine, dried (Na₂SO₄), and concentrated in vacuo. Purification of the crude by column chromatography (SiO₂, EtOAc/hexane, 50 to 100%) provided the product (127 mg, 91%).

### Step d: tert-butyl 4-[2-[2-[1-(4-aminophenyl)indol-3-yl]imidazol-1-yl]ethyl]piperidine-1-carboxylate

To a solution of tert-butyl 4-[2-[2-[1-(4-nitrophenyl)indol-3-yl]imidazol-1-yl]ethyl]piperidine-1-carboxylate (156 mmg, 0.303 mmol) in MeOH (3 mL) and satd. NH₄Cl (1 mL) was added iron (105 mg, 1.88 mmol) and iron sulfate (31 mg). The mixture was heated at 80° C. for 2 h, cooled to rt, filtered, and washed with CH₂Cl₂. After the combined filtrates were concentrated in vacuo, the residue was dissolved in CH₂Cl₂, washed with brine, dried (Na₂SO₄), filtered, and concentrated in vacuo to afford the product (130 mg) which was used for the next step without further purification.

### Step e: tert-butyl 4-[2-[2-[1-[4-(3-pyridylmethylcarbamoylamino)phenyl]indol-3-yl]imidazol-1-yl]ethyl]piperidine-1-carboxylate

The product (100 mg, 49%) was obtained using tert-butyl 4-[2-[2-[1-(4-aminophenyl)indol-3-yl]imidazol-1-yl]ethyl]piperidine-1-carboxylate (130 mg, 0.337 mmol) as a starting material in a similar manner as shown in General Synthetic Method A (step e).

### Step f: -[4-[3-[1-[2-(4-piperidyl)ethyl]imidazol-2-yl]indol-1-yl]phenyl]-3-(3-pyridylmethyl)urea (60)

To a solution of tert-butyl 4-[2-[2-[1-[4-(3-pyridylmethylcarbamoylamino)phenyl]indol-3-yl]imidazol-1-yl]ethyl]piperidine-1-carboxylate (90 mg, 0.15 mmol) in CH₂Cl₂ (2 mL) was treated with 4 M HCl (730 μL, 2.90 mmol). After stirring for 2 h, the mixture was filtered, washed with Et₂O, dried in a vacuum to afford the product (100 mg) as HCl salt.

Synthetic Method P:

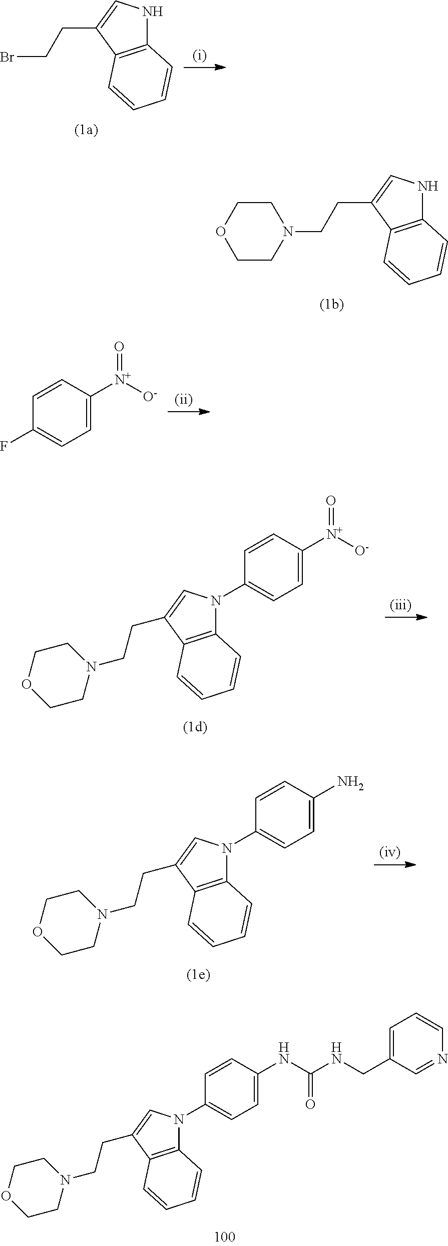 [SMILES: BrCCc1c[nH]c2ccccc12 ; c1ccc2c(CCN3CCOCC3)c[nH]c2c1 ; O=[N+]([O-])c1ccc(F)cc1 ; O=[N+]([O-])c1ccc(-n2cc(CCN3CCOCC3)c3ccccc32)cc1 ; Nc1ccc(-n2cc(CCN3CCOCC3)c3ccccc32)cc1 ; O=C(NCc1cccnc1)Nc1ccc(-n2cc(CCN3CCOCC3)c3ccccc32)cc1]

Reagents & Conditions:

(i) Morpholine, neat, 80 C; (ii) NaH, (1b), DMF; (iii) Zn (dust), Acetic acid; (iv) Diphosgene, 3-pyridylmethanamine, NEt₃, DCM.

### 4-[2-(1H-indol-3-yl)ethyl]morpholine (1b)

To stirred solid 3-(2-bromoethyl)-1H-indole (1a) (1 g) was added morpholine (neat) (2 mL) and heated to 80 C overnight. Excess morpholine from the reaction mixture was evaporated and the residue obtained was chromatographed over silica gel using dichloromethane and methanol as eluent, to get desired product (1b), as off white solid.

### 1-[4-[3-(2-morpholinoethyl)indol-1-yl]phenyl]-3-(3-pyridylmethyl)urea (100)

Employing similar conditions described in Synthetic Method A, intermediate 1b was converted to the final compound 100 in three steps.

Synthetic Method Q:

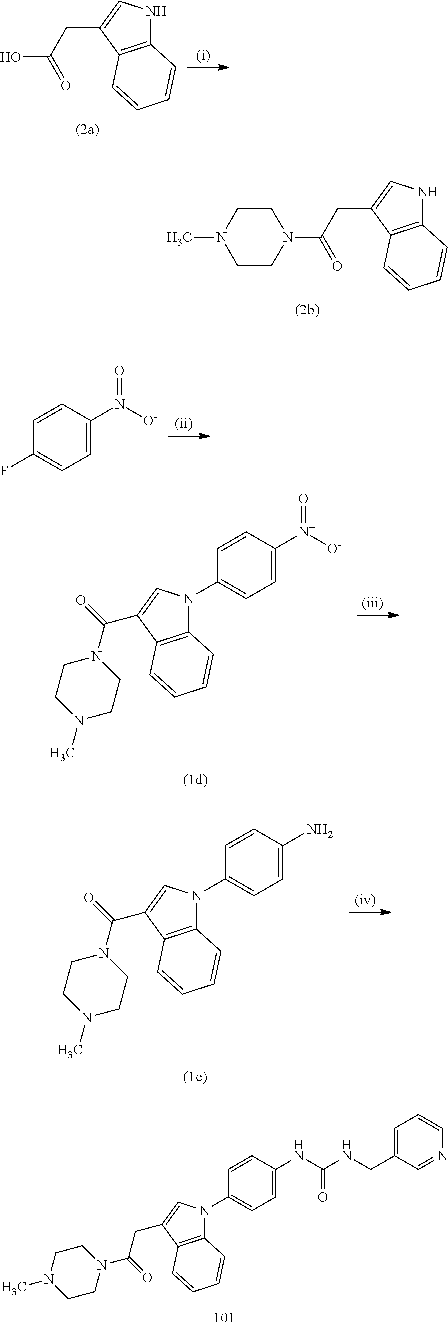 [SMILES: O=C(O)Cc1c[nH]c2ccccc12 ; CN1CCN(C(=O)Cc2c[nH]c3ccccc23)CC1 ; O=[N+]([O-])c1ccc(F)cc1 ; CN1CCN(C(=O)c2cn(-c3ccc([N+](=O)[O-])cc3)c3ccccc23)CC1 ; CN1CCN(C(=O)c2cn(-c3ccc(N)cc3)c3ccccc23)CC1 ; CN1CCN(C(=O)Cc2cn(-c3ccc(NC(=O)NCc4cccnc4)cc3)c3ccccc23)CC1]

Reagents & Conditions

(i) HATU, N-methyl piperazine, DMF; (ii) NaH, (1b), DMF; (iii) Zn (dust), Acetic acid; (iv) Diphosgene, 3-pyridylmethanamine, NEt₃, DCM.

### 2-(1H-indol-3-yl)-1-(4-methylpiperazin-1-yl)ethanone

To a stirred solution of acid (2a) (500 mg, 2.8 mmols) in DMF (15 mL) was added HATU (1 g, 2.8 mmols) and was allowed to be stirred at the room temperature for 30 min. N-methyl piperazine (560 mg, 5.6 mmols) was added and stirred for another 4 hours. The solvent was removed using rotovap and the residue thus obtained, was purified over silica gel using ethyl acetate and hexanes as eluent.

### 1-[4-[3-[2-(4-methylpiperazin-1-yl)-2-oxo-ethyl]indol-1-yl]phenyl]-3-(3-pyridylmethyl)urea (101)

Intermediate (2b) was converted to the final compound 101 in three steps employing similar conditions as described in Synthetic Method A.

Synthetic Method R:

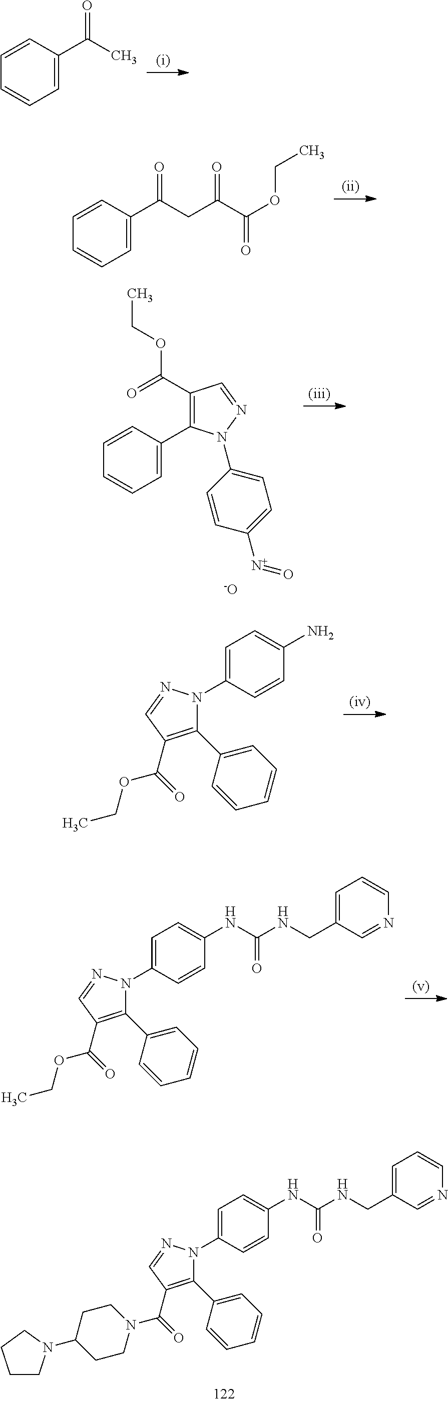

Reagents & Conditions: (i) Diethyl oxalate, K^{+ -}O^{t}Bu, EtOH; (ii) 4-nitropheyl hydrazine; (iii) Zn (dust), Acetic acid; (iv) Diphosgene, 3-pyridylmethanamine, NEt₃, DCM; (v)(a) 4N NaOH, MeOH, THF, 60 C; (b) HATU, 4-pyrrolidin-1-ylpiperidine, DMF.

### Ethyl 1-(4-nitrophenyl)-5-phenyl-pyrazole-4-carboxylate

Prepared according to the similar procedure reported in WO 2007079086. To a stirred solution of acetophenone (1 g, 8.2 mmols) and diethyl oxalate (1.3 g, 9.02 mmols) in EtOH (25 mL) was added potassium tert-butoxide (1 g, 9.02 mmols) at the room temperature. The resulting thick slurry was stirred for additional 2 hours and 4-nitrophenyl hydrazine (1.4 g, 9.03 mmols) in EtOH (10 mL) followed by acetic acid (541 mg, 9.02 mmols) was added. The mixture was stirred at room temperature overnight. Solvent was evaporated and the residue was purified over silica gel using ethyl acetate and hexanes as eluents.

### 1-[4-[5-phenyl-4-(4-pyrrolidin-1-ylpiperidine-1-carbonyl)pyrazol-1-yl]phenyl]-3-(3-pyridylmethyl)urea (122)

Intermediate X was converted in three steps to the final compound 122 employing similar conditions as described in Synthetic Method A.

Synthetic Method S:

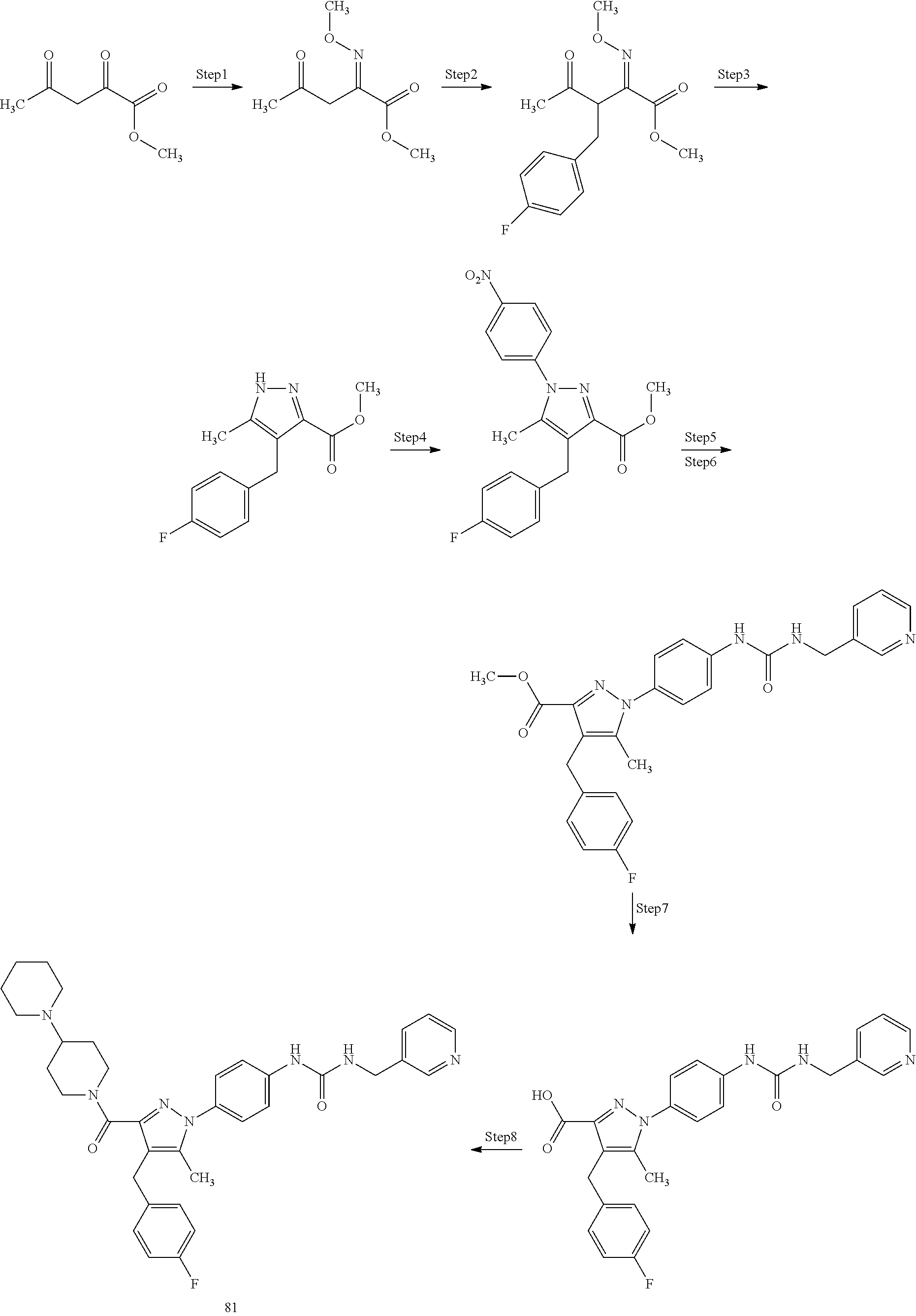

Reagents: 1) MeONH₂.HCl, EtOH, H₂O, RT; 2) BnBr, K₂CO₃, DMF, RT; 3) N₂H₂.H₂O, AcOH, 80° C. 1 hr; 4) NaH, p-F—C₆H₄—NO₂, DMF; 5) Zn, AcOH; 6) CCl₃OCOCl, 3-aminomethyl-pyridine, Et₃N, DCM; 7) aq. NaOH, MeOH, 60° C. 3 hr; 8) HATU, amine, ⁱPr₂EtN, DMF.

Step 1: To a mixture of 2,4-dioxopentanoate (34.69 mmol, 5 g), ethanlool (45 mL) and water (25 mL), a solution of O— methylhydroxylammine hydrochloride (20.81 mmol, 1.7 g) in water (20 mL) added dropwise, and the mixture was stirred at room temperature for 16 hr. The reaction mixture was concentrated under reduced pressure; the residue was diluted with water and extracted with ethyl acetate. The ethyl acetate layer was washed with saturated brine, dried over anhydrous sodium sulfate, evaporated under reduced pressure and the residue was purified by flash column chromatography. The product was eluted with 10% ethyl acetate in hexanes.

Step 2: A suspension of ethyl (2Z)-2-(methoxyimino)-4-oxopentanoate (14.45 mmol, 2.5 g), 4-fluoro-benzyl bromide (14.45 mmol, 1.71 mL), and K₂CO₃ (17.34 mmol, 2.39 g) in DMF (10 mL) was stirred at room temperature for overnight. The mixture was neutralized with dil. HCl and extracted with ethyl acetate, dried over Na₂SO₄, evaporated under reduced pressure and the residue was purified by flash column chromatography. The product was eluted with 25% ethyl acetate in hexanes.

Step 3: To a mixture of step 2 product (3.8 mmol, 1 g) in acetic acid (5 mL) at room temperature, hydrazine monohydrate (4.1 mmol, 133 mg) in 1 mL acetic acid was added, and the reaction mixture was stirred at 80° C. for 1 hr. The reaction mixture was cooled to room temperature, neutralized with aqueous NaHCO₃ solution, the extracted with ethyl acetate. The organic layer was washed with saturated NaCl solution, dried over Na₂SO₄, evaporated under reduced pressure to get the product.

Step 4: To a mixture of pyrazole product obtained in step 3 (4.34 mmol, 1 g) was reacted with 4-fluoro nitro benzene (4.34 mmol, 613 mg) in a similar method explained in Synthetic Method A to get the product.

### Step 5: Nitro reduction using Zn dust in acetic acid.

Step 6: Urea formation as explained in Synthetic Method A.

Step 7: A mixture of step 6 product (0.74 mmol, 337 mg), 10% aq. NaOH solution (7.4 mmol, 296 mg) in MeOH (3 mL) heated at 60° C. for 3 hr. The reaction mixture was cooled to room temperature neutralized with dil. HCl solution, evaporated to dryness under reduced pressure to get the crude product, which was used as such in next reaction.

Step 8: To a mixture of step 7 product (0.45 mmol, 200 mg), di-isopropyl ethyl amine (0.9 mmol, 157 μL), in DMF (5 mL) at room temperature, HATU (0.68 mmol, 258 mg) was added and stirred for 30 min. To the mixture amine (0.9 mmol, 152 mg) was added and the reaction mixture was stirred for 2 hr. The mixture then evaporated to dryness and purified by C-18 flash column.

Method T:

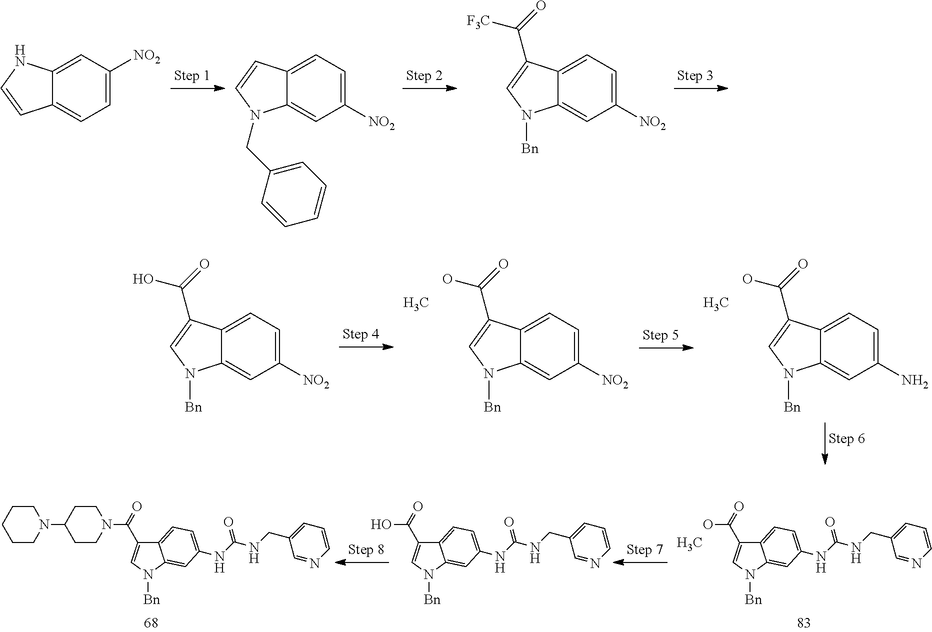

Reagents: 1) NaH, BnBr, DMF; 2) TFA, DMF, 60° C., 3 hr; 3) 20% NaOH-EtOH, reflux, 4 hr; 4) Me₃SICHN₂, MeOH, THF; 5) Zn, AcOH; 6) CCl₃OCOCl, 3-aminomethyl-pyridine, Et₃N, DCM; 7) aq. NaOH, MeOH, 60° C. 3 hr; 8) HATU, amine, ⁱPr₂EtN, DMF.

Step 1: To a mix of 6-nitro-1H-indole in DMF (5 mL) at 0° C., NaH (18.5 mmol, 444 mg) was added and stirred for 15 min. To the mixture benzyl bromide (13.57 mmol, 1.61 mL) was added drop wise and continued stirring for 4 hr. The reaction mixture diluted with water, stirred for 15 min, the solid product was collected by filtration.

Step 2: To the product from step 1 (3.96 mmol, 1 g) in DMF (5 mL), trifluoroacetic anhydride (1.65 mL) was added and the mixture heated at 60° C. for 3 hr. The reaction mixture cooled to room temperature, diluted with ethyl acetate, washed with sat. aq. NaHCO₃, saturated NaCl solution, dried over Na₂SO₄, evaporated under reduced pressure to get the product.

Step 3: The mixture of step 2 product (3.73 mmol, 1.3 g) in 20% NaOH-ethanol solution was refluxed for 4 hr. The reaction mixture diluted with water (5 mL), cooled to 0° C., acidified with con. HCl, evaporated to dryness under reduced pressure. The residue diluted with 5% MeOH in DCM and filtered. The filtrate was evaporated to get the product.

Step 4: To the mixture of step 3 product (0.67 mmol, 200 mg), MeOH (1 mL) and THF (3 mL) at room temperature, Me₃SICHN₂ (1 mL) was added drop wise and the mixture stirred for 3 hr. The reaction mixture was then evaporated to dryness and the residue purified by flash column (40% EtOAc in Hexanes).

Step 5: Nitro reduction using Zn dust in acetic acid.

Step 6: Urea formation as explained in Synthetic Method A.

Step 7: Ester hydrolysis as explained in Synthetic Method S.

Step 8: As explained in Synthetic Method S.

Synthetic Method U:

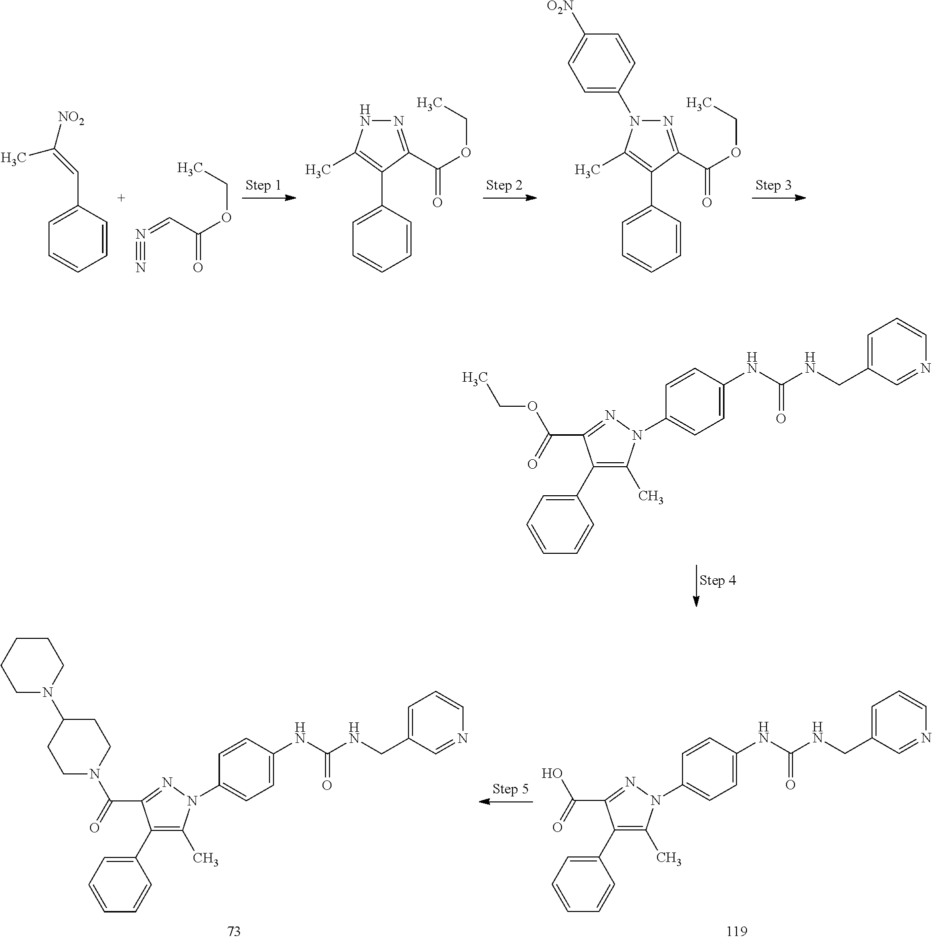

Reagents: 1) Toluene, reflux, 14 hr; 2) NaH, p-F—C₆H₄—NO₂, DMF; 3) Zn, AcOH; 4) CCl₃OCOCl, 3-aminomethyl-pyridine, Et₃N, DCM; 5) aq. NaOH, MeOH, 60° C. 3 hr; 6) HATU, amine, ⁱPr₂EtN, DMF.

Step 1: The mixture of trans-methyl-nitrostyrene (12.25 mmol, 2 g) and ethyldiazoacetate (17.16 mmol, 1.8 mL) in toluene (3 mL) under nitrogen atmosphere, heated to reflux for 14 hr. The solvent was evaporated under reduced pressure and the crude was purified by flash column chromatography (1:1 ethyl acetate and hexane).

Step 2-6: The reactions were done as explained in the above synthetic methods, such as in Synthetic Method S.

Synthetic Method V:

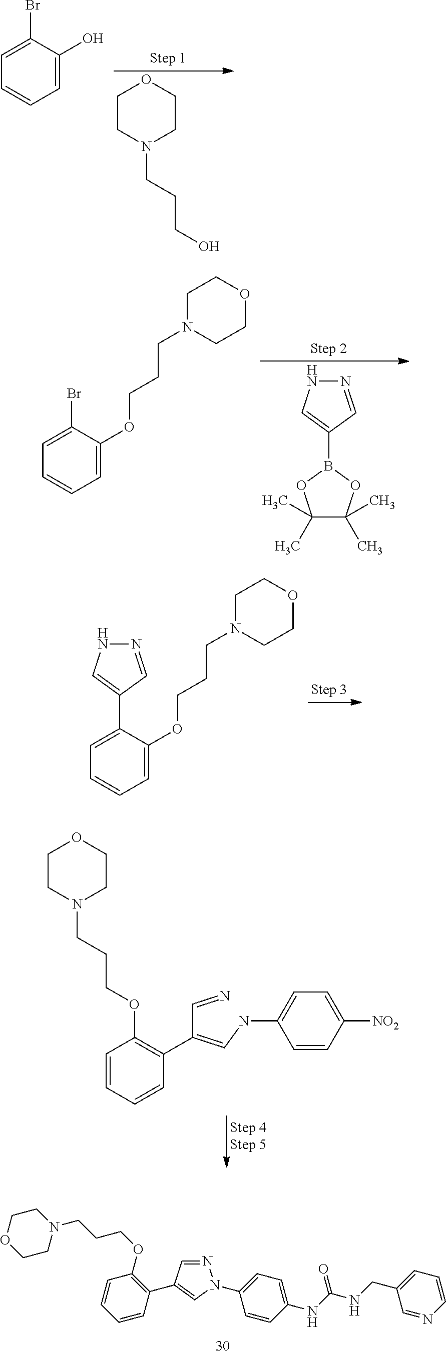

Reagents: 1) DEAD, PPh₃, THF; 2) Pd₂(PPh₃)₄, Na₂CO₃, Dioxane-water (1:1), 90° C.; 3) NaH, p-F—C₆H₄—NO₂, DMF; 4) 10% Pd—C, MeOH, H₂; 5) CCl₃OCOCl, 3-aminomethyl-pyridine, Et₃N, DCM.

Step 1: To the mixture of 2-bromo-phenol (5.78 mmol, 1 g), 3-morpholinopropan-1-ol (5.78 mmol, 838 mg), triphenyl phosphine (6.35 mmol, 1.6 g) in THF (10 mL) at 0° C., DEAD 40% in toluene (6.35 mmol, 1.1 g) was added drop wise. The reaction mixture stirred for overnight at room temperature, evaporated the solvent under reduced pressure and the crude was purified by flash column chromatography using ethyl acetate and hexane mixture. But compound coeluted with triphenyl phosphine oxide, so the mixture was treated with TFA and evaporated to dryness, and purified by reverse phase chromatography using water-AcNC mixture gradient.

Step 2: The mixture of step 1 product as TFA salt (3.62 mmol, 1.5 g), the corresponding borane ester (3.98 mmol, 773 mg), Na₂CO₃ (18.11 mmol, 1.92 g) in Dioxane-Water (1:1) (10 mL) was purged with nitrogen gas. To the mixture Pd₂(PPh₃)₄ (0.18 mmol, 209 mg) was added, the mixture was purged with nitrogen gas, stirred at 90° C. for overnight. The mixture cooled to room temperature, filtered through celite, solvents were evaporated and purified by flash column chromatography using 1:20 MeOH-DCM mixture.

Step 3-5: The reactions were done as explained in Synthetic Method A.

Synthetic Method W:

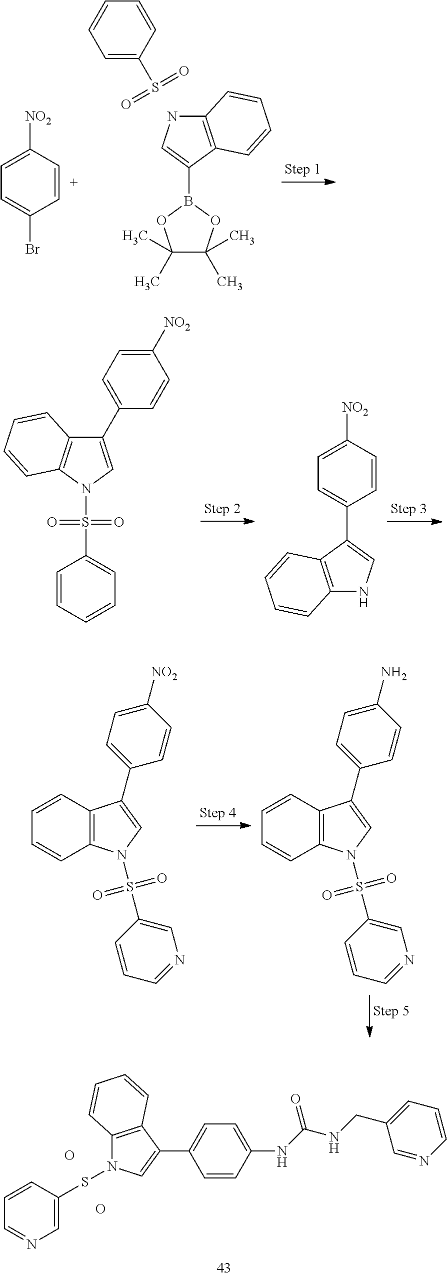

Reagents: 1) Pd₂(PPh₃)₄, Na₂CO₃, Dioxane-water (1:1), 90° C.; 2) aq. NaOH, MeOH, 60° C. 16 hr; 3) pyridine-3-sulfonyl chloride; 4) 10% Pd—C, MeOH, H₂; 5) CCl₃OCOCl, 3-aminomethyl-pyridine, Et₃N, DCM.

Step 1-5: The reactions were done similar to as explained in previous examples.

Synthetic Method X:

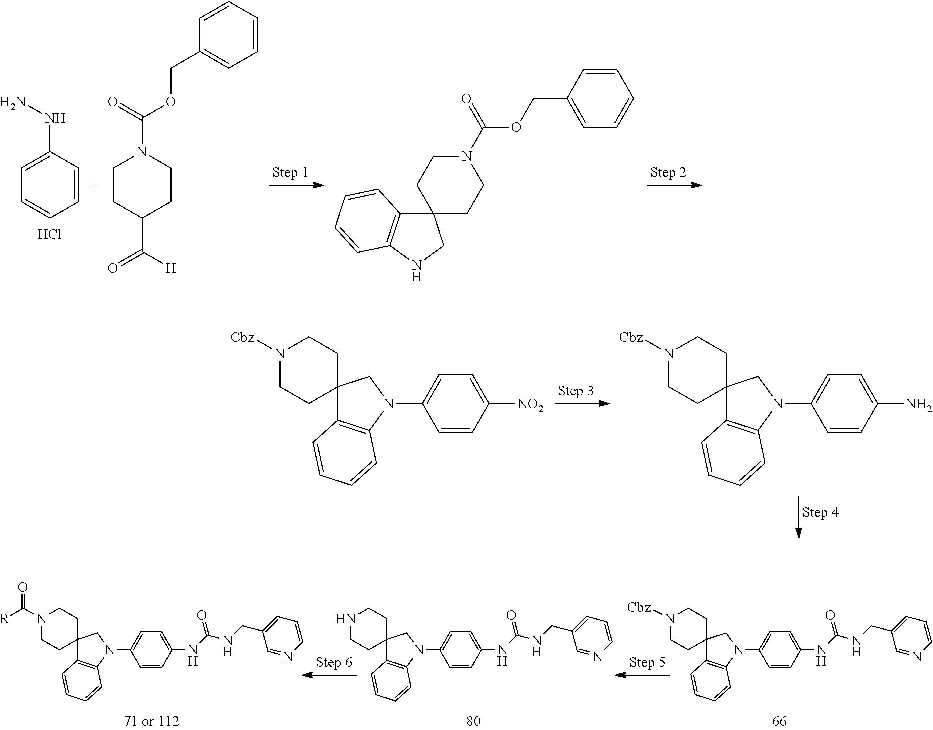

Reagents: 1) TFA, AcCN-Tol, NaBH₄, MeOH; 2) NaH, p-F—C₆H₄—NO₂, DMF; 3) Zn, AcOH; 4) CCl₃OCOCl, 3-aminomethyl-pyridine, Et₃N, DCM; 5) 10% Pd—C, MeOH, H₂; 6) RCOOH, HATU, ⁱPr₂EtN, DMF (or) RCOCl, Et₃N, THF.

Step 1: To a mixture of 4-fluorophenylhydrazine hydrochloride (13.36 mmol, 1.93 g), trifluoroacetic acid (40.06 mmol, 2.97 mL) in acetonitrile:toluene (49:1, 25 mL), a solution of benzyl 4-formylpiperidine-1-carboxylate (12.14 mmol, 3 g) in acetonitrile:toluene (49:1, 10 mL) was added drop wise at room temperature. The reaction mixture was stirred at 35° C. for 16 hr followed by 5 hr at 50° C. Then the reaction mixture cooled to 0° C., diluted with 3 mL MeOH, added NaBH₄ (18.21 mmol, 688 mg) and continued stirring at room temperature for 3 hr. The reaction mixture was then diluted with sat. aq. NaHCO₃ solution, extracted with ethyl acetate, dried over anhydrous Na₂SO₄, evaporated under reduced pressure and the residue was purified by flash column chromatography. The product eluted at 1:2 hexane:ethyl acetate solvent mixture.

Step 2-4: The reactions were done as explained for Synthetic Method S.

Step 5: The mixture of step 4 product (0.31 mmol, 170 mg), 10% Pd—C (20 mg) in ethanol (5 mL), stirred under hydrogen gas balloon atmosphere for over night. The reaction mixture filtered through celite bed, washed with MeOH, solvents evaporated to get the product.

Step 6: Method 1: Compound 71: To the mixture of step 5 product (0.25 mmol, 106 mg), triethylamine (0.64 mmol, 89 μL) in THF (3 mL) was added (S)-2-acetoxy propionyl chloride (0.3 mmol, 39 μL) and the mixture stirred for overnight. The reaction mixture was diluted with ethyl acetate, washed with sat. aq. NaHCO₃ solution, saturated NaCl solution, dried over Na₂SO₄, evaporated under reduced pressure. The residue obtained was diluted with MeOH (3 mL), added K₂CO₃ (50 mg) and stirred at room temperature for 4 hr. The solvent was evaporated and purified by reverse phase flash column.

Step 6: Method 2: Compound 112: To a mixture of step 5 product (0.26 mmol, 109 mg), diisopropyl ethyl amine (0.52 mmol, 91 μL) in DMF (2 mL), at room temperature HATU (0.39 mmol, 150 mg) was added was added and stirred for 30 min. To the mixture amine (0.9 mmol, 152 mg) was added and the reaction mixture was stirred for 2 hr. The mixture then evaporated to dryness and purified by C-18 flash column.

Synthetic Method Y:

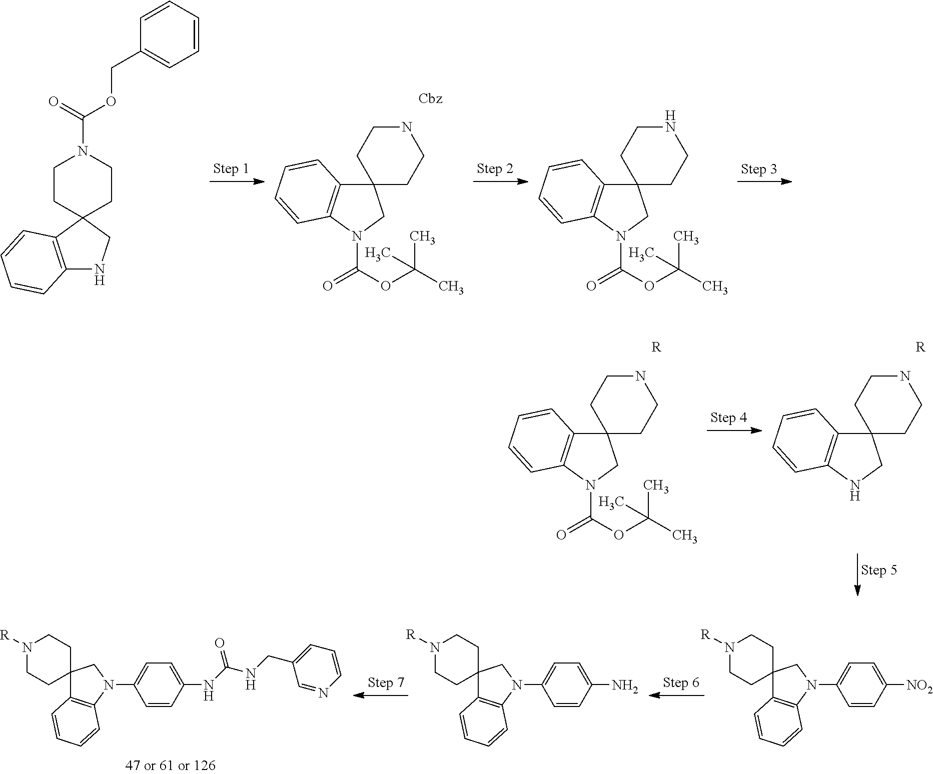

Reagents: 1) (BOC)₂O, NaH, DMF; 2) 10% Pd—C, MeOH, H₂; 3) R—Br, ⁱPr₂EtN, DMF (or) R═O, MeOH, NaCNBH₄; 4) TFA, DCM; 5) NaH, p-F—C₆H₄—NO₂, DMF; 6) Zn, AcOH; 7) CCl₃OCOCl, 3-aminomethyl-pyridine, Et₃N, DCM;

Step 1: To a solution of spiroindole (3.32 mmol, 1.07 g) in DMF (5 mL) at 0° C., NaH (4.98 mmol, 119 mg) added and stirred for 10 min. Then to the mixture boc-anhydride (4.31 mmol, 942 mg) was added and stirred for 4 hr. The reaction mixture diluted with water (50 mL), extracted with ethylacetate (2×50 mL), the ethylacetate layer was washed with brine, dried over Na₂SO₄, evaporated under reduced pressure and the crude was purified by flash column chromatography.

Step 2: CBZ-deprotection.

Step 3: Method 1: A mixture of step 2 product (1.14 mmol, 330 mg), isopropyl bromide (1.71 mmol, 161 μL) and diisopropyl ethyl amine (0.3.43 mmol, 598 μL) in DMF (2 mL) stirred over night at room temperature. The reaction mixture diluted with water (30 mL), extracted with ethylacetate (2×30 mL), the ethylacetate layer was washed with brine, dried over Na₂SO₄, evaporated under reduced pressure to get the product.

Step 3: Method 2: To a mixture of step 2 product (1.90 mmol, 550 mg), cyclopentanone (2.10 mmol, 185 μL) in MeOH (5 mL) at room temperature NaCNBH₃ (1.90 mmol, 120 mg) was added portions wise and the stirred for over night. The reaction mixture evaporated to dryness, diluted with ethylacetate (50 mL), washed with 10% aq. NaOH solution, water and brine. The ethylacetate layer dried over Na₂SO₄, evaporated under reduced pressure to get the product.

Step 4: The mixture of step 3 product (1 mmol) in DCM (2 mL) and TFA (1 mL) stirred at room temperature for 2 hr, then evaporated under reduced pressure to get the product as TFA salt.

Step 5-7: The reactions were done as explained earlier, such as in Synthetic Method A.

Synthetic Method Z:

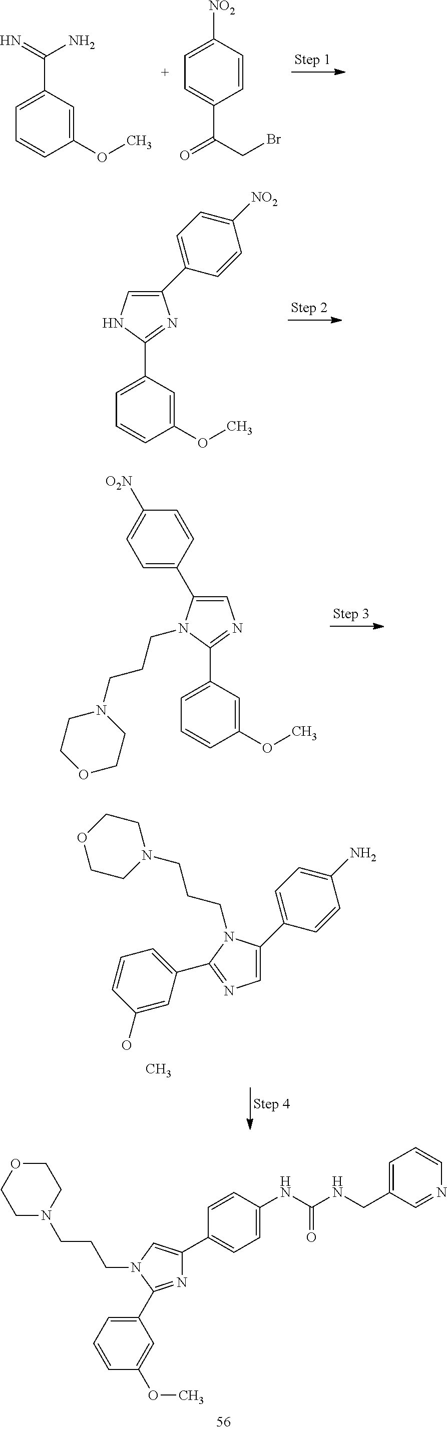

Reagents: 1) NaHCO₃, THF—H₂O (5:2), reflux; 2) 3-morpholinopropyl methanesulfonate, CsCO₃, DMF; 3) 10% Pd—C, MeOH, H₂; 4) CCl₃OCOCl, 3-aminomethyl-pyridine, Et₃N, DCM;

Step 1: To a solution of 3-methoxylbezamidine (10.71 mmol, 2 g) in THF—H₂O (5:2, 15 mL), was added NaHCO₃ (42.86 mmol, 3.6 g) and the solution was brought to a vigorous rreflux. A solution of 4-nitrophenacyl bromide (10.71 mmol, 2.61 g) in dry THF (5 mL) was added dropwise and the solution heated at reflux for 2 hr. The mixture was cooled and the THF was removed under reduced pressure, the residue was diluted with DCM, stirred for 5 min, filtered through celite bed and washed with DCM. The combined DCM was evaporated to get the product.

Step 2: To the mixture of step 1 product (1.69 mmol, 500 mg), CsCO₃ (5.08 mmol, 1.65 mg) in DMF (5 mL) at room temperature, added 3-morpholinopropyl methanesulfonate (3.38 mmol, 755 mg) and stirred for over night. The reaction mixture was diluted with ethyl acetate, washed with water, brine, dried over Na₂SO₄, evaporated under reduced pressure and the residue was purified by flash column chromatography.

Step 3-4: The reactions were done as explained in earlier, such as in Synthetic Method A.

Synthetic Method 1

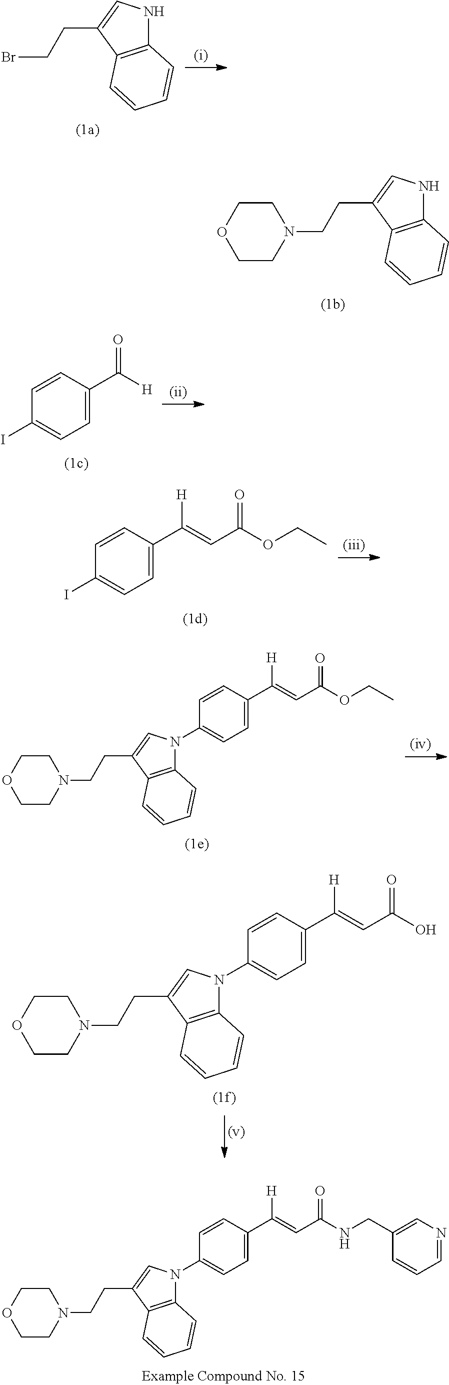

In general, many compounds of the present invention can be synthesized in a manner similar to Synthetic Method 1. Synthetic Method 1: Reagents & Conditions: (i) Morpholine (neat), 80° C.; (ii) Ethyl Phosphonoacetate, NaH, THF, RT; (iii) 4-[2-(1H-indol-3-yl)ethyl]morpholine, CuI, trans-1,2-cyclohexyldiamine, DMF, 110° C.; (iv) 1 N NaOH, THF, MeOH; and (v) HATU, DMF, 3-pyridylmethanamine.

### 4-[2-(1H-indol-3-yl)ethyl]morpholine (1b)

To stirred solid 3-(2-bromoethyl)-1H-indole (1a) was added morpholine (neat) (2 mL) and heated to 80° C. overnight. Excess morpholine from the reaction mixture was evaporated and the residue obtained was chromatographed over silica gel using dichloromethane and methanol as eluent, to get desired product (1b), as off-white solid.

### Ethyl (E)-3-(4-iodophenyl)prop-2-enoate (1d)

To a stirred suspension of sodium hydride (517 mg, 12.9 mmols) in THF (50 mL) at room temperature was added triethyl phosphonoacetate (2.9 g, 12.9 mmols) and was allowed to be stirred at the room temperature for 30 min or until the solution was clear. A solution of 4-iodobenzaldehyde (1c) (2 g, 8.62 mmol) in THF (30 mL) was added to the reaction mixture and was allowed to be stirred at the room temperature overnight. Reaction mixture was diluted with water (10 mL) was added and extracted with ethyl acetate (2×60 mL). Organic layer was washed with brine and dried over anhydrous sodium sulfate. Evaporation of the solvent yielded oily residue, which was chromatographed over silica gel using ethyl acetate and hexanes as eluent to get ethyl (E)-3-(4-iodophenyl)prop-2-enoate (1d), as an off-white solid.

### Ethyl (E)-3-[4-[3-(2-morpholinoethyl)indol-1-yl]phenyl]prop-2-enoate (1e)

To a stirred solution of 4-[2-(1H-indol-3-yl)ethyl]morpholine (411 mg, 1.78 mmols) and ethyl (E)-3-(4-iodophenyl)prop-2-enoate (539 mg, 1.78 mmols) in DMF (10 mL) was added cuprous iodide (34 mg, 0.178 mmols) and tripotassium phosphate (755 mg, 3.56 mmols) and flushed with nitrogen. Trans-cyclohexyldiamine (22 μl, 0.178 mmols) was added and the reaction mixture was allowed to be stirred at 110° C. over 12-16 hrs. The solvent was evaporated using a rotary evaporator and diluted with water and extracted with ethyl acetate. The organic layer was washed with brine and dried over anhydrous sodium sulfate. The residue obtained after evaporation of the solvent, was chromatographed over silica gel using dichloromethane and methanol as eluents to get the title compound as oil.

### (E)-3-[4-[3-(2-morpholinoethyl)indol-1-yl]phenyl]-N-(3-pyridylmethyl)prop-2-enamide (example compound no. 15)

To a stirred solution of compound (1e) (100 mg) in methanol (4 mL) and THF (4 mL) was added 2 N NaOH (2 mL) and heated at 60° C. over 3 hrs. Reaction solvent was evaporated on the rotary evaporator and the residue thus obtained, was purified over C-18 reverse phase ISCO using water (with 0.1% TFA) and MeOH (with 0.1% TFA) as eluents. Pure fractions were collected and rotary evaporated to get the desired acid (1f). The acid (70 mg, 0.186 mmols) was dissolved in DMF (5 mL) and HATU (106 mg, 0.279 mmols) was added and stirred at room temperature for 30 min. 3-pyridylmethanamine (40 mg, 0.372 mmols) was added to the reaction mixture and allowed to be stirred for another 4 hrs. Solvent from the reaction mixture was evaporated and the residue obtained was purified on C-18 reverse phase ISCO, as described for the acid above, to get the desired product (example compound no. 15) as an off-white solid.

Synthetic Method 2

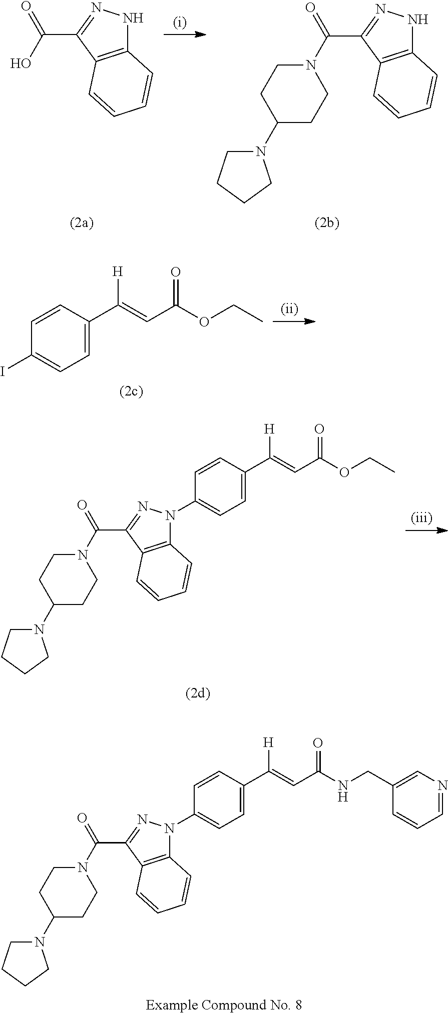 [SMILES: O=C(O)c1n[nH]c2ccccc12 ; O=C(c1n[nH]c2ccccc12)N1CCC(N2CCCC2)CC1 ; CCOC(=O)/C=C/c1ccc(I)cc1 ; CCOC(=O)/C=C/c1ccc(-n2nc(C(=O)N3CCC(N4CCCC4)CC3)c3ccccc32)cc1 ; O=C(/C=C/c1ccc(-n2nc(C(=O)N3CCC(N4CCCC4)CC3)c3ccccc32)cc1)NCc1cccnc1]

Additionally, many other compounds of the present invention can be synthesized in a manner similar to Synthetic Method 2.

Synthetic Method 2: Reagents & Conditions: (i) HATU, 4-pyrrolidin-1-ylpiperidine, DMF; (ii) 1H-indazol-3-yl-(4-pyrrolidin-1-yl-1-piperidyl)methanone, CuI, trans-1,2-cyclohexyldiamine, DMF, 110° C.; (iii) (a) 1 N NaOH, THF, MeOH; (b) HATU, DMF, 3-pyridylmethanamine.

### 1H-indazol-3-yl-(4-pyrrolidin-1-yl-1-piperidyl)methanone (2b)

To a stirred solution of commercially-available acid (2a) (500 mg, 3.06 mmols) in DMF (25 mL) was added HATU (1.74 gm, 4.56 mmols) and stirred at room temperature for 30 min. 4-pyrrolidin-1-ylpiperidine (772 mg, 4.59 mmols) was added to the reaction mixture and stirring was continued for additional 4 hours. Solvent from the reaction mixture was evaporated on a rotary evaporator and the residue thus obtained, was chromatographed on the silica gel using dichloromethane and methanol as eluents, to get the desired product (2b) as viscous oil.

Ethyl (E)-3-[4-[3-(4-pyrrolidin-1-ylpiperidine-1-carbonyl)indazol-1-yl]phenyl]prop-2-enoate (2d)

Prepared according to the procedure described for the compound (1e) (Synthetic Method 1) using (2b) and (2c).

### (E)-N-(3-pyridylmethyl)-3-[4-[3-(4-pyrrolidin-1-ylpiperidine-1-carbonyl)indazol-1-yl]phenyl]prop-2-enamide (Example Compound No. 8)

The compound (2d) was converted to (2e), employing similar conditions as described for example compound no. 8 in the Synthetic Method 1.

Exemplary compounds of the present invention are shown in Table 1, 2, 3, 4, 5, 8, and 9. Table 1 is separated into an “A” and “B”, but is referred to throughout the Specification as “Table 1”. Table 1A shows the structure, name, and synthetic method for a particular example compound. Compound names were generated using Symyx® Draw version 3.3.NET to generate IUPAC names (Accelrys, Inc., San Diego, Calif.). Table 1B shoes the High Performance Liquid Chromatography (“HPLC”) retention time, molecular weight found using High Resolution Mass Spectrometry (“HRMS”), and proton Nuclear Magnetic Resonance (“NMR”) for a particular example compound. Tables 6 and 7 provide IUPAC names, HPLC retention times, molecular weights, and NMR data for certain example compounds. In most instances, the Synthetic Method listed is similar to the procedure actually used to make a particular example compound, rather than the actual procedure used. Each of the example compounds was synthesized using commercially available starting materials that are well known in the art. Tables 3, 4, 5, and 9 include Example compounds that were never made.

Regarding Table 8, Example compound number 168 was made according to Synthetic Method 2. Example compound number 175 was made according to Synthetic Method 1. The other example compounds of Table 8 were made in a manner similar to Synthetic Methods 1 and 2.

### Example Compounds

TABLE 1A

| Example | | | |
|---|---|---|---|
| Comp'd | | | Syn. |
| Number | Structure | IUPAC Name | Method |
| 1 | 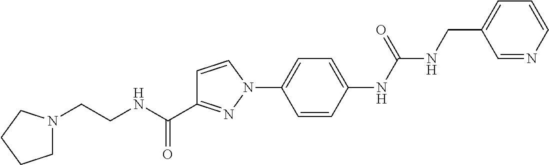 [SMILES: O=C(NCc1cccnc1)Nc1ccc(-n2ccc(C(=O)NCCN3CCCC3)n2)cc1] | 1-(4-{[(Pyridin-3- ylmethyl)carbamoyl]amino} phenyl)-N-[2- (pyrrolidin-1-yl)ethyl]- 1H-pyrazole-3- carboxamide | A |
| 2 | 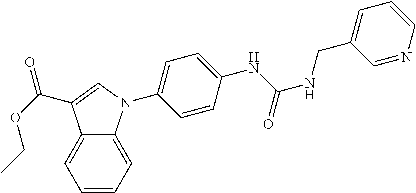 [SMILES: CCOC(=O)c1cn(-c2ccc(NC(=O)NCc3cccnc3)cc2)c2ccccc12] | Ethyl 1-(4-{[(pyridin-3- ylmethyl)carbamoyl]amino} phenyl)-1H-indole-3- carboxylate | A |
| 3 | 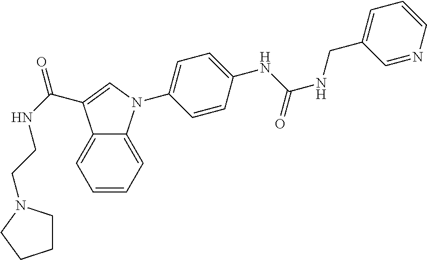 [SMILES: O=C(NCc1cccnc1)Nc1ccc(-n2cc(C(=O)NCCN3CCCC3)c3ccccc32)cc1] | 1-(4-{[(Pyridin-3- ylmethyl)carbamoyl]amino} phenyl)-N-[2- (pyrrolidin-1-yl)ethyl]- 1H-indole-3-carboxamide | A |
| 4 | 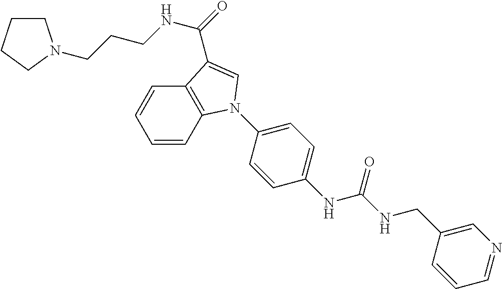 [SMILES: O=C(NCc1cccnc1)Nc1ccc(-n2cc(C(=O)NCCCN3CCCC3)c3ccccc32)cc1] | 1-[4-(3- pyridylmethylcarbamoylamino) phenyl]-N-(3- pyrrolidin-1- ylpropyl)indazole-3- carboxamide | A |
| 5 | 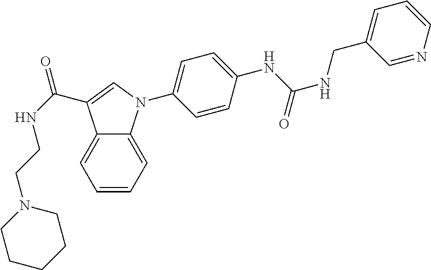 [SMILES: O=C(NCc1cccnc1)Nc1ccc(-n2cc(C(=O)NCCN3CCCCC3)c3ccccc32)cc1] | N-[2-(Piperidin-1- yl)ethyl]-1-(4-{[(pyridin- 3- ylmethyl)carbamoyl]amino} phenyl)-1H-indole-3- carboxamide | A |
| 6 | 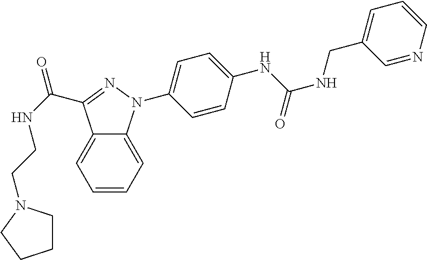 [SMILES: O=C(NCc1cccnc1)Nc1ccc(-n2nc(C(=O)NCCN3CCCC3)c3ccccc32)cc1] | 1-(4-{[(Pyridin-3- ylmethyl)carbamoyl]amino} phenyl)-N-[2- (pyrrolidin-1-yl)ethyl]- 1H-indazole-3- carboxamide | A |
| 7 | 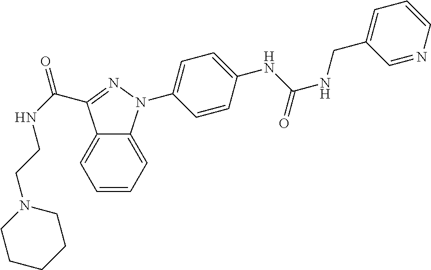 [SMILES: O=C(NCc1cccnc1)Nc1ccc(-n2nc(C(=O)NCCN3CCCCC3)c3ccccc32)cc1] | N-[2-(Piperidin-1- yl)ethyl]-1-(4-{[(pyridin- 3- ylmethyl)carbamoyl]amino} phenyl)-1H-indazole-3- carboxamide | A |
| 8 | 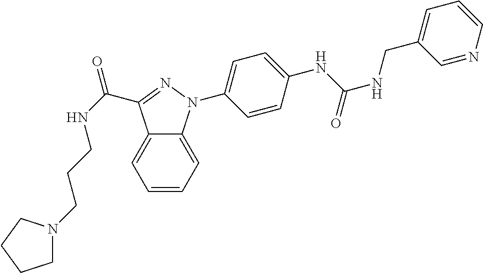 [SMILES: O=C(NCc1cccnc1)Nc1ccc(-n2nc(C(=O)NCCCN3CCCC3)c3ccccc32)cc1] | 1-(4-{[(Pyridin-3- ylmethyl)carbamoyl]amino} phenyl)-N-[3- (pyrrolidin-1-yl)propyl]- 1H-indazole-3- carboxamide | A |
| 9 | 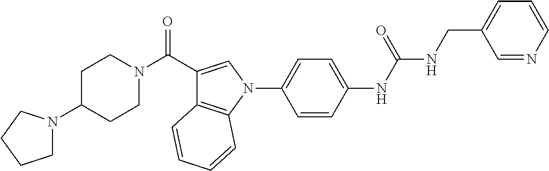 [SMILES: O=C(NCc1cccnc1)Nc1ccc(-n2cc(C(=O)N3CCC(N4CCCC4)CC3)c3ccccc32)cc1] | 1-(Pyridin-3-ylmethyl)-3- [4-(3-{[4-(pyrrolidin-1- yl)piperidin-1- yl]carbonyl}-1H-indol-1- yl)phenyl]urea | A |
| 10 | 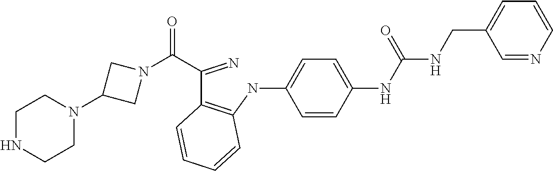 [SMILES: N=C(C(=O)N1CC(N2CCNCC2)C1)c1ccccc1Nc1ccc(NC(=O)NCc2cccnc2)cc1] | 1-[4-(3-{[3-(Piperazin-1- yl)azetidin-1- yl]carbonyl}-1H-indazol- 1-yl)phenyl]-3-(pyridin-3- ylmethyl)urea | A |
| 11 | 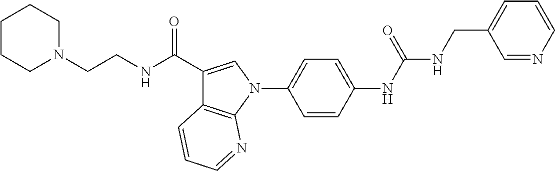 [SMILES: O=C(NCc1cccnc1)Nc1ccc(-n2cc(C(=O)NCCN3CCCCC3)c3cccnc32)cc1] | N-[2-(Piperidin-1- yl)ethyl]-1-(4-{[(pyridin- 3- ylmethyl)carbamoyl]amino} phenyl)-1H-pyrrolo[2,3- b]pyridine-3-carboxamide | A |
| 12 | 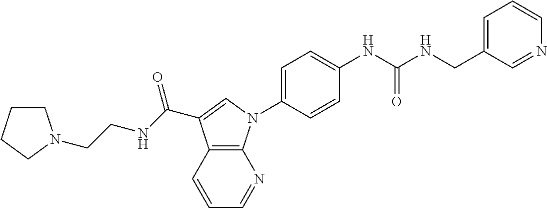 [SMILES: O=C(NCc1cccnc1)Nc1ccc(-n2cc(C(=O)NCCN3CCCC3)c3cccnc32)cc1] | 1-(4-{[(Pyridin-3- ylmethyl)carbamoyl]amino} phenyl)-N-[2- (pyrrolidin-1-yl)ethyl]- 1H-pyrrolo[2,3- b]pyridine-3-carboxamide | A |
| 13 | 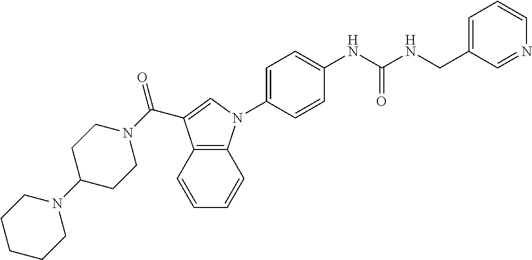 [SMILES: O=C(NCc1cccnc1)Nc1ccc(-n2cc(C(=O)N3CCC(N4CCCCC4)CC3)c3ccccc32)cc1] | 1-{4-[3-(1,4′-Bipiperidin- 1′-ylcarbonyl)-1H-indol-1- yl]phenyl}-3-(pyridin-3- ylmethyl)urea | A |
| 14 | 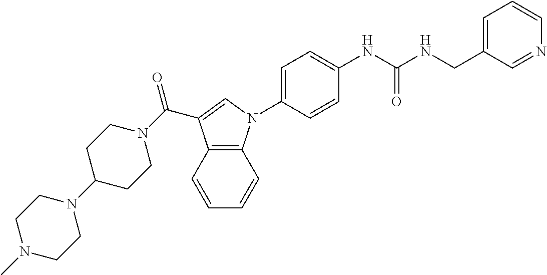 [SMILES: CN1CCN(C2CCN(C(=O)c3cn(-c4ccc(NC(=O)NCc5cccnc5)cc4)c4ccccc34)CC2)CC1] | 1-[4-(3-{[4-(4- Methylpiperazin-1- yl)piperidin-1- yl]carbonyl}-1H-indol-1- yl)phenyl]-3-(pyridin-3- ylmethyl)urea | A |
| 15 | 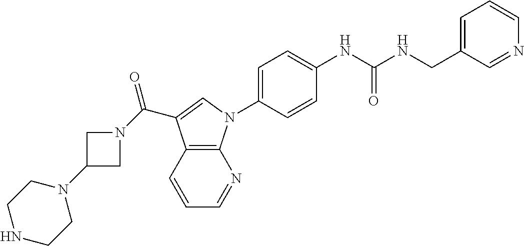 [SMILES: O=C(NCc1cccnc1)Nc1ccc(-n2cc(C(=O)N3CC(N4CCNCC4)C3)c3cccnc32)cc1] | 1-[4-(3-{[3-(Piperazin-1- yl)azetidin-1- yl]carbonyl}-1H-indol-1- yl)phenyl]-3-(pyridin-3- ylmethyl)urea | A |
| 16 | 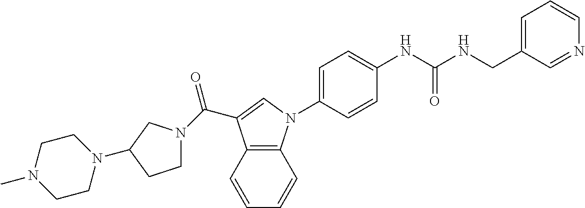 [SMILES: CN1CCN(C2CCN(C(=O)c3cn(-c4ccc(NC(=O)NCc5cccnc5)cc4)c4ccccc34)C2)CC1] | 1-[4-(3-{[3-(4- Methylpiperazin-1- yl)pyrrolidin-1- yl]carbonyl}-1H-indol-1- yl)phenyl]-3-(pyridin-3- ylmethyl)urea | A |
| 17 | 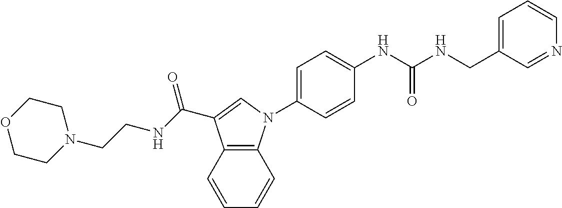 [SMILES: O=C(NCc1cccnc1)Nc1ccc(-n2cc(C(=O)NCCN3CCOCC3)c3ccccc32)cc1] | N-[2-(Morpholin-4- yl)ethyl]-1-(4-{[(pyridin- 3- ylmethyl)carbamoyl]amino} phenyl)-1H-indole-3- carboxamide | A |
| 18 | 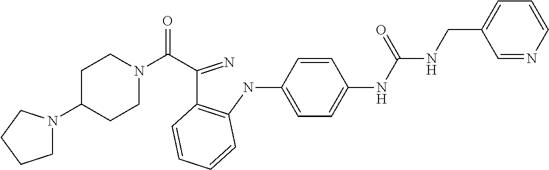 [SMILES: N=C(C(=O)N1CCC(N2CCCC2)CC1)c1ccccc1Nc1ccc(NC(=O)NCc2cccnc2)cc1] | 1-(Pyridin-3-ylmethyl)-3- [4-(3-{[4-(pyrrolidin-1- yl)piperidin-1- yl]carbonyl}-1H-indazol- 1-yl)phenyl]urea | A |
| 19 | 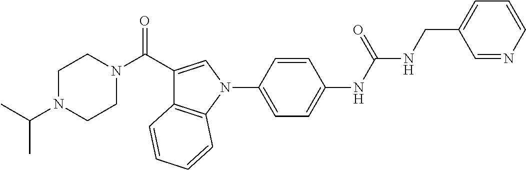 [SMILES: CC(C)N1CCN(C(=O)c2cn(-c3ccc(NC(=O)NCc4cccnc4)cc3)c3ccccc23)CC1] | 1-[4-(3-{[4-(Propan-2- yl)piperazin-1- yl]carbonyl}-1H-indol-1- yl)phenyl]-3-(pyridin-3- ylmethyl)urea | A |
| 20 | 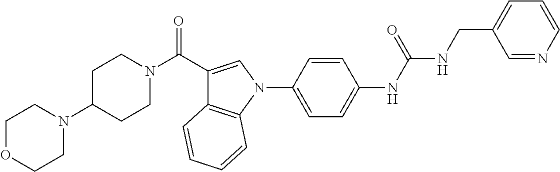 [SMILES: O=C(NCc1cccnc1)Nc1ccc(-n2cc(C(=O)N3CCC(N4CCOCC4)CC3)c3ccccc32)cc1] | 1-[4-(3-{[4-(Morpholin-4- yl)piperidin-1- yl]carbonyl}-1H-indol-1- yl)phenyl]-3-(pyridin-3- ylmethyl)urea | A |
| 21 | 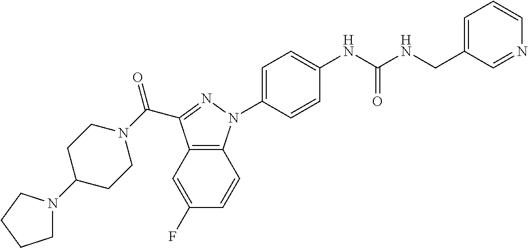 [SMILES: O=C(NCc1cccnc1)Nc1ccc(-n2nc(C(=O)N3CCC(N4CCCC4)CC3)c3cc(F)ccc32)cc1] | 1-[4-(5-Fluoro-3-{[4- (pyrrolidin-1-yl)piperidin- 1-yl]carbonyl}-1H- indazol-1-yl)phenyl]-3- (pyridin-3-ylmethyl)urea | A |
| 22 | 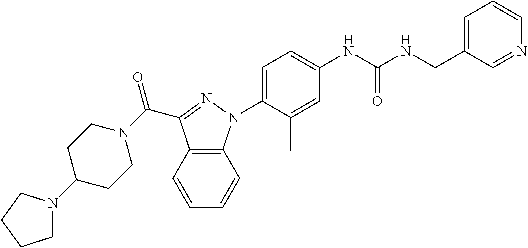 [SMILES: Cc1cc(NC(=O)NCc2cccnc2)ccc1-n1nc(C(=O)N2CCC(N3CCCC3)CC2)c2ccccc21] | 1-[3-Methyl-4-(3-{[4- (pyrrolidin-1-yl)piperidin- 1-yl]carbonyl}-1H- indazol-1-yl)phenyl]-3- (pyridin-3-ylmethyl)urea | A |
| 23 | 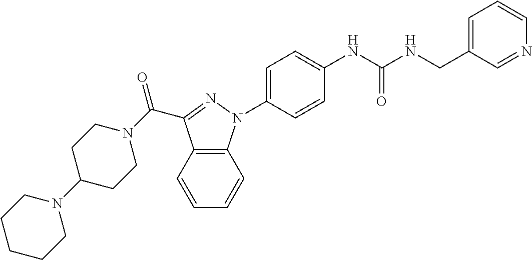 [SMILES: O=C(NCc1cccnc1)Nc1ccc(-n2nc(C(=O)N3CCC(N4CCCCC4)CC3)c3ccccc32)cc1] | 1-{4-[3-(1,4′-Bipiperidin- 1′-ylcarbonyl)-1H- indazol-1-yl]phenyl}-3- (pyridin-3-ylmethyl)urea | A |
| 24 | 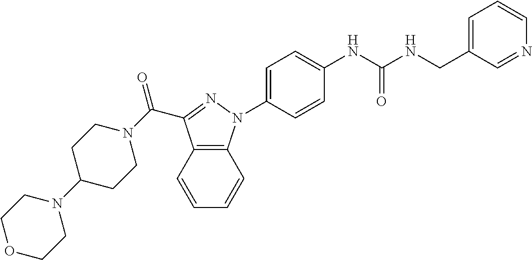 [SMILES: O=C(NCc1cccnc1)Nc1ccc(-n2nc(C(=O)N3CCC(N4CCOCC4)CC3)c3ccccc32)cc1] | 1-[4-(3-{[4-(Morpholin-4- yl)piperidin-1- yl]carbonyl}-1H-indazol- 1-yl)phenyl]-3-(pyridin-3- ylmethyl)urea | A |
| 25 | 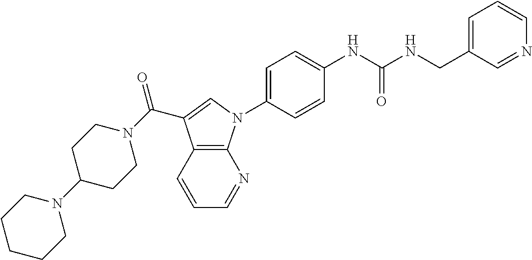 [SMILES: O=C(NCc1cccnc1)Nc1ccc(-n2cc(C(=O)N3CCC(N4CCCCC4)CC3)c3cccnc32)cc1] | 1-{4-[3-(1,4′-Bipiperidin- 1′-ylcarbonyl)-1H- pyrrolo[2,3-b]pyridin-1- yl]phenyl}-3-(pyridin-3- ylmethyl)urea | A |
| 26 | 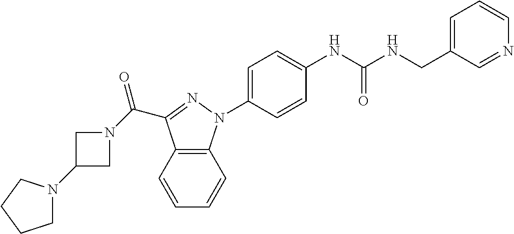 [SMILES: O=C(NCc1cccnc1)Nc1ccc(-n2nc(C(=O)N3CC(N4CCCC4)C3)c3ccccc32)cc1] | 1-(3-pyridylmethyl)-3-[4- [3-(3-pyrrolidin-1- ylazetidine-1- carbonyl)indol-1- yl]phenyl]urea | A |
| 27 | 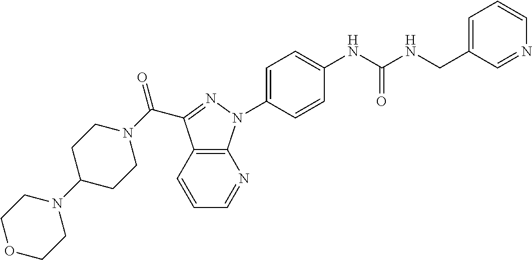 [SMILES: O=C(NCc1cccnc1)Nc1ccc(-n2nc(C(=O)N3CCC(N4CCOCC4)CC3)c3cccnc32)cc1] | 1-[4-[3-(4- morpholinopiperidine-1- carbonyl)pyrrolo[2,3- b]pyridin-1-yl]phenyl]-3- (3-pyridylmethyl)urea | A |
| 28 | 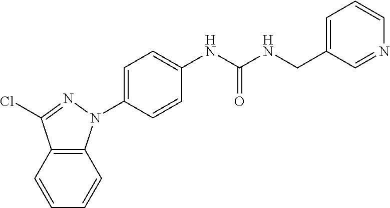 [SMILES: O=C(NCc1cccnc1)Nc1ccc(-n2nc(Cl)c3ccccc32)cc1] | 1-[4-(3-chloroindazol-1- yl)phenyl]-3-(3- pyridylmethyl)urea | A |
| 29 | 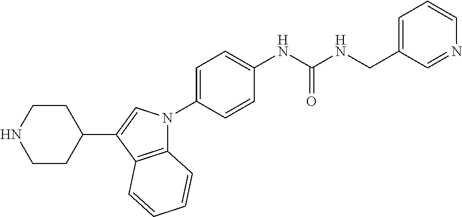 [SMILES: O=C(NCc1cccnc1)Nc1ccc(-n2cc(C3CCNCC3)c3ccccc32)cc1] | 1-[4-[3-(4-piperidyl)indol- 1-yl]phenyl]-3-(3- pyridylmethyl)urea | B |

TABLE 1B

| | HPLC | | |
|---|---|---|---|
| Example | Retention | | |
| Comp'd | Time | Mass Spec | |
| Number | (minutes) | (M + H)⁺ | H¹-NMR Data (ppm) δ |
| 1 | 4.433 | 434.2299 | 9.54 (bs, 1H), 9.19 (s, 1H), 8.70 (bs, 1H), 8.59 (t, |
| | | | 5.82 Hz, 1H), 8.48 (d, 2.38 Hz, 1H), 8.13 (d, 7.70 Hz, |
| | | | 1H), 7.77 (d, 8.47 Hz, 2H), 7.75 (bs, 1H), 7.58 (d, |
| | | | 8.47 Hz, 2H), 7.16 (t, 5.82 Hz, 1H), 6.89 (d, 2.38 Hz, |
| | | | 1H), 4.43 (d, 5.93 Hz, 2H), 3.69-3.58 (m, 4H), |
| | | | 3.38-3.31 (m, 2H), 3.10-3.00 (m, 2H), 2.06-1.97 (m, 2H), |
| | | | 1.90-1.81 (m, 2H). |
| 2 | 6.862 | 415.1745 | 8.96 (s, 1H), 8.56 (d, 1.57 Hz, 1H), 8.47 (dd, 1.57, |
| | | | 4.72 Hz, 1H), 8.22 (s, 1H), 8.13-8.10 (m, 1H), |
| | | | 7.74 (dt, 7.92, 2.13 Hz, 1H), 7.65-7.62 (m, 2H), |
| | | | 7.52-7.45 (m, 3H), 7.38 (ddd, 0.83, 4.74, 7.86 Hz, 1H), |
| | | | 7.32-7.26 (m, 2H), 6.84 (t, 5.85 Hz, 1H), 4.36 (d, 6.27 Hz, |
| | | | 2H), 4.32 (q 6.77 Hz, 2H), 1.35 (t, 6.77 Hz, 3H). |
| 3 | 5.897 | 483.2489 | 9.79 (bs, 1H), 9.23 (s, 1H), 8.39 (t, 5.52 Hz, 1H), |
| | | | 8.27-8.24 (m, 2H), 8.08 (d, 7.83 Hz, 1H), 7.78 (bs, |
| | | | 1H), 7.66 (d, 8.94 Hz, 2H), 7.47 (d, 8.94 Hz, 2H), |
| | | | 7.27-7.21 (m, 2H), 7.12 (t, 5.52 Hz, 1H), 4.64 (d, 5.63 Hz, |
| | | | 2H), 3.70-3.59 (m, 4H), 3.35 (q, 5.52 Hz, 2H), |
| | | | 3.12-3.03 (m, 2H), 2.06-1.98 (m, 2H), 1.91-1.83 (m, |
| | | | 2H). |
| 4 | 5.212 | 497.2641 | 9.87 (bs, 1H), 9.37 (s, 1H), 8.73 (bs, 1H), |
| | | | 8.28-8.23 (m, 3H), 8.12 (d, 7.90 Hz, 1H), 7.73 (bs, 1H), 7.67 (d, |
| | | | 8.78 Hz, 2H), 7.50-7.44 (m, 3H), 7.34 (t, 6.03 Hz, |
| | | | 1H), 7.27-720 (m, 2H), 4.45 (d, 5.81 Hz, 2H), |
| | | | 3.62-3.53 (m, 2H), 3.40-3.34 (m, 2H), 3.24-3.18 (m, 2H), |
| | | | 3.05-2.95 (m, 2H), 2.06-1.81 (m, 6H). |
| 5 | 5.483 | 497.2639 | 9.26 (bs, 1H), 9.20 (s, 1H), 8.75 (bs, 1H), 8.38 (t, |
| | | | 5.30 Hz, 1H), 8.27-8.22 (m, 2H), 8.07 (d, 7.48 Hz, |
| | | | 1H), 7.72 (bs, 1H), 7.66 (d, 8.30 Hz, 2H), |
| | | | 7.49-7.45 (m, 3H), 7.28-7.22 (m, 2H), 7.11 (t, 5.30 Hz, 1H), |
| | | | 4.43 (d, 5.89 Hz, 2H), 3.68-3.61 (m, 2H), 3.57 (d, |
| | | | 11.3 Hz, 2H), 3.28-3.22 (m, 2H), 3.01-2.91 (m, 2H), |
| | | | 1.87-1.80 (m, 2H), 1.73-1.60 (m, 3H), 1.49-1.34 (m, |
| | | | 1H). |
| 6 | 5.498 | 484.2446 | 9.64 (bs, 1H), 9.39 (s, 1H), 8.81 (t, 5.84 Hz, 1H), |
| | | | 8.73 (s, 1H), 8.67 (s, 1H), 8.30 (d, 8.14 Hz, 1H), 8.14 (d, |
| | | | 7.89 Hz, 1H), 7.78-7.64 (m, 5H), 7.54 (t, 7.89 Hz, |
| | | | 1H), 7.39 (t, 8.20 Hz, 1H), 7.32 (t, 5.30 Hz, 1H), |
| | | | 4.45 (d, 5.64 Hz, 2H), 3.71-3.64 (m, 4H), 3.43-3.37 (m, |
| | | | 2H), 3.12-3.03 (m, 2H), 2.06-1.98 (m, 2H), |
| | | | 1.91-1.83 (m, 2H). |
| 7 | 5.425 | 498.2603 | 9.49 (s, 1H), 9.33 (bs, 1H), 8.84 (t, 5.96 Hz, 1H), |
| | | | 8.30 (d, 8.37 Hz, 1H), 8.24 (d, 6.98 Hz, 1H), 7.90 (bs, 1H), |
| | | | 7.76 (d, 8.64 Hz, 2H), 7.69 (q, 8.47 Hz, 3H), 7.54 (t, |
| | | | 7.72 Hz, 1H), 7.47-7.42 (m, 1H), 7.39 (t, 7.46 Hz, |
| | | | 1H), 4.49 (d, 5.33 Hz, 2H), 3.72 (q, 6.05 Hz, 2H), |
| | | | 3.60 (d, 12.1 Hz, 2H), 3.34-3.27 (m, 2H), |
| | | | 3.01-2.90 (M, 2H), 1.87-1.78 (m, 2H), 1.73-1.59 (m, 2H), |
| | | | 1.45-1.35 (m, 2H). |
| 8 | 5.458 | 498.2602 | 9.70 (bs, 1H), 9.36 (s, 1H), 8.78 (bs, 1H), 8.73 (t, |
| | | | 5.86 Hz, 1H), 8.29 (d, 8.24 Hz, 1H), 8.13 (d, 7.61 Hz, |
| | | | 1H), 7.75 (d, 8.00 Hz, 1H), 7.76-7.65 (m, 5H), 7.53 (t, |
| | | | 8.00 Hz, 1H), 7.37 (t, 7.24 Hz, 1H), 7.29 (5.71 Hz, |
| | | | 1H), 4.45 (d, 5.59 Hz, 2H), 3.61-3.52 (m, 2H), |
| | | | 3.44-3.39 (m, 2H), 3.24-3.17 (m, 2H), 3.04-2.95 (m, 2H), |
| | | | 2.04-1.92 (m, 4H), 1.90-1.81 (m, 2H). |
| 9 | 6.146 | 523.2805 | 9.85 (bs, 1H), 9.20 (s, 1H), 8.70 (bs, 1H), 8.10 (d, |
| | | | 7.98 Hz, 1H), 7.89 (s, 1H), 7.76 (d, 7.98 Hz, 1H), |
| | | | 7.71 (bs, 1H), 7.64 (d, 8.92 Hz, 2H), 7.51-7.46 (m, 3H), |
| | | | 7.33-7.19 (m, 2H), 7.12 (t, 6.39 Hz, 1H), |
| | | | 4.47-4.40 (m, 4H), 3.58-3.49 (m, 2H), 3.46-3.36 (m, 1H), |
| | | | 3.16-2.96 (m, 4H), 2.15-2.08 (m, 2H), 2.04-1.96 (m, 2H), |
| | | | 1.90-1.80 (m, 2H), 1.64-1.53 (m, 2H). |
| 10 | 5.239 | 511.2592 | 9.43 (s, 1H), 8.87 (bs, 3H), 8.37 (d, 7.80 Hz, 1H), |
| | | | 8.28 (d, 7.80 Hz, 1H), 7.97-7.92 (m, 1H), 7.81 (d, |
| | | | 8.51 Hz, 1H), 7.69 (s, 3H), 7.56-7.52 (m, 1H), |
| | | | 7.40-7.36 (m, 3H), 4.76 (dd, 7.00, 11.0 Hz, 1H), 4.53 (d, |
| | | | 5.00 Hz, 1H), 4.51 (d, 6.00 Hz, 1H), 4.22 (dd, 7.00, |
| | | | 10.5 Hz, 1H), 4.05 (dd, 5.00, 10.5 Hz, 1H), |
| | | | 3.55-3.48 (m, 1H), 3.23-3.17 (m, 5H), 2.80-2.75 (m, 4H). |
| 11 | 5.234 | 498.2639 | 9.33 (bs, 1H), 9.27 (s, 1H), 8.82 (bs, 1H), |
| | | | 8.60-8.52 (m, 2H), 8.51 (s, 1H), 8.39-8.32 (m, 2H), 7.91 (bs, |
| | | | 1H), 7.71-7.61 (m, 4H), 7.33 (dd, 4.35, 7.98 Hz, 1H), |
| | | | 7.21 (t, 6.17 Hz, 1H), 4.49 (d, 5.69 Hz, 2H), |
| | | | 3.69-3.63 (m, 2H), 3.60-3.54 (m, 2H), 3.29-3.24 (m, 2H), |
| | | | 3.01-2.92 (m, 2H), 1.87-1.80 (m, 2H), 1.73-1.61 (m, 3H), |
| | | | 1.44-1.35 (m, 1H). |
| 12 | 5.206 | 484.2487 | 9.67 (bs, 1H), 9.18 (s, 1H), 8.78 (bs, 1H), 8.56 (dd, |
| | | | 1.51, 8.31 Hz, 1H), 8.52-8.49 (m, 3H), 8.37 (dd, 1.51, |
| | | | 4.15 Hz, 1H), 8.14 (d, 7.17 Hz, 1H), 7.77 (bs, 1H), |
| | | | 7.65 (q, 8.74 Hz, 3H), 7.33 (dd, 4.37, 8.15 Hz, 1H), |
| | | | 7.10 (t, 6.01 Hz, 1H), 4.45 (d, 6.32 Hz, 2H), |
| | | | 3.70-3.60 (m, 4H), 3.39-3.34 (m, 2H), 3.13-3.04 (m, 2H), |
| | | | 2.07-1.98 (m, 2H), 1.91-1.83 (m, 2H). |
| 13 | 5.476 | 537.2992 | 9.38 (bs, 1H), 9.32 (s, 1H), 8.77 (bs, 1H), 8.28 (d, |
| | | | 8.06 Hz, 1H), 7.89-7.83 (m, 2H), 7.75 (d, 7.64 Hz, |
| | | | 1H), 7.63 (d, 8.91 Hz, 2H), 7.49-7.44 (m, 3H), |
| | | | 7.29-7.017 (m, 3H), 4.50-4.43 (m, 4H), 3.51-3.36 (m, 3H), |
| | | | 3.04-2.88 (m, 4H), 2.09-2.03 (m, 2H), 1.85-1.78 (m, |
| | | | 2H), 1.72-1.60 (m, 5H), 1.43-1.32 (m, 1H). |
| 14 | 5.396 | 552.3100 | 9.30 (s, 1H), 8.80 (bs, 2H), 8.27 (d, 7.75 Hz, 1H), |
| | | | 7.91-7.85 (m, 2H), 7.74 (d, 7.75 Hz, 1H), 7.62 (d, |
| | | | 8.79 Hz, 2H), 7.49-7.44 (m, 3H), 7.26-7.17 (m, 3H), |
| | | | 4.48-4.38 (m, 4H), 3.70-3.10 (m, 8H), 3.04-2.92 (m, |
| | | | 2H), 2.83 (s, 3H), 2.04-1.99 (m, 2H), 1.60-1.48 (m, |
| | | | 2H). |
| 15 | 5.759 | 510.2622 | 9.36 (s, 1H), 8.82 (bs, 3H), 8.34 (d, 8.34 Hz, 1H), |
| | | | 8.18 (d, 6.67 Hz, 1H), 7.94-7.88 (m, 2H), 7.64 (d, |
| | | | 8.89 Hz, 2H), 7.48 (d, 8.89 Hz, 2H), 7.43 (d, 8.34 Hz, |
| | | | 1H), 7.31-7.18 (m, 3H), 4.58-4.44 (m, 3H), 4.33 (bs, |
| | | | 1H), 4.10 (bs, 1H), 3.94 (bs, 1H), 3.45-3.41 (m, 1H), |
| | | | 3.16 (bs, 4H), 2.68 (bs. 4H). |
| 16 | 5.488 | 538.2947 | 9.40 (s, 1H), 8.82 (bs, 2H), 8.32 (d, 7.82 Hz, 1H), |
| | | | 8.12 (d, 7.82 Hz, 1H), 8.02 (s, 1H), 7.90 (bs, 1H), |
| | | | 7.64 (d, 9.24 Hz, 2H), 7.48 (d, 9.24 Hz, 2H), 7.43 (d, |
| | | | 7.82 Hz, 1H), 7.38-7.32 (m, 1H), 7.24-7.16 (m, 2H), |
| | | | 4.47 (d, 5.82 Hz, 2H), 4.18-2.19 (m, 13H), 2.79 (s, |
| | | | 3H), 2.22 (bs, 1H), 1.087 (bs, 1H). |
| 17 | 6.265 | 499.2480 | 10.1 (bs, 1H), 9.43 (s, 1H), 8.79 (bs, 2H), 8.41 (t, |
| | | | 5.77 Hz, 1H), 8.30 (d, 7.42 Hz, 1H), 8.23 (d, 7.42 Hz, |
| | | | 1H), 8.21 (s, 1H), 7.88 (bs, 1H), 7.65 (d, 8.25 Hz, |
| | | | 2H), 7.45 (d, 8.25 Hz, 2H), 7.37 (t, 5.77 Hz, 1H), |
| | | | 7.26-7.19 (m, 2H), 4.47 (d, 5.33 Hz, 2H), |
| | | | 4.03-3.93 (m, 2H), 3.72-3.48 (m, 6 H), 3.32 (t, 6.36 Hz, 2H), |
| | | | 3.20-3.08 (m, 2H). |
| 18 | 4.589 | 524.2771 | 9.91 (bs, 1H), 9.27 (s, 1H), 8.72 (bs, 1H), 8.12 (d, |
| | | | 7.77 Hz, 1H), 8.04 (d, 7.77 Hz, 1H), 7.78 (d, 8.63 Hz, |
| | | | 1H), 7.73 (bs, 1H), 7.69-7.63 (m, 4H), 7.54 (t, 7.51 Hz, |
| | | | 1H), 7.35 (t, 7.51 Hz, 1H), 7.17 (t, 5.63 Hz, 1H), |
| | | | 4.86 (d, 12.8 Hz, 1H), 4.71 (d, 12.8 Hz, 1H), 4.45 (d, |
| | | | 5.96 Hz, 2H), 3.60-3.40 (m, 3H), 3.27-2.84 (m, 4H), |
| | | | 2.28-2.12 (m, 2H), 2.06-1.96 (m, 2H), 1.89-1.80 (m, |
| | | | 2H), 1.68-1.56 (m, 2H). |
| 19 | 4.629 | 497.2680 | 9.83 (bs, 1H), 9.33 (s, 1H), 8.79 (bs, 1H), 8.29 (d, |
| | | | 7.86 Hz, 1H), 8.03 (s, 1H), 7.91-7.84 (m, 2H), 7.66 (d, |
| | | | 8.64 Hz, 2H), 7.53-7.46 (m, 3H), 7.30-7.21 (m, 3H), |
| | | | 4.55 (d, 13.0 Hz, 2H), 4.55 (d, 5.69 Hz, 2H), |
| | | | 3.60-3.31 (m, 5H), 3.20-3.09 (m, 2H), 1.28 (d, 6.66 Hz, |
| | | | 6H). |
| 20 | 3.883 | 539.2776 | 10.1 (bs. 1H), 9.31 (s, 1H), 8.79 (bs, 1H), 8.30 (d, |
| | | | 7.95 Hz, 1H), 7.91-7.85 (m, 2H), 7.77 (d, 7.95 Hz, |
| | | | 1H), 7.65 (d, 8.61 Hz, 2H), 7.51-7.47 (m, 3H), |
| | | | 7.28-7.20 (m, 3H), 4.52-4.43 (m, 4H), 4.06-3.97 (m, 2H), |
| | | | 3.74-3.64 (m, 2H), 3.58-3.40 (m, 3H), 3.19-2.95 (m, |
| | | | 4H), 2.17-2.09 (m, 2H), 1.68-1.58 (m, 2H). |
| 21 | 4.089 | 542.2665 | 9.86 (bs, 1H), 9.32 (s, 1H), 8.81 (bs, 1H), 8.18 (d, |
| | | | 7.71 Hz, 3H), 7.83-7.73 (m, 3H), 7.69-7.63 (m, 4H), |
| | | | 7.45 (dt, 2.05, 6.16 Hz, 1H), 7.21 (t, 5.65 Hz, 1H), |
| | | | 4.95 (d, 11.8 Hz, 1H), 4.70 (d, 11.8 Hz, 1H), 4.46 (d, |
| | | | 5.55 Hz, 2H), 3.59-3.42 (m, 3H), 3.28-3.05 (m, 3H), |
| | | | 2.92-2.83 (m, 1H), 2.26-2.11 (m, 2H), 2.05-1.97 (m, |
| | | | 2H), 1.86-1.80 (m, 2H), 1.69-1.55 (m, 2H). |
| 22 | 4.045 | 538.2933 | 9.84 (bs, 1H), 9.28 (s, 1H), 8.81-8.68 (m, 1H), |
| | | | 8.22 (d, 8.22 Hz, 1H), 8.04 (d, 8.22 Hz, 1H), 7.83-7.78 (m, |
| | | | 1H), 7.58 (s, 1H), 7.50-7.43 (m, 2H), 7.37-7.22 (m, |
| | | | 4H), 4.86 (d, 11.7 Hz, 1H), 4.72 (d, 11.7 Hz, 1H), |
| | | | 4.46 (d, 5.83 Hz, 2H), 3.60-3.51 (m, 3H), |
| | | | 3.24-3.02 (m, 3H), 2.87 (t, 11.7 Hz, 1H), 2.27-2.11 (m, 2H), |
| | | | 2.06-1.95 (m, 5H), 1.89-1.78 (m, 2H), 1.68-1.55 (m, |
| | | | 2H). |
| 23 | 4.042 | 538.2935 | 9.38 (s, 1H), 9.34 (bs, 1H), 8.83-8.72 (m, 1H), |
| | | | 8.29 (d, 7.92 Hz, 1H), 8.05 (d, 7.92 Hz, 1H), 7.89-7.84 (m, |
| | | | 1H), 7.78 (d, 7.92 Hz, 1H), 7.70-7.64 (m, 4H), 7.54 (t, |
| | | | 7.92 Hz, 1H), 7.35 (t, 7.92 Hz, 1H), 7.31 (t, 5.28 Hz, |
| | | | 1H), 4.94 (d, 11.8 Hz, 1H), 4.78 (d, 11.8 Hz, 1H), |
| | | | 4.49 (d, 6.09 Hz, 2H), 3.58-3.48 (m, 1H), 3.44 (d, |
| | | | 11.8 Hz, 2H), 3.22 (t, 11.8 Hz, 1H), 3.01-2.83 (m, |
| | | | 3H), 2.21-2.08 (m, 2H), 1.87-1.62 (m, 7H), |
| | | | 1.45-1.35 (m, 1H). |
| 24 | 3.882 | 540.2724 | 10.1 (bs, 1H), 9.38 (s, 1H), 8.86 (bs, 1H), 8.27 (d, |
| | | | 7.92 Hz, 1H), 8.05 (d, 7.92 Hz, 1H), 7.89 (bs, 1H), |
| | | | 7.78 (d, 7.92 Hz, 1H), 7.70-7.64 (m, 4H), 7.54 (t, 7.92 Hz, |
| | | | 1H), 7.36 (t, 7.92 Hz, 1H), 7.27 (t, 6.60 Hz, 1H), |
| | | | 4.92 (d, 11.8 Hz, 1H), 4.77 (d, 11.8 Hz, 1H), 4.48 (d, |
| | | | 4.71 Hz, 2H), 4.06-3.97 (m, 2H), 3.72-3.42 (m, 5H), |
| | | | 3.27-3.07 (m, 3H), 2.88 (t, 11.8 Hz, 1H), |
| | | | 2.28-2.14 (m, 2H), 1.73-1.61 (m, 2H). |
| 25 | 3.541 | 538.2927 | 9.40-9.32 (m, 1H), 9.18 (s, 1H), 8.74 (bs, 1H), |
| | | | 8.37 (d, 4.53 Hz, 1H), 8.22-8.18 (m, 3H), 7.80 (bs, 1H), |
| | | | 7.72 (d, 9.30 Hz, 2H), 7.61 (d, 9.30 Hz, 1H), 7.30 (dd, |
| | | | 4.65, 7.76 Hz, 1H), 7.14 (t, 5.20 Hz, 1H), |
| | | | 4.56-4.45 (m, 4H), 3.55-3.44 (m, 3H), 3.11-2.91 (m, 4H), |
| | | | 2.07 (d, 10.7 Hz, 2H), 1.85 (d, 10.7 Hz, 2H), 1.76-1.63 (m, |
| | | | 5H), 1.46-1.36 (m, 1H). |
| 26 | 3.931 | 495.2494 | 10.9 (bs, 1H), 9.35 (s, 1H), 8.75 (m, 2H), 8.21, (d, 6.6 Hz, |
| | | | 2H), 8.00 (s, 1H), 7.82-7.79 (m, 1H), 7.67, (d, |
| | | | 8.25 Hz, 2H), 7.51 (d, 8.25 Hz, 2H), 7.47 (dd, 1.61, |
| | | | 6.63 Hz, 1H), 7.29-7.22 (m, 3H), 4.47 (d, 5.53 Hz, 2), |
| | | | 4.27 (bs, 1H), 3.63 (bs, 2H), 3.01 (bs, 2H), |
| | | | 2.10-2.86 (m, 4H). |
| 27 | 3.349 | 540.2729 | 10.0 (bs, 1H), 9.20 (s, 1H), 8.75, (bs, 1H), 8.37 (d, |
| | | | 4.56 Hz, 1H), 8.22 (d, 7.49 Hz, 1H), 8.20-8.16 (m, |
| | | | 2H), 7.82 (bs, 1H), 7.72 (d, 9.34 Hz, 2H), 7.61 (d, |
| | | | 9.34 Hz, 2H), 7.11 (t, 5.57 Hz, 1H), 4.54-4.44 (m, |
| | | | 4H), 4.05-3.99 (m, 4H), 3.73-3.39 (m, 6H), |
| | | | 3.21-2.94 (m, 4H), 2.13 (d, 11.2 Hz, 2H), 1.70-1.59 (m, 2H). |
| 28 | 5.941 | 378.1114 | 8.94 (s, 1H), 8.56 (s, 1H), 8.48 (d, 4.00 Hz, 1H), |
| | | | 7.80-7.74 (m, 3H), 7.65-7.56 (m, 5H), 7.41-7.35 (m, 2H), |
| | | | 6.83 (t, 5.69 Hz, 1H), 4.36 (d, 5.71 Hz, 2H). |
| 29 | 4.229 | 426.2341 | 9.16 (s, 1H), 8.73 (bs, 2H), 8.67 (bs, 1H), |
| | | | 8.55-8.45 (m, 1H), 8.15 (d, 7.93 Hz, 1H), 7.76-7.72 (m, 2H), |
| | | | 7.61 (d, 8.63 Hz, 2H), 7.47-7.38 (m, 4H), |
| | | | 7.21-7.10 (m, 3H), 4.45 (d, 5.81 Hz, 2H), 3.41 (d, 12.6 Hz, 2H), |
| | | | 3.22-3.05 (m, 3H), 2.16 (d, 12.6 Hz, 2H), |
| | | | 1.98-1.87 (m, 2H). |

TABLE 2

| Ex- | | | | Syn. |
|---|---|---|---|---|
| am- | | | HPLC; | Meth- |
| ple | Structure | IUPAC Name | LCMS | od |
| 30 | 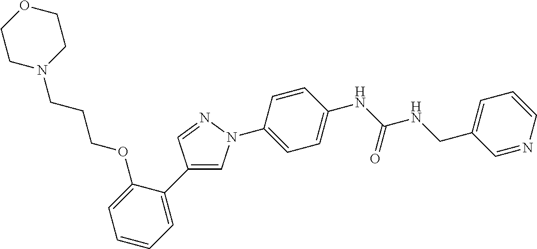 [SMILES: O=C(NCc1cccnc1)Nc1ccc(-n2cc(-c3ccccc3OCCCN3CCOCC3)cn2)cc1] | 1-[4-(4-{2-[3- (Morpholin-4- yl)propoxy] phenyl}- 1H-pyrazol-1- yl)phenyl]-3- (pyridin- 3-ylmethyl)urea | 4.293 min; 513.26331 [M + H] | V |
| 31 | 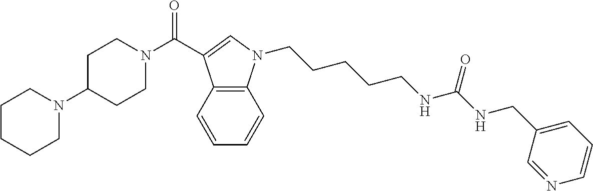 [SMILES: O=C(NCCCCCn1cc(C(=O)N2CCC(N3CCCCC3)CC2)c2ccccc21)NCc1cccnc1] | 1-{5-[3-(1,4′- Bipiperidin-1′-yl- carbonyl)-1H- indol- 1-yl]pentyl}-3- (pyridin-3- ylmethyl)urea | 3.06 min; 531.3499 [M + H] | M |
| 32 | 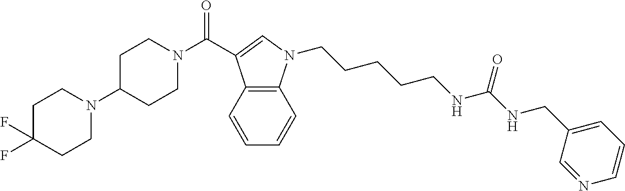 [SMILES: O=C(NCCCCCn1cc(C(=O)N2CCC(N3CCC(F)(F)CC3)CC2)c2ccccc21)NCc1cccnc1] | 1-(5-{3-[(4,4- Difluoro- 1,4′-bipiperidin-1′- yl)carbonyl]-1H- indol-1-yl}pentyl)- 3-(pyridin-3- ylmethyl)urea | 4.16 min; 567.32991 [M + H] | M |
| 33 | 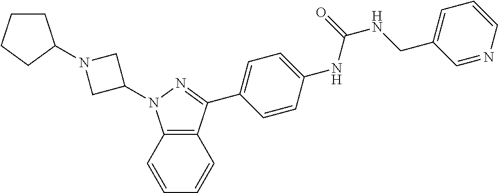 [SMILES: O=C(NCc1cccnc1)Nc1ccc(-c2nn(C3CN(C4CCCC4)C3)c3ccccc23)cc1] | 1-{4-[1-(1- Cyclopentyl- azetidin- 3-yl)-1H-indazol- 3-yl]phenyl}-3- (pyridin-3- ylmethyl)urea | 4.205 min; 467.2481 [M + H] | L |
| 34 | 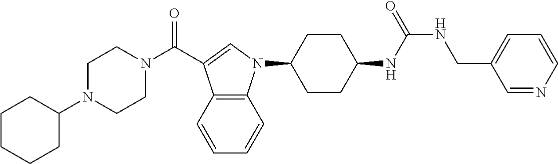 [SMILES: O=C(NCc1cccnc1)N[C@H]1CC[C@@H](n2cc(C(=O)N3CCN(C4CCCCC4)CC3)c3ccccc32)CC1] | 1-(cis-4-{3-[(4- Cyclohexyl- piperazin-1-yl) carbonyl]-1H- indol-1-yl} cyclohexyl)-3- (pyridin-3- ylmethyl)urea | 4.45 min; 543.3482 [M + H] | N |
| 35 | 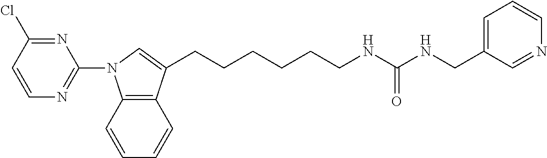 [SMILES: O=C(NCCCCCCc1cn(-c2nccc(Cl)n2)c2ccccc12)NCc1cccnc1] | 1-{6-[1-(4-Chloro- pyrimidin-2-yl)- 1H-indol-3-yl] hexyl}-3-(pyridin- 3-ylmethyl)urea | 6.20 min; 463.2028 [M + H] | M-3 |
| 36 | 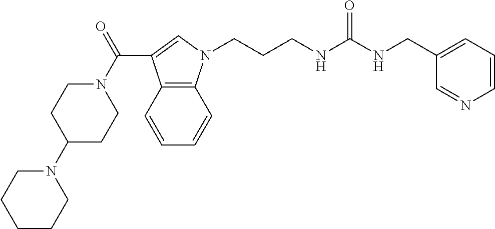 [SMILES: O=C(NCCCn1cc(C(=O)N2CCC(N3CCCCC3)CC2)c2ccccc21)NCc1cccnc1] | 1-{3-[3-(1,4′- Bipiperidin-1′- ylcarbonyl)-1H- indol-1-yl]propyl}- 3-(pyridin-3- ylmethyl)urea | 3.49 min; 503.3088 [M + H] | M |
| 37 | 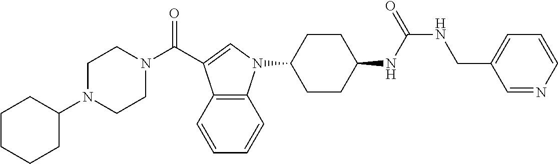 [SMILES: O=C(NCc1cccnc1)N[C@H]1CC[C@H](n2cc(C(=O)N3CCN(C4CCCCC4)CC3)c3ccccc32)CC1] | 1-(trans-4-{3-[(4- Cyclohexyl- piperazin-1-yl) carbonyl]-1H- indol-1-yl} cyclohexyl)-3- (pyridin-3- ylmethyl)urea | 3.27 min; 543.3452 [M + H] | N |
| 38 | 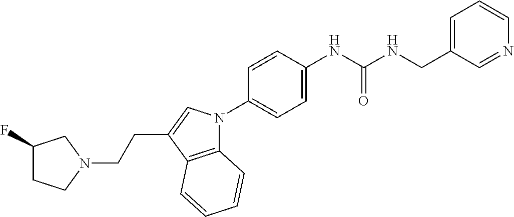 [SMILES: O=C(NCc1cccnc1)Nc1ccc(-n2cc(CCN3CC[C@@H](F)C3)c3ccccc32)cc1] | 1-[4-(3-{2-[(3R)-3- Fluoropyrrolidin-1- yl]ethyl}-1H- indol-1-yl) phenyl]-3-(pyridin- 3-ylmethyl)urea | 4.18 min; 458.23802 [M + H] | P |
| 39 | 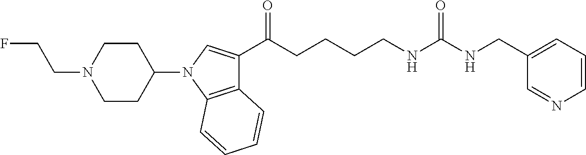 [SMILES: O=C(NCCCCC(=O)c1cn(C2CCN(CCF)CC2)c2ccccc12)NCc1cccnc1] | 1-5-{1-[1-(2- Fluoroethyl) piperidin- 4-yl]-1H-indol- 3-yl}-5-oxopentyl)- 3-(pyridin- 3-ylmethyl)urea | 3.38 min; LC-MS 480.4 [M + H] | M-2 |
| 40 | 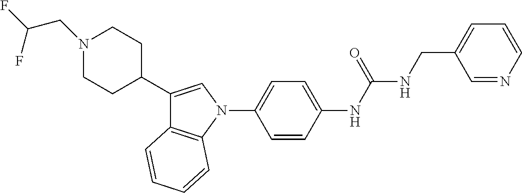 [SMILES: O=C(NCc1cccnc1)Nc1ccc(-n2cc(C3CCN(CC(F)F)CC3)c3ccccc32)cc1] | 1-(4-{3-[1-(2,2- Difluoroethyl) piperidin- 4-yl]-1H-indol-1- yl}phenyl)-3- (pyridin- 3-ylmethyl)urea | 4.069 min; 490.24781 [M + H] | K |
| 41 | 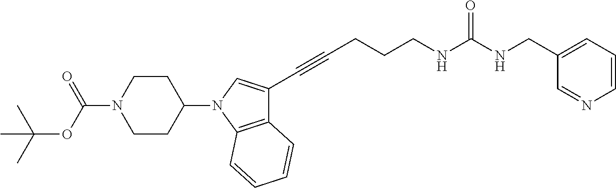 [SMILES: CC(C)(C)OC(=O)N1CCC(n2cc(C#CCCCNC(=O)NCc3cccnc3)c3ccccc32)CC1] | tert-Butyl 4-[3-(5- {[(pyridin-3- ylmethyl) carbamoyl]ami- no}pent-1-yn-1-yl)- 1H-indol-1- yl]piperidine-1- carboxylate | 6.13 min; 516.2974 [M + H] | M-2 |
| 42 | 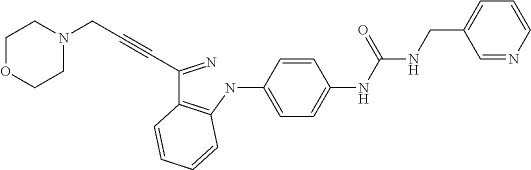 [SMILES: N=C(C#CCN1CCOCC1)c1ccccc1Nc1ccc(NC(=O)NCc2cccnc2)cc1] | 1-(4-{3-[3- (Morpholin-4- yl)prop-1-yn-1-yl]- 1H-indazol-1-yl} phenyl)-3-(pyridin- 3-ylmethyl)urea | 4.065 min; 467.2213 [M + H] | H |
| 43 | 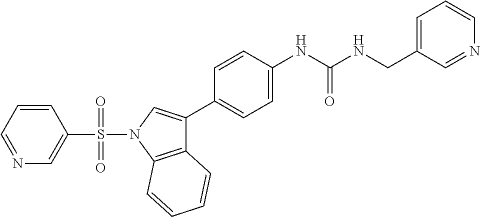 [SMILES: O=C(NCc1cccnc1)Nc1ccc(-c2cn(S(=O)(=O)c3cccnc3)c3ccccc23)cc1] | 1-(Pyridin-3- ylmethyl)-3- {4-[1-(pyridin-3- ylsulfonyl)-1H- indol-3-yl]phenyl} urea | 5.929 min; 484.1438 [M + H] | W |
| 44 | 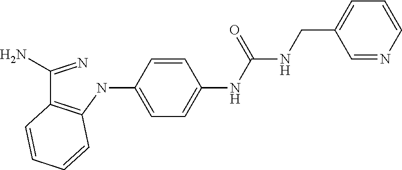 [SMILES: N=C(N)c1ccccc1Nc1ccc(NC(=O)NCc2cccnc2)cc1] | 1-[4-(3-Amino-1H- indazol-1-yl) phenyl]- 3-(pyridin-3- ylmethyl)urea | 4.6 min; 359.1666 [M + H] | C |
| 45 | 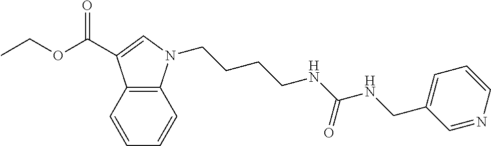 [SMILES: CCOC(=O)c1cn(CCCCNC(=O)NCc2cccnc2)c2ccccc12] | Ethyl 1-(4- {[(pyridin- 3-ylmethyl) carbamoyl]amino} butyl)-1H-indole- 3-carboxylate | 5.38 min; 395.2066 [M + H] | M |
| 46 | 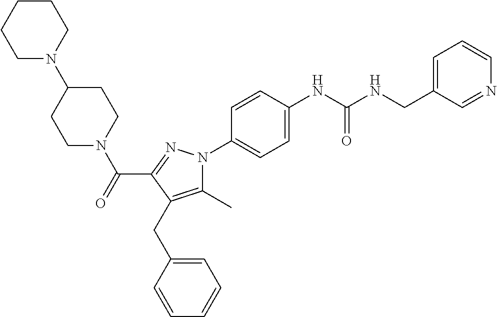 [SMILES: Cc1c(Cc2ccccc2)c(C(=O)N2CCC(N3CCCCC3)CC2)nn1-c1ccc(NC(=O)NCc2cccnc2)cc1] | 1-{4-[4-Benzyl-3- (1,4′-bipiperidin-1′- ylcarbonyl)-5- methyl- 1H-pyrazol-1-yl] phenyl}-3-(pyridin- 3-ylmethyl)urea | 3.457 min; 592.34122 [M + H] | S |
| 47 | 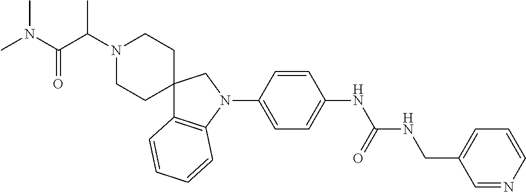 [SMILES: CC(C(=O)N(C)C)N1CCC2(CC1)CN(c1ccc(NC(=O)NCc3cccnc3)cc1)c1ccccc12] | N,N-Dimethyl-2-[1- (4-{[(pyridin-3- ylmethyl) carbamoyl]ami- no}phenyl)-1,2- dihydro-1′H- spiro[indole-3,4′- piperidin]-1′- yl]propanamide | RT = 3.807 min; 513.29497 [M + H] | Y |
| 48 | 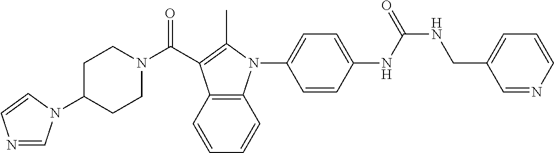 [SMILES: Cc1c(C(=O)N2CCC(n3ccnc3)CC2)c2ccccc2n1-c1ccc(NC(=O)NCc2cccnc2)cc1] | 1-[4-(3-{[4-(1H- Imidazol-1- yl)piperidin-1- yl]carbonyl}-2- methyl-1H- indol-1-yl)phenyl]- 3-(pyridin-3- ylmethyl)urea | 3.21 min; 534.2596 [M + H] | A |
| 49 | 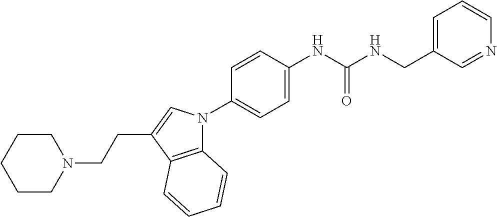 [SMILES: O=C(NCc1cccnc1)Nc1ccc(-n2cc(CCN3CCCCC3)c3ccccc32)cc1] | 1-(4-{3-[2- (Piperidin-1-yl) ethyl]-1H-indol- 1-yl}phenyl)- 3-(pyridin- 3-ylmethyl)urea | 4.39 min; 454.2593 [M + H] | P |
| 50 | 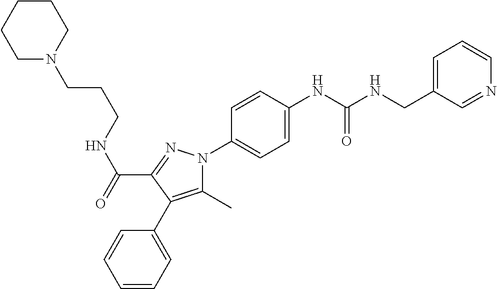 [SMILES: Cc1c(-c2ccccc2)c(C(=O)NCCCN2CCCCC2)nn1-c1ccc(NC(=O)NCc2cccnc2)cc1] | 5-Methyl-4-phenyl- N-[3-(piperidin-1- yl)propyl]-1-(4- {[(pyridin-3- ylmethyl) carbamoyl]ami- no}phenyl)-1H- pyrazole-3- carboxamide | 4.577 min; 552.30203 [M + H] | U |
| 51 | 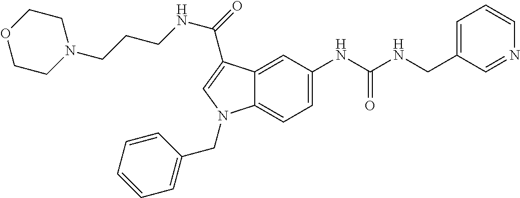 [SMILES: O=C(NCc1cccnc1)Nc1ccc2c(c1)c(C(=O)NCCCN1CCOCC1)cn2Cc1ccccc1] | 1-Benzyl-N-[3- (morpholin-4- yl)propyl]-5- {[(pyridin-3- ylmethyl) carbamoyl]ami- no}-1H-indole-3- carboxamide | 3.105 min; 527.27511 [M + H] | T |
| 52 | 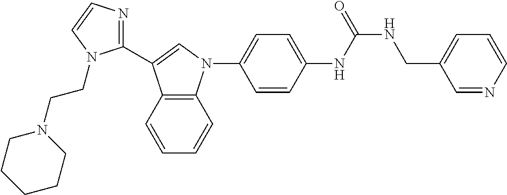 [SMILES: O=C(NCc1cccnc1)Nc1ccc(-n2cc(-c3nccn3CCN3CCCCC3)c3ccccc32)cc1] | 1-[4-(3-{1-[2- (Piperidin-1- yl)ethyl]- 1H-imidazol-2-yl}- 1H-indol-1-yl) phenyl]-3-(pyridin- 3-ylmethyl)urea | 2.96 min; 520.2764 [M + H] | O |
| 53 | 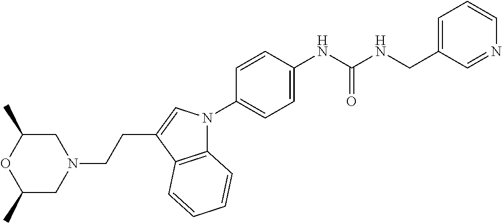 [SMILES: C[C@@H]1CN(CCc2cn(-c3ccc(NC(=O)NCc4cccnc4)cc3)c3ccccc23)C[C@H](C)O1] | 1-[4-(3-{2-[(2R,6S)- 2,6-Dimethyl- morpholin-4-yl] ethyl}-1H-indol-1- yl)phenyl]-3- (pyridin- 3-ylmethyl)urea | 3.447 min; 484.26989 [M + H] | P |
| 54 | 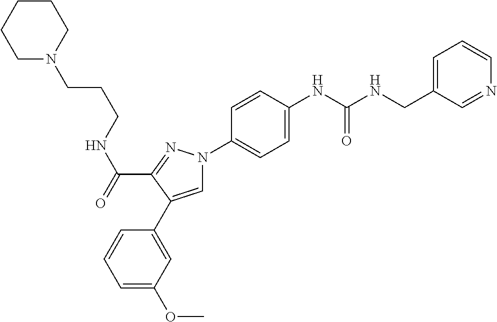 [SMILES: COc1cccc(-c2cn(-c3ccc(NC(=O)NCc4cccnc4)cc3)nc2C(=O)NCCCN2CCCCC2)c1] | 4-(3- Methoxyphenyl)- N-[3-(piperidin-1- yl)propyl]-1-(4- {[(pyridin-3- ylmethyl) carbamoyl]ami- no}phenyl)-1H- pyrazole-3- carboxamide | 4.429 min; 568.30927 [M + H] | U |
| 55 | 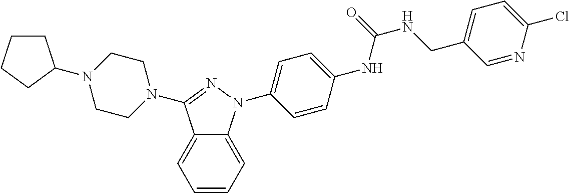 [SMILES: O=C(NCc1ccc(Cl)nc1)Nc1ccc(-n2nc(N3CCN(C4CCCC4)CC3)c3ccccc32)cc1] | 1-[(6-Chloro- pyridin-3-yl) methyl]-3-{4-[3-(4- cyclopentyl- piperazin- 1-yl)-1H-indazol-1- yl]phenyl}urea | 5.954 min; 530.2443 [M + H] | F |
| 56 | 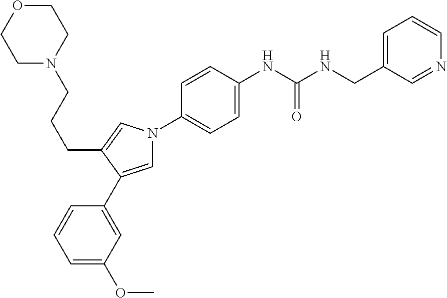 [SMILES: COc1cccc(-c2cn(-c3ccc(NC(=O)NCc4cccnc4)cc3)cc2CCCN2CCOCC2)c1] | 1-(4-{2-(3- Methoxyphenyl)-1- [3-(morpholin-4- yl)propyl]-1H- imidazol-4-yl} phenyl)-3-(pyridin- 3-ylmethyl)urea | 7.147 min; 527.27955 [M + H] | Z |
| 57 | 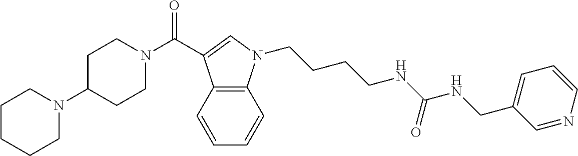 [SMILES: O=C(NCCCCn1cc(C(=O)N2CCC(N3CCCCC3)CC2)c2ccccc21)NCc1cccnc1] | 1-{4-[3-(1,4′- Bipiperidin-1′-yl- carbonyl)-1H-indol- 1-yl]butyl}-3- (pyridin-3-ylmethyl) urea | 3.64 min; 517.3305 [M + H] | M |
| 58 | 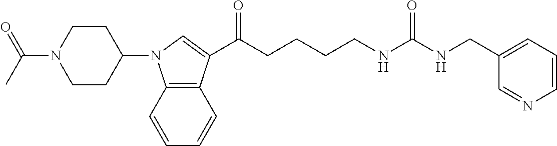 [SMILES: CC(=O)N1CCC(n2cc(C(=O)CCCCNC(=O)NCc3cccnc3)c3ccccc32)CC1] | 1-{5-[1-(1- Acetylpiperidin- 4-yl)-1H- indol-3-yl]-5-oxo- pentyl}-3-(pyridin- 3-ylmethyl)urea | 4.38 min; LC-MS 476.4 [M + H] | M-2 |
| 59 | 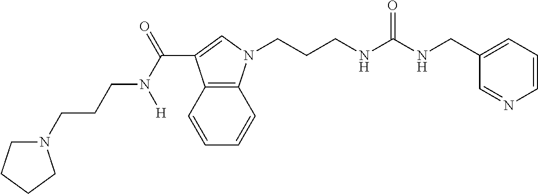 [SMILES: O=C(NCCCn1cc(C(=O)NCCCN2CCCC2)c2ccccc21)NCc1cccnc1] | 1-(3-{[(Pyridin-3- ylmethyl) carbamoyl]amino} propyl)-N-[3- (pyrrolidin-1-yl) propyl]-1H-indole- 3-carboxamide | 3.43 min; 463.2777 [M + H] | M |
| 60 | 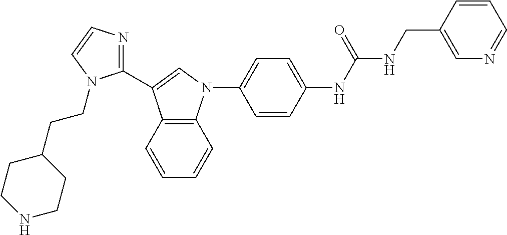 [SMILES: O=C(NCc1cccnc1)Nc1ccc(-n2cc(-c3nccn3CCC3CCNCC3)c3ccccc32)cc1] | 1-[4-(3-{1-[2- (Piperidin-4- yl)ethyl]- 1H-imidazol-2-yl}- 1H-indol-1-yl) phenyl]-3-(pyridin- 3-ylmethyl)urea | 2.87 min; 520.2844 [M + H] | O |
| 61 | 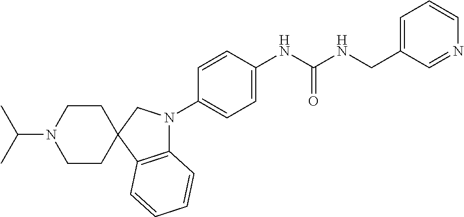 [SMILES: CC(C)N1CCC2(CC1)CN(c1ccc(NC(=O)NCc3cccnc3)cc1)c1ccccc12] | 1-{4-[1′-(Propan-2- yl)spiro[indole-3,4′- piperidin]-1(2H)- yl]phenyl}-3- (pyridin- 3-ylmethyl)urea | 3.594 min; 456.27720 [M + H] | Y |
| 62 | 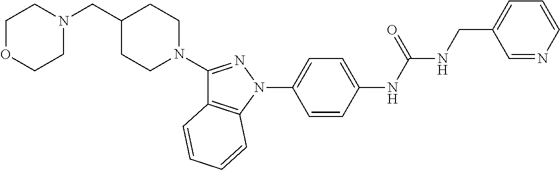 [SMILES: O=C(NCc1cccnc1)Nc1ccc(-n2nc(N3CCC(CN4CCOCC4)CC3)c3ccccc32)cc1] | 1-(4-{3-[4- (Morpholin-4- ylmethyl)piperidin- 1-yl]-1H-indazol-1- yl}phenyl)-3- (pyridin- 3-ylmethyl)urea | 3.661 min; 526.298 [M + H] | F |
| 63 | 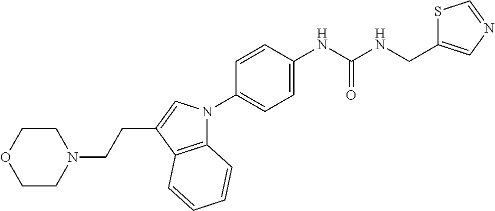 [SMILES: O=C(NCc1cncs1)Nc1ccc(-n2cc(CCN3CCOCC3)c3ccccc32)cc1] | 1-(4-{3-[2- (Morpholin-4-yl) ethyl]-1H-indol-1- yl}phenyl)-3-(1,3- thiazol-5-ylmethyl) urea | 4.89 min; 462.1969 [M + H] | P |
| 64 | 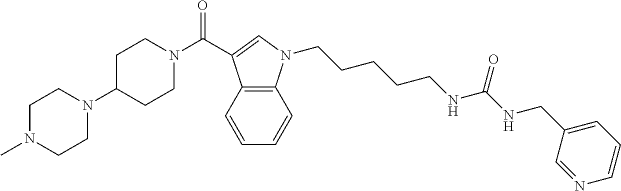 [SMILES: CN1CCN(C2CCN(C(=O)c3cn(CCCCCNC(=O)NCc4cccnc4)c4ccccc34)CC2)CC1] | 1-[5-(3-{[4-(4- Methylpiperazin-1- yl)piperidin-1-yl] carbonyl}-1H-indol- 1-yl)pentyl]-3- (pyridin-3-ylmethyl) urea | 3.59 min; 568.3393 [M + Na] | M |
| 65 | 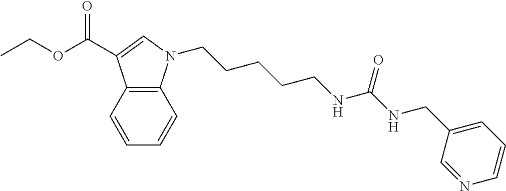 [SMILES: CCOC(=O)c1cn(CCCCCNC(=O)NCc2cccnc2)c2ccccc12] | Ethyl 1-(5- {[(pyridin- 3-ylmethyl) carbamoyl]amino} pentyl)-1H-indole- 3-carboxylate | 5.64 min; 409.2221 [M + H] | M |
| 66 | 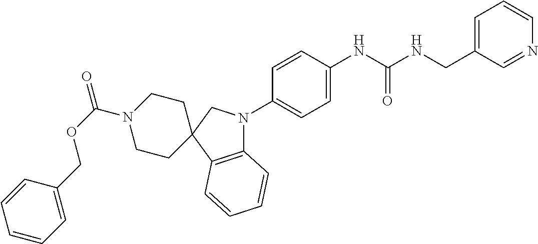 [SMILES: O=C(NCc1cccnc1)Nc1ccc(N2CC3(CCN(C(=O)OCc4ccccc4)CC3)c3ccccc32)cc1] | Benzyl 1-(4- {[(pyridin- 3-ylmethyl) carbamoyl]ami- no}phenyl)-1,2- dihydro-1′H- spiro[indole-3,4′- piperidine]-1′- carboxyylate | 7.17 min; 548.2662 (M + H) | X |
| 67 | 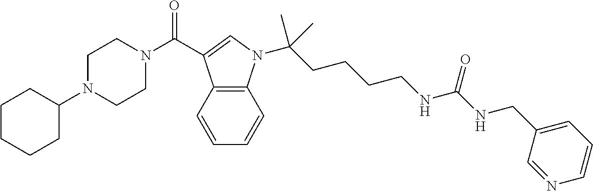 [SMILES: CC(C)(CCCCNC(=O)NCc1cccnc1)n1cc(C(=O)N2CCN(C3CCCCC3)CC2)c2ccccc21] | 1-(5-{3-[(4- Cyclohexyl- piperazin-1- yl)carbonyl]- 1H-indol-1-yl}- 5-methylhexyl)- 3-(pyridin-3- ylmethyl)urea | 4.51 min; 559.38 [M + H] | M |
| 68 | 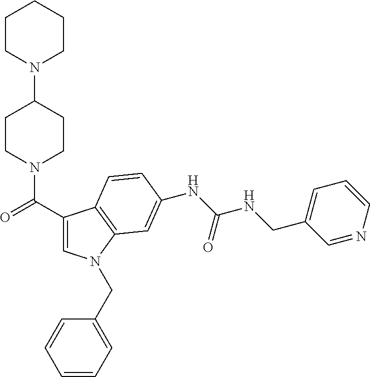 [SMILES: O=C(NCc1cccnc1)Nc1ccc2c(C(=O)N3CCC(N4CCCCC4)CC3)cn(Cc3ccccc3)c2c1] | 1-[1-Benzyl-3-(1,4′- bipiperidin-1′-yl- carbonyl)-1H-indol- 6-yl]-3-(pyridin-3- ylmethyl)urea | 2.979 min; 551.31753 [M + H] | T |
| 69 | 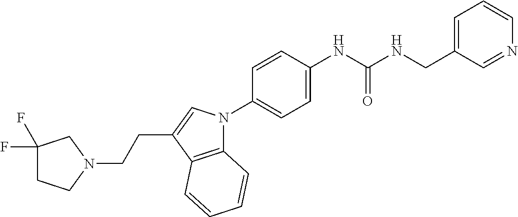 [SMILES: O=C(NCc1cccnc1)Nc1ccc(-n2cc(CCN3CCC(F)(F)C3)c3ccccc32)cc1] | 1-(4-{3-[2-(3,3- Difluoropyrrolidin- 1-yl)ethyl]- 1H-indol-1- yl}phenyl)-3- (pyridin-3-ylmethyl) urea | 3.42 min; 476.22947 [M + H] | P |
| 70 | 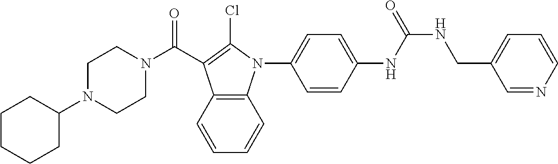 [SMILES: O=C(NCc1cccnc1)Nc1ccc(-n2c(Cl)c(C(=O)N3CCN(C4CCCCC4)CC3)c3ccccc32)cc1] | 1-(4-{2-Chloro- 3-[(4-cyclohexyl- piperazin-1-yl) carbonyl]-1H-indol- 1-yl}phenyl)-3- (pyridin-3- ylmethyl)urea | 3.56 min; 571.2709 [M + H] | A |
| 71 | 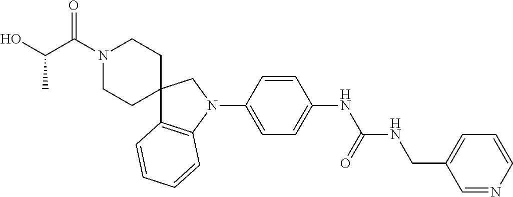 [SMILES: C[C@H](O)C(=O)N1CCC2(CC1)CN(c1ccc(NC(=O)NCc3cccnc3)cc1)c1ccccc12] | 1-(4-{1′-[(2S)-2- Hydroxypropanoyl] spiro[indole-3,4′- piperidin]-1(2H)- yl}phenyl)-3- (pyridin-3-ylmethyl) urea | 4.535 min; 486.25297 [M + H] | X |
| 72 | 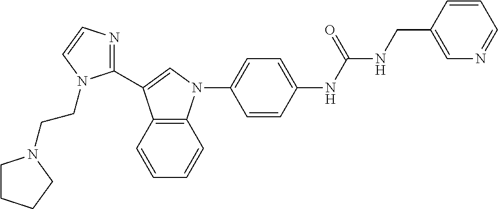 [SMILES: O=C(NCc1cccnc1)Nc1ccc(-n2cc(-c3nccn3CCN3CCCC3)c3ccccc32)cc1] | 1-(Pyridin-3- ylmethyl)-3-[4- (3-{1-[2- (pyrrolidin-1- yl)ethyl]-1H- imidazol-2-yl}-1H- indol-1-yl)phenyl] urea | 2.79 min; 506.2662 [M + H] | O |
| 73 | 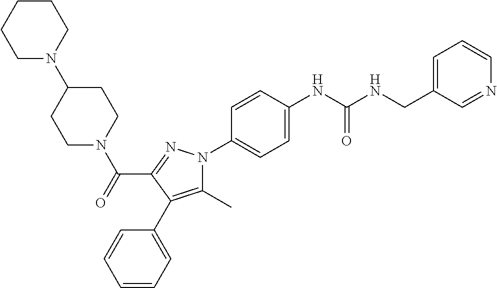 [SMILES: Cc1c(-c2ccccc2)c(C(=O)N2CCC(N3CCCCC3)CC2)nn1-c1ccc(NC(=O)NCc2cccnc2)cc1] | 1-{4-[3-(1,4′- Bipiperidin-1′- ylcarbonyl)-5- methyl-4-phenyl- 1H-pyrazol-1-yl] phenyl}-3-(pyridin- 3-ylmethyl)urea | 4.267 min; 578.31371 [M + H] | U |
| 74 | 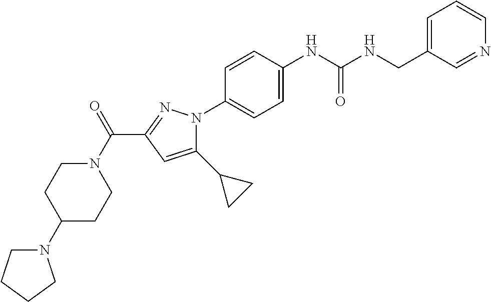 [SMILES: O=C(NCc1cccnc1)Nc1ccc(-n2nc(C(=O)N3CCC(N4CCCC4)CC3)cc2C2CC2)cc1] | 1-[4-(5- Cyclopropyl- 3-{[4-(pyrrolidin-1- yl)piperidin-1- yl]carbonyl}-1H- pyrazol-1-yl) phenyl]- 3-(pyridin-3- ylmethyl)urea | 3.71 min; 514.2918 [M + H] | R |
| 75 | 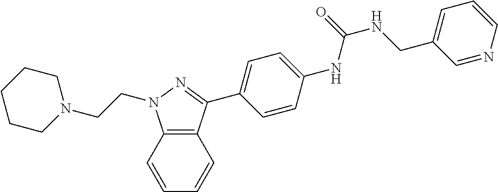 [SMILES: O=C(NCc1cccnc1)Nc1ccc(-c2nn(CCN3CCCCC3)c3ccccc23)cc1] | 1-(4-{1-[2- (Piperidin- 1-yl)ethyl]-1H- indazol-3-yl} phenyl)- 3-(pyridin-3- ylmethyl)urea | 4.111 min; 455.2568 [M + H] | D |
| 76 | 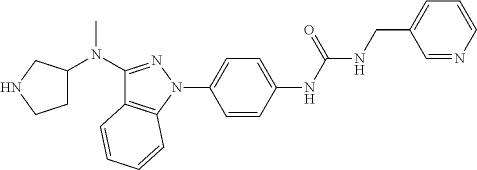 [SMILES: CN(c1nn(-c2ccc(NC(=O)NCc3cccnc3)cc2)c2ccccc12)C1CCNC1] | 1-(4-{3-[Methyl (pyrrolidin-3- yl)amino]-1H- indazol- 1-yl}phenyl)-3- (pyridin-3- ylmethyl)urea | 3.601 min; 442.23081 [M + H] | F |
| 77 | 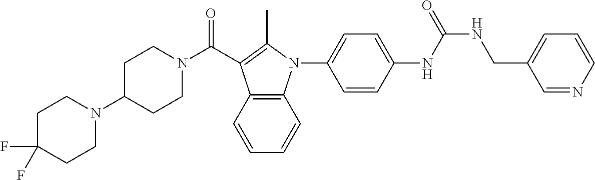 [SMILES: Cc1c(C(=O)N2CCC(N3CCC(F)(F)CC3)CC2)c2ccccc2n1-c1ccc(NC(=O)NCc2cccnc2)cc1] | 1-(4-{3- [(4,4-Difluoro- 1,4′-bipiperidin-1′- yl)carbonyl]-2- methyl-1H-indol- 1-yl}phenyl)- 3-(pyridin-3- ylmethyl)urea | 3.29 min; 587.2956 [M + H] | A |
| 78 | 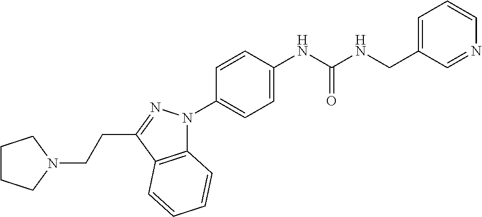 [SMILES: O=C(NCc1cccnc1)Nc1ccc(-n2nc(CCN3CCCC3)c3ccccc32)cc1] | 1-(Pyridin-3- ylmethyl)-3-(4-{3- [2-(pyrrolidin-1- yl)ethyl]-1H-indol- 1-yl}phenyl)urea | 3.254 min; 440.24501 [M + H] | P |
| 79 | 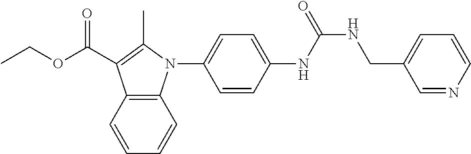 [SMILES: CCOC(=O)c1c(C)n(-c2ccc(NC(=O)NCc3cccnc3)cc2)c2ccccc12] | Ethyl 2-methyl-1- (4-{[(pyridin-3- ylmethyl) carbamoyl]amino} phenyl)-1H-indole- 3-carboxylate | 6.41 min; 429.1948 [M + H] | A |
| 80 | 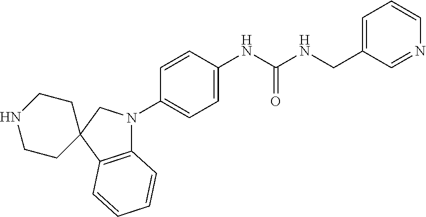 [SMILES: O=C(NCc1cccnc1)Nc1ccc(N2CC3(CCNCC3)c3ccccc32)cc1] | 1-(Pyridin-3- ylmethyl)-3-[4- (spiro[indole-3,4′- piperidin]-1(2H)- yl)phenyl]urea | 3.481 min; 414.23187 [M + H] | X |
| 81 | 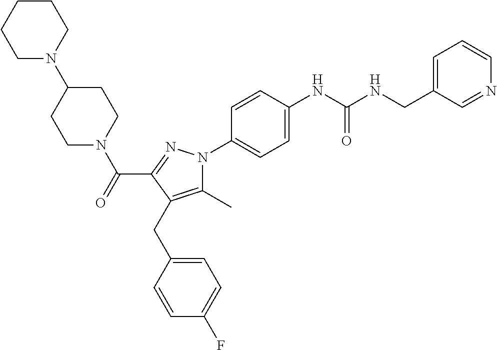 [SMILES: Cc1c(Cc2ccc(F)cc2)c(C(=O)N2CCC(N3CCCCC3)CC2)nn1-c1ccc(NC(=O)NCc2cccnc2)cc1] | 1-{4-[3-(1,4′- Bipiperidin-1′- ylcarbonyl)-4-(4- fluorobenzyl)-5- methyl-1H- pyrazol-1-yl] phenyl}-3-(pyridin- 3-ylmethyl)urea | 4.632 min; 610.32924 [M + H] | S |
| 82 | 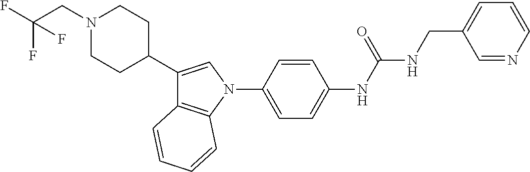 [SMILES: O=C(NCc1cccnc1)Nc1ccc(-n2cc(C3CCN(CC(F)(F)F)CC3)c3ccccc32)cc1] | 1-(Pyridin- 3-ylmethyl)-3-(4- {3-[1-(2,2,2- trifluoroethyl) piperidin- 4-yl]-1H-indol-1- yl}phenyl)urea | 4.636 min; 508.2371 [M + H] | I |
| 83 | 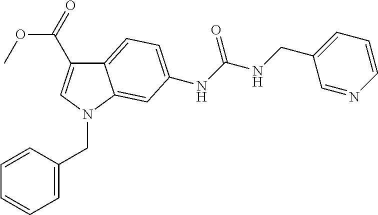 [SMILES: COC(=O)c1cn(Cc2ccccc2)c2cc(NC(=O)NCc3cccnc3)ccc12] | Methyl 1-benzyl-6- {[(pyridin-3- ylmethyl) carbamoyl]ami- no}-1H-indole-3- carboxylate | 5.562 min; 415.20894 [M + H] | T |
| 84 | 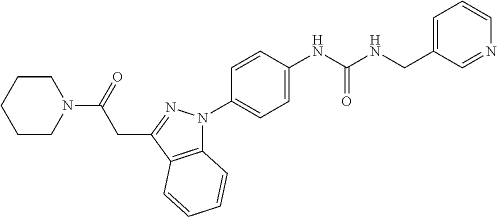 [SMILES: O=C(NCc1cccnc1)Nc1ccc(-n2nc(CC(=O)N3CCCCC3)c3ccccc32)cc1] | 1-(4-{3-[2-Oxo-2- (piperidin-1- yl)ethyl]- 1H-indazol-1-yl} phenyl)-3-(pyridin- 3-ylmethyl)urea | 5.7 min; 468.2394 [M + H] | Q |
| 85 | 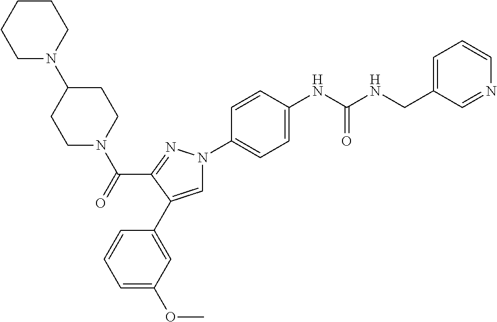 [SMILES: COc1cccc(-c2cn(-c3ccc(NC(=O)NCc4cccnc4)cc3)nc2C(=O)N2CCC(N3CCCCC3)CC2)c1] | 1-{4-[3-(1,4′- Bipiperidin-1′- ylcarbonyl)-4-(3- methoxyphenyl)- 1H-pyrazol-1-yl] phenyl}- 3-(pyridin-3- ylmethyl)urea | 4.122 min; 594.32090 [M + H] | U |
| 86 | 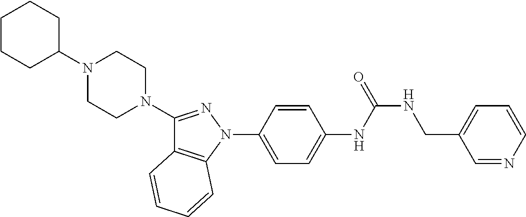 [SMILES: O=C(NCc1cccnc1)Nc1ccc(-n2nc(N3CCN(C4CCCCC4)CC3)c3ccccc32)cc1] | 1-{4-[3-(4-Cyclo- hexylpiperazin-1- yl)-1H-indazol-1- yl]phenyl}-3- (pyridin- 3-ylmethyl)urea | 3.665 min; 510.32082 [M + H] | F |
| 87 | 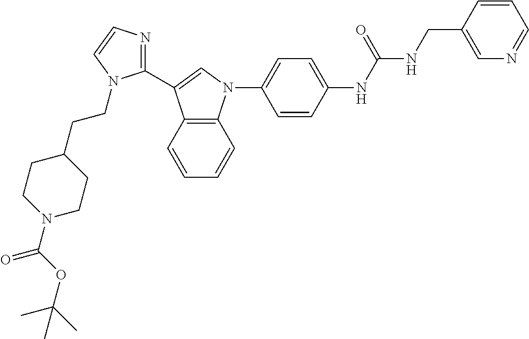 [SMILES: CC(C)(C)OC(=O)N1CCC(CCn2ccnc2-c2cn(-c3ccc(NC(=O)NCc4cccnc4)cc3)c3ccccc23)CC1] | tert-Butyl 4-(2-{2-[1- (4-{[(pyridin-3-yl- methyl)carbamoyl] amino}phenyl)- 1H-indol-3-yl]- 1H-imidazol-1- yl}ethyl)piperidine- 1-carboxylate | 4.71 min; 620.3358 [M + H] | O |
| 88 | 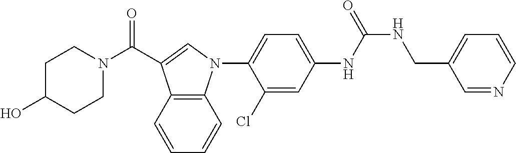 [SMILES: O=C(NCc1cccnc1)Nc1ccc(-n2cc(C(=O)N3CCC(O)CC3)c3ccccc32)c(Cl)c1] | 1-(3-Chloro-4- {3-[(4-hydroxy- piperidin-1- yl)carbonyl]-1H- indol-1-yl}phenyl)- 3-(pyridin-3- ylmethyl)urea | 4.60 min; 504.1779 [M + H] | A |
| 89 | 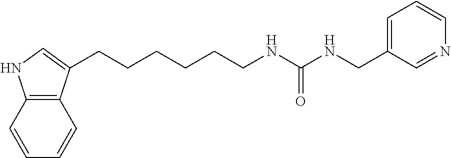 [SMILES: O=C(NCCCCCCc1c[nH]c2ccccc12)NCc1cccnc1] | 1-[6-(1H-Indol-3- yl)hexyl]-3- (pyridin- 3-ylmethyl)urea | 5.27 min; 351.2199 [M + H] | M-3 |
| 90 | 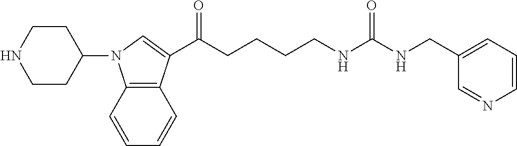 [SMILES: O=C(NCCCCC(=O)c1cn(C2CCNCC2)c2ccccc12)NCc1cccnc1] | 1-{5-Oxo-5-[1- (piperidin-4-yl)-1H- indol-3-yl]pentyl}- 3-(pyridin-3- ylmethyl)urea | 3.30 min; 434.2517 [M + H] | M-2 |
| 91 | 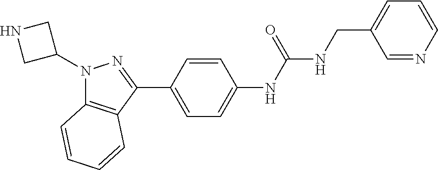 [SMILES: O=C(NCc1cccnc1)Nc1ccc(-c2nn(C3CNC3)c3ccccc23)cc1] | 1-{4-[1-(Azetidin-3- yl)-1H-indazol-3- yl]phenyl}-3- (pyridin-3-ylmethyl) urea | 3.737 min; 399.18907 [M + H] | J |
| 92 | 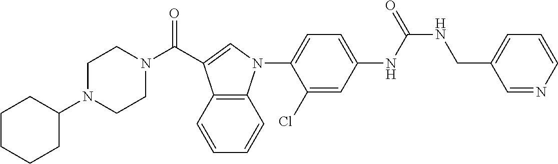 [SMILES: O=C(NCc1cccnc1)Nc1ccc(-n2cc(C(=O)N3CCN(C4CCCCC4)CC3)c3ccccc32)c(Cl)c1] | 1-(3-Chloro-4-{3- [(4-cyclohexyl- piperazin-1-yl) carbonyl]-1H-indol- 1-yl}phenyl)-3- (pyridin-3- ylmethyl)urea | 4.58 min; 571.2596 [M + H] | A |
| 93 | 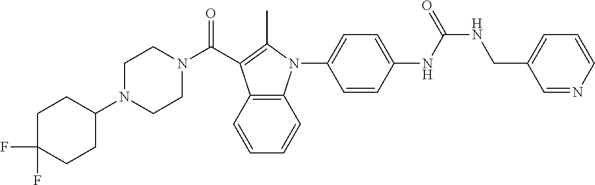 [SMILES: Cc1c(C(=O)N2CCN(C3CCC(F)(F)CC3)CC2)c2ccccc2n1-c1ccc(NC(=O)NCc2cccnc2)cc1] | 1-[4-(3-{[4-(4,4- Difluoro- cyclohexyl)pipe- razin-1-yl] carbonyl}-2- methyl-1H-indol-1- yl)phenyl]-3- (pyridin- 3-ylmethyl)urea | 3.50 min; 587.2921 [M + H] | A |
| 94 | 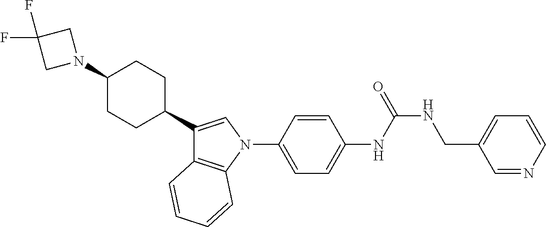 [SMILES: O=C(NCc1cccnc1)Nc1ccc(-n2cc([C@H]3CC[C@@H](N4CC(F)(F)C4)CC3)c3ccccc32)cc1] | 1-(4-{3-[cis-4-(3,3- Difluoroazetidin-1- yl)cyclohexyl]-1H- indol-1-yl}phenyl)- 3-(pyridin-3- ylmethyl)urea | 5.115 min; 516.2653 [M + H] | E |
| 95 | 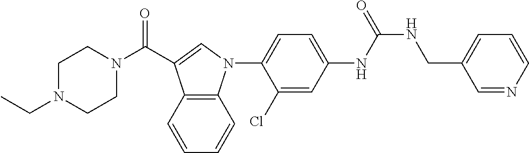 [SMILES: CCN1CCN(C(=O)c2cn(-c3ccc(NC(=O)NCc4cccnc4)cc3Cl)c3ccccc23)CC1] | 1-(3-Chloro-4-{3- [(4-ethylpiperazin- 1-yl)carbonyl]-1H- indol- 1-yl}phenyl)-3- (pyridin-3- ylmethyl)urea | 3.99 min; 517.2115 [M + H] | A |
| 96 | 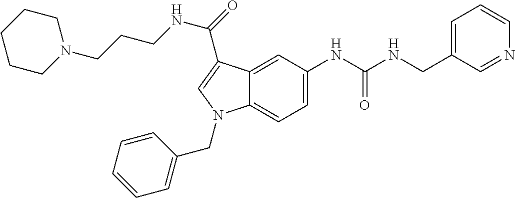 [SMILES: O=C(NCc1cccnc1)Nc1ccc2c(c1)c(C(=O)NCCCN1CCCCC1)cn2Cc1ccccc1] | 1-Benzyl-N-[3- (piperidin-1-yl) propyl]-5- {[(pyridin-3-yl- methyl)carbamoyl] amino}-1H-indole- 3-carboxamide | 3.253 min; 525.29725 [M + H] | T |
| 97 | 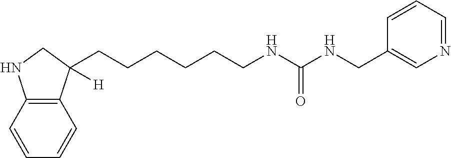 [SMILES: O=C(NCCCCCCC1CNc2ccccc21)NCc1cccnc1] | 1-[6-(2,3-Dihydro- 1H-indol-3-yl) hexyl]-3-(pyridin-3- ylmethyl)urea | 3.42 min; 353.2344 [M + H] | M-3 |
| 98 | 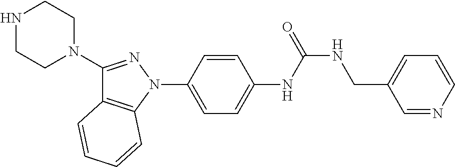 [SMILES: O=C(NCc1cccnc1)Nc1ccc(-n2nc(N3CCNCC3)c3ccccc32)cc1] | 1-{4-[3-(Piperazin- 1-yl)-1H-indazol-1- yl]phenyl}-3- (pyridin-3- ylmethyl)urea | 2.999 min; 428.2213 [M + H] | F |
| 99 | 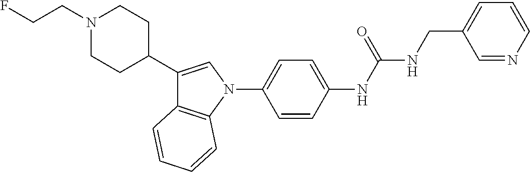 [SMILES: O=C(NCc1cccnc1)Nc1ccc(-n2cc(C3CCN(CCF)CC3)c3ccccc32)cc1] | 1-(4-{3-[1-(2- Fluoroethyl) piperidin-4-yl]- 1H-indol-1-yl} phenyl)-3-(pyridin- 3-ylmethyl)urea | 3.809 min; 472.2485 [M + H] | G |
| 100 | 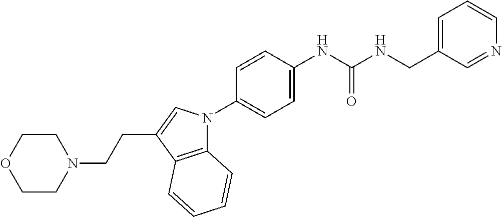 [SMILES: O=C(NCc1cccnc1)Nc1ccc(-n2cc(CCN3CCOCC3)c3ccccc32)cc1] | 1-(4-{3-[2- (Morpholin-4-yl) ethyl]-1H-indol-1- yl}phenyl)-3- (pyridin- 3-ylmethyl)urea | 4.074 min; 456.23825 [M + H] | P |
| 101 | 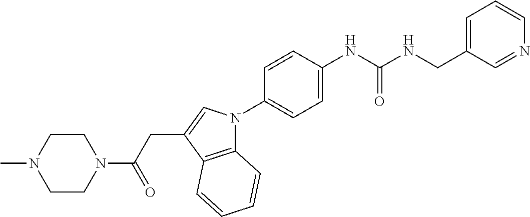 [SMILES: CN1CCN(C(=O)Cc2cn(-c3ccc(NC(=O)NCc4cccnc4)cc3)c3ccccc23)CC1] | 1-(4-{3-[2-(4- Methylpiperazin- 1-yl)-2-oxoethyl]- 1H-indol- 1-yl}phenyl)-3- (pyridin-3- ylmethyl)urea | 3.0 min; 483.2478 [M + H] | Q |
| 102 | 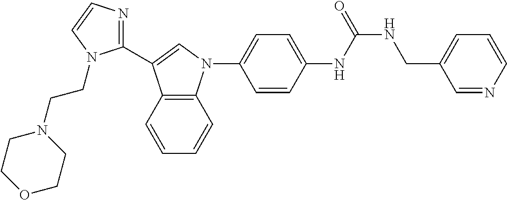 [SMILES: O=C(NCc1cccnc1)Nc1ccc(-n2cc(-c3nccn3CCN3CCOCC3)c3ccccc32)cc1] | 1-[4-(3-{1-[2- (Morpholin- 4-yl)ethyl]-1H- imidazol-2-yl}-1H- indol-1-yl)phenyl]- 3-(pyridin-3- ylmethyl)urea | 3.38 min; 522.2648 [M + H] | O |
| 103 | 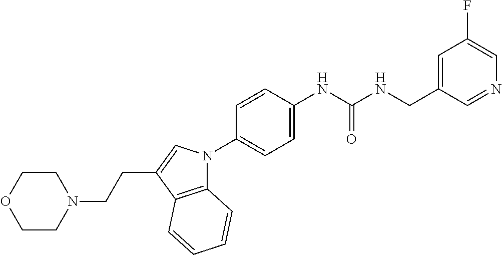 [SMILES: O=C(NCc1cncc(F)c1)Nc1ccc(-n2cc(CCN3CCOCC3)c3ccccc32)cc1] | 1-[(5-Fluoro- pyridin-3-yl) methyl]-3-(4-{3-[2- (morpholin-4-yl) ethyl]-1H-indol-1- yl}phenyl)urea | 5.16 min; 474.2346 (M + H) | P |
| 104 | 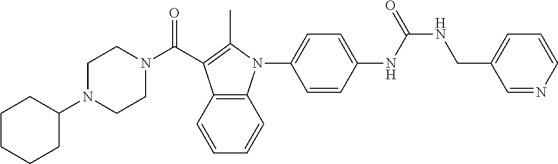 [SMILES: Cc1c(C(=O)N2CCN(C3CCCCC3)CC2)c2ccccc2n1-c1ccc(NC(=O)NCc2cccnc2)cc1] | 1-(4-{3-[(4- Cyclohexyl- piperazin-1-yl) carbonyl]-2-methyl- 1H-indol-1-yl} phenyl)-3-(pyridin- 3-ylmethyl)urea | 3.54 min; 551.317 [M + H] | A |
| 105 | 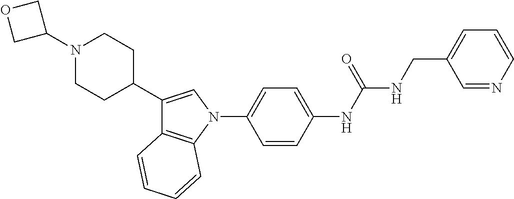 [SMILES: O=C(NCc1cccnc1)Nc1ccc(-n2cc(C3CCN(C4COC4)CC3)c3ccccc32)cc1] | 1-(4-{3-[1- (Oxetan-3-yl) piperidin-4-yl]-1H- indol-1-yl}phenyl)- 3-(pyridin-3- ylmethyl)urea | 4.114 min; 482.26307 [M + H] | K |
| 106 | 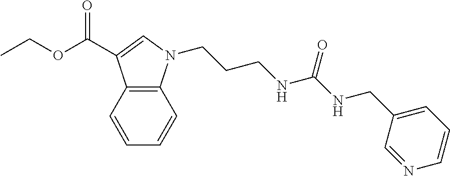 [SMILES: CCOC(=O)c1cn(CCCNC(=O)NCc2cccnc2)c2ccccc12] | Ethyl 1-(3- {[(pyridin- 3-ylmethyl) carbamoyl]ami- no}propyl)-1H- indole-3- carboxylate | 5.20 min; 403.1855 [M + Na] | M |
| 107 | 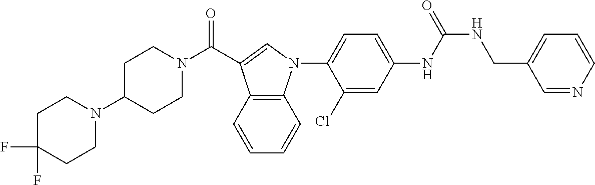 [SMILES: O=C(NCc1cccnc1)Nc1ccc(-n2cc(C(=O)N3CCC(N4CCC(F)(F)CC4)CC3)c3ccccc32)c(Cl)c1] | 1-(3-Chloro-4-{3- [(4,4-difluoro-1,4′- bipiperidin-1′-yl) carbonyl]-1H-indol- 1-yl}phenyl)-3- (pyridin-3- ylmethyl)urea | 4.36 min; 607.2387 [M + H] | A |
| 108 | 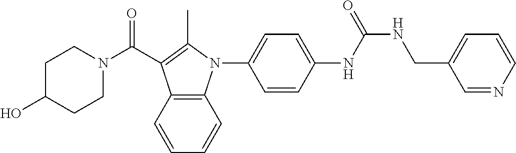 [SMILES: Cc1c(C(=O)N2CCC(O)CC2)c2ccccc2n1-c1ccc(NC(=O)NCc2cccnc2)cc1] | 1-(4-{3-[(4- Hydroxypiperidin- 1-yl)carbonyl]-2- methyl-1H-indol-1- yl}phenyl)-3- (pyridin-3-yl- methyl)urea | 3.94 min; 484.2391 [M + H] | A |
| 109 | 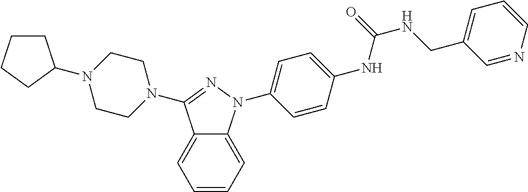 [SMILES: O=C(NCc1cccnc1)Nc1ccc(-n2nc(N3CCN(C4CCCC4)CC3)c3ccccc32)cc1] | 1-{4-[3-(4- Cyclopentyl- piperazin- 1-yl)-1H-indazol-1- yl]phenyl}-3- (pyridin-3- ylmethyl)urea | 4.352 min; 496.28193 [M + H] | F |
| 110 | 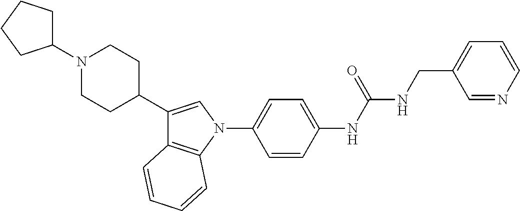 [SMILES: O=C(NCc1cccnc1)Nc1ccc(-n2cc(C3CCN(C4CCCC4)CC3)c3ccccc32)cc1] | 1-{4-[3-(1- Cyclopentyl- piperidin- 4-yl)-1H-indol-1- yl]phenyl}-3- (pyridin-3- ylmethyl)urea | 4.813 min; 494.29428 [M + H] | K |
| 111 | 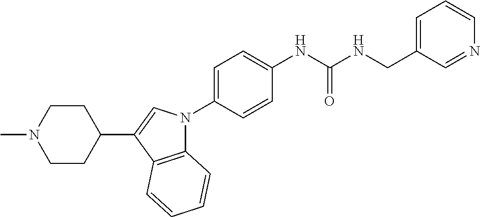 [SMILES: CN1CCC(c2cn(-c3ccc(NC(=O)NCc4cccnc4)cc3)c3ccccc23)CC1] | 1-{4-[3-(1- Methylpiperidin-4- yl)-1H-indol-1-yl] phenyl}-3-(pyridin- 3-ylmethyl)urea | 4.306 min; 440.24435 [M + H] | I |
| 112 | 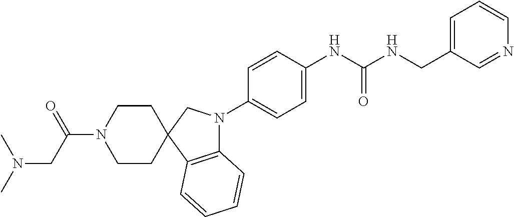 [SMILES: CN(C)CC(=O)N1CCC2(CC1)CN(c1ccc(NC(=O)NCc3cccnc3)cc1)c1ccccc12] | 1-{4-[1′-(N,N- Dimethylglycyl) spiro[indole-3,4′- piperidin]- 1(2H)-yl]phenyl}- 3-(pyridin-3- ylmethyl)urea | 3.670 min; 499.28723 [M + H] | X |
| 113 | 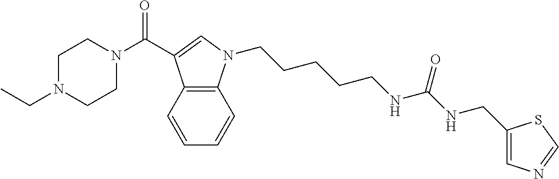 [SMILES: CCN1CCN(C(=O)c2cn(CCCCCNC(=O)NCc3cncs3)c3ccccc23)CC1] | 1-(5-{3-[(4- Ethylpiperazin-1- yl)carbonyl]-1H- indol-1-yl} pentyl)-3-(1,3- thiazol-5-ylmethyl) urea | 4.29 min; 483.2537 [M + H] | M |
| 114 | 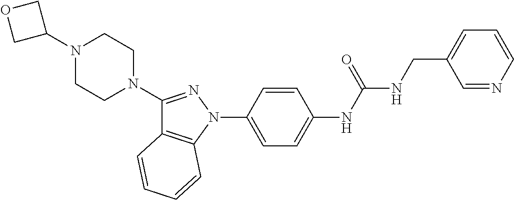 [SMILES: O=C(NCc1cccnc1)Nc1ccc(-n2nc(N3CCN(C4COC4)CC3)c3ccccc32)cc1] | 1-(4-{3-[4- (Oxetan-3-yl) piperazin-1-yl]-1H- indazol-1-yl} phenyl)-3-(pyridin- 3-ylmethyl)urea | 3.533 min, 3.752 min; 484.24714 [M + H] | F |
| 115 | 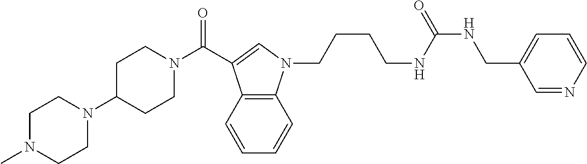 [SMILES: CN1CCN(C2CCN(C(=O)c3cn(CCCCNC(=O)NCc4cccnc4)c4ccccc34)CC2)CC1] | 1-[4-(3-{[4-(4- Methylpiperazin-1- yl)piperidin-1-yl] carbonyl}-1H- indol-1-yl)butyl]- 3-(pyridin-3- ylmethyl)urea | 3.38 min; 554.3220 [M + Na] | M |
| 116 | 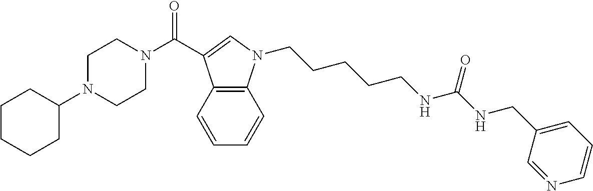 [SMILES: O=C(NCCCCCn1cc(C(=O)N2CCN(C3CCCCC3)CC2)c2ccccc21)NCc1cccnc1] | 1-(5-{3-[(4- Cyclohexyl- piperazin-1-yl) carbonyl]-1H- indol-1-yl}pentyl)- 3-(pyridin-3- ylmethyl)urea | 4.15 min; 531.343 [M + H] | M |
| 117 | 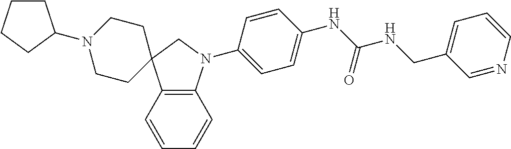 [SMILES: O=C(NCc1cccnc1)Nc1ccc(N2CC3(CCN(C4CCCC4)CC3)c3ccccc32)cc1] | 1-[4-(1′-Cyclo- pentylspiro[indole- 3,4′-piperidin]- 1(2H)-yl)phenyl]- 3-(pyridin- 3-ylmethyl)urea | 4.056 min; 482.29351 [M + H] | Y |
| 118 | 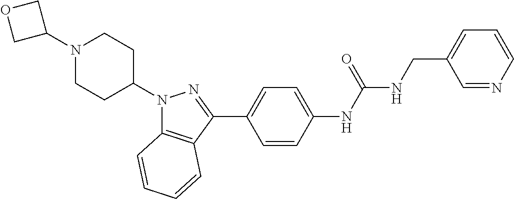 [SMILES: O=C(NCc1cccnc1)Nc1ccc(-c2nn(C3CCN(C4COC4)CC3)c3ccccc23)cc1] | 1-(4-{1-[1- (Oxetan-3-yl) piperidin-4-yl]- 1H-indazol-3-yl} phenyl)-3-(pyridin- 3-ylmethyl)urea | 3.558 min, 3.800 min; 483.24839 [M + H] | J |
| 119 | 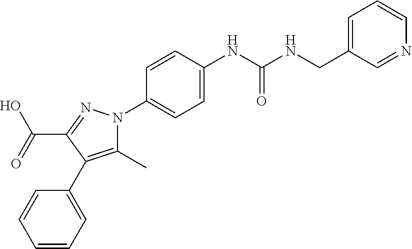 [SMILES: Cc1c(-c2ccccc2)c(C(=O)O)nn1-c1ccc(NC(=O)NCc2cccnc2)cc1] | 5-Methyl-4- phenyl-1-(4- {[(pyridin-3- ylmethyl) carbamoyl] amino}phenyl)- 1H-pyrazole-3- carboxylic acid | 4.994 min; 428.17601 [M + H] | U |
| 120 | 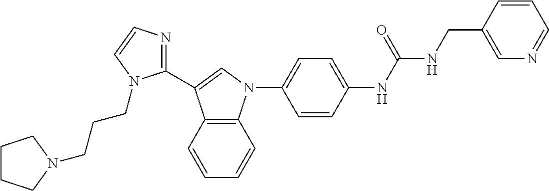 [SMILES: O=C(NCc1cccnc1)Nc1ccc(-n2cc(-c3nccn3CCCN3CCCC3)c3ccccc32)cc1] | 1-(Pyridin-3- ylmethyl)- 3-[4-(3-{1-[3- (pyrrolidin-1- yl)propyl]-1H- imidazol-2-yl}- 1H-indol- 1-yl)phenyl] urea | 2.87 min; 520.2794 [M + H] | O |
| 121 | 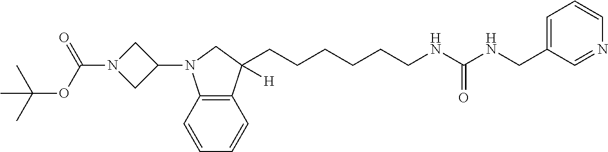 [SMILES: CC(C)(C)OC(=O)N1CC(N2CC(CCCCCCNC(=O)NCc3cccnc3)c3ccccc32)C1] | tert-Butyl 3-[3-(6- {[(pyridin-3-yl- methyl)carbamoyl] amino}hexyl)-2,3- dihydro-1H-indol- 1-yl]azetidine-1- carboxylate | 6.34 min; 508.3234 [M + H] | M-3 |
| 122 | 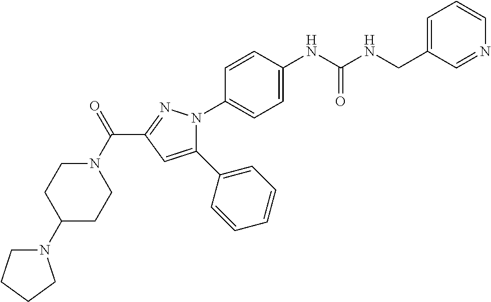 [SMILES: O=C(NCc1cccnc1)Nc1ccc(-n2nc(C(=O)N3CCC(N4CCCC4)CC3)cc2-c2ccccc2)cc1] | 1-[4-(5-Phenyl-3- {[4-(pyrrolidin-1- yl)piperidin-1- yl]carbonyl}-1H- pyrazol-1-yl) phenyl]- 3-(pyridin-3- ylmethyl)urea | 4.1 min; 550.2911 [M + H] | R |
| 123 | 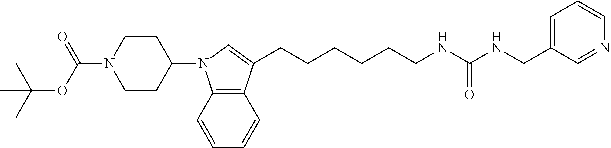 [SMILES: CC(C)(C)OC(=O)N1CCC(n2cc(CCCCCCNC(=O)NCc3cccnc3)c3ccccc32)CC1] | tert-Butyl 4-[3-(6- {[(pyridin-3- ylmethyl) carbamoyl]amino} hexyl)-1H-indol-1- yl]piperidine-1- carboxylate | 6.56 min; 534.3475 [M + H] | M-2 |
| 124 | 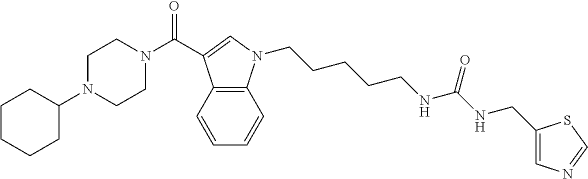 [SMILES: O=C(NCCCCCn1cc(C(=O)N2CCN(C3CCCCC3)CC2)c2ccccc21)NCc1cncs1] | 1-(5-{3-[(4- Cyclohexyl- piperazin-1-yl) carbonyl]-1H- indol-1-yl} pentyl)-3-(1,3- thiazol-5-ylmethyl) urea | 4.95 min; 537.298 [M + H] | M |
| 125 | 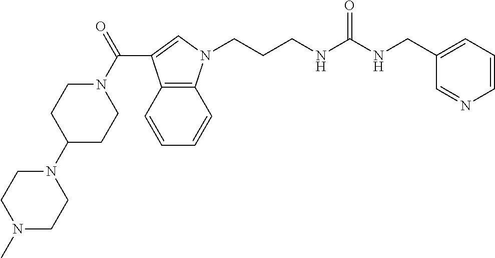 [SMILES: CN1CCN(C2CCN(C(=O)c3cn(CCCNC(=O)NCc4cccnc4)c4ccccc34)CC2)CC1] | 1-[3-(3-{[4-(4- Methylpiperazin- 1-yl)piperidin-1- yl]carbonyl}-1H- indol-1-yl)propyl]- 3-(pyridin-3- ylmethyl)urea | 3.23 min; 518.3248 [M + H] | M |
| 126 | 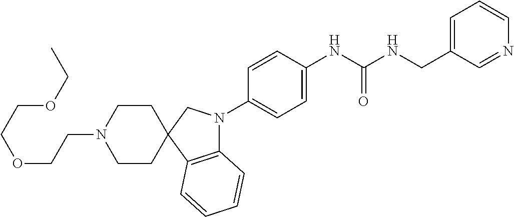 [SMILES: CCOCCOCCN1CCC2(CC1)CN(c1ccc(NC(=O)NCc3cccnc3)cc1)c1ccccc12] | 1-(4-{1′-[2-(2- Ethoxy- ethoxy)ethyl] spiro[indole-3,4′- piperidin]-1(2H)- yl}phenyl)-3- (pyridin-3- ylmethyl)urea | 3.920; 530.31169 [M + H] | Y |
| 127 | 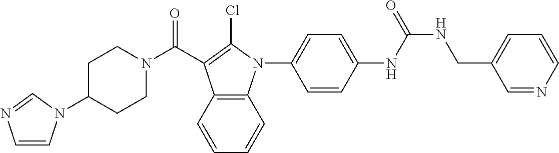 [SMILES: O=C(NCc1cccnc1)Nc1ccc(-n2c(Cl)c(C(=O)N3CCC(n4ccnc4)CC3)c3ccccc32)cc1] | 1-[4-(2-Chloro-3- {[4-(1H-imidazol- 1-yl)piperidin-1- yl]carbonyl}-1H- indol-1-yl)phenyl]- 3-(pyridin-3- ylmethyl)urea | 3.28 min; 554.2063 [M + H] | A |
| 128 | 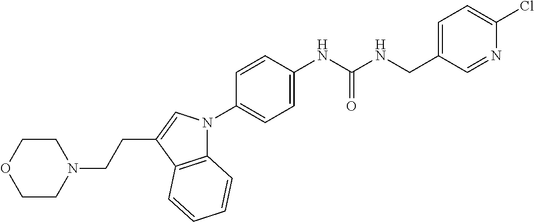 [SMILES: O=C(NCc1ccc(Cl)nc1)Nc1ccc(-n2cc(CCN3CCOCC3)c3ccccc32)cc1] | 1-[(6-Chloro- pyridin-3-yl) methyl]-3-(4-{3- [2-(morpholin-4- yl)ethyl]-1H-indol- 1-yl}phenyl)urea | 5.63 min; 490.1998 [M + H] | P |
| 129 | 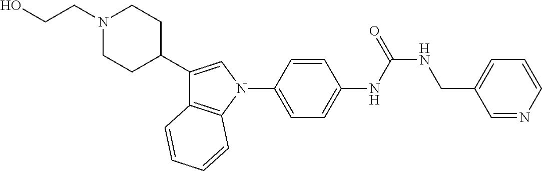 [SMILES: O=C(NCc1cccnc1)Nc1ccc(-n2cc(C3CCN(CCO)CC3)c3ccccc32)cc1] | 1-(4-{3-[1-(2- Hydroxy- ethyl)piperidin- 4-yl]-1H-indol-1- yl}phenyl)-3- (pyridin-3- ylmethyl)urea | 3.364 min; 470.25206 [M + H] | G |
| 129-A | 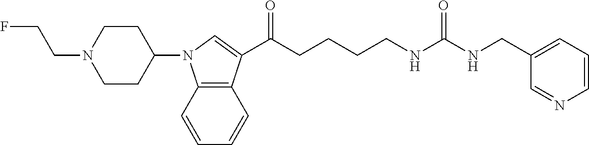 [SMILES: O=C(NCCCCC(=O)c1cn(C2CCN(CCF)CC2)c2ccccc12)NCc1cccnc1] | 1-[5-[1-[1-(2- fluoroethyl)-4- piperidyl]indol- 3-yl]-5-oxo- pentyl]-3-(3- pyridylmethyl) urea | 3.38 min; 480.4 [M + H] | M-2 |
| 129-B | 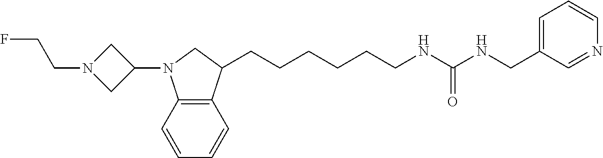 [SMILES: O=C(NCCCCCCC1CN(C2CN(CCF)C2)c2ccccc21)NCc1cccnc1] | 1-[6-[1-[1-(2- fluoro- ethyl)azetidin-3- yl]indolin-3-yl] hexyl]-3-(3- pyridylmethyl)urea | 4.1 min; 454.3035 [M + H] | M-3 |
| 129-C | 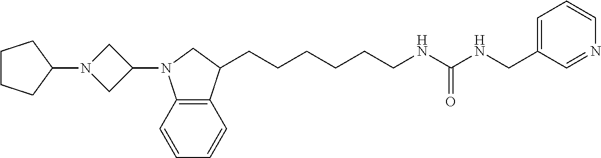 [SMILES: O=C(NCCCCCCC1CN(C2CN(C3CCCC3)C2)c2ccccc21)NCc1cccnc1] | 1-[6-[1-(1-cyclo- pentylazetidin-3- yl)indolin-3-yl] hexyl]-3-(3- pyridylmethyl)urea | 4.4 min; 476.3444 [M + H] | M-3 |

TABLE 3

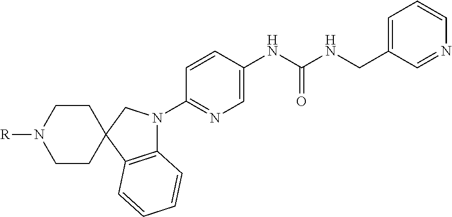

| # | R |
|---|---|
| 130 | 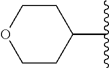 [SMILES: *C1CCOCC1] |
| 131 | 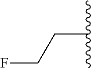 [SMILES: *CCF] |
| 132 | 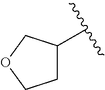 [SMILES: *C1CCOC1] |
| 133 | 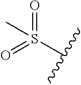 [SMILES: *S(C)(=O)=O] |
| 134 | 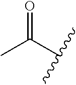 [SMILES: *C(C)=O] |
| 135 | 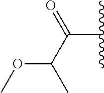 [SMILES: *C(=O)C(C)OC] |
| 136 | 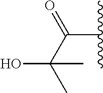 [SMILES: *C(=O)C(C)(C)O] |
| 137 | 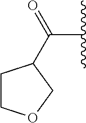 [SMILES: *C(=O)C1CCOC1] |
| 138 | 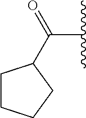 [SMILES: *C(=O)C1CCCC1] |
| 139 | 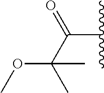 [SMILES: *C(=O)C(C)(C)OC] |
| 143 | 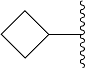 [SMILES: *C1CCC1] |
| 144 | 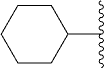 [SMILES: *C1CCCCC1] |
| 145 | 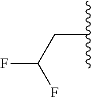 [SMILES: *CC(F)F] |
| 146 | 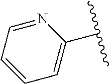 [SMILES: *c1ccccn1] |

TABLE 4

| 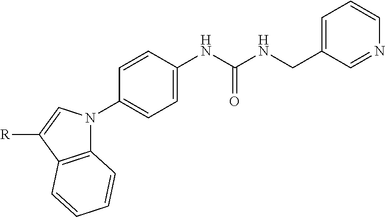 | |
|---|---|
| # | R |
| 147 | 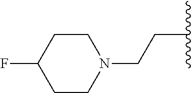 [SMILES: *CCN1CCC(F)CC1] |
| 148 | 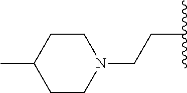 [SMILES: *CCN1CCC(C)CC1] |
| 149 | 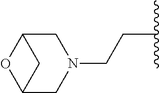 [SMILES: *CCN1CC2CC(C1)O2] |
| 150 | 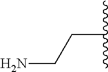 [SMILES: *CCN] |
| 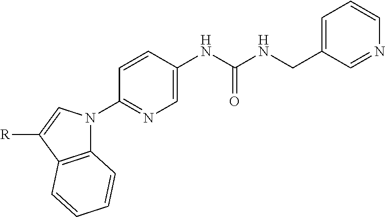 | |
| # | R |
| 151 | 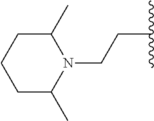 [SMILES: *CCN1C(C)CCCC1C] |
| 152 | 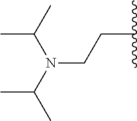 [SMILES: *CCN(C(C)C)C(C)C] |

TABLE 5

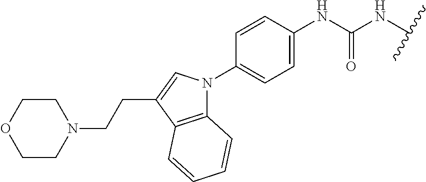 [SMILES: *NC(=O)Nc1ccc(-n2cc(CCN3CCOCC3)c3ccccc32)cc1]

| # | R |
|---|---|
| 153 | 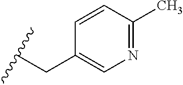 [SMILES: *Cc1ccc(C)nc1] |
| 154 | 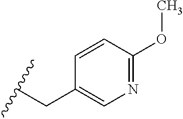 [SMILES: *Cc1ccc(OC)nc1] |
| 155 | 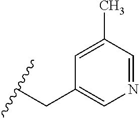 [SMILES: *Cc1cncc(C)c1] |
| 156 | 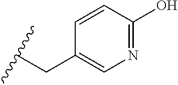 [SMILES: *Cc1ccc(O)nc1] |

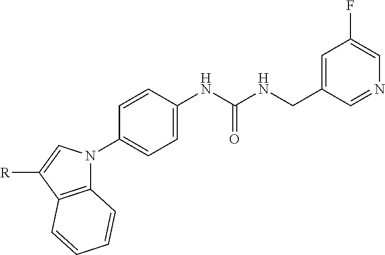

| # | R |
|---|---|
| 157 | 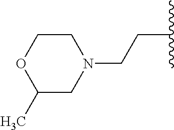 [SMILES: *CCN1CCOC(C)C1] |
| 158 | 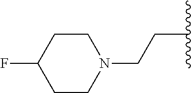 [SMILES: *CCN1CCC(F)CC1] |
| 159 | 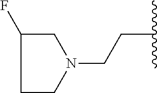 [SMILES: *CCN1CCC(F)C1] |
| 160 | 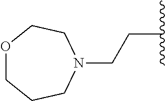 [SMILES: *CCN1CCCOCC1] |

TABLE 6

| | | | HPLC: | Syn. |
|---|---|---|---|---|
| Example | Structure | IUPAC Name | LCMS | Method |
| 150 | 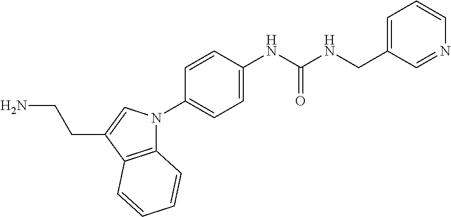 [SMILES: NCCc1cn(-c2ccc(NC(=O)NCc3cccnc3)cc2)c2ccccc12] | 1-{4-[3-(2- Aminoethyl)-1H-indol- 1-yl]phenyl}-3- (pyridin-3- ylmethyl)urea | 3.364 min; 386.4 [M + H] | P |
| 151 | 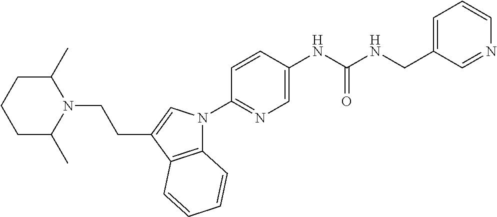 [SMILES: CC1CCCC(C)N1CCc1cn(-c2ccc(NC(=O)NCc3cccnc3)cn2)c2ccccc12] | l-(6-{3-[2-(2,6- Dimethylpiperidin-1- yl)ethyl]-1H-indol-1- yl}pyridin-3-yl)-3- (pyridin-3- ylmethyl)urea | 3.711 min; 483.2 [M + H] | P |
| 152 | 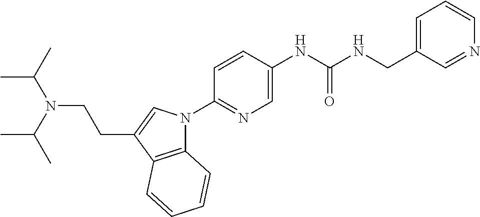 [SMILES: CC(C)N(CCc1cn(-c2ccc(NC(=O)NCc3cccnc3)cn2)c2ccccc12)C(C)C] | 1-(6-{3-[2-(Dipropan- 2-ylamino)ethyl]-1H- indol-1-yl}pyridin-3- yl)-3-(pyridin-3- ylmethyl)urea | 3.661 min; 471.2 [M + H] | P |
| 147 | 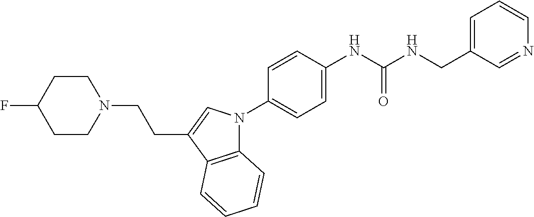 [SMILES: O=C(NCc1cccnc1)Nc1ccc(-n2cc(CCN3CCC(F)CC3)c3ccccc32)cc1] | 1-(4-{3-[2-(4- Fluoropiperidin-1- yl)ethyl]-1H-indol-1- yl}phenyl)-3-(pyridin- 3-ylmethyl)urea | 2.913 min; 472.3 [M + H] | P |
| 148 | 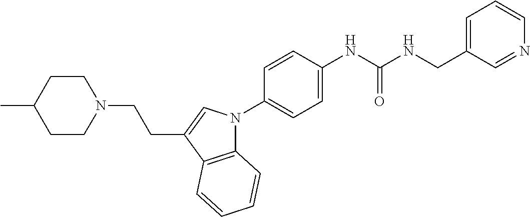 [SMILES: CC1CCN(CCc2cn(-c3ccc(NC(=O)NCc4cccnc4)cc3)c3ccccc23)CC1] | 1-(4-{3-[2-(4- Methylpiperidin-1- yl)ethyl]-1H-indol-1- yl}phenyl)-3-(pyridin- 3-ylmethyl)urea | 3.838 min; 468.2 [M + H] | P |
| 153 | 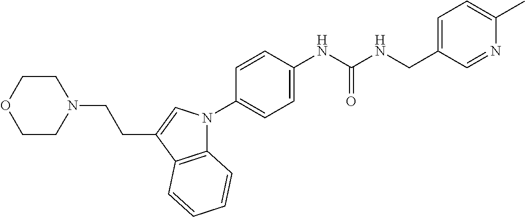 [SMILES: Cc1ccc(CNC(=O)Nc2ccc(-n3cc(CCN4CCOCC4)c4ccccc43)cc2)cn1] | 1-[(6-Methylpyridin-3- yl)methyl]-3-(4-{3-[2- (morpholin-4-yl)ethyl]- 1H-indol-1- yl}phenyl)urea | 2.899 min; 470.2 [M + H] | P |
| 154 | 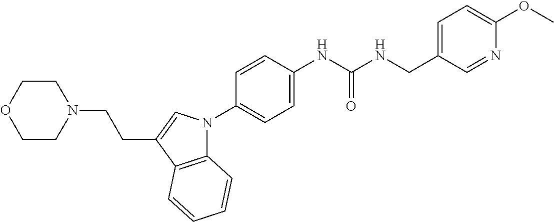 [SMILES: COc1ccc(CNC(=O)Nc2ccc(-n3cc(CCN4CCOCC4)c4ccccc43)cc2)cn1] | 1-[(6-Methoxypyridin- 3-yl)methyl]-3-(4-{3- [2-(morpholin-4- yl)ethyl]-1H-indol-1- yl}phenyl)urea | 4.287 min: 486.1 [M + H] | P |
| 155 | 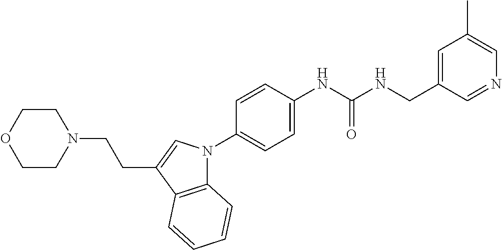 [SMILES: Cc1cncc(CNC(=O)Nc2ccc(-n3cc(CCN4CCOCC4)c4ccccc43)cc2)c1] | 1-[(5-Methylpyridin-3- yl)methyl]-3-(4-{3-[2- (morpholin-4-yl)ethyl]- 1H-indol-1- yl}phenyl)urea | 3.581 min; 470.1 [M + H] | P |
| 156 | 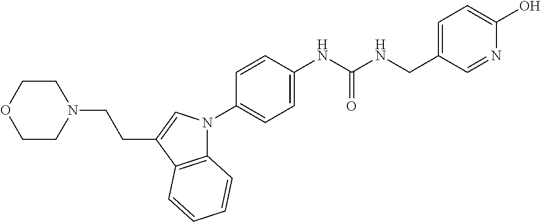 [SMILES: O=C(NCc1ccc(O)nc1)Nc1ccc(-n2cc(CCN3CCOCC3)c3ccccc32)cc1] | 1-(4-{3-[2-(Morpholin- 4-yl)ethyl]-1H-indol-1- yl}phenyl)-3-[(6-oxo- 1,6-dihydropyridin-3- yl)methyl]urea | 2.912 min; 472.3 [M + H] | P |

TABLE 7

| | HPLC | | |
|---|---|---|---|
| Example | Retention | | |
| Comp'd | Time | Mass Spec | |
| Number | (minutes) | (M + H)⁺ | H¹-NMR Data (ppm) δ |
| 150 | 3.364 | 386.4 | ¹H NMR (DMSO-d₆) δ 8.85 (s, 1H), 8.5 (s, 1H), |
| | | | 8.45 (d, 1H), 7.7 (d, 1H), 7.6 (m, 3H), 7.4 (m, 5H), 7.2 (m, |
| | | | 2H), 6.8 (t, 1H), 4.35 (d, 2H), 2.9 (m, 4H), 2.2 (m, |
| | | | 2H). |
| 151 | 3.711 | 483.2 | ¹H NMR (DMSO-d₆) δ 9.4 (bs, 1H), 9.0 (s, 1H), 8.6 (s, |
| | | | 2H), 8.45 (d, 1H), 8.2 (m, 1H), 8.15 (m, 1H), 8.0 (m, |
| | | | 1H), 7.71 (m, 1H), 7.6 (m, 2H), 7.4 (m, 1H), 7.2 (m, |
| | | | 2H), 6.9 (t, 1H), 5.7 (s, 1H), 4.4 (d, 2H), 3.5 (m, 4H), |
| | | | 3.15 (m, 2H), 1.9 (m, 2H), 1.6 (m, 5H), 1.4 (m, 4H), |
| | | | 1.3 (m, 3H), 1.2 (m, 3H). |
| 152 | 3.661 | 471.2 | ¹H NMR (DMSO-d₆) δ 9.0 (s, 1H), 8.7 (m, 1H), |
| | | | 8.55 (m, 1H), 8.45 (m, 1H), 8.25 (m, 1H), 8.1 (m, 1H), |
| | | | 8.0 (s, 1H), 7.7 (d, 1H), 7.6 (m, 2H), 7.4 (m, 1H), 7.2 (m, |
| | | | 2H), 6.9 (m, 1H), 4.4 (m, 2H), 4.2 (m, 1H), 3.7 (m, |
| | | | 2H), 3.4 (m, 3H), 3.2 (m, 2H), 1.4 (m, 11H), 1.2 (m, |
| | | | 6H), 0.8 (m, 1H). |
| 147 | 2.913 | 472.3 | ¹H NMR (CDCl₃) δ 8.5 (bd, 2H), 7.8 (d, 1H), 7.6 (d, |
| | | | 2H), 7.5 (d, 2H), 7.4 (d, 1H), 7.2 (m, 4H), 6.9 (m, 1H), |
| | | | 5.9 (bs, 1H), 4.8 (bd, 1H), 4.4 (d, 2H), 3.2 (m, 2H), |
| | | | 3.0 (m, 6H), 2.0-2.3 (m, 8H), 1.3 (m, 3H). |
| 148 | 3.838 | 468.2 | ¹H NMR (DMSO-d₆) δ 8.9 (s, 1H), 8.5 (s, 1H), 8.4 (d, |
| | | | 1H), 7.7 (t, 2H), 7.6 (m, 2H), 7.4 (m, 3H), 7.2 (m, 2H), |
| | | | 6.8 (t, 1H), 4.4 (d, 2H), 3.6 (m, 2H), 3.4 (m, 1H), |
| | | | 3.2 (m, 2H), 3.0 (m, 3H), 1.8 (m, 2H), 1.6 (m, 1H), 1.4 (m, |
| | | | 2H), 1.2 (m, 2H), 0.9 (m, 3H). |
| 153 | 2.899 | 470.2 | ¹H NMR (CDCl₃) δ 8.4 (s, 1H), 7.6 (t, 2H), 7.4 (m, |
| | | | 4H), 7.2 (m, 4H), 5.1 (t, 1H), 4.5 (m, 2H), 3.8 (t, 4H), |
| | | | 3.1 (t, 2H), 2.8 (m, 2H), 2.7 (s, 3H), 2.5 (s, 3H), |
| | | | 1.4 (m, 1H), 1.3 (m, 2H). |
| 154 | 4.287 | 486.1 | ¹H NMR (CDCl₃) δ 8.1 (m, 1H), 7.6 (m, 2H), 7.4 (m, |
| | | | 5H), 7.2 (m, 3H), 6.7 (m, 1H), 6.5 (m, 1H), 5.1 (bm, |
| | | | 1H), 4.4 (dd, 2H), 3.9 (s, 3H), 3.8 (m, 4H), 3.0 (t, 2H), |
| | | | 2.8 (t, 2H), 2.6 (m, 4H), 1.3 (m, 4H). |
| 155 | 3.581 | 470.1 | ¹H NMR (CDCl₃) δ 8.4 (s, 2H), 7.6 (d, 1H), 7.5 (s, |
| | | | 1H), 7.4 (m, 5H), 7.1 (m, 3H), 7.0 (s, 1H), 5.4 (bt, |
| | | | 1H), 4.5 (d, 2H), 3.8 (t, 4H), 3.0 (t, 2H), 2.8 (m, 2H), |
| | | | 2.6 (s, 4H), 2.3 (s, 3H), 1.4 (s, 1H), 1.3 (s, 2H). |
| 156 | 2.912 | 472.3 | ¹H NMR (DMSO-d₆) δ 11.4 (s, 1H), 8.8 (s, 1H), |
| | | | 7.6 (m, 3H), 7.4 (m, 5H), 7.3 (s, 1H), 7.1 (m, 2H), 6.5 (t, |
| | | | 1H), 6.3 (s, 1H), 4.0 (d, 2H), 3.6 (s, 3H), 2.9 (m, 2H), |
| | | | 2.6 (m, 2H), 1.2 (m, 10H), 0.8 (m, 1H). |

TABLE 8

| Ex- | | | HPLC |
|---|---|---|---|
| am- | | | Retention |
| ple | | | Time; Mass |
| No. | IUPAC Name | Structure | Spec |
| 161 | (2E)-3-(4-{3-[2-(2- Methylpyrrolidin-1- yl)ethyl]-1H-indol-1- yl)phenyl)-N-(pyridin-3- ylmethyl)prop-2-enamide | 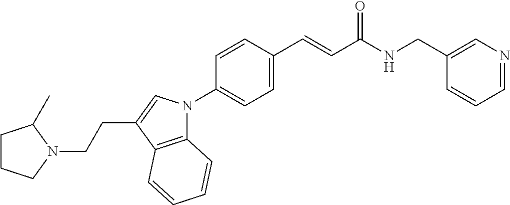 [SMILES: CC1CCCN1CCc1cn(-c2ccc(/C=C/C(=O)NCc3cccnc3)cc2)c2ccccc12] | 4.05 min; 465.2649 |
| 162 | (2E)-3-(4-{3-[2- (Diethylamio)ethyl]-1H- indol-1-yl}phenyl)-N- (pyridin-3-ylmethyl)prop-2- enamide | 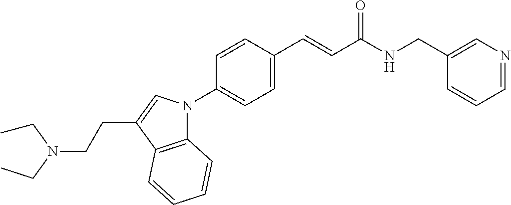 [SMILES: CCN(CC)CCc1cn(-c2ccc(/C=C/C(=O)NCc3cccnc3)cc2)c2ccccc12] | 4.0 min; 453.2679 [M + H] |
| 163 | (2E)-3-(4-{3-[2-(Dipropan- 2-ylamino)ethyl]-1H-indol- 1-yl(phenyl)-N-(pyridin-3- ylmethyl)prop-2-enamide | 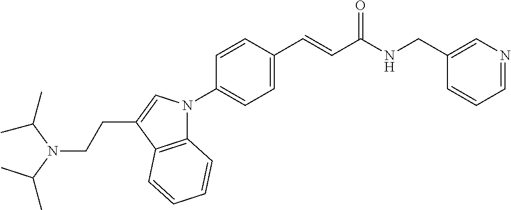 [SMILES: CC(C)N(CCc1cn(-c2ccc(/C=C/C(=O)NCc3cccnc3)cc2)c2ccccc12)C(C)C] | 4.26 min; 481.3000 [M + H] |
| 164 | (2E)-3-(4-{3-[2-(Morpholin- 4-yl)ethyl]-1H-indol-1- yl}phenyl)-N-(pyridin-3- ylmethyl)but-2-enamide | 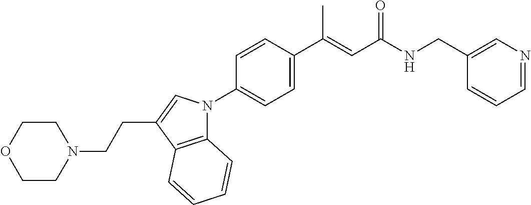 [SMILES: C/C(=C\C(=O)NCc1cccnc1)c1ccc(-n2cc(CCN3CCOCC3)c3ccccc32)cc1] | 4.0 min; 481.2593 [M + H] |
| 165 | (2E)-3-[4-(3-{[3- (Dimethylamino)azetidin-1- yl]carbonyl}-1H-indazol-1- yl)phenyl]-N-(pyridin-3- ylmethyl)prop-2-enamide | 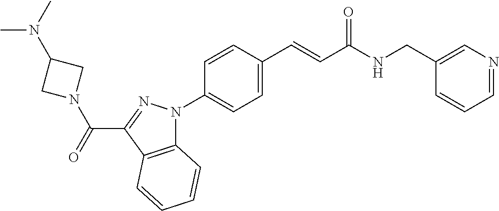 [SMILES: CN(C)C1CN(C(=O)c2nn(-c3ccc(/C=C/C(=O)NCc4cccnc4)cc3)c3ccccc23)C1] | 3.56 min; 481.23199 [M + H] |
| 166 | (2E)-3-[4-(3-{[3- (Dimethylamino)pyrrolidin- 1-yl]carbonyl}-1H-indazol- 1-yl)phenyl]-N-(pyridin-3- ylmethyl)prop-2-enamide | 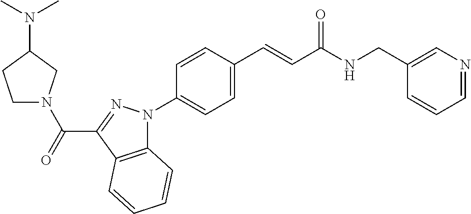 [SMILES: CN(C)C1CCN(C(=O)c2nn(-c3ccc(/C=C/C(=O)NCc4cccnc4)cc3)c3ccccc23)C1] | 3.56 min; 495.2484 [M + H] |
| 167 | (2E)-3-(4-{3-[(4- Cyclobutylpiperazin-1- yl)carbonyl]-1H-indazol-1- yl}phenyl)-N-(pyridin-3- ylmethyl)prop-2-enamide | 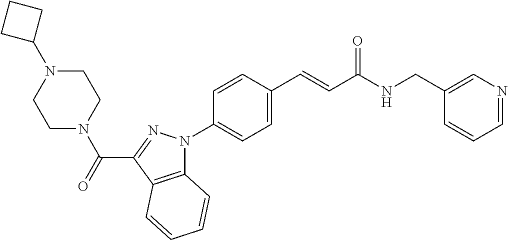 [SMILES: O=C(/C=C/c1ccc(-n2nc(C(=O)N3CCN(C4CCC4)CC3)c3ccccc32)cc1)NCc1cccnc1] | 3.77 min; 521.2628 [M + H] |
| 168 | (2E)-N-(Pyridin-3- ylmethyl)-3-[4-(3-{[4- (pyrrolidin-1-yl)piperidin-1- yl]carbonyl}-1H-indazol-1- yl)phenyl]prop-2-enamide | 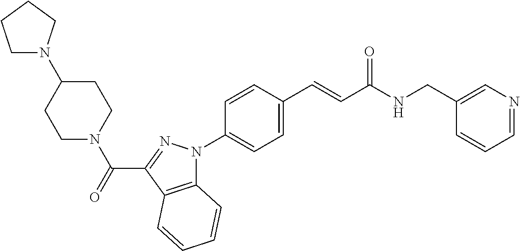 [SMILES: O=C(/C=C/c1ccc(-n2nc(C(=O)N3CCC(N4CCCC4)CC3)c3ccccc32)cc1)NCc1cccnc1] | 3.68 min; 535.2828 [M + H] |
| 169 | (2E)-3-[4-(3-{[3- (Morpholin-4-yl)azetidin-1- yl]carbonyl}-1H-indazol-1- yl)phenyl]-N-(pyridin-3- ylmethyl)prop-2-enamide | 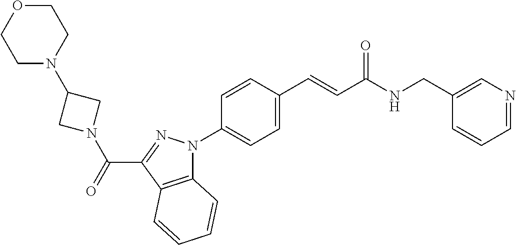 [SMILES: O=C(/C=C/c1ccc(-n2nc(C(=O)N3CC(N4CCOCC4)C3)c3ccccc32)cc1)NCc1cccnc1] | 3.78 min; 523.2456 [M + H] |
| 170 | (2E)-3-[4-(3-{[3- (Morpholin-4-yl)pyrrolidin- 1-yl]carbonyl}-1H-indazol- 1-yl)phenyl]-N-(pyridin-3- ylmethyl)prop-2-enamide | 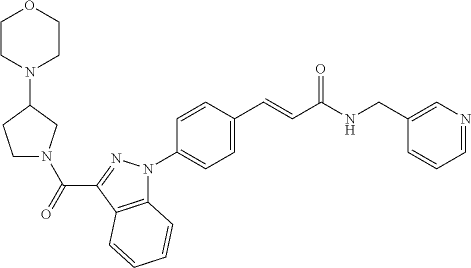 [SMILES: O=C(/C=C/c1ccc(-n2nc(C(=O)N3CCC(N4CCOCC4)C3)c3ccccc32)cc1)NCc1cccnc1] | 3.68 min; 537.2679 [M + H] |
| 171 | (2E)-3-[4-(3-{[4- (Morpholin-4-yl)piperidin- 1-yl]carbonyl}-1H-indazol- 1-yl)phenyl]-N-(pyridin-3- ylmethyl)prop-2-enamide | 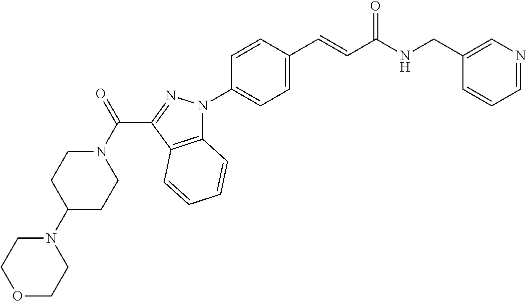 [SMILES: O=C(/C=C/c1ccc(-n2nc(C(=O)N3CCC(N4CCOCC4)CC3)c3ccccc32)cc1)NCc1cccnc1] | 3.69 min; 551.2846 [M + H] |
| 172 | (2E)-3-(4-{3-[2-(Pyrrolidin- 1-yl)ethyl]-1H-indol-1- yl}phenyl)-N-(1,3-thiazol-5- ylmethyl)prop-2-enamide | 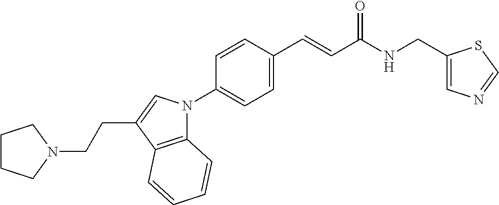 [SMILES: O=C(/C=C/c1ccc(-n2cc(CCN3CCCC3)c3ccccc32)cc1)NCc1cncs1] | 5.3 min; 457.2091 (M + H) |
| 173 | (2E)-3-(4-{3-[(4- Cyclohexylpiperazin-1- yl)carbonyl]-1H-indazol-1- yl}phenyl)-N-(pyridin-3- ylmethyl)prop-2-enamide | 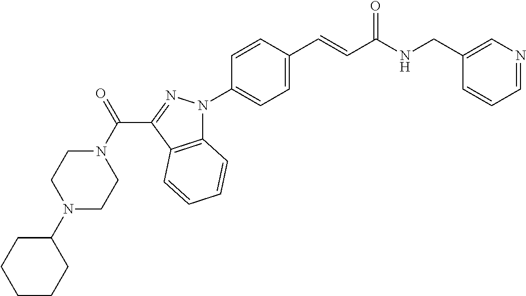 [SMILES: O=C(/C=C/c1ccc(-n2nc(C(=O)N3CCN(C4CCCCC4)CC3)c3ccccc32)cc1)NCc1cccnc1] | 4.53 min; 549.2991 (M + H) |
| 174 | (2E)-N-(Pyridin-3- ylmethyl)-3-(4-{3-[2- (pyrrolidin-1-yl)ethyl]-1H- indol-1-yl}phenyl)prop-2- enamide | 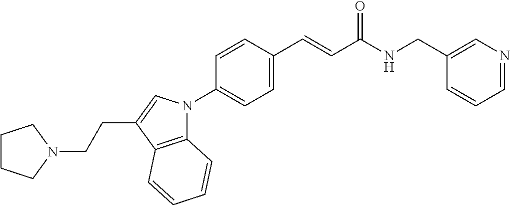 [SMILES: O=C(/C=C/c1ccc(-n2cc(CCN3CCCC3)c3ccccc32)cc1)NCc1cccnc1] | 4.39 min; 451.2529 (M + H) |
| 175 | (2E)-3-(4-{3-[2-(Morpholin- 4-yl)ethyl]-1H-indol-1- yl}phenyl)-N-(pyridin-3- ylmethyl)prop-2-enamide | 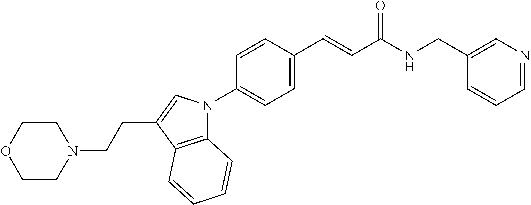 [SMILES: O=C(/C=C/c1ccc(-n2cc(CCN3CCOCC3)c3ccccc32)cc1)NCc1cccnc1] | 4.21 min; 467.2452 (M + H) |
| 176 | (2E)-3-{4-[3-(1,4′- Bipiperidin-1′-ylcarbonyl)- 1H-indazol-1-yl]phenyl}-N- (pyridin-4-yl)prop-2- enamide | 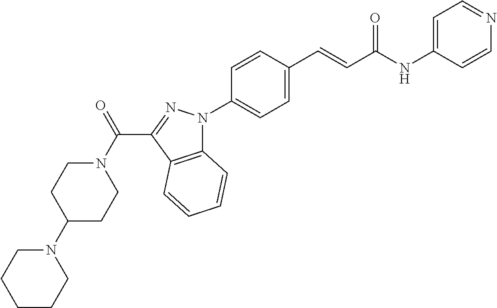 [SMILES: O=C(/C=C/c1ccc(-n2nc(C(=O)N3CCC(N4CCCCC4)CC3)c3ccccc32)cc1)Nc1ccncc1] | 4.449 min; 535.28160 (M + H) |
| 177 | (2E)-3-{4-[3-(1,4′- Bipiperidin-1′-ylcarbonyl)- 1H-indazol-1-yl]phenyl}-N- (pyridin-3-ylmethyl)prop-2- enamide | 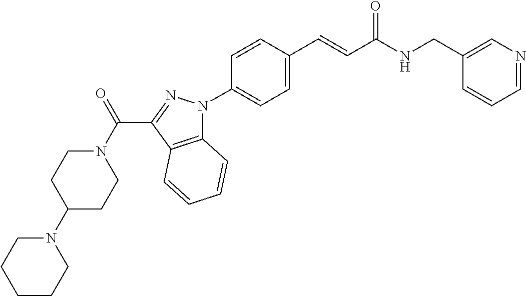 [SMILES: O=C(/C=C/c1ccc(-n2nc(C(=O)N3CCC(N4CCCCC4)CC3)c3ccccc32)cc1)NCc1cccnc1] | 4.18 min; 549.29739 (M + H) |
| 178 | 2-(4-{3-[2-(Morpholin-4- yl)ethyl]-1H-indol-1- yl}phenyl)-N-(pyridin-3- ylmethyl) cyclopropanecarboxamide | 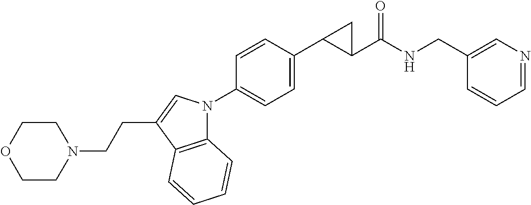 [SMILES: O=C(NCc1cccnc1)C1CC1c1ccc(-n2cc(CCN3CCOCC3)c3ccccc32)cc1] | 3.89 min; 481.2589 [M + H] |
| 179 | (2E)-3-(6-{3-[2-(Morpholin- 4-yl)ethyl]-1H-indol-1- yl}pyridin-3-yl)-N-(pyridin- 3-ylmethyl)prop-2-enamide | 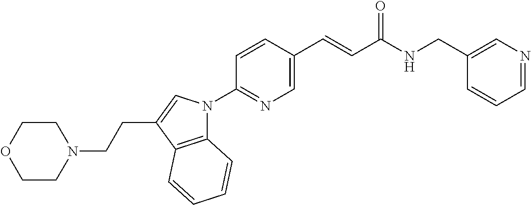 [SMILES: O=C(/C=C/c1ccc(-n2cc(CCN3CCOCC3)c3ccccc32)nc1)NCc1cccnc1] | 3.7 min; 468.2455 [M + H] |
| 180 | 3-(4-{3-[2-(Morpholin-4- yl)ethyl]-1H-indol-1- yl}phenyl)-N-(pyridin-3- ylmethyl)propanamide | 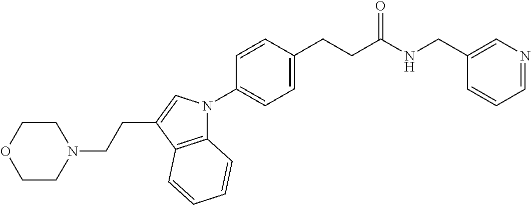 [SMILES: O=C(CCc1ccc(-n2cc(CCN3CCOCC3)c3ccccc32)cc1)NCc1cccnc1] | 3.84 min; 469.2677 [M + H] |
| 181 | (2E)-3-(4-{3-[2-(1,4- Oxazepan-4-yl)ethyl]-1H- indol-1-yl}phenyl)-N- (pyridin-3-ylmethyl)prop-2- enamide | 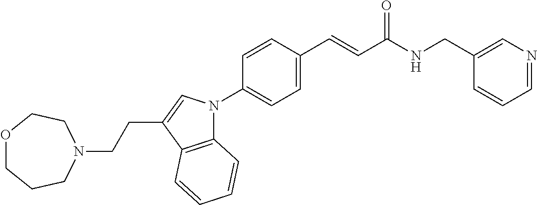 [SMILES: O=C(/C=C/c1ccc(-n2cc(CCN3CCCOCC3)c3ccccc32)cc1)NCc1cccnc1] | 3.88 min; 481.2595 [M + H] |
| 182 | (2E)-3-(4-{3-[2-(Piperidin- 1-yl)ethyl]-1H-indol-1- yl}phenyl)-N-(pyridin-3- ylmethyl)prop-2-enamide | 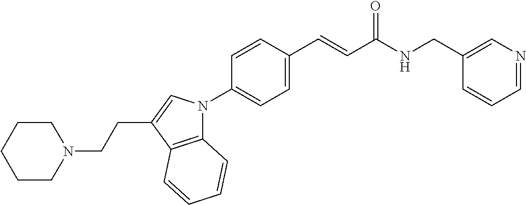 [SMILES: O=C(/C=C/c1ccc(-n2cc(CCN3CCCCC3)c3ccccc32)cc1)NCc1cccnc1] | 4.09 min; 465.22698 [M + H] |
| 183 | 2-{4-[3-(1,4′-Bipiperidin-1′- ylcarbonyl)-1H-indazol-1- yl]phenyl}-N-(pyridin-3- ylmethyl)acetamide | 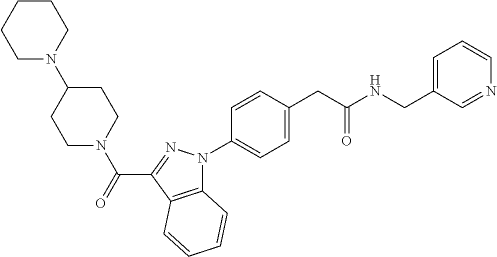 [SMILES: O=C(Cc1ccc(-n2nc(C(=O)N3CCC(N4CCCCC4)CC3)c3ccccc32)cc1)NCc1cccnc1] | 3.9 min; 537.2981 [M + H] |
| 184 | (2E)-3-(4-{3-[2-(2,6- Dimethylpiperidin-1- yl)ethyl]-1H-indol-1- yl}phenyl)-N-(pyridin-3- ylmethyl)prop-2-enamide | 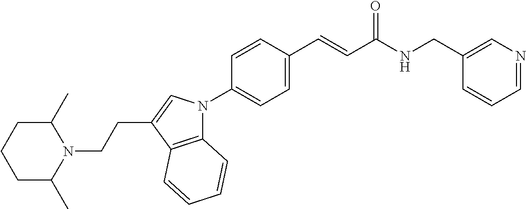 [SMILES: CC1CCCC(C)N1CCc1cn(-c2ccc(/C=C/C(=O)NCc3cccnc3)cc2)c2ccccc12] | 4.37 min; 493.3028 [M + H] |
| 185 | (2E)-3-(4-{3-[2-(4- Fluoropiperidin-1-yl)ethyl]- 1H-indol-1-yl}phenyl)-N- (pyridin-3-ylmethyl)prop-2- enamide | 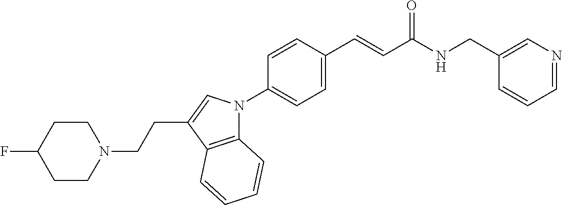 [SMILES: O=C(/C=C/c1ccc(-n2cc(CCN3CCC(F)CC3)c3ccccc32)cc1)NCc1cccnc1] | 4.13 min; 483.2575 [M + H] |
| 186 | (2E)-3-(4-{3-[2-(3- Hydroxypiperidin-1- yl)ethyl]-1H-indol-1- yl}phenyl)-N-(pyridin-3- ylmethyl)prop-2-enamide | 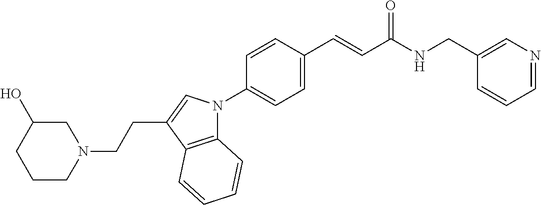 [SMILES: O=C(/C=C/c1ccc(-n2cc(CCN3CCCC(O)C3)c3ccccc32)cc1)NCc1cccnc1] | 3.9 min; 481.2605 [M + H] |

TABLE 9

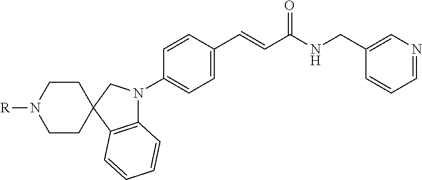

| # | R |
|---|---|
| 187 | 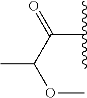 [SMILES: *C(=O)C(C)OC] |
| 188 | 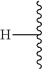 [SMILES: *[H]] |

### Biochemical and Biological Examples

### Cytotoxicity Assay

HCT116 cells were seeded in 96 well plates (Greiner Bio-One, Monroe, N.C.) and allowed to settle overnight. Test compound dissolved in dimethyl sulfoxide (DMSO) was added and drug incubation proceeded for 72 hours. When applicable, a 1000× solution of nicotinic acid (NA; Sigma-Aldrich, St. Louis, Mo.) dissolved in water was generated, and 1×NA (10 μM final concentration) was added at the same time as the test compound. After 72 hour, 50 μL of CellTiter-Glo Luminescent Cell Viability Assay reagent (Promega Corporation, Madison, Wis.) was added to cells in 200 μL of cellular media. After a proscribed incubation period, luminescence was measured using a TopCount NXT plate reader (PerkinElmer, Waltham, Mass.).

Example compounds I-28 were tested in this assay. Many of those compounds exhibited HCT116 cell cytotoxicity with an IC₅₀ of less than 100 nM. For example, example compound number 4 exhibited an IC₅₀ of about 8 nM, example compound number 6 exhibited an IC₅₀ of about 18 nM, example compound number 15 exhibited an IC₅₀ of about 60 nM, and example compound number 27 exhibited an IC₅₀ of about 4 nM.

Example compounds 30-33, 37-40, 42, 43, 45-47, 49, 50, 52-54, 56, 57, 61, 62, 64-67, 69-73, 75-78, 80, 82, 84-87, 90-95, 97-105, 107-117, 119, 121, 124, 125, 127, 128, and 130 were tested in this assay and exhibited HCT116 cell cytotoxicity with an IC₅₀ of less than 100 nM. For example, example compound number 33 exhibited an IC₅₀ of about 1 nM, example compound number 47 exhibited an IC₅₀ of less than 1 nM, example compound number 61 exhibited an IC₅₀ of less than 1 nM, example compound number 78 exhibited an IC₅₀ of about 1 nM, example compound number 109 exhibited an IC₅₀ of about 1 nM, and example compound number 119 exhibited an in vitro IC₅₀ of about 4 nM.

Example compound 34, 36, 41, 44, 48, 51, 55, 58-60, 63, 68, 74, 79, 81, 83, 88, 89, 96, 106, 120, 123, 126, and 129 were tested in this assay and exhibited HCT116 cell cytotoxicity with an IC₅₀ of greater than or equal to 100 nM.

All of the example compounds of Table 8, except for example compound numbers 164 and 184-186, were tested in this assay. Compounds 161-163, 165-171, 173-175, 177, and 179-183 exhibited HCT116 cell cytotoxicity with an IC₅₀ of less than 100 nM. For example, example compound number 169 exhibited an IC₅₀ of about 10 nM and example compound number 175 exhibited an IC₅₀ of about 7 nM.

### Liquid Chromatograph-Mass Spectrometry

Bound proteins were digested by treating the beads with trypsin as follows. After the final wash, beads were resuspended in an equal volume of trypsin digest buffer (50 mM ammonium bicarbonate, (pH 8.0), 5% acetonitrile, 1 mM calcium chloride). Samples were reduced with 5 mM DTT at 65° C. for 15 minutes and alkylated with 10 mM iodoacetamide in the dark at 30° C. for 30 minutes. Sequencing grade modified trypsin (Promega Corporation, Madison, Wis.) was added and samples digested for 1.5 hours at 37° C.

### Nampt Activity Assays

5-phosphoribosyl-1-pyrophosphate (PRPP), ATP, NaM, NaMN, Triton X-100, UDP-glucose and diaphorase were purchased from Sigma-Aldrich, St. Louis, Mo. Human NAMPT, NMN adenylyltransferase (NMNAT1) and UDP-glucose dehydrogenase (UGDH) encoding DNAs were each inserted into a house-modified E. Coli expression vector such that the expressed proteins carried an N-terminal 6×His tag. The His-tagged proteins were expressed in the BL21-AI E. Coli expression strain (Invitrogen Corporation, Carlsbad, Calif.) following induction by 0.2% L-arabinose and 0.5 mM IPTG at 30° C. Proteins were purified on Ni-NTA resin (Qiagen, Germantown, Md.).

The assay for Nampt catalytic activity was constructed based on a previously published coupled enzyme fluorometric technique, which employs NADH as ultimate analyte (Revollo, J. R. et al. Biol. Chem. 279, 50754-50763 (2004)). A substantial improvement in assay sensitivity was achieved by switching from direct detection to a resazurin/diaphorase-based fluorometric detection system for NADH (Guilbault, G. G., and Kramer, D. N. Anal. Chem. 37, 1219-1221 (1965)). The standard inhibition analyses were performed in a real-time mode in 96-well microtiter plates using 50 mM Tris-HCl, pH 7.5, 1% DMSO (v/v), 0.01% Triton X-100 (v/v), 10 mM MgCl₂, 2 mM ATP, 3 μM NAM, 8 μM PRPP, 50 μM Nampt, as well as the following detection reagents: 5 nM Nmnat, 200 nM Ugdh, 200 μM UDP-glucose, 0.02 U/mL diaphorase and 0.25 μM resazurin. Incubation of samples at room temperature for up to 3 hours was followed by quantification of fluorescence intensities at excitation and emission wavelengths of 510 nm and 590 nm, respectively, using Gemini XS plate reader (Molecular Devices, Sunnyvale, Calif.). The counter-assay intended to disqualify false positives, such as inhibitors of detection enzymes or fluorescence quenchers, was carried out essentially as described above with an exception that 1 μM NaMN was substituted for Nampt. A preparation of catalytically inactive Nampt-D313A mutant enzyme was used as a negative control for assay development.

All of the compounds of Table 1 were tested using this assay. For example, example compound number 1 exhibited an in vitro IC₅₀ of about 10 nM, example compound number 4 exhibited an in vitro IC₅₀ of about 1 nM, example compound number 6 exhibited an in vitro IC₅₀ of about 2 nM, example compound number 15 exhibited an in vitro IC₅₀ of about 1 nM, example compound number 27 exhibited an in vitro IC₅₀ of about 1 nM, and example compound number 29 exhibited an in vitro IC₅₀ of about 1 nM.

All of the compounds of Table 2 were tested using this assay except for example compounds 35, 71, and 122. For example, example compound number 33 exhibited an in vitro IC₅₀ of less than 1 nM, example compound number 47 exhibited an in vitro IC₅₀ of less than 1 nM, example compound number 61 exhibited an in vitro IC₅₀ of less than 1 nM, example compound number 78 exhibited an in vitro IC₅₀ of about 1 nM, example compound number 109 exhibited an in vitro IC₅₀ of about 1 nM, and example compound number 119 exhibited an in vitro IC₅₀ of about 2 nM.

Example compound numbers 147-148 and 150-156 were tested in this assay. Each of example compound numbers 147, 148, and 150-152 exhibited an in vitro IC₅₀ of less than 1 nM. Each of example compound numbers exhibited an in vitro IC₅₀ of about 10 nM or less.

All of the compounds of Table 8, except for example compound numbers 184-186, were tested using this assay. For example, example compound number 169 exhibited an in vitro IC₅₀ of about 3 nM and example compound number 175 exhibited an in vitro IC₅₀ of about 1 nM.

### Assay to Measure NAD⁺ in Cellular Lysates

NAD⁺ in cells was measured by modification of existing protocols (Lee, H. I., et al. Exp. Mol. Med. 40, 246-253 (2008)). MCF-10A cells stably transduced with the PIK3CA(H1047R) oncogene were seeded in 96 well plates at very high density (100% confluence) and allowed to settle overnight. Test compound dissolved in DMSO was added and drug incubation proceeded for 20-24 hours. Cells were washed with PBS and harvested by incubation in 25 μL 0.5 M perchloric acid (HClO₄) followed by vigorous shaking at 4° C. for 15 minutes. Acidic cell lysates were neutralized by adding 8 μL of 2 M KOH/0.2 M K₂HPO₄. The entire lysate volume was transferred to a centrifuge plate and spun at 3000 rpm in a table top centrifuge (4° C.) for 5 minutes to clear the precipitate. Lysate was assayed for both NAD⁺ and ATP. For NAD⁺ measurement, 10 μL lysate from the centrifuged plate was added to 90 μL of reaction solution in Costar 96 half-well plates (Corning, Corning, N.Y.). The final concentration of the reaction mixture was 120 μM Tris-HCl, pH 7.5, 0.01% Triton X-100, 35 μM UDP-Glucose, 50 nM UGDH, 0.5 μM resazurin, and 0.1 unit/mL Diaphorase. Reactions were allowed to proceed for 1 hour at room temperature, after which time fluorescence was read on a Gemini plate reader as described above. For ATP measurement, 5 μL of cleared lysate was added to 195 μL PBS. 50 μL CellTiter-Glo reagent (Promega Corporation, Madison, Wis.) was added and ATP measured as described in the cytotoxicity assay methods.

### PAR Assay

To measure Poly (ADP-Ribose) Polymerase (PARP) activity, an imaging-based cellular assay was developed. MCF-10A cells stably transduced with the PIK3CA(H1047R) oncogene were seeded in 96 well plates and allowed to settle overnight. Test compound dissolved in DMSO was added and drug incubation proceeded for 20-24 hours. Under these conditions, Nampt inhibitors showed no evidence of toxicity. The next morning, hydrogen peroxide was added to the cells to a final concentration of 500 μM. After 8 minutes of hydrogen peroxide treatment, cells were fixed in 100%, −20° C. methanol. After re-hydrating and washing with PBS, cells were incubated in blocking buffer (HBSS, 1% BSA, 0.1% Tween20), and were then stained overnight with an anti-PAR mouse monoclonal antibody (Trevigen, Gaithersburg, Md.; 1:2000 dilution in blocking buffer). Cells were washed with PBS and incubated with 1:1000 of anti-mouse-Alexa488 (Invitrogen Corporation, Carlsbad, Calif.), 5 μg/mL Hoechst 33342 (Invitrogen), and 0.1 μg/mL HCS CellMask deep red (Invitrogen). Cells were washed with PBS and then stored in blocking buffer).

Images were acquired on a Pathway 855 instrument (BD Biosciences, San Jose, Calif.) using a 10× objective. Using Attovision software (BD Biosciences, San Jose, Calif.), the Hoechst signal was used to segment nuclei and the PAR signal for each nuclei in a well was subsequently averaged to generate a single value. After background subtraction using samples that were not incubated with the anti-PAR primary antibody, PAR intensity per well was graphed (Prism; GraphPad Software, Inc.; La Jolla, Calif.).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be understood that certain changes and modifications can be practiced within the scope of the appended claims.

1. A compound having a structure according to Formula I
J-K-L-E-Q-P Formula I
and pharmaceutically-acceptable salts and solvates thereof; wherein:
J is selected from: alkyl, nitro, cyano, alkoxy, C-amido, N-amido, haloalkyl, C-carboxy, O-carboxy, sulfonamide, amino, hydroxyl, mercapto, alkylthio, sulfonyl, sulfinyl, carbocycle, spiro-linked (i.e., two adjacent atoms of J are linked to one atom of K) carbocycle, cycloalkyl, spiro-linked cycloalkyl, cycloalkenyl, spiro-linked cycloalkenyl, heterocycle, spiro-linked heterocycle, heterocyclonoyl, aryl, spiro-linked aryl, heteroaryl, spiro-linked heteroaryl, carbocycloalkyl, heterocyclylalkyl, arylalkyl, arylalkenyl, heteroarylalkyl, heteroarylalkenyl, heteroarylalkynyl, or arylalkynyl, wherein any of the foregoing groups are optionally substituted at least once with alkyl, alkylene, alkenyl, alkenylene, alkynyl, alkynylene, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyamino carbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide, wherein any of the foregoing optional substituents are themselves optionally substituted;
K is an optionally further substituted 5-membered heteroaryl or heterocyclic ring;
L is either (i) an optionally-substituted phenyl or an optionally-substituted 5- or 6-membered heteroaryl ring, (ii) optionally-substituted 5- or 6-membered cycloalkyl, (iii) optionally-substituted alkyl, (iv) optionally-substituted alkenyl, or (v) optionally-substituted alkynyl;
E is either (i) —C₀₋₂ alkylene-N(H)—C(═X)—N(H)— or (ii) -M-C(═X′)—N(H)—, wherein X is O, S, or N—C≡N, wherein M is optionally-substituted ethenylene or optionally-substituted ethylene, and wherein X′ is O or S;
Q is optionally present and if present is optionally-substituted ethylene or optionally-substituted methylene;
P is an optionally-substituted pyridinyl ring;
with the proviso that when L is optionally-substituted alkyl, then K is an optionally-substituted 5-membered bicyclic heteroaryl or bicyclic heterocyclic ring (i.e., K comprises a 5-membered heteroaryl or heterocyclic ring fused to a second ring, wherein attachment to J and L is via the 5-membered heteroaryl or heterocyclic ring); and
with the proviso that when E is -M-C(═X′)—N(H)—, then K is not xanthine; and also with the proviso that when E is —C₀₋₂ alkylene-N(H)—C(═X)—N(H)—, then either K is an optionally-substituted 5-membered bicyclic heteroaryl or bicyclic heterocyclic ring (i.e., K comprises a 5-membered heteroaryl or heterocyclic ring fused to a second ring, wherein attachment to J and L is via the 5-membered heteroaryl or heterocyclic ring) or J is a spiro-linked moiety (i.e., two adjacent atoms of J are linked to one atom of K), such as, for example, spiro-linked carbocycle, spiro-linked cycloalkyl, spiro-linked cycloalkenyl, spiro-linked heterocycle, spiro-linked aryl, and spiro-linked heteroaryl; and
with the proviso that the compound is not:
Urea, N-(6-chloro-3-pyridinyl)-N′-[2-[4-(5-methyl-3-oxo-1H-imidazo[1,5-c]imidazol-2(3H)-yl)-1-piperidinyl]-2-oxo-1-phenylethyl]-;
Urea, N-[2-(3′-chloro[1,1′-biphenyl]-4-yl)-2-(1-cyclopentyl-4-piperidinyl)ethyl]-N′-3-pyridinyl-;
Urea, N-[2-(3′-cyano[1,1′-biphenyl]-4-yl)-2-(1-cyclopentyl-4-piperidinyl)ethyl]-N′-3-pyridinyl-;
2H-Pyrazino[2,1-c][1,2,4]triazine-1(6H)-carboxamide,hexahydro-6-[(4-hydroxyphenyl)methyl]-8-[[1-methyl-3-[4-[[[[6-(4-methyl-1-piperazinyl)-3-pyridinyl]amino]carbonyl]amino]phenyl]-1H-indol-7-yl]methyl]-4,7-dioxo-N-(phenylmethyl)-2-(2-propen-1-yl)-,(6S,9aS)-; or
2H-Pyrazino[2,1-c][1,2,4]triazine-1(6H)-carboxamide,hexahydro-6-[(4-hydroxyphenyl)methyl]-8-[[3-[4-[[[(6-methoxy-3-pyridinyl)amino]carbonyl]amino]phenyl]-1-methyl-1H-indol-7-yl]methyl]-4,7-dioxo-N-(phenylmethyl)-2-(2-propen-1-yl)-(6S,9aS)-.

2-5. (canceled)

6. The compound of claim 1, wherein the compound has a structure according to Formula II 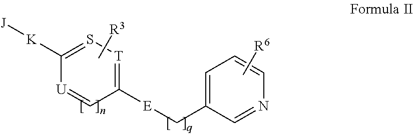
and pharmaceutically-acceptable salts and solvates thereof; wherein:
J and K are each as defined for Formula I;
S, T, and U are each independently carbon or nitrogen, provided that when any of S, T, or U is nitrogen, then there is no substituent on the nitrogen;
n is 0 or 1;
R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
E is either (i) —C₀₋₂ alkylene-N(H)—C(═X)—N(H)— or (ii) -M-C(═X′)—N(H)—, wherein X is O, S, or N—C≡N, wherein M is optionally-substituted ethenylene or optionally-substituted ethylene, and wherein X′ is O or S;
q is 0, 1, or 2, wherein any methylene group of the q region is optionally independently substituted with C₁₋₄ alkyl, halo, C₁₋₄ haloalkyl, or C₃ or C₄ cycloalkyl;
R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl; and
with the proviso that when E is -M-C(═X′)—N(H)—, then K is not xanthine; and also
with the proviso that when E is —C₀₋₂ alkylene-N(H)—C(═X)—N(H)—, then either K is an optionally-substituted 5-membered bicyclic heteroaryl or bicyclic heterocyclic ring (i.e., K comprises a 5-membered heteroaryl or heterocyclic ring fused to a second ring, wherein attachment to J and L is via the 5-membered heteroaryl or heterocyclic ring) or J is a spiro-linked moiety (i.e., two adjacent atoms of J are linked to one atom of K), such as, for example, spiro-linked carbocycle, spiro-linked cycloalkyl, spiro-linked cycloalkenyl, spiro-linked heterocycle, spiro-linked aryl, and spiro-linked heteroaryl; and,
with the proviso that the compound is not:
2H-Pyrazino[2,1-c][1,2,4]triazine-1(6H)-carboxamide, hexahydro-6-[(4-hydroxyphenyl)methyl]-8-[[3-[4-[[[(6-methoxy-3-pyridinyl)amino]carbonyl]amino]phenyl]-1-methyl-1H-indol-7-yl]methyl]-4,7-dioxo-N-(phenylmethyl)-2-(2-propen-1-yl)-, (6S,9aS)-;
Benzenepropanamide, 4-(2-methyl-3H-imidazo[4,5-b]pyridin-3-yl)-N-(3-pyridinylmethyl)-;
Pentanamide, 5-chloro-N-[(5-chloro-2-methyl-3-pyridinyl)methyl]-2-[[3-methoxy-4-(4-methyl-1H-imidazol-1-yl)phenyl]methylene]-,(2E)-; or
Pentanamide, 5-chloro-2-[[3-methoxy-4-(4-methyl-1H-imidazol-1-yl)phenyl]methylene]-N-[[6-(4-morpholinyl)-3-pyridinyl]methyl]-,(2E)-.

7-22. (canceled)

23. The compound of claim 1, wherein the compound has a structure according to Formula III 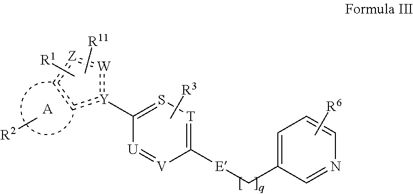
and pharmaceutically-acceptable salts and solvates thereof; wherein:
R¹ substitutes for a hydrogen and is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, and optionally-substituted sulfinyl;
R¹¹ is optionally present, and if present, substitutes a hydrogen and together with R¹ forms a spiro-linked heterocycle (i.e., R¹ and R¹¹ both attach to the same ring carbon atom) optionally substituted at a heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, or sulfonyl;
A is optionally present and when present is cycloalkyl, heterocycle, aryl, or heteroaryl;
R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl; with the proviso that R² is only present if A is present;
W, Y, and Z are each independently carbon or nitrogen, provided that at least one, but not both, of Y and Z is nitrogen;
S, T, U, and V are each independently carbon or nitrogen, provided that when any of S, T, U, or V is nitrogen, then there is no substituent on the nitrogen;
R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
E′ is either —C₀₋₂ alkylene-N(H)—C(═O)—N(H)— or 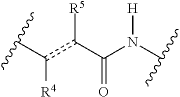
wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;
q is 0, 1, or 2, wherein any methylene group of the q region is optionally independently substituted with C₁₋₄ alkyl, halo, C₁₋₄ haloalkyl, or C₃ or C₄ cycloalkyl;
R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl;
with the proviso that when E′ is —C₀₋₂ alkylene-N(H)—C(═O)—N(H)—, then A is present; and
with the proviso that the compound is not:
Benzenepropanamide, 4-(2-methyl-3H-imidazo[4,5-b]pyridin-3-yl)-N-(3-pyridinylmethyl)-; and
with the proviso that when E is -M-C(═X′)—N(H)—, then K is not xanthine.

24-50. (canceled)

51. The compound of claim 1, wherein the compound has a structure according to Formula IV 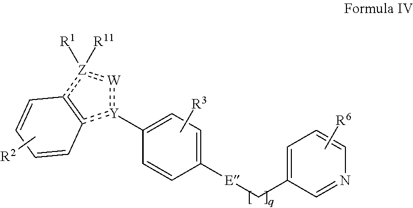
and pharmaceutically-acceptable salts and solvates thereof; wherein:
R¹ is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl;
R¹¹ is optionally present, and if present, substitutes a hydrogen and together with R¹ forms a spiro-linked heterocycle (i.e., R¹ and R¹¹ both attach to the same ring carbon atom) optionally substituted at a heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, or sulfonyl;
R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
W, Y, and Z are each independently carbon or nitrogen, provided that at least one, but not both, of Y and Z is nitrogen;
R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
E″ is either —N(H)—C(═O)—N(H)— or 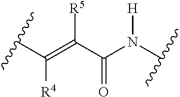
wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;
q is 0, 1, or 2, wherein any methylene group of the q region is optionally independently substituted with C₁₋₄ alkyl, halo, C₁₋₄ haloalkyl, or C₃ or C₄ cycloalkyl; and
R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl.

52-54. (canceled)

55. The compound of claim 1, wherein the compound has a structure according to Formula IVa 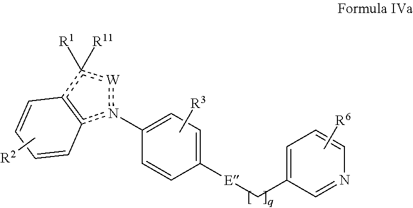
and pharmaceutically-acceptable salts and solvates thereof; wherein:
R¹ is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl;
R¹¹ is optionally present, and if present, substitutes a hydrogen and together with R¹ forms a spiro-linked heterocycle (i.e., R¹ and R¹¹ both attach to the same ring carbon atom) optionally substituted at a heteroatom of the heterocycle with alkyl, haloalkyl, cycloalkyl, heterocyclo, aryl, heteroaryl, halo, hydroxyl, alkoxy, alkoxyalkoxy, alkoxyalkanoyl, hydroxyalkanoyl, mercapto, arylalkyl, heteroarylalkyl, aldehyde, thiocarbonyl, heterocyclonoyl, cycloalkylcarbonyl, O-carboxy, C-carboxy, carboxylic acid, ester, C-carboxy salt, carboxyalkyl, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, C-amidoalkyl, N-amido, aminothio, hydroxyaminocarbonyl, alkoxyaminocarbonyl, cyano, nitrile, cyanato, isocyanato, thiocyanato, isothiocyanato, sulfinyl, or sulfonyl;
R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
W is carbon or nitrogen;
R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl; 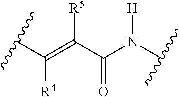
E″ is either —N(H)—C(═O)—N(H)— or wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;
q is 1 or 2, wherein any methylene group of the q region is optionally independently substituted with C₁₋₄ alkyl, halo, C₁₋₄ haloalkyl, or C₃ or C₄ cycloalkyl; and
R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl.
In some embodiments of the compounds of Formula IV, the ring comprising W is aromatic. In some embodiments of the compounds of Formula IV, the ring comprising W is alicyclic. In some of such embodiments, the ring comprising W contains only single bonds.

56. The compound of claim 1, wherein the compound has a structure according to Formula IVb 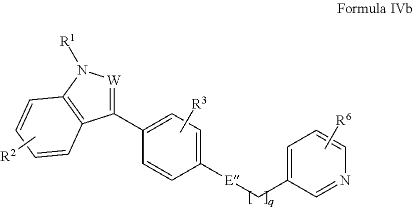
and pharmaceutically-acceptable salts and solvates thereof; wherein:
R¹ is selected from halo, hydroxyl, C₁₋₅ alkyl, C₁₋₅ haolalkyl, C₂₋₅ alkanoyl, C₂₋₅ hydroxyalkanoyl, C₃₋₆ heterocyclic, C₃₋₆ carbocycle, C₃₋₆ heterocyclonoyl, C₃₋₆ heterocycloalkyl, heteroaryl, aryl, nitro, cyano, C₁₋₅ alkoxy, C-amido, ester, N-amido, trihalomethyl, C-carboxy, O-carboxy, sulfonamide, amino, aminoalkyl, hydroxyl, mercapto, alkylthio, sulfonyl, or sulfinyl, wherein any of the foregoing are each optionally substituted one or more times with halo, hydroxyl, C₁₋₅ alkyl, C₁₋₅ haolalkyl, C₂₋₅ alkanoyl, C₂₋₅ hydroxyalkanoyl, optionally-substituted C₃₋₆ heterocyclic, optionally-substituted C₃₋₆ carbocycle, optionally-substituted C₃₋₆ heterocyclonoyl, optionally-substituted C₃₋₆ heterocycloalkyl, optionally-substituted heteroaryl, optionally-substituted aryl, nitro, cyano, optionally-substituted optionally-substituted C₁₋₅ alkoxy, optionally-substituted optionally-substituted C-amido, optionally-substituted ester, optionally-substituted N-amido, trihalomethyl, optionally-substituted C-carboxy, optionally-substituted O-carboxy, optionally-substituted sulfonamide, optionally-substituted amino, optionally-substituted aminoalkyl, hydroxyl, mercapto, alkylthio, optionally-substituted sulfonyl, or optionally-substituted sulfinyl;
R² is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
W is carbon or nitrogen;
R³ is optionally present, and if present, substitutes one, two, three, or four hydrogens, and in each instance is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, or sulfinyl;
E″ is either —N(H)—C(═O)—N(H)— or 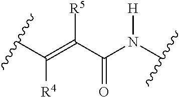
wherein R⁴ is hydro, hydroxyl, C₁₋₄ alkyl, C₁₋₄ alkoxy, halo, C₁₋₄ haloalkyl, or C₃ or C₄ cycloalkyl; and R⁵ is hydro, hydroxyl, C₁₋₄ alkyl, C₁₋₄ alkoxy, halo, C₁₋₄ haloalkyl, C≡N, or C₃ or C₄ cycloalkyl;
q is 1 or 2, wherein any methylene group of the q region is optionally independently substituted with C₁₋₄ alkyl, halo, C₁₋₄ haloalkyl, or C₃ or C₄ cycloalkyl; and
R⁶ is optionally present, and if present, substitutes one, two, three, or four hydrogens, and in each instance is independently selected from halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl.

57-90. (canceled)

91. A compound selected from any one of Tables 1-9.

92. A pharmaceutical composition comprising a compound of claim 1 and a pharmaceutically acceptable excipient.

93. A method of treating cancer, comprising administering a therapeutically effective amount of a compound of claim 1 to a patient in need of such treatment.

94. The method of claim 93, further comprising administering a therapeutically effective amount of a PARD activator to said patient.

95-116. (canceled)

117. A pharmaceutical composition comprising a compound of claim 6 and a pharmaceutically acceptable excipient.

118. A pharmaceutical composition comprising a compound of claim 23 and a pharmaceutically acceptable excipient.

119. A pharmaceutical composition comprising a compound of claim 51 and a pharmaceutically acceptable excipient.

120. A pharmaceutical composition comprising a compound of claim 55 and a pharmaceutically acceptable excipient.

121. A pharmaceutical composition comprising a compound of claim 56 and a pharmaceutically acceptable excipient.

122. A method of treating cancer, comprising administering a therapeutically effective amount of a compound of claim 6 to a patient in need of such treatment.

123. A method of treating cancer, comprising administering a therapeutically effective amount of a compound of claim 23 to a patient in need of such treatment.

124. A method of treating cancer, comprising administering a therapeutically effective amount of a compound of claim 51 to a patient in need of such treatment.

125. A method of treating cancer, comprising administering a therapeutically effective amount of a compound of claim 55 to a patient in need of such treatment.

126. A method of treating cancer, comprising administering a therapeutically effective amount of a compound of claim 56 to a patient in need of such treatment.
